(19) United States
(12) Patent Application Publication
(10) Pub. No.: US 2024/0182487 A1
(43) Pub. Date: Jun. 6, 2024

(54) MACROCYCLES AND THEIR USE

(71) Applicant: BLOSSOMHILL THERAPEUTICS, INC., San Diego, CA (US)

(72) Inventors: Jingrong J. CUI, San Diego, CA (US); Eugene RUI, San Diego, CA (US); Evan W. ROGERS, San Diego, CA (US)

(21) Appl. No.: 18/257,166

(22) PCT Filed: Dec. 16, 2021
(86) PCT No.: PCT/US2021/063721

§ 371 Date: Jun. 13, 2023

Related U.S. Application Data
(60) Provisional application No. 63/126,722, filed on Dec. 17, 2020.
(60) Provisional application No. 63/279,850, filed on Nov. 16, 2021.
(60) Provisional application No. 63/284,789, filed on Dec. 1, 2021.
(60) Provisional application No. 63/289,014, filed on Dec. 13, 2021.

(51) Int. Cl. C07D 491/22; C07D 498/22

(52) U.S. Cl. CPC C07D 491/22; C07D 498/22

(57) ABSTRACT

The present disclosure relates to macrocyclic compounds, pharmaceutical compositions containing macrocyclic compounds, and methods of using macrocyclic compounds to treat disease, such as cancer.

### CROSS-REFERENCE TO RELATED APPLICATIONS

This application claims priority under 35 U.S.C. § 119(e) to U.S. Provisional Application Ser. No. 63/126,722 filed on Dec. 17, 2020, U.S. Provisional Application Ser. No. 63/279,850 filed on Nov. 16, 2021, U.S. Provisional Application Ser. No. 63/284,789 filed on Dec. 1, 2021, and U.S. Provisional Application Ser. No. 63/289,014 filed on Dec. 13, 2021, the entire disclosures of all of which are incorporated herein by reference.

### TECHNICAL FIELD

The present disclosure relates to macrocyclic compounds, pharmaceutical compositions containing macrocyclic compounds, and methods of using macrocyclic compounds to treat disease, such as cancer.

### BACKGROUND

Protein kinases are tightly regulated signaling proteins that orchestrate the activation of signaling cascades by phosphorylating target proteins in response to extracellular and intracellular stimuli. The human genome encodes approximately 518 protein kinases (Manning G, et al The protein kinase complement of the human genome. Science. 2002, 298:1912-34). Dysregulation of kinase activity is associated with many diseases, including cancers, and cardiovascular, degenerative, immunological, infectious, inflammatory, and metabolic diseases (Levitzki, A. Protein kinase inhibitors as a therapeutic modality. Acc. Chem. Res. 2003, 36:462-469). The molecular bases leading to various diseases include kinase gain- and loss-of-function mutations, gene amplifications and deletions, splicing changes, and translocations (Wilson L J, et al New Perspectives, Opportunities, and Challenges in Exploring the Human Protein Kinome. Cancer Res. 2018, 78:15-29). The critical role of kinases in cancer and other diseases makes them attractive targets for drug inventions with 52 small molecule kinase inhibitors have been approved and 46 of them for cancer targeted therapies (Roskoski R Jr, Properties of FDA-approved Small Molecule Protein Kinase Inhibitors: A 2020 Update. Pharmacol Res 2020, 152:104609). Although kinase inhibitors have achieved dramatic success in cancer-targeted therapies, the development of treatment resistance has remained as a challenge for small molecule kinase inhibitors. Acquired secondary mutations within kinase domain during the treatment often lead to treatment resistance to kinase inhibitors (Pottier C, et al Tyrosine Kinase Inhibitors in Cancer: Breakthrough and Challenges of Targeted Therapy. Cancers (Basel), 2020, 12:731). Therefore, it is necessary to invent kinase inhibitors that can target not only the kinase oncogenic drivers, and also overcome most frequent resistance mutations for better efficacy and longer disease control.

Non-small-cell lung cancer (NSCLC) is the leading cause of cancer mortality worldwide (World Health Organisation. Cancer Fact Sheet 2017). Activating EGFR mutations have been reported in approximately 10% to 15% of cases of adenocarcinoma in white patients and 50% of cases in Asian patients (Chan B A, Hughes B G. Targeted therapy for non-small cell lung cancer: current standards and the promise of the future. Transl Lung Cancer Res 2015; 4:36-54). The two most frequent EGFR alterations found in NSCLC tumors are short in-frame deletions in exon 19 (del19) of the EGFR gene and L858R, a single missense mutation in exon 21 (Konduri K. et al. EGFR Fusions as Novel Therapeutic Targets in Lung Cancer. Cancer Discovery 2016, 6:601-11).

The first-generation reversible EGFR inhibitors, erlotinib and gefitinib are superior to chemotherapy in patients with advanced EGFR mutation-positive (Del19 or L858R) NSCLC and have been used as first-line standard of care in this setting. However, most patients will develop resistance to gefitinib or erlotinib with 50% to 70% of tumors developing EGFR T790M gatekeeper mutation with time of treatment (Sequist L V, et al. Genotypic and histological evolution of lung cancers acquiring resistance to EGFR inhibitors. Sci Transl Med 2011; 3:75ra26). The second generation of EGFR inhibitors afatinib and dacomitinib are covalent, irreversible EGFR inhibitors that also inhibit HER2 and ERB4 of the ERB family (Li D, et al. BIBW2992, an irreversible EGFR/HER2 inhibitor highly effective in preclinical lung cancer models. Oncogene 2008; 27: 4702-11; Ou S H, Soo R A. Dacomitinib in lung cancer: a “lost generation” EGFR tyrosine-kinase inhibitor from a bygone era? Drug Des Devel Ther 2015; 9:5641-53).

Although afatinib and dacomitinib are more potent EGFR inhibitors approved as first-line therapy for advanced EGFR mutation-positive (Del19 or L858R) NSCLC with longer progression free survival time (PFS) in comparison with gefitinib and erlotinib, EGFR T790M has been developed with time of treatment with afatinib (Tanaka K, et al. Acquisition of the T790M resistance mutation during afatinib treatment in EGFR tyrosine kinase inhibitor-naive patients with non-small cell lung cancer harboring EGFR mutations. Onco-target 2017; 8:68123-30). EGFR T790M confers resistance to dacomitinib In vitro studies (Kobayashi Y, et al. EGFR T790M and C797S mutations as mechanisms of acquired resistance to dacomitinib. J Thorac Oncol 2018; 13: 727-31). The third-generation EGFR inhibitor Osimertinib is also an irreversible inhibitor targeting both EGFR activating mutations (Del19 and L858R) and T790M resistant double mutations, with selectivity over the wild-type EGFR (Finlay M R, et al. Discovery of a potent and selective EGFR inhibitor (AZD9291) of both sensitizing and T790M resistance mutations that spares the wild type form of the receptor. J Med Chem 2014; 57:8249-67). Osimertinib was first approved for patients with metastatic EGFR T790M mutation-positive NSCLC after failure of first-line EGFR inhibitors, and later approved in the first-line setting for patients with EGFR mutation-positive NSCLC following the phase III FLAURA trial with head-to-head trials comparing with erlotinib or gefitinib (Soria J C, et al. Osimertinib in untreated EGFR-mutated advanced non-small-cell lung cancer. N Engl J Med 2018; 378:113-25). The mutation C797S at the EGFR covalent binding residue with irreversible EGFR inhibitor Osimertinib has been detected in Osimertinib-resistant patients (Ramalingam S S, et al. Mechanisms of acquired resistance to first-line osimertinib: preliminary data from the phase III FLAURA study. Presented at the ESMO 2018).

Therefore, it is necessary to develop a new generation reversible EGFR inhibitors that are potent against oncogenic driver EGFR mutations, such as L858R, Del19, Δ746-750, Δ746-750/T790M, Δ746-750/C979S, L858R/T790M, Del19/T790M, L858R/C979S, Del19/C979S, L858R/T790M/C979S, and Δ746-750/T790M/C979S, as well as other emerging and established resistance mutations, while maintaining good selectivity over wild-type EGFR.

### SUMMARY

In one aspect, the disclosure relates to a compound of the formula Ia, or a pharmaceutically acceptable salt thereof,

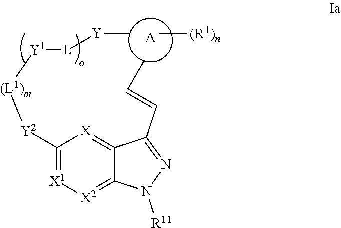

wherein
A is a ring selected from 5- to 10-membered heteroarylene or C₆-C₁₀ arylene;
X is C(R²) or N;
X¹ is C(R³) or N;
X² is C(R⁴) or N;
Y is O, N(R⁵)C(O), C(O)N(R⁵), N(R⁶), N(R⁵)S(O), S(O)N(R⁵), N(R⁵)S(O)₂, S(O)₂N(R⁵), S, S(O), S(O)₂, or Y is absent;
each Y¹ is independently O, C(O)N(R⁷), N(R⁷)C(O), N(R⁸), N(R⁷)S(O), S(O)N(R⁷), N(R⁷)S(O)₂, S(O)₂N(R⁷), S, S(O), S(O)₂, or absent;
Y² is O, C(O)N(R⁹), N(R⁹)C(O), N(R¹⁰), N(R⁹)S(O), S(O)N(R⁹), N(R⁹)S(O)₂, S(O)₂N(R⁹), S, S(O), S(O)₂, or Y² is absent;
each L is independently a C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or optionally two hydrogen atoms on one carbon atom in C₁-C₆ alkylene are optionally substituted by a C₂-C₅ alkylene to provide a C₃-C₆ cycloalkylene; or optionally two hydrogen atoms on two carbon atoms in C₁-C₆ alkylene are optionally substituted by a C₁-C₄ alkylene to provide a C₃-C₆ cycloalkylene; or optionally one hydrogen atom on one carbon atom in C₁-C₆ alkylene and one of R⁵, R⁶, R⁷, or R⁸ taken together with the atoms to which they are attached combine to form a 3- to 7-membered heterocycloalkylene;
each L¹, when present, is independently C₁-C₆ alkylene or a ring B selected from the group consisting of 5- to 10-membered heteroarylene, 3- to 10-membered heterocycloalkylene, 3- to 6-membered cycloalkylene, and C₆-C₁₀ arylene, wherein each hydrogen atom in 5- to 10-membered heteroarylene, 3- to 10-membered heterocycloalkylene, C₆-C₁₀ arylene, and C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R², R³, and R⁴, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, R⁸, R⁹, R¹⁰, and R¹¹, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, and 5- to 10-membered heteroaryl;
m is 0, 1, 2, 3, or 4;
n is 0, 1, 2, 3, or 4, and
is 0, 1, 2, or 3.

In another aspect, the disclosure relates to a compound of the formula Ia, or a pharmaceutically acceptable salt thereof,

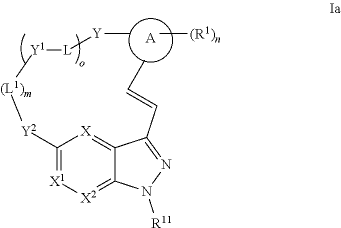

wherein
A is a ring selected from 5- to 10-membered heteroarylene or C₆-C₁₀ arylene;
X is C(R²) or N;
X¹ is C(R³) or N;
X² is C(R⁴) or N;
Y is O, N(R⁵)C(O), C(O)N(R⁵), N(R⁶), N(R⁵)S(O), S(O)N(R⁵), N(R⁵)S(O)₂, S(O)₂N(R⁵), S, S(O), S(O)₂, or Y is absent;
each Y¹ is independently O, C(O)N(R⁷), N(R⁷)C(O), N(R⁸), N(R⁷)S(O), S(O)N(R⁷), N(R⁷)S(O)₂, S(O)₂N(R⁷), S, S(O), S(O)₂, or absent;
Y² is O, C(O)N(R⁹), N(R⁹)C(O), N(R¹⁰), N(R⁹)S(O), S(O)N(R⁹), N(R⁹)S(O)₂, S(O)₂N(R⁹), S, S(O), S(O)₂, or Y² is absent;
each L is independently a C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or optionally two hydrogen atoms on one carbon atom in C₁-C₆ alkylene are optionally substituted by a C₂-C₅ alkylene to provide a C₃-C₆ cycloalkylene; or optionally two hydrogen atoms on two carbon atoms in C₁-C₆ alkylene are optionally substituted by a C₁-C₄ alkylene to provide a C₃-C₆ cycloalkylene; or optionally one hydrogen atom on one carbon atom in C₁-C₆ alkylene and one of R⁵, R⁶, R⁷, or R⁸ taken together with the atoms to which they are attached combine to form a 3- to 7-membered heterocycloalkylene;
each L¹, when present, is independently C₁-C₆ alkylene or a ring B selected from the group consisting of 5- to 10-membered heteroarylene, 3- to 10-membered heterocycloalkylene, 3- to 6-membered cycloalkylene, and C₆-C₁₀ arylene, wherein each hydrogen atom in 5- to 10-membered heteroarylene, 3- to 10-membered heterocycloalkylene, C₆-C₁₀ arylene, and C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R², R³, and R⁴, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, R⁸, R⁹, R¹⁰, and R¹¹, when present, is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{c} and R^{d} or R^{e} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m is 0, 1, 2, 3, or 4;
n is 0, 1, 2, 3, or 4; and
is 0, 1, 2, or 3.

In another aspect, the disclosure relates to a compound of the formula I, or a pharmaceutically acceptable salt thereof,

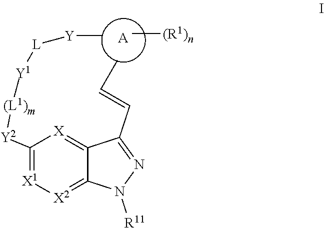

wherein
A is a ring selected from 5- to 10-membered heteroarylene or C₆-C₁₀ arylene;
X is C(R²) or N;
X¹ is C(R³) or N;
X² is C(R⁴) or N;
Y is O, N(R⁵)C(O), N(R⁶), S, S(O), S(O)₂, or Y is absent;
Y¹ is O, C(O)N(R⁷), N(R⁸), S, S(O), S(O)₂, or Y¹ is absent;
Y² is O, C(O)N(R⁹), N(R¹⁰), S, S(O), S(O)₂, or Y² is absent;
L is a C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂;
each L¹, when present, is independently C₁-C₆ alkylene or a ring B selected from the group consisting of 5- to 10-membered heteroarylene, 3- to 10-membered heterocycloalkylene, 3- to 6-membered cycloalkylene, and C₆-C₁₀ arylene, wherein each hydrogen atom in 5- to 10-membered heteroarylene, 3- to 10-membered heterocycloalkylene, C₆-C₁₀ arylene, and C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R², R³, and R⁴, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, R⁸, R⁹, R¹⁰, and R¹¹, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, and 5- to 10-membered heteroaryl;
m is 0, 1, 2, 3, or 4; and
n is 0, 1, 2, 3, or 4.

In another aspect, the disclosure relates to a compound of the formula I, or a pharmaceutically acceptable salt thereof,

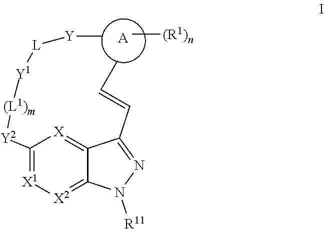

wherein
A is a ring selected from 5- to 10-membered heteroarylene or C₆-C₁₀ arylene;
X is C(R²) or N;
X¹ is C(R³) or N;
X² is C(R⁴) or N;
Y is O, N(R⁵)C(O), N(R⁵), N(R⁶), S, S(O), S(O)₂, or Y is absent;
Y¹ is O, C(O)N(R⁷), N(R⁸), S, S(O), S(O)₂, or Y¹ is absent;
Y² is O, C(O)N(R⁹), N(R¹⁰), S, S(O), S(O)₂, or Y² is absent;
L is a C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂;
each L¹, when present, is independently C₁-C₆ alkylene or a ring B selected from the group consisting of 5- to 10-membered heteroarylene, 3- to 10-membered heterocycloalkylene, 3- to 6-membered cycloalkylene, and C₆-C₁₀ arylene, wherein each hydrogen atom in 5- to 10-membered heteroarylene, 3- to 10-membered heterocycloalkylene, C₆-C₁₀ arylene, and C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R², R³, and R⁴, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, R⁸, R⁹, R¹⁰, and R¹¹, when present, is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{c} and R^{d} or R^{e} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m is 0, 1, 2, 3, or 4; and
n is 0, 1, 2, 3, or 4.

In another aspect, the disclosure provides a compound of the formula IIa, or a pharmaceutically acceptable salt thereof,

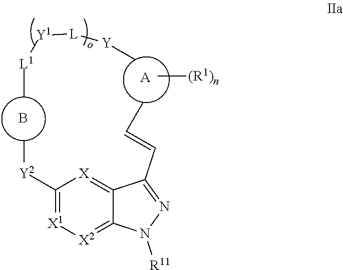

wherein R¹, R¹¹, A, L, X, X¹, X², Y, Y¹, Y², n, and o are as described herein;
ring B is a 5- to 10-membered heteroarylene, 3- to 10-membered heterocycloalkylene, or C₆-C₁₀ arylene, wherein each hydrogen atom in 3- to 10-membered heteroarylene, 5- to 10-membered heterocycloalkylene, and C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂; and
L¹ is C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In another aspect, the disclosure provides a compound of the formula II, or a pharmaceutically acceptable salt thereof,

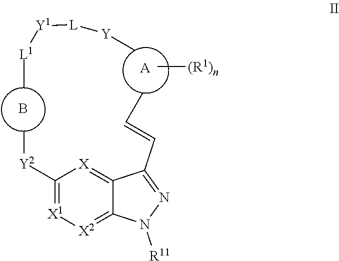

wherein R¹, R¹¹, A, L, X, X¹, X², Y, Y¹, Y², and n are as described herein;
ring B is a 5- to 10-membered heteroarylene, 3- to 10-membered heterocycloalkylene, or C₆-C₁₀ arylene, wherein each hydrogen atom in 3- to 10-membered heteroarylene, 5- to 10-membered heterocycloalkylene, and C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂; and
L¹ is C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In another aspect, the disclosure provides a compound of the formula IIIa, or a pharmaceutically acceptable salt thereof,

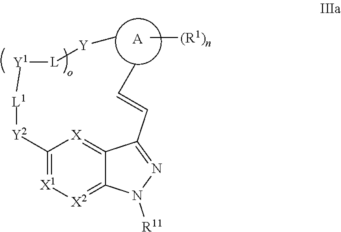

wherein R¹, R¹¹, A, L, X, X¹, X², Y, Y¹, Y², n, and o are as described herein; and.
L¹ is C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In another aspect, the disclosure provides a compound of the formula III, or a pharmaceutically acceptable salt thereof,

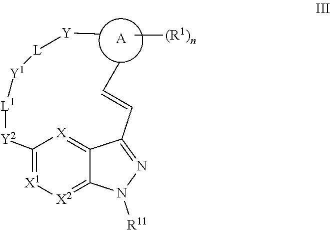

wherein R¹, R¹¹, A, L, X, X¹, X², Y, Y¹, Y², and n are as described herein; and.
L¹ is C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In another aspect, the disclosure relates to a compound of the formula IVa, or a pharmaceutically acceptable salt thereof,

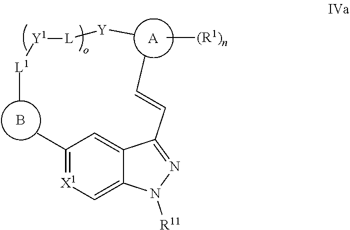

wherein
ring A is a ring selected from 5- to 10-membered heteroarylene or C₆-C₁₀ arylene;
X¹ is C(R³) or N;
Y is O, N(R⁵)C(O), S, S(O), or S(O)₂;
each Y¹ is independently O, S, S(O), or S(O)₂;
each L is independently a C₁-C₆ alkylene, wherein each hydrogen atom in C₁-C₆ alkylene is independently optionally substituted by deuterium;
L¹ is absent, or L¹ is C₁-C₆ alkylene, wherein each hydrogen atom in C₁-C₆ alkylene is independently optionally substituted by deuterium or C₁-C₆ alkyl;
ring B is a 5- to 10-membered heteroarylene or C₆-C₁₀ arylene, wherein each hydrogen atom in 3- to 10-membered heteroarylene or C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, and R¹¹, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{c} and R^{d} or R^{e} and R^{f}, taken together with the atom to which they are attached, form a a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
n is 0, 1, 2, 3, or 4; and
o is 0, 1, 2, or 3.

In another aspect, the disclosure relates to a compound of the formula IV, or a pharmaceutically acceptable salt thereof,

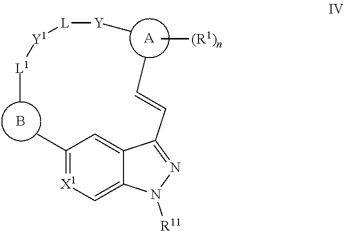

wherein
ring A is a ring selected from 5- to 10-membered heteroarylene or C₆-C₁₀ arylene;
Y is O, N(R⁵)C(O), S, S(O), or S(O)₂;
Y¹ is O, S, S(O), or S(O)₂;
L is a C₁-C₆ alkylene, wherein each hydrogen atom in C₁-C₆ alkylene is independently optionally substituted by deuterium;
L¹ is absent, or L¹ is C₁-C₆ alkylene, wherein each hydrogen atom in C₁-C₆ alkylene is independently optionally substituted by deuterium or C₁-C₆ alkyl;
ring B is a 5- to 10-membered heteroarylene or C₆-C₁₀ arylene, wherein each hydrogen atom in 3- to 10-membered heteroarylene or C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵ and R¹¹, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{c} and R^{d} or R^{e} and R^{f}, taken together with the atom to which they are attached, form a a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂; and
n is 0, 1, 2, 3, or 4.

In certain embodiments of the above aspects, the compound of Formula (Ia)-(IXa) or (I)-(IX) is a compound selected from those species described or exemplified in the detailed description below.

In further aspects, the disclosure relates to a pharmaceutical composition comprising at least one compound of Formula (Ia)-(IXa) or (I)-(IX) or a pharmaceutically acceptable salt thereof. Pharmaceutical compositions according to the disclosure may further comprise a pharmaceutically acceptable excipient.

In further aspects, the disclosure relates to a compound of Formula (Ia)-(IXa) or (I)-(IX), or a pharmaceutically acceptable salt thereof, for use as a medicament.

In further aspects, the disclosure relates to a method of treating disease, such as cancer comprising administering to a subject in need of such treatment an effective amount of at least one compound of Formula (Ia)-(IXa) or (I)-(IX), or a pharmaceutically acceptable salt thereof.

In further aspects, the disclosure relates to use of a compound of Formula (Ia)-(IXa) or (I)-(IX), or a pharmaceutically acceptable salt thereof, in the preparation of a medicament for the treatment of disease, such as cancer, and the use of such compounds and salts for treatment of such diseases.

In further aspects, the disclosure relates to a method of inhibiting a tyrosine kinase, such as EGFR, comprising contacting a cell comprising one or more of kinase with an effective amount of at least one compound of Formula (Ia)-(IXa) or (I)-(IX), or a pharmaceutically acceptable salt thereof, and/or with at least one pharmaceutical composition of the disclosure, wherein the contacting is in vitro, ex vivo, or in vivo.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A compound of the formula Ia, or a pharmaceutically acceptable salt thereof,

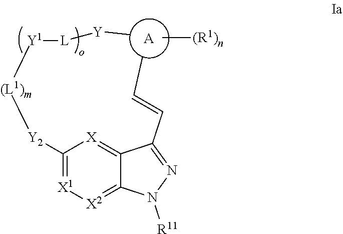

wherein
A is a ring selected from 5- to 10-membered heteroarylene or C₆-C₁₀ arylene;
X is C(R²) or N;
X¹ is C(R³) or N;
X² is C(R⁴) or N;
Y is O, N(R⁵)C(O), C(O)N(R⁵), N(R⁶), N(R⁵)S(O), S(O)N(R⁵), N(R⁵)S(O)₂, S(O)₂N(R⁵), S, S(O), S(O)₂, or Y is absent;
each Y¹ is independently O, C(O)N(R⁷), N(R⁷)C(O), N(R⁸), N(R⁷)S(O), S(O)N(R⁷), N(R⁷)S(O)₂, S(O)₂N(R⁷), S, S(O), S(O)₂, or absent;
Y² is O, C(O)N(R⁹), N(R⁹)C(O), N(R¹⁰), N(R⁹)S(O), S(O)N(R⁹), N(R⁹)S(O)₂, S(O)₂N(R⁹), S, S(O), S(O)₂, or Y² is absent;
each L is independently a C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or optionally two hydrogen atoms on one carbon atom in C₁-C₆ alkylene are optionally substituted by a C₂-C₅ alkylene to provide a C₃-C₆ cycloalkylene; or optionally two hydrogen atoms on two carbon atoms in C₁-C₆ alkylene are optionally substituted by a C₁-C₄ alkylene to provide a C₃-C₆ cycloalkylene; or optionally one hydrogen atom on one carbon atom in C₁-C₆ alkylene and one of R⁵, R⁶, R⁷, or R⁸ taken together with the atoms to which they are attached combine to form a 3- to 7-membered heterocycloalkylene;
each L¹, when present, is independently C₁-C₆ alkylene or a ring B selected from the group consisting of 5- to 10-membered heteroarylene, 3- to 10-membered heterocycloalkylene, 3- to 6-membered cycloalkylene, and C₆-C₁₀ arylene, wherein each hydrogen atom in 5- to 10-membered heteroarylene, 3- to 10-membered heterocycloalkylene, C₆-C₁₀ arylene, and C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R², R³, and R⁴, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, R⁸, R⁹, R¹⁰, and R¹¹, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, and 5- to 10-membered heteroaryl;
m is 0, 1, 2, 3, or 4;
n is 0, 1, 2, 3, or 4, and
is 0, 1, 2, or 3;
or a pharmaceutically acceptable salt thereof.

1a. A compound of the formula Ia, or a pharmaceutically acceptable salt thereof,

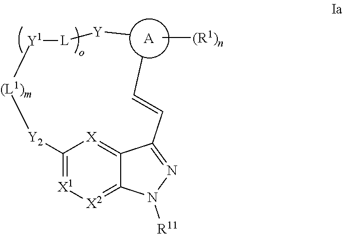

wherein
A is a ring selected from 5- to 10-membered heteroarylene or C₆-C₁₀ arylene;
X is C(R²) or N;
X¹ is C(R³) or N;
X² is C(R⁴) or N;
Y is O, N(R⁵)C(O), C(O)N(R⁵), N(R⁶), N(R⁵)S(O), S(O)N(R⁵), N(R⁵)S(O)₂, S(O)₂N(R⁵), S, S(O), S(O)₂, or Y is absent;
each Y¹ is independently O, C(O)N(R⁷), N(R⁷)C(O), N(R⁸), N(R⁷)S(O), S(O)N(R⁷), N(R⁷)S(O)₂, S(O)₂N(R⁷), S, S(O), S(O)₂, or absent;
Y² is O, C(O)N(R⁹), N(R⁹)C(O), N(R¹⁰), N(R⁹)S(O), S(O)N(R⁹), N(R⁹)S(O)₂, S(O)₂N(R⁹), S, S(O), S(O)₂, or Y² is absent;
each L is independently a C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or optionally two hydrogen atoms on one carbon atom in C₁-C₆ alkylene are optionally substituted by a C₂-C₅ alkylene to provide a C₃-C₆ cycloalkylene; or optionally two hydrogen atoms on two carbon atoms in C₁-C₆ alkylene are optionally substituted by a C₁-C₄ alkylene to provide a C₃-C₆ cycloalkylene; or optionally one hydrogen atom on one carbon atom in C₁-C₆ alkylene and one of R⁵, R⁶, R⁷, or R⁸ taken together with the atoms to which they are attached combine to form a 3- to 7-membered heterocycloalkylene;
each L¹, when present, is independently C₁-C₆ alkylene or a ring B selected from the group consisting of 5- to 10-membered heteroarylene, 3- to 10-membered heterocycloalkylene, 3- to 6-membered cycloalkylene, and C₆-C₁₀ arylene, wherein each hydrogen atom in 5- to 10-membered heteroarylene, 3- to 10-membered heterocycloalkylene, C₆-C₁₀ arylene, and C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R², R³, and R⁴, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, R⁸, R⁹, R¹⁰, and R¹¹, when present, is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{c} and R^{d} or R^{e} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m is 0, 1, 2, 3, or 4;
n is 0, 1, 2, 3, or 4; and
is 0, 1, 2, or 3;
or a pharmaceutically acceptable salt thereof.

1b. A compound of the formula I

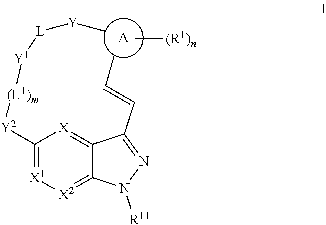

wherein
A is a ring selected from 5- to 10-membered heteroarylene or C₆-C₁₀ arylene;
X is C(R²) or N;
X¹ is C(R³) or N;
X² is C(R⁴) or N;
Y is O, N(R⁵)C(O), N(R⁶), S, S(O), S(O)₂, or Y is absent;
Y¹ is O, C(O)N(R⁷), N(R⁸), S, S(O), S(O)₂, or Y¹ is absent;
Y² is O, C(O)N(R⁹), N(R¹⁰), S, S(O), S(O)₂, or Y² is absent;
L is a C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂;
each L¹, when present, is independently C₁-C₆ alkylene or a ring B selected from the group consisting of 5- to 10-membered heteroarylene, 3- to 10-membered heterocycloalkylene, 3- to 6-membered cycloalkylene, and C₆-C₁₀ arylene, wherein each hydrogen atom in 5- to 10-membered heteroarylene, 3- to 10-membered heterocycloalkylene, C₆-C₁₀ arylene, and C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R², R³, and R⁴, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, R⁸, R⁹, R¹⁰, and R¹¹, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, and 5- to 10-membered heteroaryl;
m is 0, 1, 2, 3, or 4; and
n is 0, 1, 2, 3, or 4;
or a pharmaceutically acceptable salt thereof.

1c. A compound of the formula I

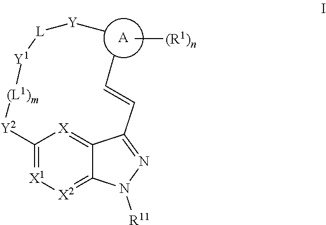

wherein
A is a ring selected from 5- to 10-membered heteroarylene or C₆-C₁₀ arylene;
X is C(R²) or N;
X¹ is C(R³) or N;
X² is C(R⁴) or N;
Y is O, N(R⁵)C(O), N(R⁶), S, S(O), S(O)₂, or Y is absent;
Y¹ is O, C(O)N(R⁷), N(R⁸), S, S(O), S(O)₂, or Y¹ is absent;
Y² is O, C(O)N(R⁹), N(R¹⁰), S, S(O), S(O)₂, or Y² is absent;
L is a C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂;
each L¹, when present, is independently C₁-C₆ alkylene or a ring B selected from the group consisting of 5- to 10-membered heteroarylene, 3- to 10-membered heterocycloalkylene, 3- to 6-membered cycloalkylene, and C₆-C₁₀ arylene, wherein each hydrogen atom in 5- to 10-membered heteroarylene, 3- to 10-membered heterocycloalkylene, C₆-C₁₀ arylene, and C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R², R³, and R⁴, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, R⁸, R⁹, R¹⁰, and R¹¹, when present, is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{c} and R^{d} or R^{e} and R^{f}, taken together with the atom to which they are attached, form a a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m is 0, 1, 2, 3, or 4; and
n is 0, 1, 2, 3, or 4;
or a pharmaceutically acceptable salt thereof.

2. The compound of clause 1 or 1a, or a pharmaceutically acceptable salt thereof, having the formula IIa

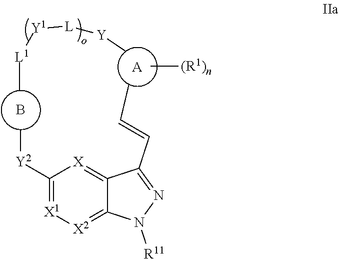

wherein
ring B is a 5- to 10-membered heteroarylene, 3- to 10-membered heterocycloalkylene, or C₆-C₁₀ arylene, wherein each hydrogen atom in 3- to 10-membered heteroarylene, 5- to 10-membered heterocycloalkylene, and C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂; and
L¹ is C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

2a. The compound of clause 1, 1a, 1b, or 1c, or a pharmaceutically acceptable salt thereof, having the formula II

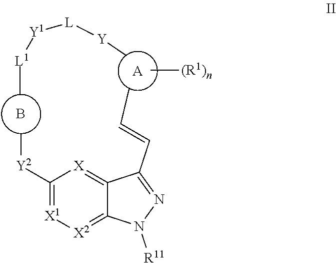

wherein
ring B is a 5- to 10-membered heteroarylene, 3- to 10-membered heterocycloalkylene, or C₆-C₁₀ arylene, wherein each hydrogen atom in 3- to 10-membered heteroarylene, 5- to 10-membered heterocycloalkylene, and C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂; and
L¹ is C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

3. The compound of clause 1 or 1a, or a pharmaceutically acceptable salt thereof, having the formula IIIa

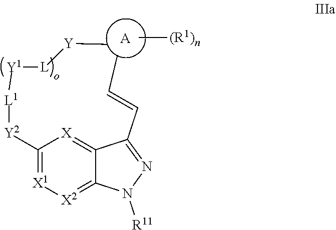

wherein
L¹ is C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

3a. The compound of clause 1, 1a, 1b, or 1c, or a pharmaceutically acceptable salt thereof, having the formula III

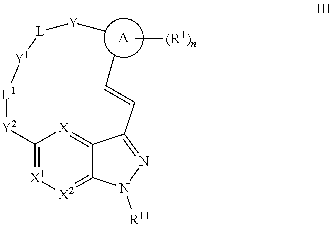

wherein
L¹ is C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

4. The compound of clause 1 or 1a, or a pharmaceutically acceptable salt thereof, having the formula IVa

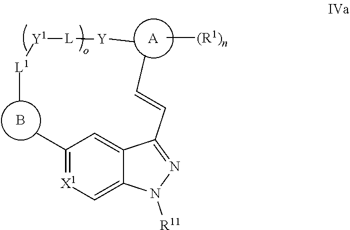

wherein
ring A is a ring selected from 5- to 10-membered heteroarylene or C₆-C₁₀ arylene;
X¹ is C(R³) or N;
Y is O, N(R⁵)C(O), S, S(O), or S(O)₂;
Y¹ is O, S, S(O), or S(O)₂;
L is a C₁-C₆ alkylene, wherein each hydrogen atom in C₁-C₆ alkylene is independently optionally substituted by deuterium;
L¹ is absent, or L¹ is C₁-C₆ alkylene, wherein each hydrogen atom in C₁-C₆ alkylene is independently optionally substituted by deuterium or C₁-C₆ alkyl;
ring B is a 5- to 10-membered heteroarylene or C₆-C₁₀ arylene, wherein each hydrogen atom in 3- to 10-membered heteroarylene or C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, and R¹¹, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{c} and R^{d} or R^{e} and R^{f}, taken together with the atom to which they are attached, form a a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂; and
n is 0, 1, 2, 3, or 4.

4a. The compound of clause 1, 1a, 1b, or 1c, or a pharmaceutically acceptable salt thereof, having the formula IV

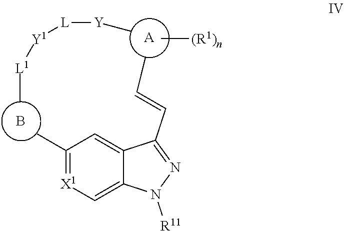

wherein
ring A is a ring selected from 5- to 10-membered heteroarylene or C₆-C₁₀ arylene;
Y is O, N(R⁵)C(O), S, S(O), or S(O)₂;
Y¹ is O, S, S(O), or S(O)₂;
L is a C₁-C₆ alkylene, wherein each hydrogen atom in C₁-C₆ alkylene is independently optionally substituted by deuterium;
L¹ is absent, or L¹ is C₁-C₆ alkylene, wherein each hydrogen atom in C₁-C₆ alkylene is independently optionally substituted by deuterium or C₁-C₆ alkyl;
ring B is a 5- to 10-membered heteroarylene or C₆-C₁₀ arylene, wherein each hydrogen atom in 3- to 10-membered heteroarylene or C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵ and R¹¹, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{c} and R^{d} or R^{e} and R^{f}, taken together with the atom to which they are attached, form a a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂; and
n is 0, 1, 2, 3, or 4.

4b. The compound of claim 1, 1a, 2, or 4, having the formula Va, V, Via, VI, VIIa, VII, VIIIa, VIII, IXa, or IX

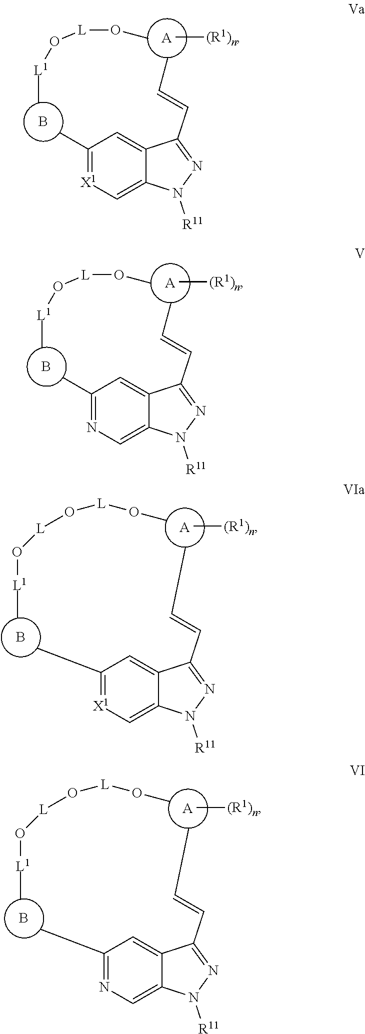

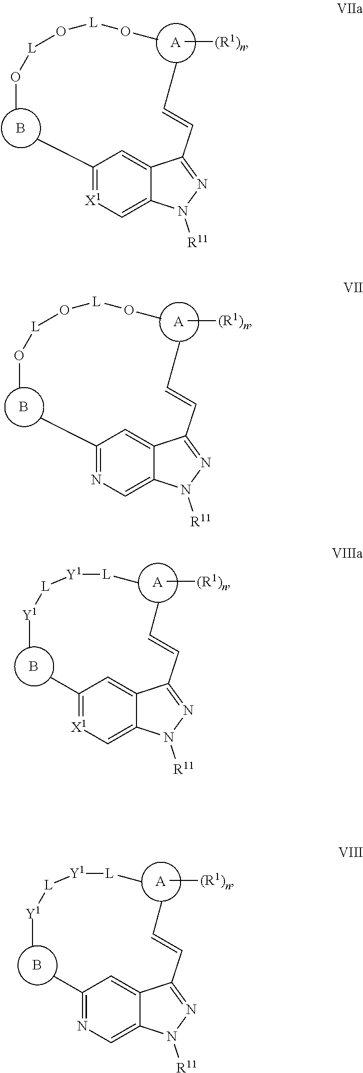

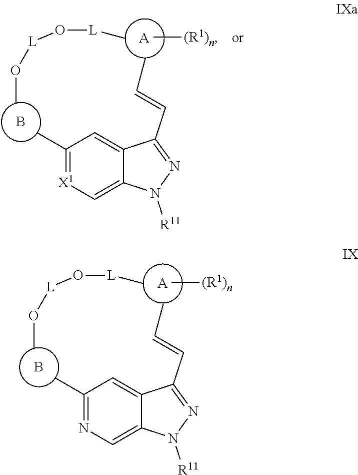

or a pharmaceutically acceptable salt thereof.

5. The compound of clause 1, 1a, 1b, 1c, 2, 2a, 4, 4a, or 4b, or a pharmaceutically acceptable salt thereof, wherein ring B is a 5- to 10-membered heteroarylene, wherein each hydrogen atom in 5- to 10-membered heteroarylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

6. The compound of any one of clauses 1, 1a, 1b, 1c, 2, 2a, 4, 4a, 4b, or 5, or a pharmaceutically acceptable salt thereof, wherein ring B is a 5- to 10-membered heteroarylene selected from the group consisting of

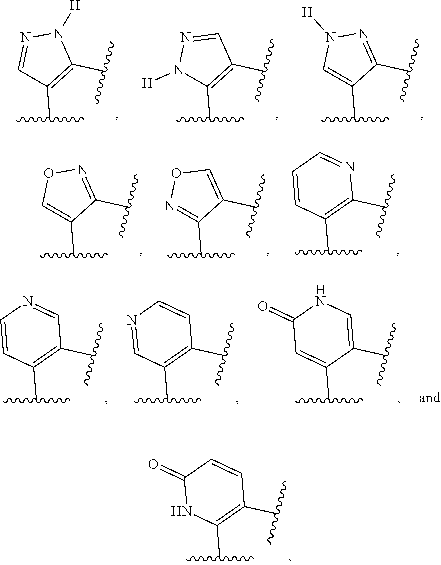

wherein each hydrogen atom in ring B is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

7. The compound of any one of clauses 1, 2, 4, 4a, 4b, 5, or 6, or a pharmaceutically acceptable salt thereof, wherein ring B is

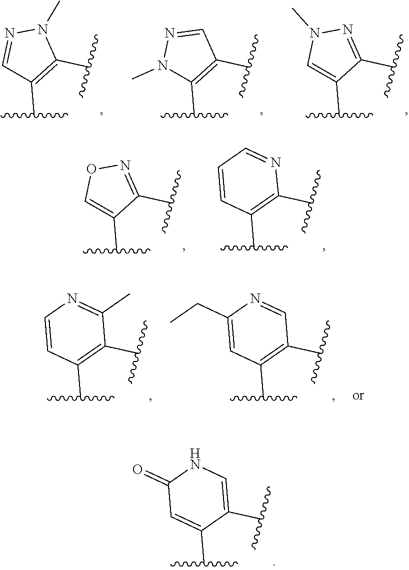

8. The compound of clause 1, 1a, 1b, 1c, 2, 2a, 4, 4a, or 4b, or a pharmaceutically acceptable salt thereof, wherein ring B is a 3- to 10-membered heterocycloalkylene, wherein each hydrogen atom in 3- to 10-membered heterocycloalkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

9. The compound of any one of clauses 1, 1a, 1b, 1c, 2, 2a, 4, 4a, or 4b, or 8, or a pharmaceutically acceptable salt thereof, wherein ring B is a 3- to 10-membered heterocycloalkylene selected from the group consisting of

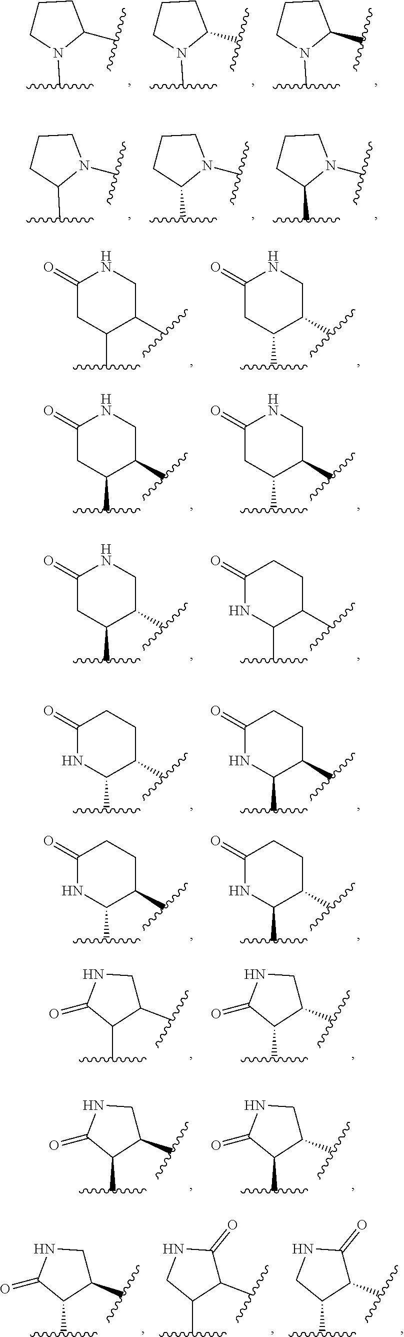

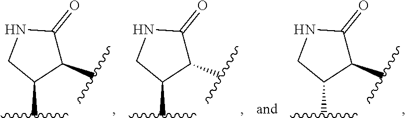

wherein each hydrogen atom in 3- to 10-membered heterocycloalkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

10. The compound of any one of clauses 1, 1a, 1b, 1c, 2, 2a, 4, 4a, 4b, 8, or 9, or a pharmaceutically acceptable salt thereof, wherein ring B is a 3- to 10-membered heterocycloalkylene selected from the group consisting of

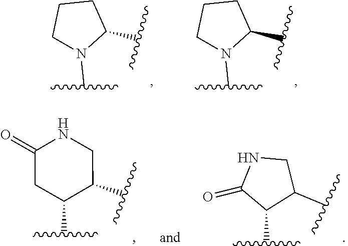 [SMILES: *[C@H]1CCCN1* ; *[C@@H]1CCCN1* ; *[C@@H]1CC(=O)NC[C@@H]1* ; *C1CNC(=O)[C@H]1*]

11. The compound of clause 1, 1a, 1b, 1c, 2, 2a, 4, 4a, or 4b, or a pharmaceutically acceptable salt thereof, wherein ring B is a C₆-C₁₀ arylene, wherein each hydrogen atom in C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

12. The compound of any one of clauses 1, 1a, 1b, 1c, 2, 2a, 4, 4a, 4b, or 11, or a pharmaceutically acceptable salt thereof, wherein ring B is a phenylene optionally substituted with one or more deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

13. The compound of any one of clauses 1, 1a, 1b, 1c, 2, 2a, 4, 4a, 4b, 11, or 12, or a pharmaceutically acceptable salt thereof, wherein ring B is selected from the group consisting of

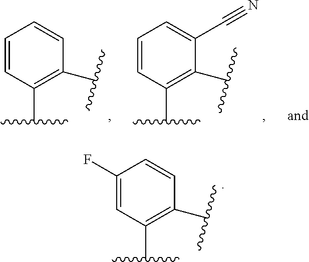

14. The compound of any one of the preceding clauses, or a pharmaceutically acceptable salt thereof, wherein

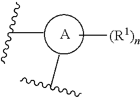

is a 5- to 10-membered heteroarylene, and n is 0, 1, 2, 3, or 4.

15. The compound of any one of the preceding clauses, or a pharmaceutically acceptable salt thereof, wherein

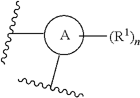

is a 5- to 10-membered heteroarylene selected from the group consisting of

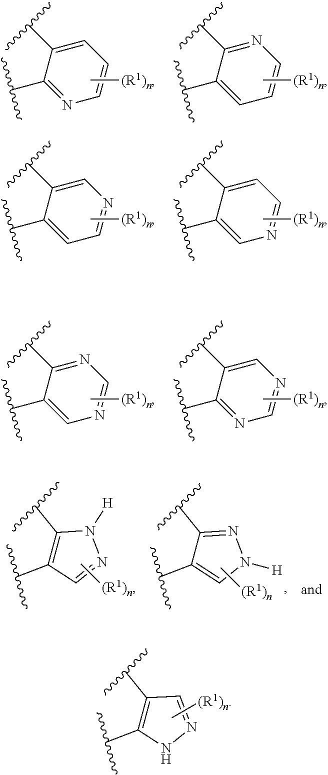

and n is 0, 1, or 2.

16. The compound of any one of the preceding clauses, or a pharmaceutically acceptable salt thereof, and n is 0, 1, or 2.

17. The compound of any one of the preceding clauses, or a pharmaceutically acceptable salt thereof, wherein each R¹, when present, is independently fluoro, chloro, methyl, ethyl, methoxy, ethoxy, —C(O)OR^{a}, —C(O)NR^{a}R^{b}, —CN, or 4-piperidinyl.

18. The compound of any one of clauses 1 to 17, or a pharmaceutically acceptable salt thereof, wherein

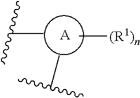

is a 5- to 10-membered heteroarylene selected from the group consisting of

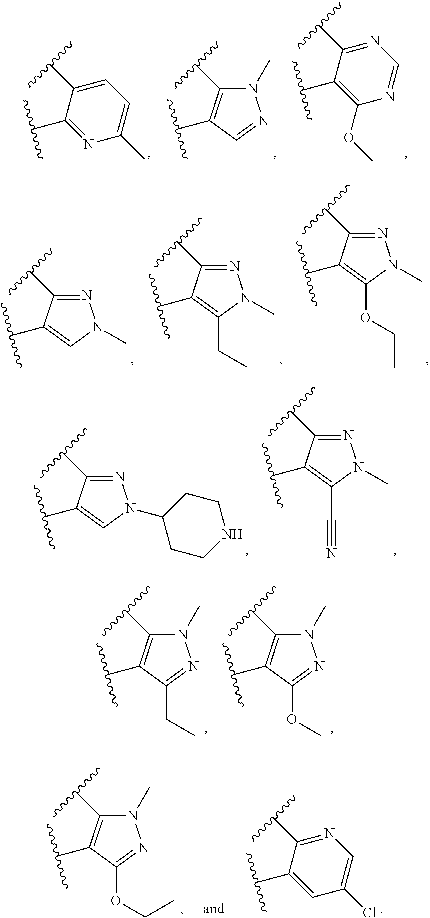

19. The compound of any one of clauses 1, 1a, 1b, 1c, 2, 2a, 3, 3a, 4, 4a, or 4b, or a pharmaceutically acceptable salt thereof, wherein

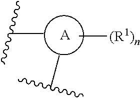

is a C₆-C₁₀ arylene, and n is 0, 1, 2, 3, or 4.

20. The compound of any one of clauses 1, 1a, 1b, 1c, 2, 2a, 3, 3a, 4, 4a, 4b, or 19, or a pharmaceutically acceptable salt thereof, wherein

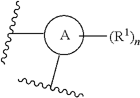

is a phenylene, and n is 0, 1, 2, 3, or 4.

21. The compound of any one of clauses 1, 1a, 1b, 1c, 2, 2a, 3, 3a, 4, 4a, 4b, 19, or 20, or a pharmaceutically acceptable salt thereof, wherein n is 0, 1, or 2.

22. The compound of any one of the preceding clauses, or a pharmaceutically acceptable salt thereof, wherein each R¹, when present, is independently fluoro, chloro, methyl, ethyl, methoxy, ethoxy, —C(O)OR^{a}, —C(O)NR^{a}R^{b}, —CN, or 4-piperidinyl.

23. The compound of any one of clauses 1, 1a, 1b, 1c, 2, 2a, 3, 3a, 4, 4a, 4b, or 19 to 22, or a pharmaceutically acceptable salt thereof, wherein

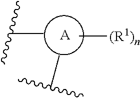

is selected from the group consisting of

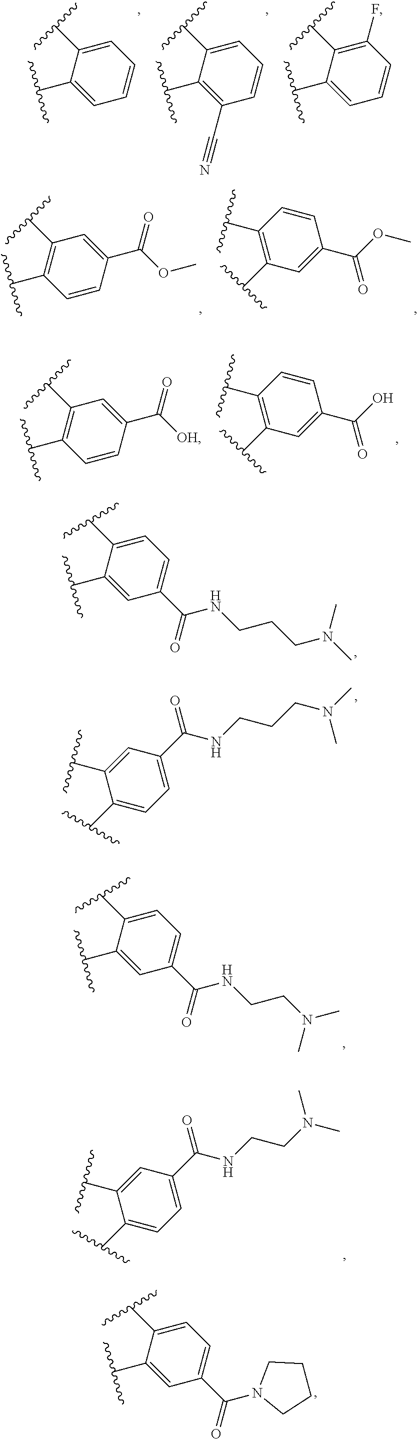

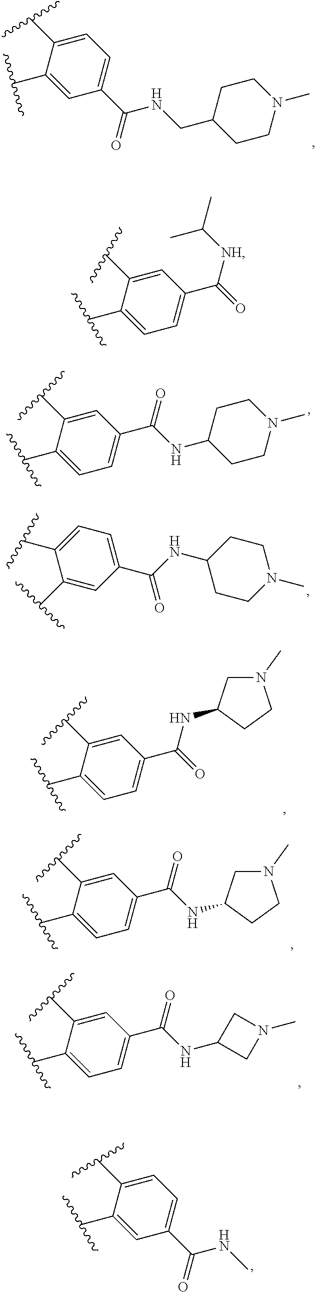

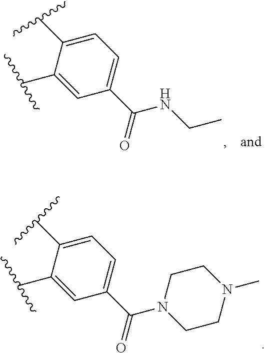 [SMILES: *c1ccc(C(=O)NCC)cc1* ; *c1ccc(C(=O)N2CCN(C)CC2)cc1*]

24. The compound of any one of the preceding clauses, or a pharmaceutically acceptable salt thereof, wherein L is an ethylene, propylene, or butylene, wherein each hydrogen atom in ethylene, propylene, and butylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂.

25. The compound of any one of the preceding clauses, or a pharmaceutically acceptable salt thereof, wherein L is an ethylene, propylene, or butylene, each of which is optionally substituted by a C₁-C₆ alkyl.

26. The compound of any one of the preceding clauses, or a pharmaceutically acceptable salt thereof, wherein Y is O.

27. The compound of any one of clauses 1, 1a, 1b, 1c, 2, 2a, 3, 3a, 4, 4a, 4b, or 5 to 25, or a pharmaceutically acceptable salt thereof, wherein Y is N(R⁵)C(O).

28. The compound of any one of the preceding clauses, or a pharmaceutically acceptable salt thereof, wherein R⁵, when present, is H or methyl.

29. The compound of any one of clauses 1, 1a, 1b, 1c, 2, 2a, 3, 3a, 4, 4a, 4b, or 5 to 25, or a pharmaceutically acceptable salt thereof, wherein Y is absent.

30. The compound of any one of the preceding clauses, or a pharmaceutically acceptable salt thereof, wherein Y¹ is O.

31. The compound of any one of clauses 1, 1a, 1b, 1c, 2, 2a, 3, 3a, 4, 4a, 4b, or 5 to 29, or a pharmaceutically acceptable salt thereof, wherein Y¹ is C(O)N(R⁷).

32. The compound of any one of the preceding clauses, or a pharmaceutically acceptable salt thereof, wherein R⁷, when present, is H or methyl.

33. The compound of any one of clauses 1, 1a, 1b, 1c, 2, 2a, 3, 3a, 4, 4a, 4b, or 5 to 29, or a pharmaceutically acceptable salt thereof, wherein Y¹ is N(R⁸).

34. The compound of any one of the preceding clauses, or a pharmaceutically acceptable salt thereof, wherein R⁸, when present, is H or methyl.

35. The compound of any one of clauses 1, 1a, 1b, 1c, 2, 2a, 3, 3a, 4, 4a, 4b, or 5 to 29, or a pharmaceutically acceptable salt thereof, wherein Y¹ is absent.

36. The compound of any one of the preceding clauses, or a pharmaceutically acceptable salt thereof, wherein Y² is O.

37. The compound of any one of clauses 1, 1a, 1b, 1c, 2, 2a, 3, 3a, 4, 4a, 4b, or 5 to 35, or a pharmaceutically acceptable salt thereof, wherein Y² is C(O)N(R⁹).

38. The compound of clause 37, or a pharmaceutically acceptable salt thereof, wherein R⁹ is H, methyl, ethyl, or cyclopropyl.

39. The compound of any one of clauses 1, 1a, 1b, 1c, 2, 2a, 3, 3a, 4, 4a, 4b, or 5 to 35, or a pharmaceutically acceptable salt thereof, wherein Y² is N(R¹⁰).

40. The compound of clause 39, or a pharmaceutically acceptable salt thereof, wherein R¹⁰ is H, methyl, or phenyl.

41. The compound of any one of clauses 1, 1a, 1b, 1c, 2, 2a, 3, 3a, 4, 4a, 4b, or 5 to 35, or a pharmaceutically acceptable salt thereof, wherein Y² is S(O)₂.

42. The compound of any one of clauses 1, 1a, 1b, 1c, 2, 2a, 3, 3a, 4, 4a, 4b, or 5 to 35, or a pharmaceutically acceptable salt thereof, wherein Y² is absent.

43. The compound of any one of the preceding clauses, or a pharmaceutically acceptable salt thereof, wherein m is 1.

44. The compound of any one of the preceding clauses, or a pharmaceutically acceptable salt thereof, wherein m is 2.

45. The compound of any one of the preceding clauses, or a pharmaceutically acceptable salt thereof, wherein at least one L¹ is methylene, ethylene, or propylene, wherein each hydrogen atom in methylene, ethylene, and propylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

46. The compound of any one of the preceding clauses, or a pharmaceutically acceptable salt thereof, wherein at least one L¹ is methylene, ethylene, or propylene, each of which is substituted with a C₁-C₆ alkyl or a —C(O)NR^{c}R^{d}.

47. The compound of any one of the preceding clauses, or a pharmaceutically acceptable salt thereof, wherein at least one L¹ is methylene, ethylene, or propylene, each of which is substituted with a methyl or a —C(O)NR^{c}R^{d}, wherein R^{c} and R^{d} are each H.

48. The compound of any one of clauses 1, 1a, 1b, 1c, 2, 2a, 3, 3a, 4, 4a, 4b, or 5 to 42, or a pharmaceutically acceptable salt thereof, wherein m is 0.

49. The compound of any one of the preceding clauses, or a pharmaceutically acceptable salt thereof, wherein X is C(R²).

50. The compound of any one of the preceding clauses, or a pharmaceutically acceptable salt thereof, wherein X¹ is CH or N.

51. The compound of any one of clauses 1, 1a, 1b, 1c, 2, 2a, 3, 3a, 4, 4a, 4b, or 5 to 49, or a pharmaceutically acceptable salt thereof, wherein X¹ is C(R³).

52. The compound of any one of the preceding clauses, or a pharmaceutically acceptable salt thereof, wherein X² is C(R⁴).

53. The compound of any one of the clauses 1, 1a, 1b, 1c, 2, 2a, 3, 3a, 4, 4a, 4b, or 5 to 51, or a pharmaceutically acceptable salt thereof, wherein X² is N.

54. The compound of any one of clauses 1, 1a, 1b, 1c, 2, 2a, 3, 3a, 4, 4a, 4b, or 5 to 48, or a pharmaceutically acceptable salt thereof, wherein X is N.

55. The compound of any one of clauses 1, 1a, 1b, 1c, 2, 2a, 3, 3a, 4, 4a, 4b, or 5 to 48, or 54, or a pharmaceutically acceptable salt thereof, wherein X¹ is C(R³).

56. The compound of any one of clauses 1, 1a, 1b, 1c, 2, 2a, 3, 3a, 4, 4a, 4b, or 5 to 48, or 54, or a pharmaceutically acceptable salt thereof, wherein X¹ is N.

57. The compound of any one of clauses 1, 1a, 1b, 1c, 2, 2a, 3, 3a, 4, 4a, 4b, 5 to 48, or 54 to 56, or a pharmaceutically acceptable salt thereof, wherein X² is C(R⁴).

58. The compound of any one of clauses 1, 1a, 1b, 1c, 2, 2a, 3, 3a, 4, 4a, 4b, 5 to 48, or 54 to 56, or a pharmaceutically acceptable salt thereof, wherein X² is N.

59. The compound of any one of the preceding clauses, or a pharmaceutically acceptable salt thereof, wherein R², when present, is H.

60. The compound of any one of the preceding clauses, or a pharmaceutically acceptable salt thereof, wherein R³, when present, is H.

61. The compound of any one of the preceding clauses, or a pharmaceutically acceptable salt thereof, wherein R⁴, when present, is H, fluoro, chloro, or methyl.

62. The compound of any one of the preceding clauses, or a pharmaceutically acceptable salt thereof, wherein R¹¹ is H.

63. The compound of clause 1, selected from the group consisting of

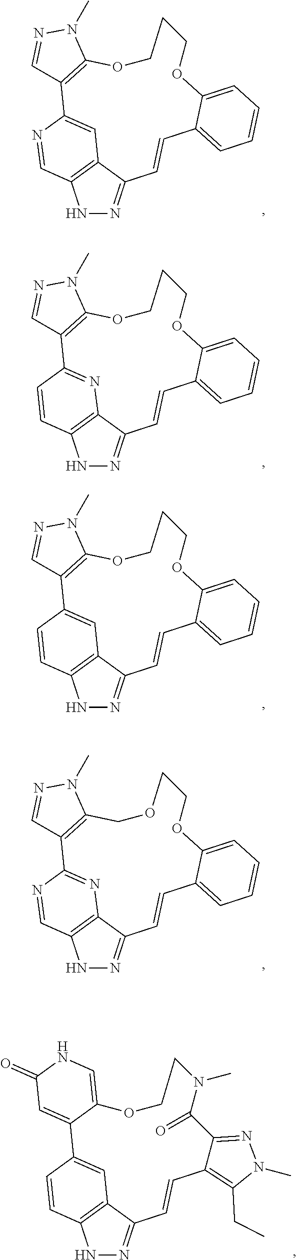

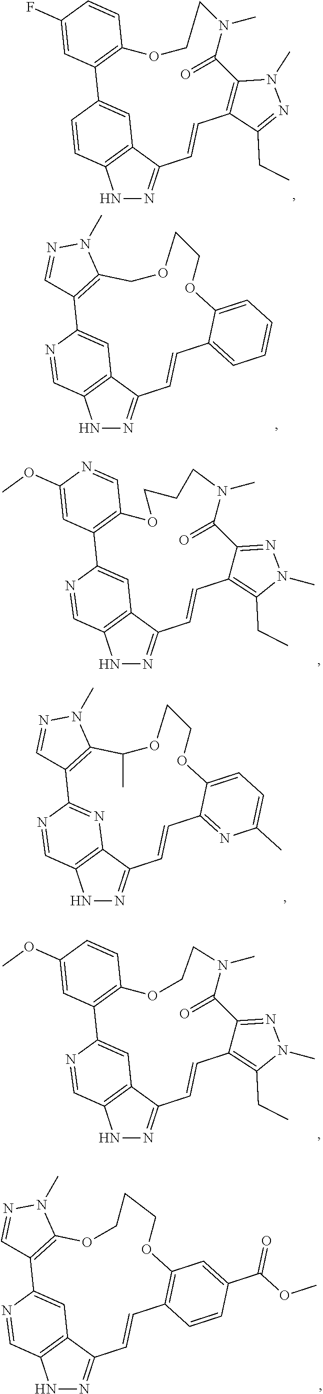

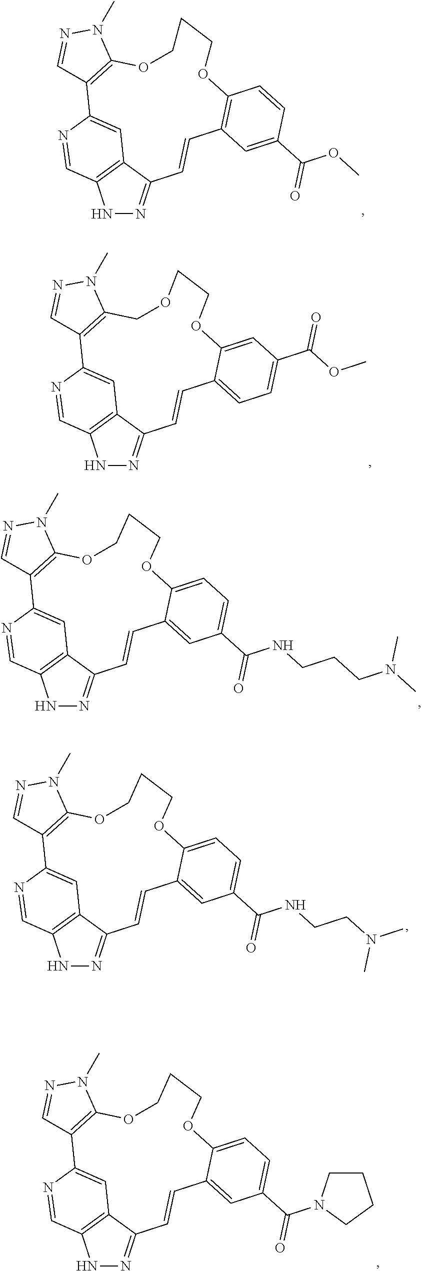

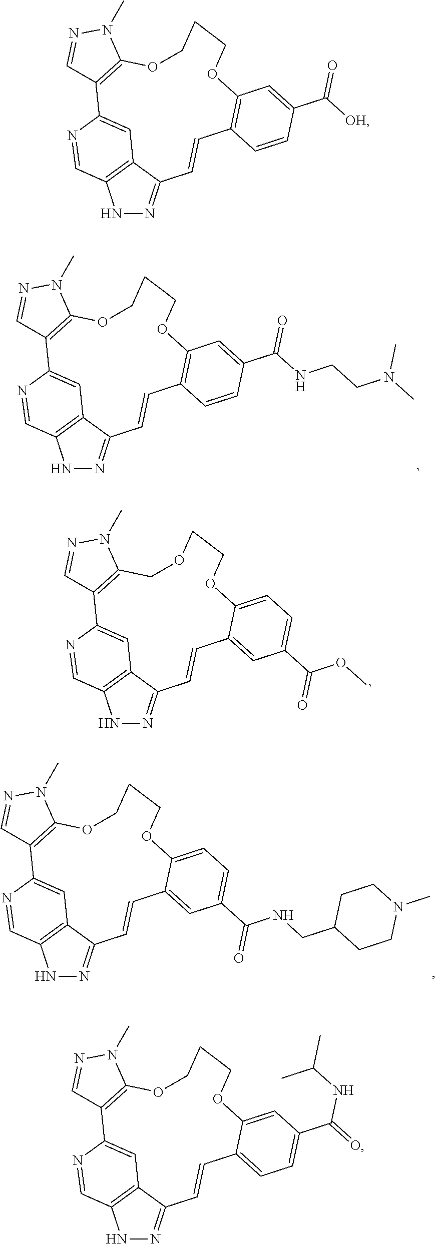 [SMILES: Cn1ncc2c1OCCCOc1cc(C(=O)O)ccc1/C=C/c1n[nH]c3cnc-2cc13 ; CN(C)CCNC(=O)c1ccc2c(c1)OCCCOc1c(cnn1C)-c1cc3c(n[nH]c3cn1)/C=C/2 ; COC(=O)c1ccc2c(c1)/C=C/c1n[nH]c3cnc(cc13)-c1cnn(C)c1COCCO2 ; CN1CCC(CNC(=O)c2ccc3c(c2)/C=C/c2n[nH]c4cnc(cc24)-c2cnn(C)c2OCCCO3)CC1 ; CC(C)NC(=O)c1ccc2c(c1)OCCCOc1c(cnn1C)-c1cc3c(n[nH]c3cn1)/C=C/2]

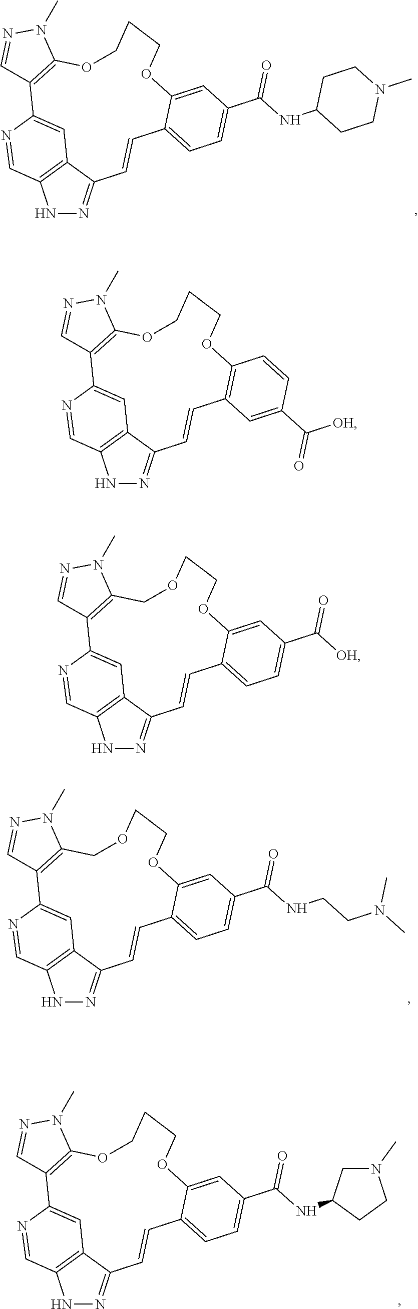 [SMILES: CN1CCC(NC(=O)c2ccc3c(c2)OCCCOc2c(cnn2C)-c2cc4c(n[nH]c4cn2)/C=C/3)CC1 ; Cn1ncc2c1OCCCOc1ccc(C(=O)O)cc1/C=C/c1n[nH]c3cnc-2cc13 ; Cn1ncc2c1COCCOc1cc(C(=O)O)ccc1/C=C/c1n[nH]c3cnc-2cc13 ; CN(C)CCNC(=O)c1ccc2c(c1)OCCOCc1c(cnn1C)-c1cc3c(n[nH]c3cn1)/C=C/2 ; CN1CC[C@@H](NC(=O)c2ccc3c(c2)OCCCOc2c(cnn2C)-c2cc4c(n[nH]c4cn2)/C=C/3)C1]

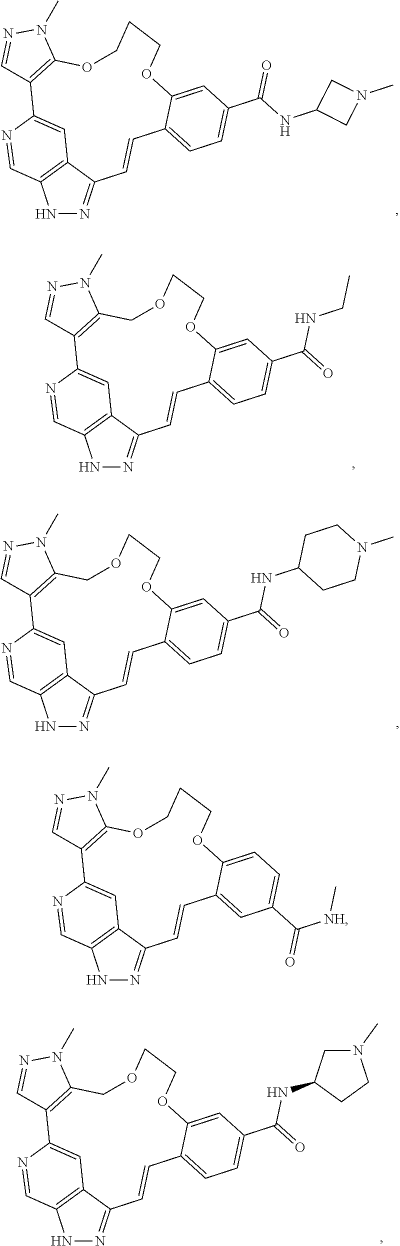

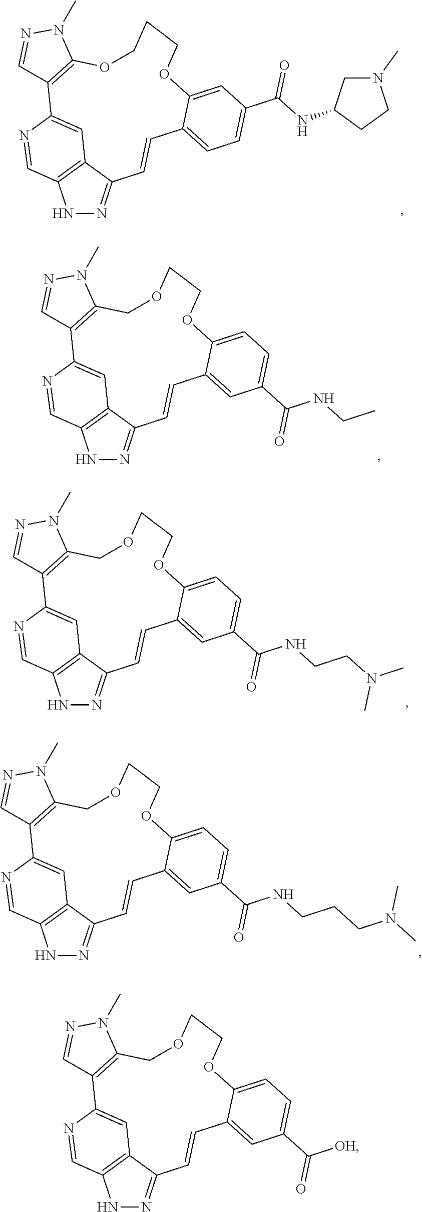

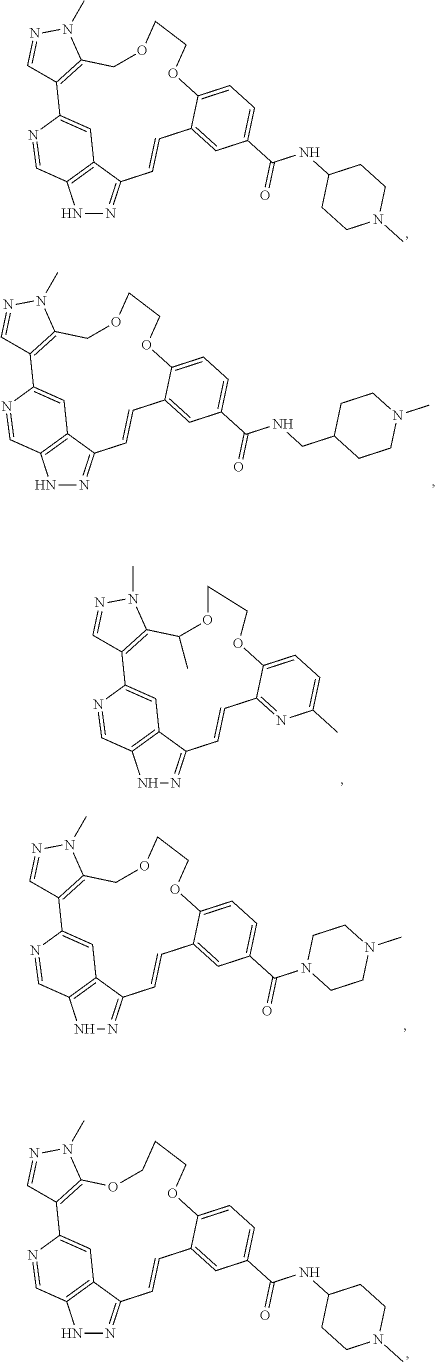 [SMILES: CN1CCC(NC(=O)c2ccc3c(c2)/C=C/c2n[nH]c4cnc(cc24)-c2cnn(C)c2COCCO3)CC1 ; CN1CCC(CNC(=O)c2ccc3c(c2)/C=C/c2n[nH]c4cnc(cc24)-c2cnn(C)c2COCCO3)CC1 ; Cc1ccc2c(n1)/C=C/c1n[nH]c3cnc(cc13)-c1cnn(C)c1C(C)OCCO2 ; CN1CCN(C(=O)c2ccc3c(c2)/C=C/c2n[nH]c4cnc(cc24)-c2cnn(C)c2COCCO3)CC1 ; CN1CCC(NC(=O)c2ccc3c(c2)/C=C/c2n[nH]c4cnc(cc24)-c2cnn(C)c2OCCCO3)CC1]

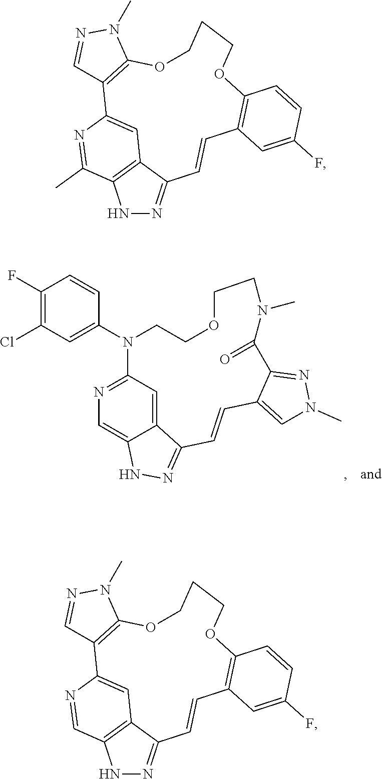 [SMILES: Cc1nc2cc3c(n[nH]c13)/C=C/c1cc(F)ccc1OCCCOc1c-2cnn1C ; CN1CCOCCN(c2ccc(F)c(Cl)c2)c2cc3c(n[nH]c3cn2)/C=C/c2cn(C)nc2C1=O ; Cn1ncc2c1OCCCOc1ccc(F)cc1/C=C/c1n[nH]c3cnc-2cc13]

or a pharmaceutically acceptable salt thereof.

64. The compound of clause 1, selected from the group consisting of

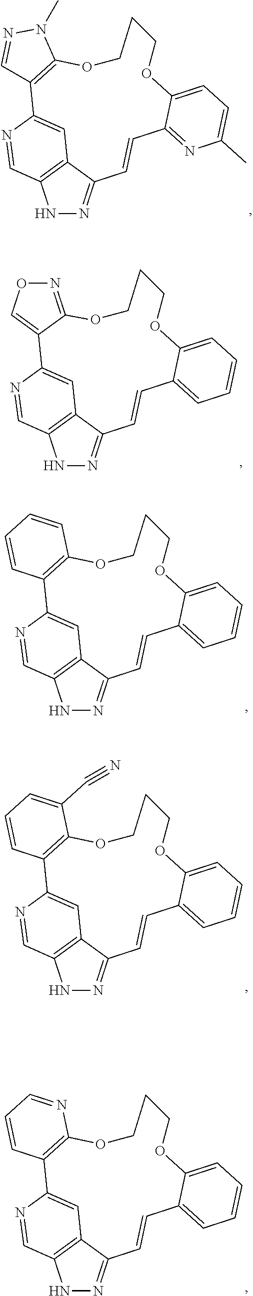 [SMILES: Cc1ccc2c(n1)/C=C/c1n[nH]c3cnc(cc13)-c1cnn(C)c1OCCCO2 ; C1=C/c2n[nH]c3cnc(cc23)-c2conc2OCCCOc2ccccc2/1 ; C1=C/c2n[nH]c3cnc(cc23)-c2ccccc2OCCCOc2ccccc2/1 ; N#Cc1cccc2c1OCCCOc1ccccc1/C=C/c1n[nH]c3cnc-2cc13 ; C1=C/c2n[nH]c3cnc(cc23)-c2cccnc2OCCCOc2ccccc2/1]

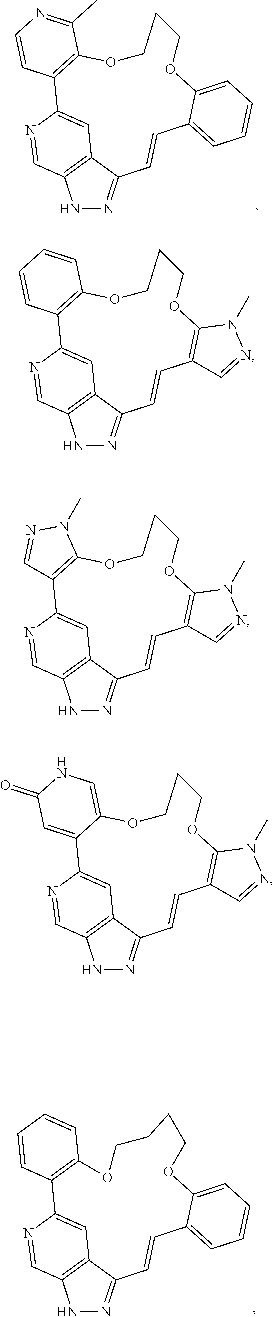 [SMILES: Cc1nccc2c1OCCCOc1ccccc1/C=C/c1n[nH]c3cnc-2cc13 ; Cn1ncc2c1OCCCOc1ccccc1-c1cc3c(n[nH]c3cn1)/C=C/2 ; Cn1ncc2c1OCCCOc1c(cnn1C)-c1cc3c(n[nH]c3cn1)/C=C/2 ; Cn1ncc2c1OCCCOc1c[nH]c(=O)cc1-c1cc3c(n[nH]c3cn1)/C=C/2 ; C1=C/c2n[nH]c3cnc(cc23)-c2ccccc2OCCCCOc2ccccc2/1]

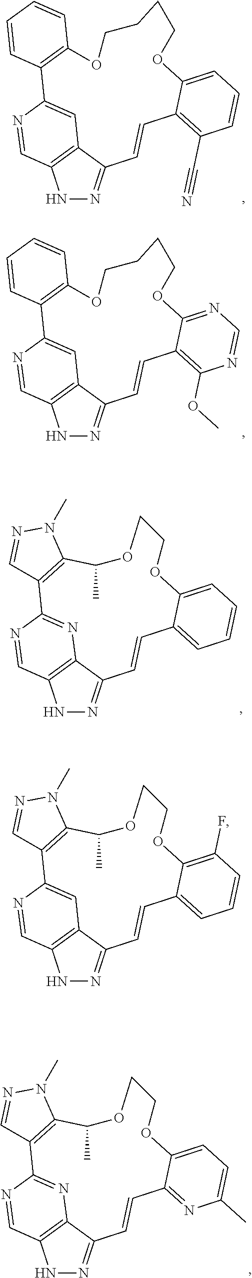 [SMILES: N#Cc1cccc2c1/C=C/c1n[nH]c3cnc(cc13)-c1ccccc1OCCCCO2 ; COc1ncnc2c1/C=C/c1n[nH]c3cnc(cc13)-c1ccccc1OCCCCO2 ; C[C@H]1OCCOc2ccccc2/C=C/c2n[nH]c3cnc(nc23)-c2cnn(C)c21 ; C[C@H]1OCCOc2c(F)cccc2/C=C/c2n[nH]c3cnc(cc23)-c2cnn(C)c21 ; Cc1ccc2c(n1)/C=C/c1n[nH]c3cnc(nc13)-c1cnn(C)c1[C@@H](C)OCCO2]

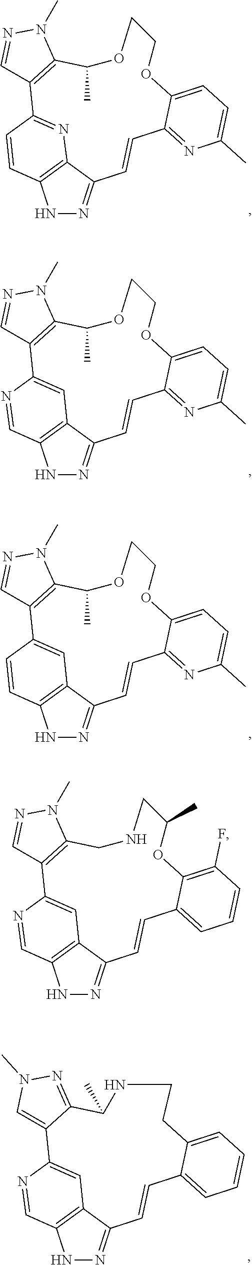

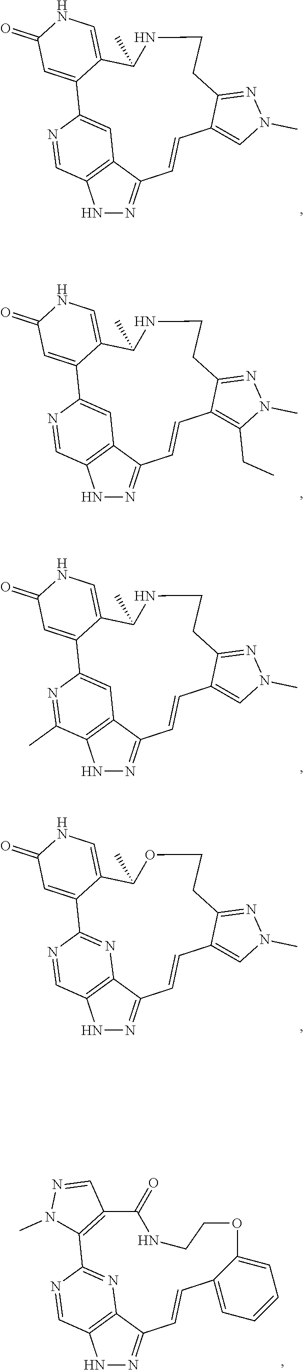

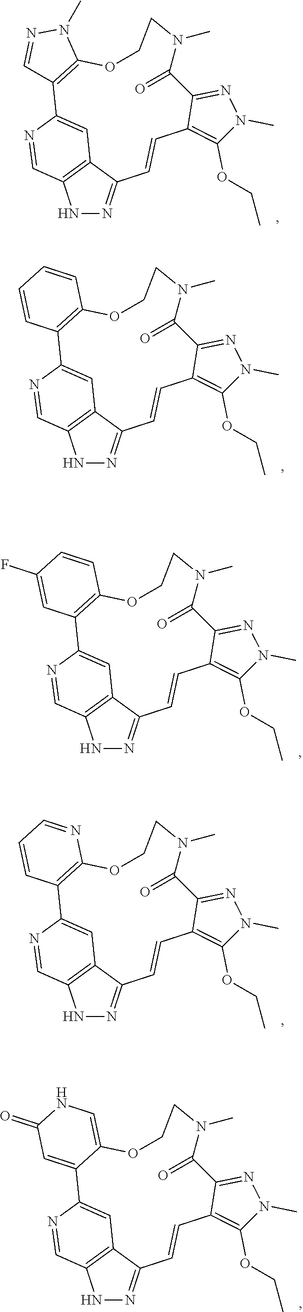 [SMILES: CCOc1c2c(nn1C)C(=O)N(C)CCOc1c(cnn1C)-c1cc3c(n[nH]c3cn1)/C=C/2 ; CCOc1c2c(nn1C)C(=O)N(C)CCOc1ccccc1-c1cc3c(n[nH]c3cn1)/C=C/2 ; CCOc1c2c(nn1C)C(=O)N(C)CCOc1ccc(F)cc1-c1cc3c(n[nH]c3cn1)/C=C/2 ; CCOc1c2c(nn1C)C(=O)N(C)CCOc1ncccc1-c1cc3c(n[nH]c3cn1)/C=C/2 ; CCOc1c2c(nn1C)C(=O)N(C)CCOc1c[nH]c(=O)cc1-c1cc3c(n[nH]c3cn1)/C=C/2]

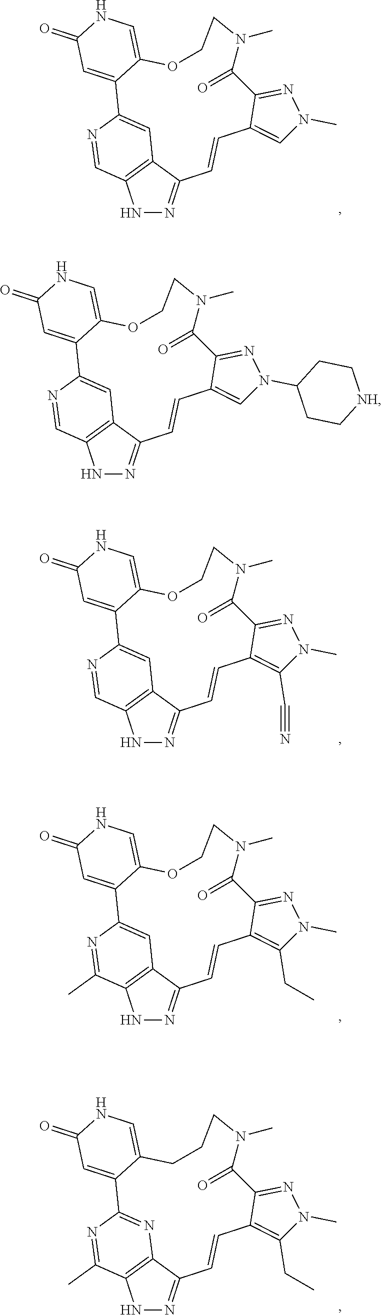 [SMILES: CN1CCOc2c[nH]c(=O)cc2-c2cc3c(n[nH]c3cn2)/C=C/c2cn(C)nc2C1=O ; CN1CCOc2c[nH]c(=O)cc2-c2cc3c(n[nH]c3cn2)/C=C/c2cn(C3CCNCC3)nc2C1=O ; CN1CCOc2c[nH]c(=O)cc2-c2cc3c(n[nH]c3cn2)/C=C/c2c(nn(C)c2C#N)C1=O ; CCc1c2c(nn1C)C(=O)N(C)CCOc1c[nH]c(=O)cc1-c1cc3c(n[nH]c3c(C)n1)/C=C/2 ; CCc1c2c(nn1C)C(=O)N(C)CCCc1c[nH]c(=O)cc1-c1nc(C)c3[nH]nc(c3n1)/C=C/2]

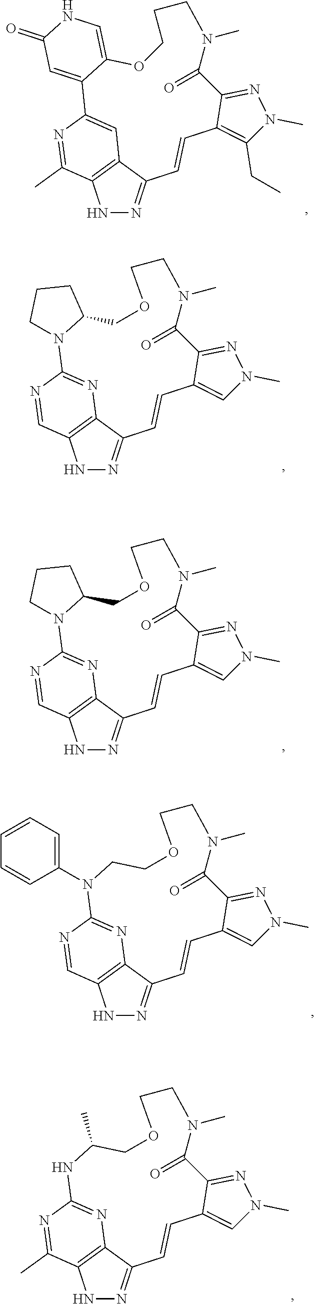

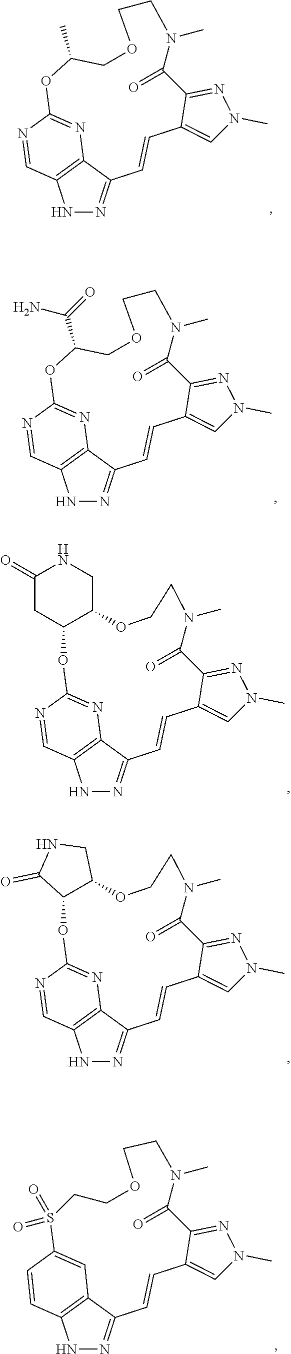 [SMILES: C[C@@H]1COCCN(C)C(=O)c2nn(C)cc2/C=C/c2n[nH]c3cnc(nc23)O1 ; CN1CCOC[C@@H](C(N)=O)Oc2ncc3[nH]nc(c3n2)/C=C/c2cn(C)nc2C1=O ; CN1CCO[C@H]2CNC(=O)C[C@H]2Oc2ncc3[nH]nc(c3n2)/C=C/c2cn(C)nc2C1=O ; CN1CCO[C@H]2CNC(=O)[C@H]2Oc2ncc3[nH]nc(c3n2)/C=C/c2cn(C)nc2C1=O ; CN1CCOCCS(=O)(=O)c2ccc3[nH]nc(c3c2)/C=C/c2cn(C)nc2C1=O]

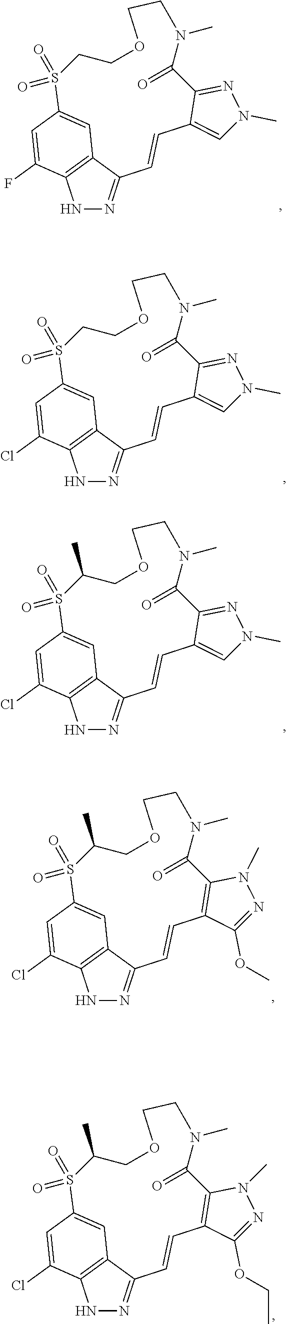 [SMILES: CN1CCOCCS(=O)(=O)c2cc(F)c3[nH]nc(c3c2)/C=C/c2cn(C)nc2C1=O ; CN1CCOCCS(=O)(=O)c2cc(Cl)c3[nH]nc(c3c2)/C=C/c2cn(C)nc2C1=O ; C[C@H]1COCCN(C)C(=O)c2nn(C)cc2/C=C/c2n[nH]c3c(Cl)cc(cc23)S1(=O)=O ; COc1nn(C)c2c1/C=C/c1n[nH]c3c(Cl)cc(cc13)S(=O)(=O)[C@@H](C)COCCN(C)C2=O ; CCOc1nn(C)c2c1/C=C/c1n[nH]c3c(Cl)cc(cc13)S(=O)(=O)[C@@H](C)COCCN(C)C2=O]

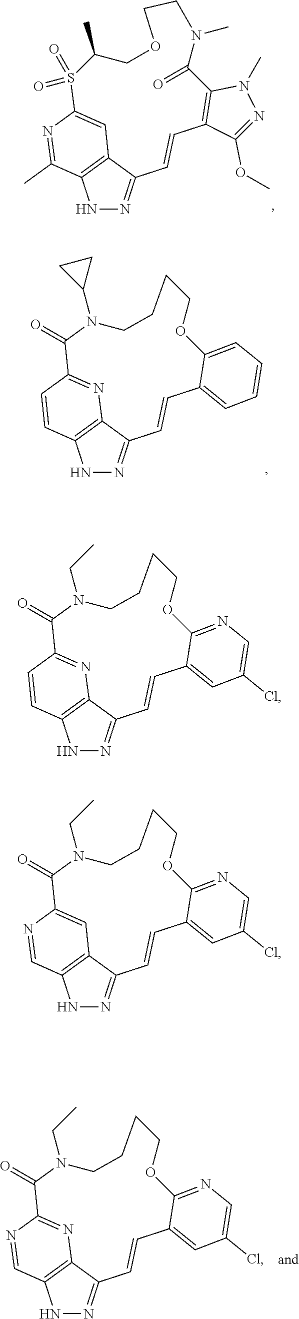

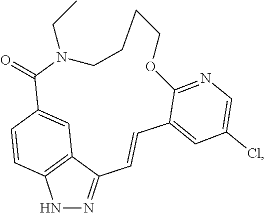 [SMILES: CCN1CCCCOc2ncc(Cl)cc2/C=C/c2n[nH]c3ccc(cc23)C1=O]

or a pharmaceutically acceptable salt thereof.

65. A pharmaceutical composition comprising a compound of any one of the preceding clauses, and optionally one or more excipients.

66. A method of treating disease, such as cancer, such as a cancer having one or more EGFR mutations, such as L858R, Del19, Δ746-750, Δ746-750/T790M, Δ746-750/C979S, L858R/T790M, Del19/T790M, L858R/C979S, Del19/C979S, L858R/T790M/C979S, and Δ746-750/T790M/C979S, in a subject comprising, administering a therapeutically effective amount of a compound of any one of clauses 1, 1a, 1b, 1c, 2, 2a, 3, 3a, 4, 4a, 4b, or 5 to 64, or a pharmaceutical composition of clause 65.

67. A compound according to any one of clauses 1, 1a, 1b, 1c, 2, 2a, 3, 3a, 4, 4a, 4b, or 5 to 64, for use in a method of treating disease, such as cancer, such as a cancer having one or more EGFR mutations, such as L858R, Del19, Δ746-750, Δ746-750/T790M, A746-750/C979S, L858R/T790M, Del19/T790M, L858R/C979S, Del19/C979S, L858R/T790M/C979S, and Δ746-750/T790M/C979S, in a subject.

68. Use of a compound according to any one of clauses 1, 1a, 1b, 1c, 2, 2a, 3, 3a, 4, 4a, 4b, or 5 to 64 in the manufacture of a medicament for the treatment of disease, such as cancer, such as a cancer having one or more EGFR mutations, such as L858R, Del19, Δ746-750, A746-750/T790M, Δ746-750/C979S, L858R/T790M, Del19/T790M, L858R/C979S, Del19/C979S, L858R/T790M/C979S, and Δ746-750/T790M/C979S, in a subject.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2020 (ACD/Labs) or ChemBioDraw Ultra 13.0 (Perkin Elmer).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Chemical Definitions

The term “alkyl” refers to a straight- or branched-chain mono-valent hydrocarbon group. The term “alkylene” refers to a straight- or branched-chain di-valent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be combined with another group as described herein or an atom, such as a N, O, or S. For example, an O can be combined with an alkyl to provide a mono-valent —O-alkyl group, such as —O—C₁-C₆ alkyl, having an open valence on only one end for connection with another structure. Alternatively, an O can be combined with an alkylene to provide an di-valent —O-alkylene- group, such as —O—C₁-C₆ alkylene-, —O—(C₁-C₆ alkylene)-, or —O(C₁-C₆ alkylene)-, having open valences on both ends of the group for covalent attachment to two different structures. It will be appreciated that an alkyl or alkylene group can be unsubstituted or substituted as described herein. An alkyl or alkylene group can be substituted with any of the substituents in the various embodiments described herein, including one or more of such substituents.

The term “alkenyl” refers to a straight- or branched-chain mono-valent hydrocarbon group having one or more double bonds. The term “alkenylene” refers to a straight- or branched-chain di-valent hydrocarbon group having one or more double bonds. In some embodiments, it can be advantageous to limit the number of atoms in an “alkenyl” or “alkenylene” to a specific range of atoms, such as C₂-C₂₀ alkenyl or C₂-C₂₀ alkenylene, C₂-C₁₂ alkenyl or C₂-C₁₂ alkenylene, or C₂-C₆ alkenyl or C₂-C₆ alkenylene. Examples of alkenyl groups include ethenyl (or vinyl), allyl, and but-3-en-1-yl. Examples of alkenylene groups include ethenylene (or vinylene) (—CH═CH—), n-propenylene (—CH═CHCH₂—), iso-propenylene (—CH═CH(CH₃)—), and the like. Included within this term are cis and trans isomers and mixtures thereof. It will be appreciated that an alkenyl or alkenylene group can be combined with another group as described herein or an atom, such as a N, O, or S. For example, an O can be combined with an alkyl to provide a mono-valent —O-alkenyl group, such as —O—C₁-C₆ alkenyl, having an open valence on only one end for connection with another structure. Alternatively, an O can be combined with an alkenylene to provide an di-valent —O-alkenylene-group, such as —O—C₁-C₆ alkenylene-, —O—(C₁-C₆ alkenylene)-, or —O(C₁-C₆ alkenylene)-, having open valences on both ends of the group for covalent attachment to two different structures. It will be appreciated that an alkenyl or alkenylene group can be unsubstituted or substituted as described herein. An alkenyl or alkenylene group can be substituted with any of the substituents in the various embodiments described herein, including one or more of such substituents.

The term “alkynyl” refers to a straight- or branched-chain mono-valent hydrocarbon group having one or more triple bonds. The term “alkynylene” refers to a straight- or branched-chain di-valent hydrocarbon group having one or more triple bonds. In some embodiments, it can be advantageous to limit the number of atoms in an “alkynyl” or “alkynylene” to a specific range of atoms, such as C₂-C₂₀ alkynyl or C₂-C₂₀ alkynylene, C₂-C₁₂ alkynyl or C₂-C₁₂ alkynylene, or C₂C₆ alkynyl or C₂-C₆ alkynylene. Examples of alkynyl groups include acetylenyl (—C≡CH) and propargyl (—CH₂C≡CH), butynyl (—C≡C—CH₂CH₃), and the like. Examples of alkynylene groups include acetylenylene (—C≡C—) and propargylene (—CH₂C≡C—), but-3-yn-1,4-diyl (—C≡C—CH₂CH₂—), and the like. It will be appreciated that an alkyl or alkylene group can be combined with another group as described herein or an atom, such as a N, O, or S. For example, an O can be combined with an alkyl to provide a mono-valent —O-alkynyl group, such as —O—C₁-C₆ alkynyl, having an open valence on only one end for connection with another structure. Alternatively, an O can be combined with an alkynylene to provide an di-valent —O-alkynylene- group, such as —O—C₁-C₆ alkynylene-, —O—(C₁-C₆ alkynylene)-, or —O(C₁-C₆ alkynylene)-, having open valences on both ends of the group for covalent attachment to two different structures. It will be appreciated that an alkynyl or alkynylene group can be unsubstituted or substituted as described herein. An alkynyl or alkynylene group can be substituted with any of the substituents in the various embodiments described herein, including one or more of such substituents.

The term “cycloalkyl” refers to a saturated or partially saturated, monocyclic or polycyclic mono-valent carbocycle. The term “cycloalkylene” refers to a saturated or partially saturated, monocyclic or polycyclic di-valent carbocycle. In some embodiments, it can be advantageous to limit the number of atoms in a “cycloalkyl” or “cycloalkylene” to a specific range of atoms, such as having 3 to 12 ring atoms. Polycyclic carbocycles include fused, bridged, and spiro polycyclic systems. Illustrative examples of cycloalkyl groups include mono-valent radicals of the following entities, while cycloalkylene groups include di-valent radicals of the following entities, in the form of properly bonded moieties:

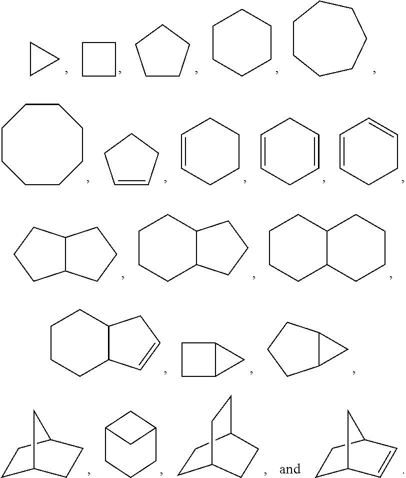

In particular, a cyclopropyl moiety can be depicted by the structural formula

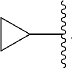 [SMILES: *C1CC1]

In particular, a cyclopropylene moiety can be depicted by the structural formula

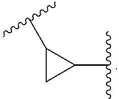 [SMILES: *C1CC1*]

It will be appreciated that a cycloalkyl or cycloalkylene group can be unsubstituted or substituted as described herein. A cycloalkyl or cycloalkylene group can be substituted with any of the substituents in the various embodiments described herein, including one or more of such substituents.

The term “halogen” or “halo” represents chlorine, fluorine, bromine, or iodine.

The term “haloalkyl” refers to an alkyl group with one or more halo substituents. Examples of haloalkyl groups include —CF₃, —(CH₂)F, —CHF₂, —CH₂Br, —CH₂CF₃, and —CH₂CH₂F. The term “haloalkylene” refers to an alkyl group with one or more halo substituents. Examples of haloalkyl groups include —CF₂—, —C(H)(F)—, —C(H)(Br)—, —CH₂CF₂—, and —CH₂C(H)(F)—.

The term “aryl” refers to a mono-valent all-carbon monocyclic or fused-ring polycyclic group having a completely conjugated pi-electron system. The term “arylene” refers to a di-valent all-carbon monocyclic or fused-ring polycyclic group having a completely conjugated pi-electron system. In some embodiments, it can be advantageous to limit the number of atoms in an “aryl” or “arylene” to a specific range of atoms, such as mono-valent all-carbon monocyclic or fused-ring polycyclic groups of 6 to 14 carbon atoms (C₆-C₁₄ aryl), mono-valent all-carbon monocyclic or fused-ring polycyclic groups of 6 to 10 carbon atoms (C₆-C₁₀ aryl), di-valent all-carbon monocyclic or fused-ring polycyclic groups of 6 to 14 carbon atoms (C₆-C₁₄ arylene), and di-valent all-carbon monocyclic or fused-ring polycyclic groups of 6 to 10 carbon atoms (C₆-C₁₀ arylene). Examples, without limitation, of aryl groups are phenyl, naphthalenyl and anthracenyl. Examples, without limitation, of arylene groups are phenylene, naphthalenylene and anthracenylene. It will be appreciated that an aryl or arylene group can be unsubstituted or substituted as described herein. An aryl or arylene group can be substituted with any of the substituents in the various embodiments described herein, including one or more of such substituents.

The term “heterocycloalkyl” refers to a mono-valent monocyclic or polycyclic ring structure that is saturated or partially saturated having one or more non-carbon ring atoms. The term “heterocycloalkylene” refers to a di-valent monocyclic or polycyclic ring structure that is saturated or partially saturated having one or more non-carbon ring atoms. In some embodiments, it can be advantageous to limit the number of atoms in a “heterocycloalkyl” or “heterocycloalkylene” to a specific range of ring atoms, such as from 3 to 12 ring atoms (3- to 12-membered), or 3 to 7 ring atoms (3- to 7-membered), or 3 to 6 ring atoms (3- to 6-membered), or 4 to 6 ring atoms (4- to 6-membered), or 5 to 7 ring atoms (5- to 7-membered). In some embodiments, it can be advantageous to limit the number and type of ring heteroatoms in “heterocycloalkyl” or “heterocycloalkylene” to a specific range or type of heteroatoms, such as 1 to 5 ring heteroatoms selected from nitrogen, oxygen, and sulfur. Polycyclic ring systems include fused, bridged, and spiro systems. The ring structure may optionally contain an oxo group on a carbon ring member or up to two oxo groups on sulfur ring members. Illustrative examples of heterocycloalkyl groups include mono-valent radicals of the following entities, while heterocycloalkylene groups include di-valent radicals of the following entities, in the form of properly bonded moieties:

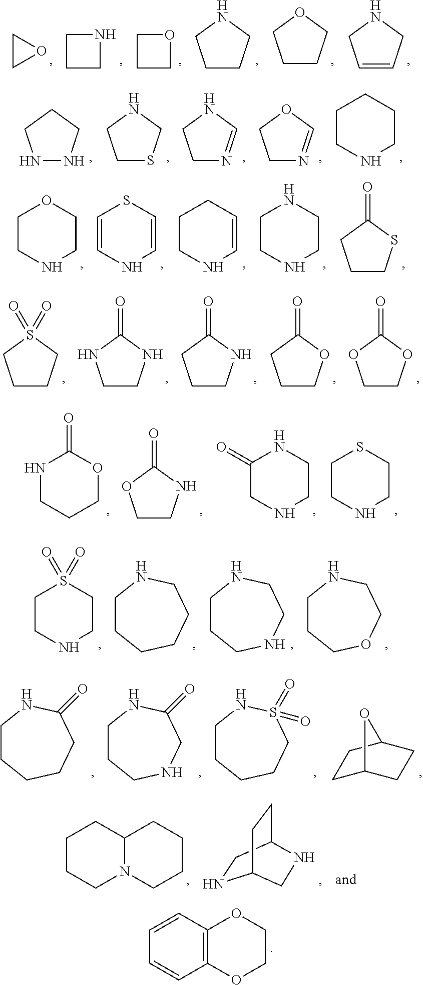

It will be appreciated that a nitrogen containing heterocycloalkyl can be represented by the formula —N(alkylene), where the alkylene group is described by a parenthetical with no indicated open valence, in which case the alkylene group is understood to occupy two valence positions on the nitrogen atom to provide a heterocycloalkyl structure. For example, the group —N(C₂-C₆ alkylene) is within the scope of the term 3- to 7-membered heterocycloalkyl, where the 3- to 7-heterocycloalkyl has one nitrogen atom in the ring that represents the point of attachment. In particular, —N(C₂-C₆ alkylene) includes each of the following heterocycloalkyl structures.

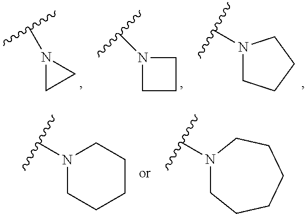

It will be appreciated that a heterocycloalkyl or heterocycloalkylene group can be unsubstituted or substituted as described herein. A heterocycloalkyl or heterocycloalkylene group can be substituted with any of the substituents in the various embodiments described herein, including one or more of such substituents.

The term “heteroaryl” refers to a mono-valent monocyclic, fused bicyclic, or fused polycyclic aromatic heterocycle (ring structure having ring atoms or members selected from carbon atoms and up to four heteroatoms selected from nitrogen, oxygen, and sulfur) that is fully unsaturated and having from 3 to 12 ring atoms per heterocycle. The term “heteroarylene” refers to a di-valent monocyclic, fused bicyclic, or fused polycyclic aromatic heterocycle (ring structure having ring atoms or members selected from carbon atoms and up to four heteroatoms selected from nitrogen, oxygen, and sulfur) having from 3 to 12 ring atoms per heterocycle. In some embodiments, it can be advantageous to limit the number of ring atoms in a “heteroaryl” or “heteroarylene” to a specific range of atom members, such as 5- to 10-membered heteroaryl or 5- to 10-membered heteroarylene. In some instances, a 5- to 10-membered heteroaryl can be a monocyclic ring or fused bicyclic rings having 5- to 10-ring atoms wherein at least one ring atom is a heteroatom, such as N, O, or S. In some instances, a 5- to 10-membered heteroarylene can be a monocyclic ring or fused bicyclic rings having 5- to 10-ring atoms wherein at least one ring atom is a heteroatom, such as N, O, or S. Illustrative examples of 5- to 10-membered heteroaryl groups include mono-valent radicals of the following entities, while examples of 5- to 10-membered heteroarylene groups include di-valent radicals of the following entities, in the form of properly bonded moieties:

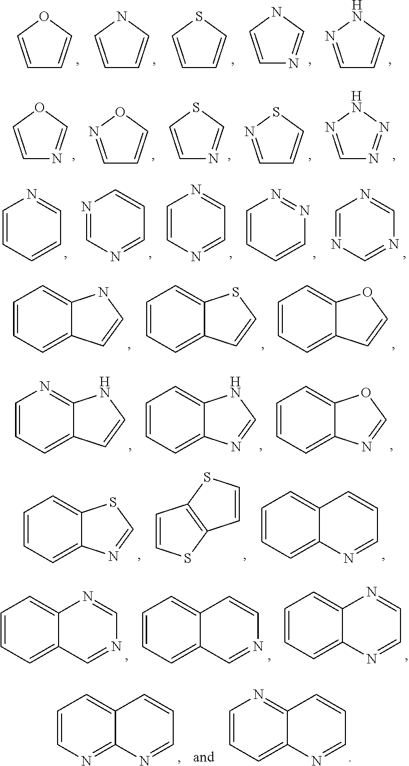

In some embodiments, a “monocyclic” heteroaryl can be an aromatic five- or six-membered heterocycle. A five-membered heteroaryl or heteroarylene can contain up to four heteroatom ring atoms, where (a) at least one ring atom is oxygen or sulfur and zero, one, two, or three ring atoms are nitrogen, or (b) zero ring atoms are oxygen or sulfur and up to four ring atoms are nitrogen. Non-limiting examples of five-membered heteroaryl groups include mono-valent radicals of furan, thiophene, pyrrole, oxazole, isoxazole, thiazole, isothiazole, pyrazole, imidazole, oxadiazole, thiadiazole, triazole, or tetrazole. Non-limiting examples of five-membered heteroarylene groups include di-valent radicals of furan, thiophene, pyrrole, oxazole, isoxazole, thiazole, isothiazole, pyrazole, imidazole, oxadiazole, thiadiazole, triazole, or tetrazole. A six-membered heteroaryl or heteroarylene can contain up to four heteroatom ring atoms, where (a) at least one ring atom is oxygen or sulfur and zero, one, two, or three ring atoms are nitrogen, or (b) zero ring atoms are oxygen or sulfur and up to four ring atoms are nitrogen. Non-limiting examples of six-membered heteroaryl groups include mono-valent radicals of pyridine, pyrazine, pyrimidine, pyridazine, or triazine. Non-limiting examples of six-membered heteroarylene groups include di-valent radicals of pyridine, pyrazine, pyrimidine, pyridazine, or triazine. In particular, a pyrazolyl moiety can be depicted by the structural formula

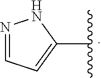 [SMILES: *c1ccn[nH]1]

In particular, a pyrazolylene moiety can be depicted by the structural formula

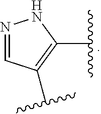 [SMILES: *c1cn[nH]c1*]

A “bicyclic heteroaryl” or “bicyclic heteroarylene” is a fused bicyclic system comprising one heteroaryl ring fused to a phenyl or another heteroaryl ring.

It will be appreciated that a heteroaryl or heteroarylene group can be unsubstituted or substituted as described herein. A heteroaryl or heteroarylene group can be substituted with any of the substituents in the various embodiments described herein, including one or more of such substituents.

The term “oxo” represents a carbonyl oxygen. For example, a cyclopentyl substituted with oxo is cyclopentanone.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The nomenclature “(ATOM)ᵢ₋ⱼ” with j>i, when applied herein to a class of substituents, is meant to refer to embodiments of this disclosure for which each and every one of the number of atom members, from i to j including i and j, is independently realized. By way of example, the term C₁₋₃ or C₁-C₃ refers independently to embodiments that have one carbon member (C₁), embodiments that have two carbon members (C₂), and embodiments that have three carbon members (C₃).

Any disubstituent referred to herein is meant to encompass the various attachment possibilities when more than one of such possibilities are allowed. For example, reference to disubstituent -A-B-, where A≠B, refers herein to such disubstituent with A attached to a first substituted member and B attached to a second substituted member, and it also refers to such disubstituent with A attached to the second substituted member and B attached to the first substituted member. For example, in certain embodiments, where applicable, a compound portion -(L)ₙ- having the formula —CH(CH₃)—CH₂NH—(CH₂)₂—, connecting two groups, A and B, will be understood that —CH(CH₃)—CH₂NH—(CH₂)₂—, can include both of the embodiments A-CH(CH₃)—CH₂NH—(CH₂)₂-B and B-CH(CH₃)—CH₂NH—(CH₂)₂-A. More particularly in the present case, compounds of the formula (Ia)-(IXa) or (I)-(IX) having a compound portion -(L)ₙ- of the formula —CH(CH₃)—CH₂NH—(CH₂)₂— connecting groups —Z— and —NR²— will be understood to include both embodiments —Z—CH(CH₃)—CH₂NH—(CH₂)₂—NR²— and —NR²—CH(CH₃)—CH₂NH—(CH₂)₂-A.

The disclosure also includes pharmaceutically acceptable salts of the compounds represented by Formula (Ia)-(IXa) or (I)-(IX), preferably of those described above and of the specific compounds exemplified herein, and pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula (Ia)-(IXa) or (I)-(IX) that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula (Ia)-(IXa) or (I)-(IX), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (Ia)-(IXa) or (I)-(IX)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of compounds of Formula (Ia)-(IXa) or (I)-(IX), and uses of such metabolites in the methods of the disclosure. A “pharmaceutically active metabolite” means a pharmacologically active product of metabolism in the body of a compound of Formula (Ia)-(IXa) or (I)-(IX) or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

As used herein, the term “protecting group” or “PG” refers to any group as commonly known to one of ordinary skill in the art that can be introduced into a molecule by chemical modification of a functional group, such as an amine or hydroxyl, to obtain chemoselectivity in a subsequent chemical reaction. It will be appreciated that such protecting groups can be subsequently removed from the functional group at a later point in a synthesis to provide further opportunity for reaction at such functional groups or, in the case of a final product, to unmask such functional group. Protecting groups have been described in, for example, Wuts, P. G. M., Greene, T. W., Greene, T. W., & John Wiley & Sons. (2006). Greene's protective groups in organic synthesis. Hoboken, N.J: Wiley-Interscience. One of skill in the art will readily appreciate the chemical process conditions under which such protecting groups can be installed on a functional group. Suitable amine protecting groups useful in connection with the present disclosure include, but are not limited to, 9-fluorenylmethyl-carbonyl (FMOC), t-butylcarbonyl (Boc), benzyloxycarbonyl (Cbz), acetyl (Ac), trifluoroacetyl, phthalimide, benzyl (Bn), triphenylmethyl (trityl, Tr), benzylidene, and p-toluenesulfonyl (tosylamide, Ts).

As used herein, the term “leaving group” or “LG” refers to any group as commonly known to one of ordinary skill in the art that can be introduced into a molecule by chemical modification of a functional group, such as a hydroxyl, to selectivity react at that position in a subsequent chemical reaction. Leaving groups can be a halogen, a mesylate group, a tosylate group, a triflate group, and the like. A person having ordinary skill in the art will appreciate what leaving groups can be used in connection with the preparation of the compounds described herein.

### Representative Embodiments

In some embodiments, the disclosure provides a compound of the formula Ia, or a pharmaceutically acceptable salt thereof,

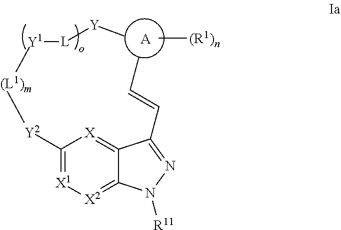

wherein R¹, R¹¹, A, L, L¹, X, X¹, X², Y, Y¹, Y², m, n, and o are as described herein.

In some embodiments, the disclosure provides a compound of the formula I, or a pharmaceutically acceptable salt thereof,

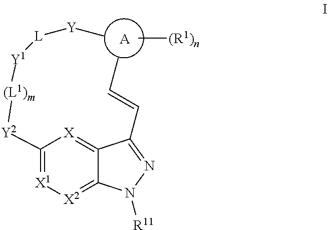

wherein R¹, R¹¹, A, L, L¹, X, X¹, X², Y, Y¹, Y², m, and n are as described herein.

In some embodiments, the disclosure provides a compound of the formula IIa, or a pharmaceutically acceptable salt thereof,

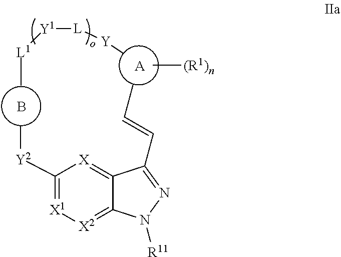

wherein R¹, R¹¹, A, B, L, L¹, X, X¹, X², Y, Y¹, Y², n, and o are as described herein.

In some embodiments, the disclosure provides a compound of the formula II, or a pharmaceutically acceptable salt thereof,

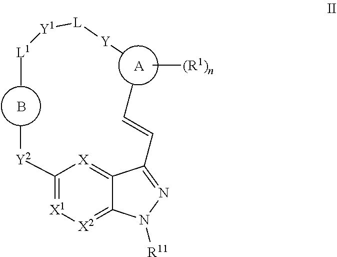

wherein R¹, R¹¹, A, B, L, L¹, X, X¹, X², Y, Y¹, Y², and n are as described herein.

In some embodiments, the disclosure provides a compound of the formula IIIa, or a pharmaceutically acceptable salt thereof,

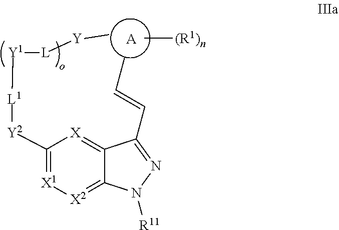

wherein R¹, R¹¹, A, L, L¹, X, X¹, X², Y, Y¹, Y², n, and o are as described herein.

In some embodiments, the disclosure provides a compound of the formula III, or a pharmaceutically acceptable salt thereof,

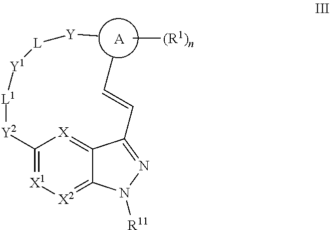

wherein R¹, R¹¹, A, L, L¹, X, X¹, X², Y¹, Y², and n are as described herein.

In some embodiments, the disclosure provides a compound of the formula IVa, or a pharmaceutically acceptable salt thereof,

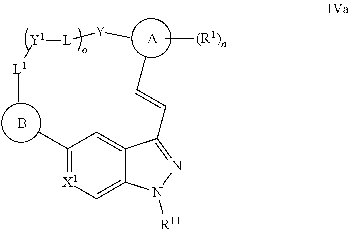

wherein R¹, R¹¹, A, B, L, L¹, X¹, Y, Y¹, n, and o are as described herein.

In some embodiments, the disclosure provides a compound of the formula IV, or a pharmaceutically acceptable salt thereof,

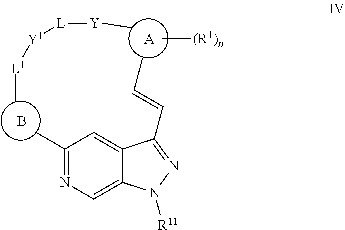

wherein R¹, R¹¹, A, B, L, L¹, Y, Y¹, and n are as described herein.

In some embodiments, the disclosure provides a compound of the formula Va, or a pharmaceutically acceptable salt thereof,

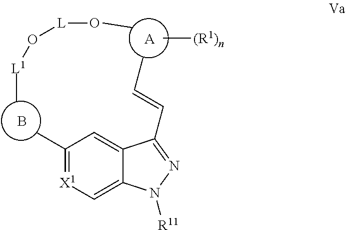

wherein R¹, R¹¹, A, B, L, L¹, X¹, and n are as described herein.

In some embodiments, the disclosure provides a compound of the formula V, or a pharmaceutically acceptable salt thereof,

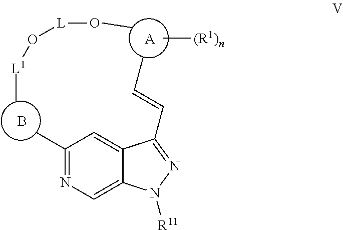

wherein R¹, R¹¹, A, B, L, L¹, and n are as described herein.

In some embodiments, the disclosure provides a compound of the formula VIa, or a pharmaceutically acceptable salt thereof,

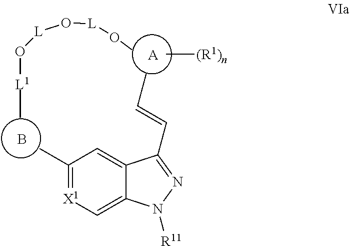

wherein R¹, R¹¹, A, B, L, L¹, X¹, and n are as described herein.

In some embodiments, the disclosure provides a compound of the formula VI, or a pharmaceutically acceptable salt thereof,

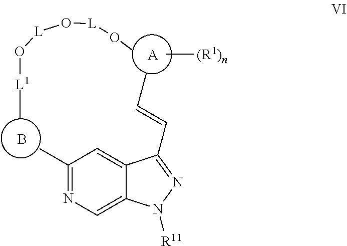

wherein R¹, R¹¹, A, B, L, L¹, and n are as described herein.

In some embodiments, the disclosure provides a compound of the formula VIIa, or a pharmaceutically acceptable salt thereof,

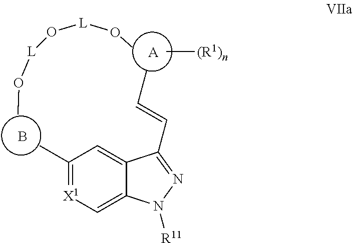

wherein R¹, R¹¹, A, B, L, X¹, and n are as described herein.

In some embodiments, the disclosure provides a compound of the formula VII, or a pharmaceutically acceptable salt thereof,

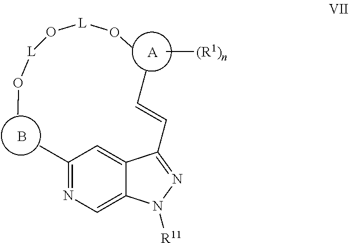

wherein R¹, R¹¹, A, B, L, and n are as described herein.

In some embodiments, the disclosure provides a compound of the formula Villa, or a pharmaceutically acceptable salt thereof,

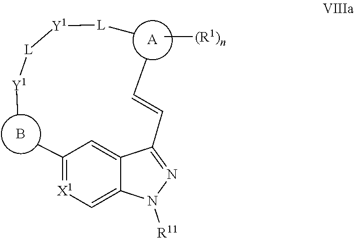

wherein R¹, R¹¹, A, B, L, Y¹, X¹, and n are as described herein.

In some embodiments, the disclosure provides a compound of the formula VIII, or a pharmaceutically acceptable salt thereof,

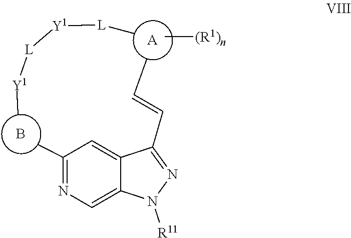

wherein R¹, R¹¹, A, B, L, Y¹, and n are as described herein.

In some embodiments, the disclosure provides a compound of the formula IXa, or a pharmaceutically acceptable salt thereof,

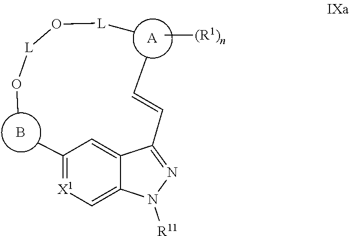

wherein R¹, R¹¹, A, B, L, X¹, and n are as described herein.

In some embodiments, the disclosure provides a compound of the formula IX, or a pharmaceutically acceptable salt thereof,

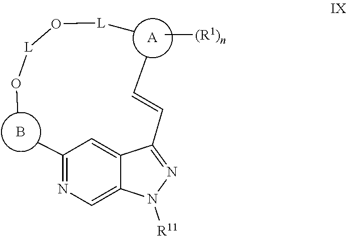

wherein R¹, R¹¹, A, B, L, and n are as described herein.

In some embodiments,

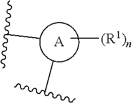

is a 5- to 10-membered heteroarylene, and n is 0, 1, 2, 3, or 4. In some embodiments,

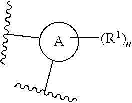

is a 5- to 10-membered heteroarylene selected from the group consisting of pyridinylene, pyrazolylene, and pyrimidinylene, and n is 0, 1, or 2. In some embodiments,

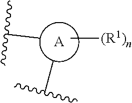

is a 5- to 10-membered heteroarylene selected from the group consisting of

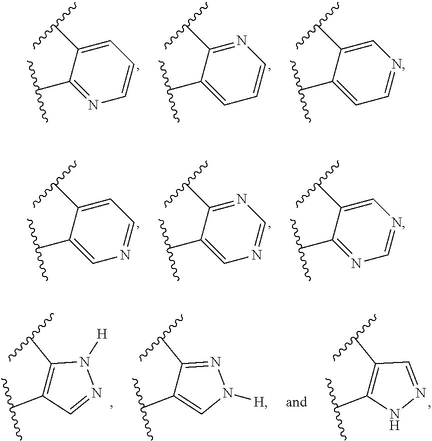

wherein n is 0, 1, 2, 3, or 4.

In some embodiments,

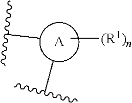

is selected from the group consisting of

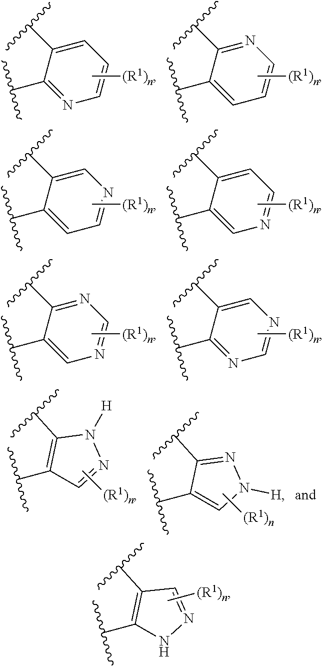

wherein n is 0, 1, 2, 3, or 4.

In some embodiments, n is 0, 1, or 2. In some embodiments, n is 0. In some embodiments, n is 1. In some embodiments, n is 2. In some embodiments, n is 3. In some embodiments, n is 4.

In some embodiments, R¹, when present, is independently fluoro, chloro, methyl, ethyl, methoxy, ethoxy, —C(O)OR^{a}, —C(O)NR^{a}R^{b}, —CN, or 4-piperidinyl.

In some embodiments,

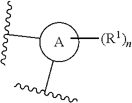

is a 5- to 10-membered heteroarylene selected from the group consisting of

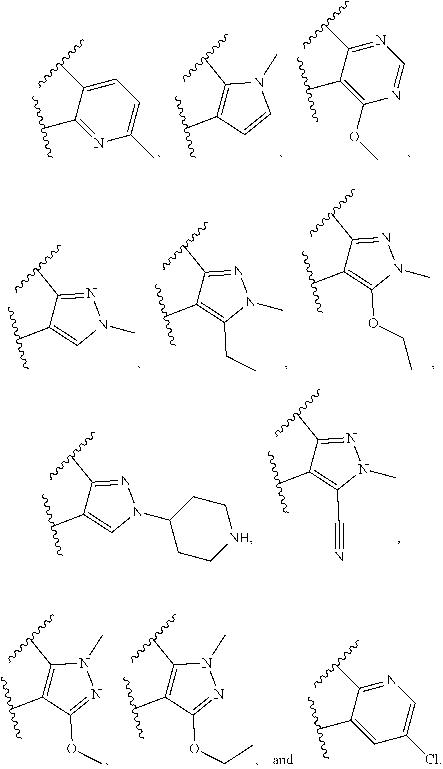

In some embodiments,

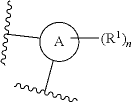

is a C₆-C₁₀ arylene, and n is 0, 1, 2, 3, or 4. In some embodiments,

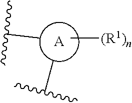

is a phenylene, and n is 0, 1, 2, 3, or 4. In some embodiments, n is 0, 1, or 2. In some embodiments, n is 0. In some embodiments, n is 1. In some embodiments, n is 2. In some embodiments, n is 3. In some embodiments, n is 4.

In some embodiments, R¹, when present, is independently fluoro, chloro, methyl, ethyl, methoxy, ethoxy, —C(O)OR^{a}, —C(O)NR^{a}R^{b}, —CN, or 4-piperidinyl.

In some embodiments,

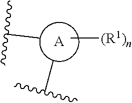

is selected from the group consisting of

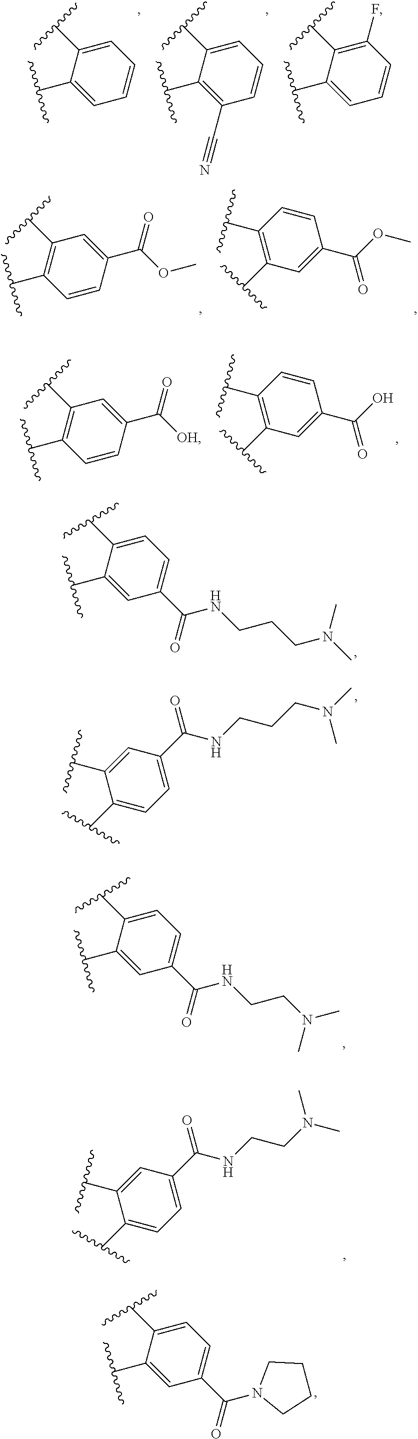

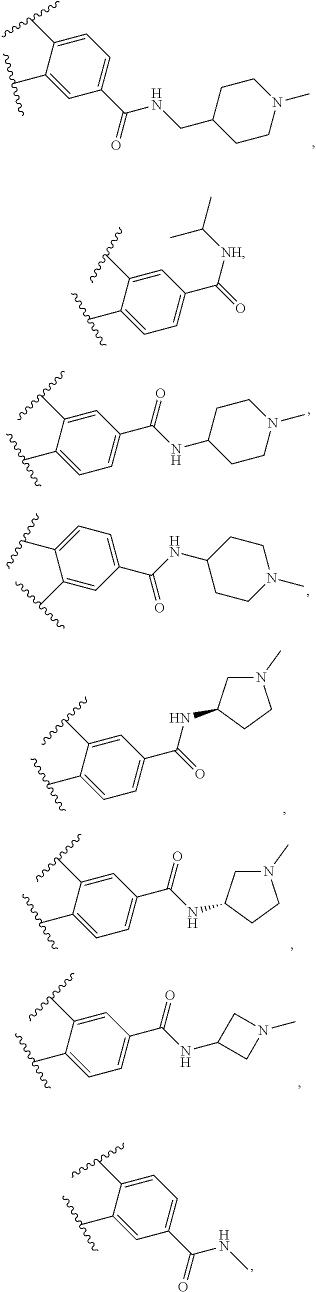

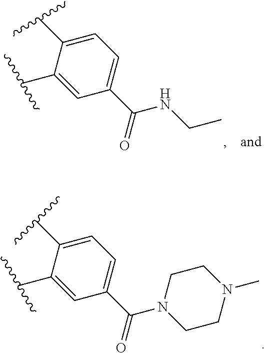 [SMILES: *c1ccc(C(=O)NCC)cc1* ; *c1ccc(C(=O)N2CCN(C)CC2)cc1*]

In some embodiments, L¹, when present, can be independently C₁-C₆ alkylene or a ring B selected from the group consisting of 5- to 10-membered heteroarylene, 3- to 10-membered heterocycloalkylene, 3- to 6-membered cycloalkylene, and C₆-C₁₀ arylene, wherein each hydrogen atom in 5- to 10-membered heteroarylene, 3- to 10-membered heterocycloalkylene, C₆-C₁₀ arylene, and C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, L¹, when present, can be C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, at least one L¹, when present, is methylene, ethylene, or propylene, wherein each hydrogen atom in methylene, ethylene, and propylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, at least one L¹, when present, is methylene, ethylene, or propylene, each of which is substituted with a C₁-C₆ alkyl or a —C(O)NR^{c}R^{d}. In some embodiments, at least one L¹, when present, is methylene, ethylene, or propylene, each of which is substituted with a methyl or a —C(O)NR^{c}R^{d}, wherein R^{c} and R^{d} are each H.

In some embodiments, ring B, when present, can be a 5- to 10-membered heteroarylene, 3- to 10-membered heterocycloalkylene, or C₆-C₁₀ arylene, wherein each hydrogen atom in 3- to 10-membered heteroarylene, 5- to 10-membered heterocycloalkylene, and C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring B, when present, can be a 5- to 10-membered heteroarylene, wherein each hydrogen atom in 5- to 10-membered heteroarylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring B, when present, can be a 5- to 10-membered heteroarylene selected from the group consisting of pyrazolylene, isoxazolylene, pyridinylene, and pyridin-2(1H)-onylene, wherein each hydrogen atom in pyrazolylene, isoxazolylene, pyridinylene, and pyridin-2(1H)-onylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring B, when present, can be a 5- to 10-membered heteroarylene selected from the group consisting of

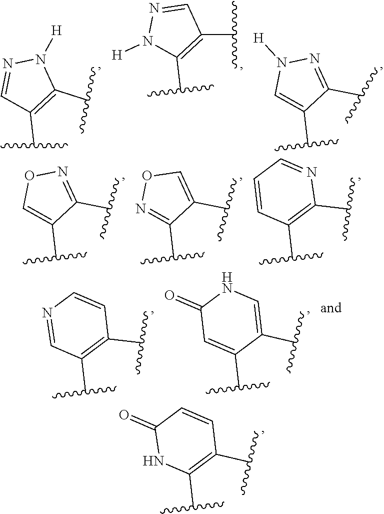

wherein each hydrogen atom in 5- to 10-membered heteroarylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, each ring B, when present, is

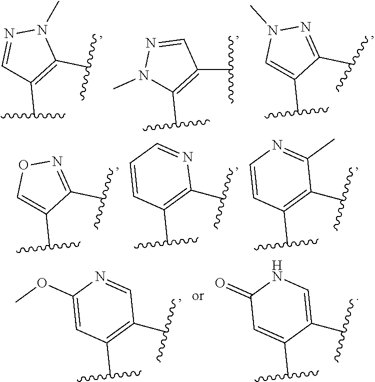

In some embodiments, ring B, when present, can be a 3- to 10-membered heterocycloalkylene, wherein each hydrogen atom in 3- to 10-membered heterocycloalkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring B, when present, can be a 3- to 10-membered heterocycloalkylene selected from the group consisting of pyrrolidinylene, piperidin-2-onylene, and pyrrolidin-2-onylene, wherein each hydrogen atom in pyrrolidinylene, piperidin-2-onylene, and pyrrolidin-2-onylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring B, when present, can be a 3- to 10-membered heterocycloalkylene selected from the group consisting of

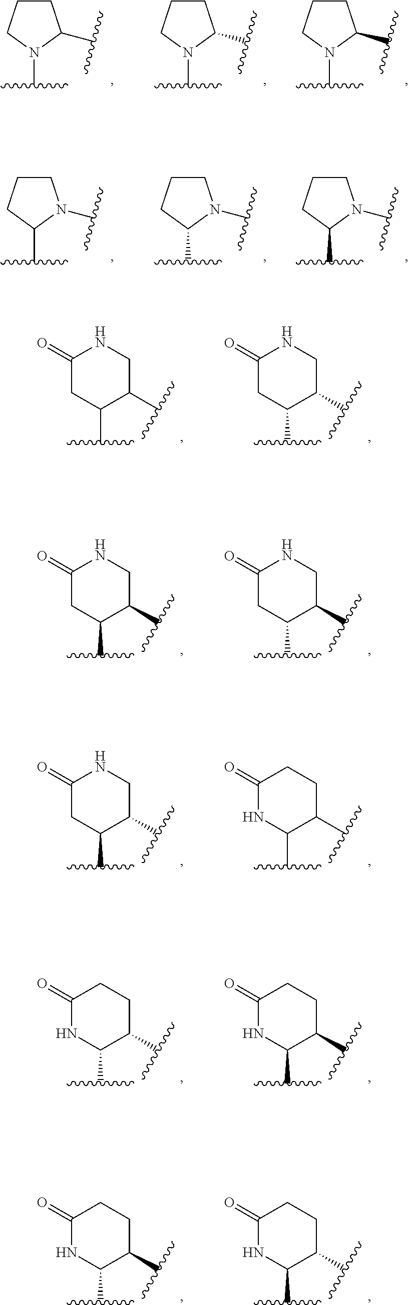

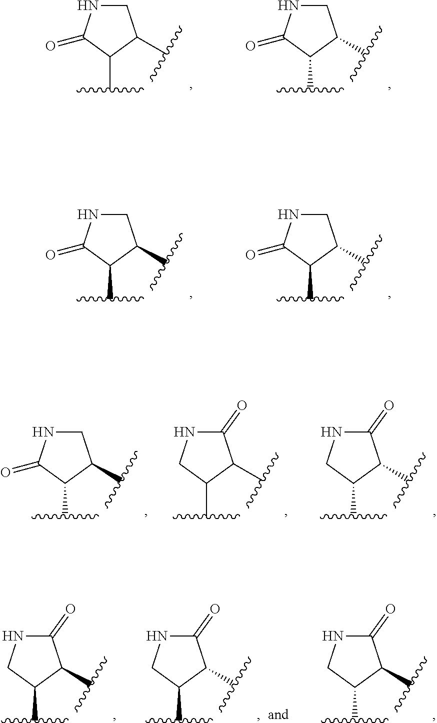

wherein each hydrogen atom in 3- to 10-membered heterocycloalkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, ring B is a 3- to 10-membered heterocycloalkylene selected from the group consisting of

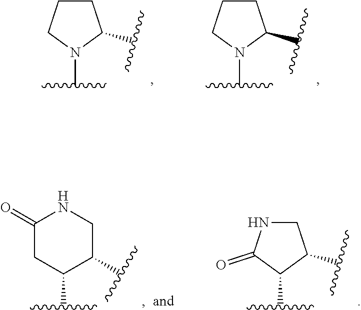 [SMILES: *[C@H]1CCCN1* ; *[C@@H]1CCCN1* ; *[C@@H]1CC(=O)NC[C@@H]1* ; *[C@@H]1C(=O)NC[C@@H]1*]

In some embodiments, ring B, when present, can be a C₆-C₁₀ arylene, wherein each hydrogen atom in C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring B, when present, can be a phenylene optionally substituted with one or more deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, ring B, when present, can be selected from the group consisting of

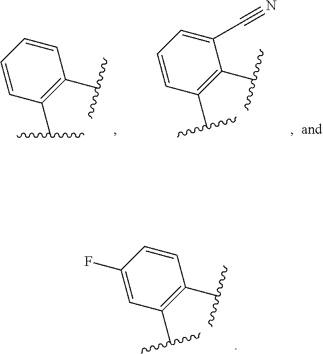 [SMILES: *c1ccccc1* ; *c1cccc(C#N)c1* ; *c1ccc(F)cc1*]

In some embodiments, ring B is absent.

In some embodiments, ring A is a 5- to 10-membered heteroarylene, and ring B is a 5- to 10-membered heteroarylene, wherein ring A and ring B are each optionally substituted as described herein. In some embodiments, ring A is a 5- to 10-membered heteroarylene, and ring B is a 3- to 10-membered heterocycloalkylene, wherein ring A and ring B are each optionally substituted as described herein. In some embodiments, ring A is a 5- to 10-membered heteroarylene, and ring B is a C₆-C₁₀ arylene, wherein ring A and ring B are each optionally substituted as described herein. In some embodiments, ring A is a C₆-C₁₀ arylene, and ring B is a 5- to 10-membered heteroarylene, wherein ring A and ring B are each optionally substituted as described herein. In some embodiments, ring A is a C₆-C₁₀ arylene, and ring B is a 3- to 10-membered heterocycloalkylene, wherein ring A and ring B are each optionally substituted as described herein. In some embodiments, ring A is a C₆-C₁₀ arylene, and ring B is a C₆-C₁₀ arylene, wherein ring A and ring B are each optionally substituted as described herein. In some embodiments, ring A is a 5- to 10-membered heteroarylene, and ring B is absent, wherein ring A is optionally substituted as described herein. In some embodiments, ring A is a C₆-C₁₀ arylene, and ring B is absent, wherein ring A is optionally substituted as described herein.

In some embodiments, ring A is a 5- to 10-membered heteroarylene selected from the group consisting of pyridinylene, pyrazolylene, and pyrimidinylene, and ring B is a 5- to 10-membered heteroarylene, wherein ring A and ring B are each optionally substituted as described herein. In some embodiments, ring A is a 5- to 10-membered heteroarylene selected from the group consisting of pyridinylene, pyrazolylene, and pyrimidinylene, and ring B is a 3- to 10-membered heterocycloalkylene, wherein ring A and ring B are each optionally substituted as described herein. In some embodiments, ring A is a 5- to 10-membered heteroarylene selected from the group consisting of pyridinylene, pyrazolylene, and pyrimidinylene, and ring B is a C₆-C₁₀ arylene, wherein ring A and ring B are each optionally substituted as described herein. In some embodiments, ring A is a phenylene, and ring B is a 5- to 10-membered heteroarylene, wherein ring A and ring B are each optionally substituted as described herein. In some embodiments, ring A is a phenylene, and ring B is a 3- to 10-membered heterocycloalkylene, wherein ring A and ring B are each optionally substituted as described herein. In some embodiments, ring A is a phenylene, and ring B is a C₆-C₁₀ arylene, wherein ring A and ring B are each optionally substituted as described herein. In some embodiments, ring A is a 5- to 10-membered heteroarylene selected from the group consisting of pyridinylene, pyrazolylene, and pyrimidinylene, and ring B is absent, wherein ring A is optionally substituted as described herein. In some embodiments, ring A is a phenylene, and ring B is absent, wherein ring A is optionally substituted as described herein.

In some embodiments, ring A is a 5- to 10-membered heteroarylene, and ring B is a 5- to 10-membered heteroarylene selected from the group consisting of pyrazolylene, isoxazolylene, pyridinylene, and pyridin-2(1H)-onylene, wherein ring A and ring B are each optionally substituted as described herein. In some embodiments, ring A is a 5- to 10-membered heteroarylene, and ring B is a 3- to 10-membered heterocycloalkylene selected from the group consisting of pyrrolidinylene, piperidin-2-onylene, and pyrrolidin-2-onylene, wherein ring A and ring B are each optionally substituted as described herein. In some embodiments, ring A is a 5- to 10-membered heteroarylene, and ring B is a phenylene, wherein ring A and ring B are each optionally substituted as described herein. In some embodiments, ring A is a C₆-C₁₀ arylene, and ring B is a 5- to 10-membered heteroarylene selected from the group consisting of pyrazolylene, isoxazolylene, pyridinylene, and pyridin-2(1H)-onylene, wherein ring A and ring B are each optionally substituted as described herein. In some embodiments, ring A is a C₆-C₁₀ arylene, and ring B is a 3- to 10-membered heterocycloalkylene selected from the group consisting of pyrrolidinylene, piperidin-2-onylene, and pyrrolidin-2-onylene, wherein ring A and ring B are each optionally substituted as described herein. In some embodiments, ring A is a C₆-C₁₀ arylene, and ring B is a phenylene, wherein ring A and ring B are each optionally substituted as described herein. In some embodiments, ring A is a 5- to 10-membered heteroarylene, and ring B is absent, wherein ring A is optionally substituted as described herein. In some embodiments, ring A is a C₆-C₁₀ arylene, and ring B is absent, wherein ring A is optionally substituted as described herein.

In some embodiments, ring A is a 5- to 10-membered heteroarylene selected from the group consisting of pyridinylene, pyrazolylene, and pyrimidinylene, and ring B is a 5- to 10-membered heteroarylene selected from the group consisting of pyrazolylene, isoxazolylene, pyridinylene, and pyridin-2(1H)-onylene, wherein ring A and ring B are each optionally substituted as described herein. In some embodiments, ring A is a 5- to 10-membered heteroarylene selected from the group consisting of pyridinylene, pyrazolylene, and pyrimidinylene, and ring B is a 3- to 10-membered heterocycloalkylene selected from the group consisting of pyrrolidinylene, piperidin-2-onylene, and pyrrolidin-2-onylene, wherein ring A and ring B are each optionally substituted as described herein. In some embodiments, ring A is a 5- to 10-membered heteroarylene selected from the group consisting of pyridinylene, pyrazolylene, and pyrimidinylene, and ring B is a phenylene, wherein ring A and ring B are each optionally substituted as described herein.

In some embodiments, ring A is a phenylene, and ring B is a 5- to 10-membered heteroarylene selected from the group consisting of pyrazolylene, isoxazolylene, pyridinylene, and pyridin-2(1H)-onylene, wherein ring A and ring B are each optionally substituted as described herein. In some embodiments, ring A is a phenylene, and ring B is a 3- to 10-membered heterocycloalkylene selected from the group consisting of pyrrolidinylene, piperidin-2-onylene, and pyrrolidin-2-onylene, wherein ring A and ring B are each optionally substituted as described herein. In some embodiments, ring A is a phenylene, and ring B is a phenylene, wherein ring A and ring B are each optionally substituted as described herein.

In some embodiments, each R¹, when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, each L is independently a C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂.

In some embodiments, each L is independently an ethylene, propylene, or butylene, wherein each hydrogen atom in ethylene, propylene, and butylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂.

In some embodiments, each L is independently an ethylene, propylene, or butylene, each of which is optionally substituted by a C₁-C₆ alkyl.

In some embodiments, L is a C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂.

In some embodiments, L is an ethylene, propylene, or butylene, wherein each hydrogen atom in ethylene, propylene, and butylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂.

In some embodiments, L is an ethylene, propylene, or butylene, each of which is optionally substituted by a C₁-C₆ alkyl.

In some embodiments, two hydrogen atoms on one carbon atom in one or more L are independently optionally substituted by a C₂-C₅ alkylene to provide a C₃-C₆ cycloalkylene. In some embodiments, two hydrogen atoms on two carbon atoms in one or more L are independently optionally substituted by a C₁-C₄ alkylene to provide a C₃-C₆ cycloalkylene. In some embodiments, one hydrogen atom on one carbon atom in L and one of R⁵, R⁶, R⁷, or R⁸ taken together with the atoms to which they are attached combine to form a 3- to 7-membered heterocycloalkylene.

In some embodiments, Y is O, N(R⁵)C(O), C(O)N(R⁵), N(R⁶), N(R⁵)S(O), S(O)N(R⁵), N(R⁵)S(O)₂, S(O)₂N(R⁵), S, S(O), S(O)₂, or Y is absent. In some embodiments, Y is O, N(R⁵)C(O), N(R⁵), N(R⁶), S, S(O), S(O)₂, or Y is absent. In some embodiments, Y is O, N(R⁵)C(O), N(R⁵), N(R⁶), S, S(O), or S(O)₂.

In some embodiments, Y is —O—. In some embodiments, Y is —N(R⁵)C(O)—, wherein R⁵ is as defined in any of the embodiments described herein. In some embodiments, Y is —N(R⁵)C(O)— and R⁵ is H or methyl. In some embodiments, Y is not —N(R⁵)C(O)—. In some embodiments, Y is —C(O)N(R⁵)—, wherein R⁵ is as defined in any of the embodiments described herein. In some embodiments, Y is —C(O)N(R⁵)— and R⁵ is H or methyl. In some embodiments, Y is not —C(O)N(R⁵)—. In some embodiments, Y is —N(R⁶)—, wherein R⁶ is as defined in any of the embodiments described herein. In some embodiments, Y is —N(R⁶)—, wherein R⁶ is H or methyl. In some embodiments, Y is not —N(R⁶)—. In some embodiments, Y is —N(R⁵)S(O)—, wherein R⁵ is as defined in any of the embodiments described herein. In some embodiments, Y is —N(R⁵)S(O)— and R⁵ is H or methyl. In some embodiments, Y is not —N(R⁵)S(O)—. In some embodiments, Y is —S(O)N(R⁵)—, wherein R⁵ is as defined in any of the embodiments described herein. In some embodiments, Y is —S(O)N(R⁵)— and R⁵ is H or methyl. In some embodiments, Y is not —S(O)N(R⁵)—. In some embodiments, Y is —N(R⁵)S(O)₂—, wherein R⁵ is as defined in any of the embodiments described herein. In some embodiments, Y is —N(R⁵)S(O)₂— and R⁵ is H or methyl. In some embodiments, Y is not —N(R⁵)S(O)₂—. In some embodiments, Y is —S(O)₂N(R⁵)—, wherein R⁵ is as defined in any of the embodiments described herein. In some embodiments, Y is —S(O)₂N(R⁵)— and R⁵ is H or methyl. In some embodiments, Y is not —S(O)₂N(R⁵)—. In some embodiments, Y is —S—. In some embodiments, Y is not —S—. In some embodiments, Y is —S(O)—. In some embodiments, Y is not —S(O)—. In some embodiments, Y is —S(O)₂—. In some embodiments, Y is not —S(O)₂—. In some embodiments, Y is absent.

In some embodiments, each Y¹ is independently O, C(O)N(R⁷), N(R⁷)C(O), N(R⁸), N(R⁷)S(O), S(O)N(R⁷), N(R⁷)S(O)₂, S(O)₂N(R⁷), S, S(O), S(O)₂, or absent. In some embodiments, each Y¹ is O, C(O)N(R⁷), N(R⁸), S, S(O), S(O)₂, or Y¹ is absent. In some embodiments, each Y¹ is O, C(O)N(R⁷), N(R⁸), S, S(O), or S(O)₂.

In some embodiments, one or more Y¹ is —O—. In some embodiments, one or more Y¹ is —C(O)N(R⁷)—, wherein R⁷ is as defined in any of the embodiments described herein. In some embodiments, one or more Y¹ is —C(O)N(R⁷)—, wherein R⁷ is H or methyl. In some embodiments, one or more Y¹ is not —C(O)N(R⁷)—. In some embodiments, each Y¹ is not —C(O)N(R⁷)—. In some embodiments, one or more Y¹ is —N(R⁷)C(O)—, wherein R⁷ is as defined in any of the embodiments described herein. In some embodiments, one or more Y¹ is —N(R⁷)C(O)—, wherein R⁷ is H or methyl. In some embodiments, one or more Y¹ is not —N(R⁷)C(O)—. In some embodiments, each Y¹ is not —N(R⁷)C(O)—. In some embodiments, one or more Y¹ is —N(R⁸)—, wherein R⁸ is as defined in any of the embodiments described herein. In some embodiments, one or more Y¹ is —N(R⁸)—, wherein R⁸ is H or methyl. In some embodiments, one or more Y¹ is not —N(R⁸)—. In some embodiments, each Y¹ is not —N(R⁸)—. In some embodiments, one or more Y¹ is —N(R⁷)S(O)—, wherein R⁷ is as defined in any of the embodiments described herein. In some embodiments, one or more Y¹ is —N(R⁷)S(O)— and R⁷ is H or methyl. In some embodiments, one or more Y¹ is not —N(R⁷)S(O)—. In some embodiments, each Y¹ is not —N(R⁷)S(O)—. In some embodiments, one or more Y¹ is —S(O)N(R⁷)—, wherein R⁷ is as defined in any of the embodiments described herein. In some embodiments, Y¹ is —S(O)N(R⁷)— and R⁷ is H or methyl. In some embodiments, Y¹ is not —S(O)N(R⁷)—. In some embodiments, each Y¹ is not —S(O)N(R⁷)—. In some embodiments, one or more Y¹ is —N(R⁷)S(O)₂—, wherein R⁷ is as defined in any of the embodiments described herein. In some embodiments, one or more Y¹ is —N(R⁷)S(O)₂— and R⁷ is H or methyl. In some embodiments, one or more Y¹ is not —N(R⁷)S(O)₂—. In some embodiments, each Y¹ is not —N(R⁷)S(O)₂—. In some embodiments, one or more Y¹ is —S(O)₂N(R⁷)—, wherein R⁷ is as defined in any of the embodiments described herein. In some embodiments, one or more Y¹ is —S(O)₂N(R⁷)— and R⁷ is H or methyl. In some embodiments, one or more Y¹ is not —S(O)₂N(R⁷)—. In some embodiments, each Y¹ is not —S(O)₂N(R⁷)—. In some embodiments, one or more Y¹ is —S—. In some embodiments, one or more Y¹ is not —S—. In some embodiments, each Y¹ is not —S—. In some embodiments, one or more Y¹ is —S(O)—. In some embodiments, one or more Y¹ is not —S(O)—. In some embodiments, each Y¹ is not —S(O)—. In some embodiments, one or more Y¹ is —S(O)₂—. In some embodiments, one or more Y¹ is not —S(O)₂—. In some embodiments, each Y¹ is not —S(O)₂—. In some embodiments, one or more Y¹ is absent.

In some embodiments, Y¹ is —O—. In some embodiments, Y¹ is —C(O)N(R⁷)—, wherein R⁷ is as defined in any of the embodiments described herein. In some embodiments, Y¹ is —C(O)N(R⁷)—, wherein R⁷ is H or methyl. In some embodiments, Y¹ is not —C(O)N(R⁷)—. In some embodiments, Y is —N(R⁵)C(O)—, wherein R⁵ is as defined in any of the embodiments described herein. In some embodiments, Y is —N(R⁵)C(O)— and R⁵ is H or methyl. In some embodiments, Y is not —N(R⁵)C(O)—. In some embodiments, Y¹ is —N(R⁸)—, wherein R⁸ is as defined in any of the embodiments described herein. In some embodiments, Y¹ is —N(R⁸)—, wherein R⁸ is H or methyl. In some embodiments, Y¹ is not —N(R⁸)—. In some embodiments, Y¹ is —S—. In some embodiments, Y¹ is not —S—. In some embodiments, Y¹ is —S(O)—. In some embodiments, Y¹ is not —S(O)—. In some embodiments, Y¹ is —S(O)₂—. In some embodiments, Y¹ is not —S(O)₂—. In some embodiments, Y¹ is absent.

In some embodiments, Y² is O, C(O)N(R⁹), N(R⁹)C(O), N(R¹⁰), N(R⁹)S(O), S(O)N(R⁹), N(R⁹)S(O)₂, S(O)₂N(R⁹), S, S(O), S(O)₂, or Y² is absent. In some embodiments, Y² is O, C(O)N(R⁹), N(R¹⁰), S, S(O), S(O)₂, or Y² is absent. In some embodiments, Y² is O, C(O)N(R⁹), N(R¹⁰), S, S(O), or S(O)₂.

In some embodiments, Y² is —O—. In some embodiments, Y² is —C(O)N(R⁹)—, wherein R⁹ is as defined in any of the embodiments described herein. In some embodiments, Y² is —C(O)N(R⁹)—, wherein R⁹ is H, methyl, ethyl, or cyclopropyl. In some embodiments, Y² is not —C(O)N(R⁹)—. In some embodiments, Y² is —N(R⁹)C(O)—, wherein R⁹ is as defined in any of the embodiments described herein. In some embodiments, Y² is —N(R⁹)C(O)— and R⁹ is H, methyl, ethyl, or cyclopropyl. In some embodiments, Y² is not —N(R⁹)C(O)—. In some embodiments, Y² is —N(R¹⁰)—, R¹⁰ is as defined in any of the embodiments described herein. In some embodiments, Y² is —N(R¹⁰)—, R¹⁰ is H, methyl, or phenyl. In some embodiments, Y² is not —N(R¹⁰)—. In some embodiments, Y² is —N(R⁹)S(O)—, wherein R⁹ is as defined in any of the embodiments described herein. In some embodiments, Y² is —N(R⁹)S(O)— and R⁹ is H, methyl, ethyl, or cyclopropyl. In some embodiments, Y² is not —N(R⁹)S(O)—. In some embodiments, Y² is —S(O)N(R⁹)—, wherein R⁹ is as defined in any of the embodiments described herein. In some embodiments, Y² is —S(O)N(R⁹)— and R⁹ is H, methyl, ethyl, or cyclopropyl. In some embodiments, Y² is not —S(O)N(R⁹)—. In some embodiments, Y² is —N(R⁹)S(O)₂—, wherein R⁹ is as defined in any of the embodiments described herein. In some embodiments, Y² is —N(R⁹)S(O)₂— and R⁹ is H, methyl, ethyl, or cyclopropyl. In some embodiments, Y² is not —N(R⁹)S(O)₂—. In some embodiments, Y² is —S(O)₂N(R⁹)—, wherein R⁹ is as defined in any of the embodiments described herein. In some embodiments, Y² is —S(O)₂N(R⁹)— and R⁹ is H, methyl, ethyl, or cyclopropyl. In some embodiments, Y² is not —S(O)₂N(R⁹)—. In some embodiments, Y² is —S—. In some embodiments, Y² is not —S—. In some embodiments, Y² is —S(O)—. In some embodiments, Y² is not —S(O)—. In some embodiments, Y² is —S(O)₂—. In some embodiments, Y² is not —S(O)₂—. In some embodiments, Y² is absent.

In some embodiments, m is 0, 1, 2, 3, or 4. In some embodiments, m is 0, 1, or 2. In some embodiments, m is 0. In some embodiments, m is 1. In some embodiments, m is 2. In some embodiments, m is 3. In some embodiments, m is 4.

In some embodiments, o is 0, 1, 2, or 3. In some embodiments, o is 0, 1, or 2. In some embodiments, o is 1 or 2. In some embodiments, o is 0. In some embodiments, o is 1. In some embodiments, o is 2. In some embodiments, o is 3.

In some embodiments, each R¹, when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, each R¹, when present, is independently fluoro, chloro, methyl, ethyl, methoxy, ethoxy, —CN, or 4-piperidinyl.

In some embodiments, X is C(R²). In some embodiments, X¹ is N. In some embodiments, X is C(R²) and X¹ is N. In some embodiments, X¹ is C(R³). In some embodiments, X is C(R²) and X¹ is C(R³). In some embodiments, X² is C(R⁴). In some embodiments, X is C(R²) and X² is C(R⁴). In some embodiments, X¹ is N and X² is C(R⁴). In some embodiments, X¹ is C(R³) and X² is C(R⁴). In some embodiments, X is C(R²), X¹ is N, and X² is C(R⁴). In some embodiments, X is C(R²), X¹ is C(R³), and X² is C(R⁴). In some embodiments, X² is N. In some embodiments, X is C(R²) and X² is N. In some embodiments, X¹ is N and X² is N. In some embodiments, X is C(R²), X¹ is N, and X² is N. In some embodiments, X is C(R²), X¹ is C(R³), and X² is N.

In some embodiments, X is N. In some embodiments, X is N and X¹ is C(R³). In some embodiments, X is N and X¹ is N. In some embodiments, X is N and X² is C(R⁴). In some embodiments, X is N, X¹ is N, and X² is C(R⁴). In some embodiments, X is N, X¹ is C(R³), and X² is C(R⁴). In some embodiments, X is N, X¹ is N, and X² is N. In some embodiments, X is N, X¹ is C(R³), and X² is N. In some embodiments, X is not N. In some embodiments, X² is not N.

In some embodiments, each of R², R³, and R⁴, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R², when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R², when present, is H.

In some embodiments, R³, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R³, when present, is H.

In some embodiments, R⁴, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R⁴, when present, is H, fluoro, chloro, or methyl.

In some embodiments, each of R⁵, R⁶, R⁷, R⁸, R⁹, R¹⁰, and R¹¹, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, R⁵, when present, is H, methyl, ethyl, cyclopropyl or phenyl. In some embodiments, R⁵, when present, is H or methyl. In some embodiments, R⁶, when present, is H, methyl, ethyl, cyclopropyl or phenyl. In some embodiments, R⁶, when present, is H, methyl, or ethyl. In some embodiments, R⁷, when present, is H, methyl, ethyl, cyclopropyl or phenyl. In some embodiments, R⁷, when present, is H, methyl, or ethyl. In some embodiments, R⁸, when present, is H, methyl, ethyl, cyclopropyl or phenyl. In some embodiments, R⁸, when present, is H, methyl, or ethyl. In some embodiments, R⁹, when present, is H, methyl, ethyl, cyclopropyl or phenyl. In some embodiments, R⁹, when present, is H, methyl, ethyl, or cyclopropyl. In some embodiments, R¹⁰, when present, is H, methyl, ethyl, cyclopropyl or phenyl. In some embodiments, R⁹, when present, is H, methyl, ethyl, or phenyl. In some embodiments, R¹¹, is H, methyl, ethyl, cyclopropyl or phenyl. In some embodiments, R¹¹, is H.

In some embodiments, the disclosure provides a compound selected from the group consisting of (11E)-1-methyl-19,20-dihydro-1H,8H,18H-10,7,4-(ethan[1]yl[1,2]diylidene)pyrazolo[3,4-f][1,5,9,12,13]benzodioxatriazacyclooctadecine;
(18E)-8,16-dimethyl-2,8,11,12-tetrahydro-10H-5,3-(azenometheno)dipyrazolo[3′,4′:10,11;4″,3″:14,15][1,5]dioxacyclopentadecino[7,6-b]pyridine;
(18E)-11,12-dihydro-2H,10H-5,3-(azenometheno)pyrazolo[4′,3′:10,11][1,5]benzodioxacyclopentadecino[6,7-c][1,2]oxazole;
(19E)-12,13-dihydro-2H,11H-5,3-(azenometheno)dibenzo[14,15:6,7][1,5]dioxacyclopentadecino[10,11-c]pyrazole;
(19E)-12,13-dihydro-2H,11H-5,3-(azenometheno)dibenzo[14,15:6,7][1,5]dioxacyclopentadecino[10,11-c]pyrazole-9-carbonitrile;
(19E)-9-methyl-12,13-dihydro-2H,11H-5,3-(azenometheno)pyrazolo[4′,3′:10,11][1,5]benzodioxacyclopentadecino[6,7-c]pyridine;

(4E)-1-methyl-1,8,19,20-tetrahydro-18H-6,9,12-(ethan[1]yl[1,2]diylidene)pyrazolo[3,4-f][1,5,11,12,15]benzodioxatriazacyclooctadecine;
(17E)-8,14-dimethyl-2,11,12,14-tetrahydro-8H,10H-5,3-(azenometheno)[1,5]dioxacyclopentadecino[10,11-c:15,14-c′:6,7-c″]tripyrazole;
(18E)-15-methyl-2,12,13,15-tetrahydro-11H-5,3-(azenometheno)dipyrazolo[4′,3′:10,11;4″,3″:14,15][1,5]dioxacyclopentadecino[6,7-c]pyridin-7(8H)-one;
(20E)-11,12,13,14-tetrahydro-2H-5,3-(azenometheno)dibenzo[15,16:7,8][1,6]dioxacyclohexadecino[11,12-c]pyrazole;
(20E)-11,12,13,14-tetrahydro-2H-5,3-(azenometheno)dibenzo[15,16:7,8][1,6]dioxacyclohexadecino[11,12-c]pyrazole-19-carbonitrile;
(20E)-19-methoxy-11,12,13,14-tetrahydro-2H-5,3-(azenometheno)pyrazolo[3′,4′:11,12][1,6]benzodioxacyclohexadecino[7,8-d]pyrimidine;
(9R,18E)-8,9,16-trimethyl-8,9,11,12-tetrahydro-2H-5,3-(azenometheno)dipyrazolo[3′,4′:9,10;4″,3″:13,14][1,4]dioxacyclopentadecino[6,5-b]pyridine;
(12R,18E)-14-fluoro-8,12-dimethyl-2,8,9,10,11,12-hexahydro-5,3-(azenometheno)dipyrazolo[3,4-f:4′,3′-j][1,4]benzoxazacyclopentadecine;
(9R,17E)-7,9-dimethyl-2,7,9,10,11,12-hexahydro-5,3-(azenometheno)dipyrazolo[3,4-e:4′,3′-i][3]benzazacyclotetradecine;
(4E,15R)-2,15-dimethyl-2,13,15,16,17,18-hexahydro-10,8-(azenometheno)dipyrazolo[4,3-g:4′,3′-k]pyrido[3,4-c]azacyclotetradecin-12(7H)-one;
(4E,15R)-3-ethyl-2,15-dimethyl-2,13,15,16,17,18-hexahydro-10,8-(azenometheno)dipyrazolo[4,3-g:4′,3′-k]pyrido[3,4-c]azacyclotetradecin-12(7H)-one;
(4E,15R)-2,15,20-trimethyl-2,13,15,16,17,18-hexahydro-10,8-(azenometheno)dipyrazolo[4,3-g:4′,3′-k]pyrido[3,4-c]azacyclotetradecin-12(7H)-one;
(17E)-16-ethoxy-8,12,15-trimethyl-2,11,12,15-tetrahydro-8H-5,3-(azenometheno)tripyrazolo[3,4-f:3′,4′-j:4″,3″-n][1,4]oxazacyclopentadecin-13(10H)-one;
(18E)-17-ethoxy-13,16-dimethyl-2,12,13,16-tetrahydro-5,3-(azenometheno)dipyrazolo[3,4-f:3′,4′-j][1,4]benzoxazacyclopentadecin-14(11H)-one;
(18E)-17-ethoxy-7-fluoro-13,16-dimethyl-2,12,13,16-tetrahydro-5,3-(azenometheno)dipyrazolo[3,4-f:3′,4′-j][1,4]benzoxazacyclopentadecin-14(11H)-one;
(18E)-17-ethoxy-13,16-dimethyl-2,12,13,16-tetrahydro-5,3-(azenometheno)dipyrazolo[3,4-f:3′,4′-j]pyrido[3,2-n][1,4]oxazacyclopentadecin-14(11H)-one;
(18E)-17-ethoxy-13,16-dimethyl-2,12,13,16-tetrahydro-5,3-(azenometheno)dipyrazolo[3,4-f:3′,4′-j]pyrido[4,3-n][1,4]oxazacyclopentadecine-7,14(8H,11H)-dione;
(18E)-13,16-dimethyl-2,12,13,16-tetrahydro-5,3-(azenometheno)dipyrazolo[3,4-f:3′,4′-j]pyrido[4,3-n][1,4]oxazacyclopentadecine-7,14(8H,11H)-dione;
(18E)-13-methyl-16-(piperidin-4-yl)-2,12,13,16-tetrahydro-5,3-(azenometheno)dipyrazolo[3,4-f:3′,4′-j]pyrido[4,3-n][1,4]oxazacyclopentadecine-7,14(8H,11H)-dione;
(18E)-13,16-dimethyl-7,14-dioxo-2,7,8,11,12,13,14,16-octahydro-5,3-(azenometheno)dipyrazolo[3,4-f:3′,4′-j]pyrido[4,3-n][1,4]oxazacyclopentadecine-17-carbonitrile;
(18E)-17-ethyl-13,16-dimethyl-2,12,13,16-tetrahydro-5,3-(azenometheno)dipyrazolo[3,4-f:3′,4′-j]pyrido[4,3-n][1,4]oxazacyclopentadecine-7,14(8H,11H)-dione;
(18E)-17-ethyl-13,16,21-trimethyl-2,12,13,16-tetrahydro-5,3-(azenometheno)dipyrazolo[3,4-f:3′,4′-j]pyrido[4,3-n][1,4]oxazacyclopentadecine-7,14(8H,11H)-dione;
(19E)-18-ethyl-14,17,22-trimethyl-11,12,13,14-tetrahydro-2H-5,3-(azenometheno)dipyrazolo[3,4-g:3′,4′-k]pyrido[4,3-o][1,5]oxazacyclohexadecine-7,15(8H,17H)-dione;
(7S,17E)-16-methoxy-7,12,14,20-tetramethyl-2,7,8,11,12,14-hexahydro-6H-5,3-(azenometheno)-6% i-dipyrazolo[4,3-g:4′,3′-k][1,4,14]oxathiazacyclohexadecine-6,6,13(10H)-trione;
(17E)-15-chloro-7-ethyl-8,9,10,11-tetrahydro-2H-5,3-(azenometheno)pyrazolo[4,3-j]pyrido[3,2-n][1,6]oxazacyclopentadecin-6(7H)-one;
(18E)-17-ethyl-7-fluoro-13,15-dimethyl-2,12,13,15-tetrahydro-5,3-(azenometheno)dipyrazolo[3,4-f:3′,4′-j][1,4]benzoxazacyclopentadecin-14(11H)-one;
(11E)-1-methyl-1,18,19,21-tetrahydro-8H-10,7,4-(ethan[1]yl[1,2]diylidene)pyrazolo[3,4-f][1,4,9,12,13]benzodioxatriazacyclooctadecine;
(19E)-18-ethyl-7-methoxy-14,17-dimethyl-2,11,12,13,14,17-hexahydro-15H-5,3-(azenometheno)dipyrazolo[3,4-g:3′,4′-k]pyrido[4,3-o][1,5]oxazacyclohexadecin-15-one;
(18E)-17-ethyl-7-methoxy-13,16-dimethyl-2,12,13,16-tetrahydro-5,3-(azenometheno)dipyrazolo[3,4-f:3′,4′-j]pyrido[4,3-n][1,4]oxazacyclopentadecin-14(11H)-one;
methyl (11E)-1-methyl-19,20-dihydro-1H,8H,18H-10,7,4-(ethan[1]yl[1,2]diylidene)pyrazolo[3,4-f][1,5,9,12,13]benzodioxatriazacyclooctadecine-15-carboxylate;
methyl (11E)-1-methyl-19,20-dihydro-1H,8H,18H-10,7,4-(ethan[1]yl[1,2]diylidene)pyrazolo[3,4-f][1,5,9,12,13]benzodioxatriazacyclooctadecine-14-carboxylate;
methyl (11E)-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-15-carboxylate;
(11E)-N-[3-(dimethylamino)propyl]-1-methyl-19,20-dihydro-1H,8H,18H-10,7,4-(ethan[1]yl[1,2]diylidene)pyrazolo[3,4-f][1,5,9,12,13]benzodioxatriazacyclooctadecine-14-carboxamide;
(11E)-N-[2-(dimethylamino)ethyl]-1-methyl-19,20-dihydro-1H,8H,18H-10,7,4-(ethan[1]yl[1,2]diylidene)pyrazolo[3,4-f][1,5,9,12,13]benzodioxatriazacyclooctadecine-14-carboxamide;
[(11E)-1-methyl-19,20-dihydro-1H,8H,18H-10,7,4-(ethan[1]yl[1,2]diylidene)pyrazolo[3,4-f][1,5,9,12,13]benzodioxatriazacyclooctadecin-14-yl](pyrrolidin-1-yl)methanone;
(11E)-1-methyl-19,20-dihydro-1H,8H,18H-10,7,4-(ethan[1]yl[11,2]diylidene)pyrazolo[3,4-f][1,5,9,12,13]benzodioxatriazacyclooctadecine-15-carboxylic acid;
(11E)-N-[2-(dimethylamino)ethyl]-1-methyl-19,20-dihydro-1H,8H,18H-10,7,4-(ethan[1]yl[1,2]diylidene)pyrazolo[3,4-f][1,5,9,12,13]benzodioxatriazacyclooctadecine-15-carboxamide;
methyl (11E)-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-14-carboxylate;
(11E)-1-methyl-N-[(1-methylpiperidin-4-yl)methyl]-19,20-dihydro-1H,8H,18H-10,7,4-(ethan[1]yl[1,2]diylidene)pyrazolo[3,4-f][1,5,9,12,13]benzodioxatriazacyclooctadecine-14-carboxamide;
(11E)-1-methyl-N-(propan-2-yl)-19,20-dihydro-1H,8H,18H-10,7,4-(ethan[1]yl[1,2]diylidene)pyrazolo[3,4-f][1,5,9,12,13]benzodioxatriazacyclooctadecine-15-carboxamide;
(11E)-1-methyl-N-(1-methylpiperidin-4-yl)-19,20-dihydro-1H,8H,18H-10,7,4-(ethan[1]yl[1,2]diylidene)pyrazolo[3,4-f][1,5,9,12,13]benzodioxatriazacyclooctadecine-15-carboxamide;
(11E)-1-methyl-19,20-dihydro-1H,8H,18H-10,7,4-(ethan[1]yl[1,2]diylidene)pyrazolo[3,4-f][1,5,9,12,13]benzodioxatriazacyclooctadecine-14-carboxylic acid;
(11E)-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-15-carboxylic acid;
(11E)-N-[2-(dimethylamino)ethyl]-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-15-carboxamide;
(11E)-1-methyl-N-[(3R)-1-methylpyrrolidin-3-yl]-19,20-dihydro-1H,8H,18H-10,7,4-(ethan[1]yl[1,2]diylidene)pyrazolo[3,4-f][1,5,9,12,13]benzodioxatriazacyclooctadecine-15-carboxamide;
(11E)-1-methyl-N-(1-methylazetidin-3-yl)-19,20-dihydro-1H,8H,18H-10,7,4-(ethan[1]yl[1,2]diylidene)pyrazolo[3,4-f][1,5,9,12,13]benzodioxatriazacyclooctadecine-15-carboxamide;
(11E)-N-ethyl-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-15-carboxamide;
(11E)-1-methyl-N-(1-methylpiperidin-4-yl)-1,18,19,21-tetrahydro-8H-10,7,4-(ethan[1]yl[1,2]diylidene)pyrazolo[3,4-f][1,4,9,12,13]benzodioxatriazacyclooctadecine-15-carboxamide;
(11E)-N,1-dimethyl-19,20-dihydro-1H,8H,18H-10,7,4-(ethan[1]yl[1,2]diylidene)pyrazolo[3,4-f][1,5,9,12,13]benzodioxatriazacyclooctadecine-14-carboxamide;
(11E)-1-methyl-N-[(3R)-1-methylpyrrolidin-3-yl]-1,18,19,21-tetrahydro-8H-10,7,4-(ethan[1]yl[1,2]diylidene)pyrazolo[3,4-f][1,4,9,12,13]benzodioxatriazacyclooctadecine-15-carboxamide;
(11E)-1-methyl-N-[(3S)-1-methylpyrrolidin-3-yl]-19,20-dihydro-1H,8H,18H-10,7,4-(ethan[1]yl[1,2]diylidene)pyrazolo[3,4-f][1,5,9,12,13]benzodioxatriazacyclooctadecine-15-carboxamide;
(11E)-N-ethyl-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-14-carboxamide;
(11E)-N-[2-(dimethylamino)ethyl]-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-14-carboxamide;
(11E)-N-[3-(dimethylamino)propyl]-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-14-carboxamide;
(11E)-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-14-carboxylic acid;
(11E)-1-methyl-N-(1-methylpiperidin-4-yl)-1,18,19,21-tetrahydro-8H-10,7,4-(ethan[1]yl[1,2]diylidene)pyrazolo[3,4-f][1,4,9,12,13]benzodioxatriazacyclooctadecine-14-carboxamide;
(11E)-1-methyl-N-[(1-methylpiperidin-4-yl)methyl]-1,18,19,21-tetrahydro-8H-10,7,4-(ethan[1]yl[1,2]diylidene)pyrazolo[3,4-f][1,4,9,12,13]benzodioxatriazacyclooctadecine-14-carboxamide;
(18E)-8,9,16-trimethyl-8,9,11,12-tetrahydro-2H-5,3-(azenometheno)dipyrazolo[3′,4′:9,10;4″,3″:13,14][1,4]dioxacyclopentadecino[6,5-b]pyridine;
(4-methylpiperazin-1-yl)[(11E)-1-methyl-1,18,19,21-tetrahydro-8H-10,7,4-(ethan[1]yl[1,2]diylidene)pyrazolo[3,4-f][1,4,9,12,13]benzodioxatriazacyclooctadecin-14-yl]methanone;
(11E)-1-methyl-N-(1-methylpiperidin-4-yl)-19,20-dihydro-1H,8H,18H-10,7,4-(ethan[1]yl[1,2]diylidene)pyrazolo[3,4-f][1,5,9,12,13]benzodioxatriazacyclooctadecine-14-carboxamide;
(11E)-14-fluoro-1,6-dimethyl-19,20-dihydro-1H,8H,18H-10,7,4-(ethan[1]yl[1,2]diylidene)pyrazolo[3,4-f][1,5,9,12,13]benzodioxatriazacyclooctadecine; and
(17E)-6-(3-chloro-4-fluorophenyl)-12,15-dimethyl-2,7,8,11,12,15-hexahydro-6H-5,3-(azenometheno)dipyrazolo[3,4-f:3′,4′-j][1,4,14]oxadiazacyclohexadecin-13(10H)-one;

or a pharmaceutically acceptable salt thereof.

In other embodiments, the disclosure provides a compound selected from the group consisting of (18E)-8-methyl-8,9,11,12-tetrahydro-2H-5,3-(azenometheno)dipyrazolo[3,4-f:4′,3′-j][1,4,9]benzodioxazacyclopentadecine;
(9R,18E)-8,9-dimethyl-8,9,11,12-tetrahydro-2H-5,3-(azenometheno)dipyrazolo[3,4-f:4′,3′-j][1,4,9]benzodioxazacyclopentadecine;
(9R,18E)-14-fluoro-8,9-dimethyl-8,9,11,12-tetrahydro-2H-5,3-(azenometheno)dipyrazolo[3,4-f:4′,3′-j][1,4,9]benzodioxazacyclopentadecine;
(9R,18E)-8,9,16-trimethyl-8,9,11,12-tetrahydro-2H-5,3-(azenometheno)dipyrazolo[3,4-f:4′,3′-j]pyrido[2,3-n][1,4,9]dioxazacyclopentadecine;
(10R,17E)-10,15-dimethyl-2,8,10,12,13,15-hexahydro-7H-5,3-(azenometheno)dipyrazolo[4,3-g:4′,3′-k]pyrido[3,4-c][1,6]oxazacyclotetradecin-7-one;
(18E)-6-methyl-2,6,11,12-tetrahydro-5,3-(azenometheno)dipyrazolo[4,3-f:4′,3′-j][1,4,9]benzoxadiazacyclopentadecin-9(10H)-one;
(18E)-17-ethyl-13,16,21-trimethyl-10,11,12,13-tetrahydro-2H-5,3-(azenometheno)dipyrazolo[3,4-j:3′,4′-n]pyrido[4,3-c][1,8]diazacyclopentadecine-7,14(8H,16H)-dione;
(9aR,19E)-14,17-dimethyl-2,8,9,9a,10,13,14,17-octahydro-7H-5,3-(azenometheno)dipyrazolo[4,3-g:4′,3′-k]pyrrolo[2,1-c][1,4,6,14]oxatriazacyclohexadecin-15(12H)-one;
(9aS,19E)-14,17-dimethyl-2,8,9,9a,10,13,14,17-octahydro-7H-5,3-(azenometheno)dipyrazolo[4,3-g:4′,3′-k]pyrrolo[2,1-c][1,4,6,14]oxatriazacyclohexadecin-15(12H)-one;
(17E)-12,15-dimethyl-6-phenyl-2,7,8,11,12,15-hexahydro-6H-5,3-(azenometheno)dipyrazolo[4,3-g:4′,3′-k][1,4,6,14]oxatriazacyclohexadecin-13(10H)-one;
(7R,17E)-7,12,15,20-tetramethyl-2,7,8,11,12,15-hexahydro-6H-5,3-(azenometheno)dipyrazolo[4,3-g:4′,3′-k][1,4,6,14]oxatriazacyclohexadecin-13(10H)-one;
(7R,17E)-7,12,15-trimethyl-2,7,8,11,12,15-hexahydro-5,3-(azenometheno)dipyrazolo[4,3-d:4′,3′-h][1,14,3,11]dioxadiazacyclohexadecin-13(10H)-one;
(7S,17E)-12,15-dimethyl-13-oxo-2,7,8,10,11,12,13,15-octahydro-5,3-(azenometheno)dipyrazolo[4,3-d:4′,3′-h][1,14,3,11]dioxadiazacyclohexadecine-7-carboxamide;
(6aR,10aS,19E)-14,17-dimethyl-2,6a,9,10,10a,13,14,17-octahydro-5,3-(azenometheno)dipyrazolo[4,3-d:4′,3′-h]pyrido[3,4-o][1,14,3,11]dioxadiazacyclohexadecine-8,15(7H,12H)-dione;
(6aS,9aS,18E)-13,16-dimethyl-2,9,9a,12,13,16-hexahydro-6aH-5,3-(azenometheno)dipyrazolo[4,3-d:4′,3′-h]pyrrolo[3,4-o][1,14,3,11]dioxadiazacyclohexadecine-7,14(8H,11H)-dione;
and (17E)-15-chloro-7-ethyl-8,9,10,11-tetrahydro-2H-5,3-(azenometheno)pyrazolo[4,3-j]pyrido[3,2-n][1,6,9]oxadiazacyclopentadecin-6(7H)-one; and
(18E)-8,9,16-trimethyl-8,9,11,12-tetrahydro-2H-5,3-(azenometheno)dipyrazolo[3,4-f:4′,3′-j]pyrido[2,3-n][1,4,9]dioxazacyclopentadecine;

or a pharmaceutically acceptable salt thereof.
In other embodiments, the disclosure provides a compound selected from the group consisting of (11E)-1-methyl-19,20-dihydro-1H,8H,18H-10,7,4-(ethan[1]yl[1,2]diylidene)pyrazolo[3,4-f][1,5,12,13]benzodioxadiazacyclooctadecine;
(19E)-12,13-dihydro-2H,11H-3,5-ethenopyrazolo[4′,3′:10,11][1,5]benzodioxacyclopentadecino[6,7-b]pyridine;
(9R,18E)-8,9,16-trimethyl-8,9,11,12-tetrahydro-2H-3,5-ethenodipyrazolo[3,4-f:4′,3′-j]pyrido[2,3-n][1,4,9]dioxazacyclopentadecine;
(9R,18E)-8,9,16-trimethyl-8,9,11,12-tetrahydro-2H-3,5-ethenodipyrazolo[3′,4′:9,10;4″,3″:13,14][1,4]dioxacyclopentadecino[6,5-b]pyridine;
(17E)-12,15-dimethyl-2,7,8,11,12,15-hexahydro-6H-3,5-etheno-6λ⁶-dipyrazolo[4,3-g:4′,3′-k][1,4,14]oxathiazacyclohexadecine-6,6,13(10H)-trione;
(17E)-20-fluoro-12,15-dimethyl-2,7,8,11,12,15-hexahydro-6H-3,5-etheno-6λ⁶-dipyrazolo[4,3-g:4′,3′-k][1,4,14]oxathiazacyclohexadecine-6,6,13(10H)-trione;
(17E)-20-chloro-12,15-dimethyl-2,7,8,11,12,15-hexahydro-6H-3,5-etheno-6λ⁶-dipyrazolo[4,3-g:4′,3′-k][1,4,14]oxathiazacyclohexadecine-6,6,13(10H)-trione;
(7S,17E)-20-chloro-7,12,14-trimethyl-2,7,8,11,12,14-hexahydro-6H-3,5-etheno-6λ⁶-dipyrazolo[4,3-g:4′,3′-k][1,4,14]oxathiazacyclohexadecine-6,6,13(10H)-trione;
(7S,17E)-20-chloro-16-methoxy-7,12,14-trimethyl-2,7,8,11,12,14-hexahydro-6H-3,5-etheno-6% i-dipyrazolo[4,3-g:4′,3′-k][1,4,14]oxathiazacyclohexadecine-6,6,13(10H)-trione;
(7S,17E)-20-chloro-16-ethoxy-7,12,14-trimethyl-2,7,8,11,12,14-hexahydro-6H-3,5-etheno-6% i-dipyrazolo[4,3-g:4′,3′-k][1,4,14]oxathiazacyclohexadecine-6,6,13(10H)-trione; and
(17E)-15-chloro-7-ethyl-8,9,10,11-tetrahydro-2H-3,5-ethenopyrazolo[4,3-j]pyrido[3,2-n][1,6]oxazacyclopentadecin-6(7H)-one;

or a pharmaceutically acceptable salt thereof.

In other embodiments, the disclosure provides a compound selected from the group consisting of (18E)-8-methyl-2,8,11,12-tetrahydro-10H-3,5-ethenodipyrazolo[3,4-f:4′,3′-j][1,5,9]benzodioxazacyclopentadecine;
(17E)-7-cyclopropyl-8,9,10,11-tetrahydro-2H-3,5-ethenopyrazolo[4,3-j][1,6,9]benzoxadiazacyclopentadecin-6(7H)-one; and
(17E)-15-chloro-7-ethyl-8,9,10,11-tetrahydro-2H-3,5-ethenopyrazolo[4,3-j]pyrido[3,2-n][1,6,9]oxadiazacyclopentadecin-6(7H)-one;

or a pharmaceutically acceptable salt thereof.

The following represent illustrative embodiments of compounds of Formula (Ia)-(IXa) or (I)-(IX):

| Cpd. | Structure | Name (ACD/Name 2020) |
|---|---|---|
| 1 | 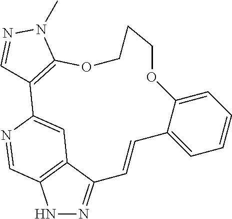 [SMILES: Cn1ncc2c1OCCCOc1ccccc1/C=C/c1n[nH]c3cnc-2cc13] | (11E)-1-methyl-19,20-dihydro-1H,8H,18H- 10,7,4-(ethan[1]yl[1,2]diylidene)pyrazolo[3,4- f][1,5,9,12,13]benzodioxatriazacyclooctadecine |
| 2 | 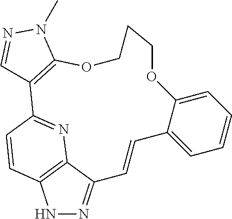 [SMILES: Cn1ncc2c1OCCCOc1ccccc1/C=C/c1n[nH]c3ccc-2nc13] | (18E)-8-methyl-2,8,11,12-tetrahydro-10H-3,5- ethenodipyrazolo[3,4-f:4′,3′- j][1,5,9]benzodioxazacyclopentadecine |
| 3 | 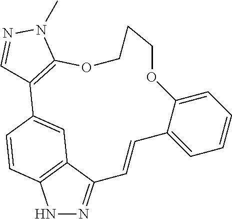 [SMILES: Cn1ncc2c1OCCCOc1ccccc1/C=C/c1n[nH]c3ccc-2cc13] | (11E)-1-methyl-19,20-dihydro-1H,8H,18H- 10,7,4-(ethan[1]yl[1,2]diylidene)pyrazolo[3,4- f][1,5,12,13]benzodioxadiazacyclooctadecine |
| 4 | 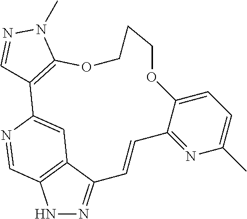 [SMILES: Cc1ccc2c(n1)/C=C/c1n[nH]c3cnc(cc13)-c1cnn(C)c1OCCCO2] | (18E)-8,16-dimethyl-2,8,11,12-tetrahydro-10H- 5,3- (azenometheno)dipyrazolo[3′,4′:10,11;4″,3″:14, 15][1,5]dioxacyclopentadecino[7,6-b]pyridine |
| 5 | 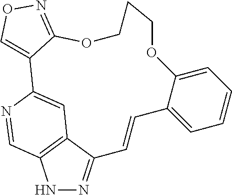 [SMILES: C1=C/c2n[nH]c3cnc(cc23)-c2conc2OCCCOc2ccccc2/1] | (18E)-11,12-dihydro-2H,10H-5,3- (azenometheno)pyrazolo[4′,3′:10,11][1,5] benzodioxacyclopentadecino [6,7-c][1,2]oxazole |
| 7 | 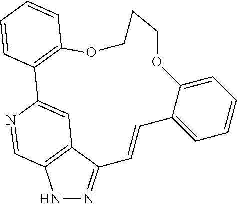 [SMILES: C1=C/c2n[nH]c3cnc(cc23)-c2ccccc2OCCCOc2ccccc2/1] | (19E)-12,13-dihydro-2H,11H-5,3- (azenometheno)dibenzo[14,15:6,7][1,5] dioxacyclopentadecino[10,11-c]pyrazole |
| 8 | 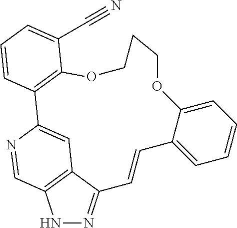 [SMILES: N#Cc1cccc2c1OCCCOc1ccccc1/C=C/c1n[nH]c3cnc-2cc13] | (19E)-12,13-dihydro-2H,11H-5,3- (azenometheno)dibenzo[14,15:6,7][1,5] dioxacyclopentadecino[10,11-c]pyrazole-9- carbonitrile |
| 9 | 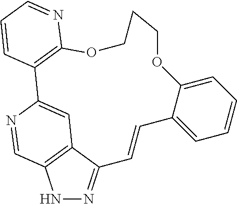 [SMILES: C1=C/c2n[nH]c3cnc(cc23)-c2cccnc2OCCCOc2ccccc2/1] | (19E)-12,13-dihydro-2H,11H-3,5- ethenopyrazolo[4′,3′:10,11][1,5] benzodioxacyclopentadecino[6,7-b]pyridine |
| 10 | 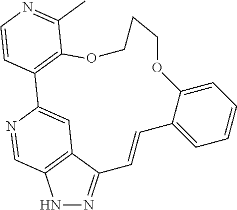 [SMILES: Cc1nccc2c1OCCCOc1ccccc1/C=C/c1n[nH]c3cnc-2cc13] | (19E)-9-methyl-12,13-dihydro-2H,11H-5,3- (azenometheno)pyrazolo[4′,3′:10,11][1,5] benzodioxacyclopentadecino[6,7-c]pyridine |
| 11 | 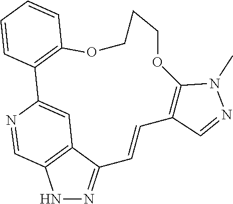 [SMILES: Cn1ncc2c1OCCCOc1ccccc1-c1cc3c(n[nH]c3cn1)/C=C/2] | (4E)-1-methyl-1,8,19,20-tetrahydro-18H- 6,9,12-(ethan[1]y][1,2]diylidene)pyrazolo[3,4- f][1,5,11,12,15]benzodioxatriazacyclooctadecine |
| 12 | 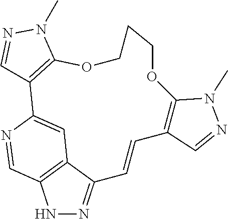 [SMILES: Cn1ncc2c1OCCCOc1c(cnn1C)-c1cc3c(n[nH]c3cn1)/C=C/2] | (17E)-8,14-dimethyl-2,11,12,14-tetrahydro- 8H,10H-5,3- (azenometheno)[1,5]dioxacyclopentadecino[10, 11-c:15,14-c′:6,7-c″]tripyrazole |
| 13 | 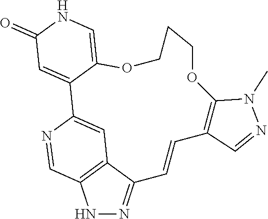 [SMILES: Cn1ncc2c1OCCCOc1c[nH]c(=O)cc1-c1cc3c(n[nH]c3cn1)/C=C/2] | (18E)-15-methyl-2,12,13,15- tetrahydro-11H-5,3- (azenometheno)dipyrazolo[4′,3′:10,11;4″,3″:14, 15][1,5]dioxacyclopentadecino[6,7-c]pyridin- 7(8H)-one |
| 14 | 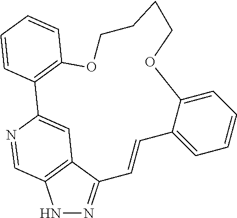 [SMILES: C1=C/c2n[nH]c3cnc(cc23)-c2ccccc2OCCCCOc2ccccc2/1] | (20E)-11,12,13,14-tetrahydro-2H-5,3- (azenometheno)dibenzo[15,16:7,8][1,6] dioxacyclohexadecino[11,12-c]pyrazole |
| 15 | 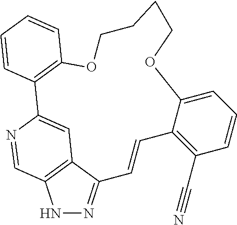 [SMILES: N#Cc1cccc2c1/C=C/c1n[nH]c3cnc(cc13)-c1ccccc1OCCCCO2] | (20E)-11,12,13,14-tetrahydro-2H-5,3- (azenometheno)dibenzo[15,16:7,8][1,6] dioxacyclohexadecino[11,12-c] pyrazole-19-carbonitrile |
| 16 | 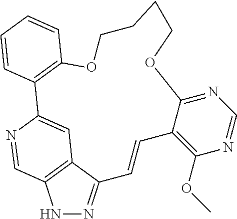 [SMILES: COc1ncnc2c1/C=C/c1n[nH]c3cnc(cc13)-c1ccccc1OCCCCO2] | (20E)-19-methoxy-11,12,13,14-tetrahydro-2H- 5,3- (azenometheno)pyrazolo[3′,4′:11,12][1,6] benzodioxacyclohexadecino[7,8-d]pyrimidine |
| 17 | 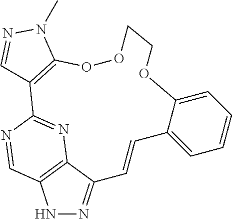 [SMILES: Cn1ncc2c1OOCCOc1ccccc1/C=C/c1n[nH]c3cnc-2nc13] | (18E)-8-methyl-8,9,11,12-tetrahydro-2H-5,3- (azenometheno)dipyrazolo[3,4-f:4′,3′- j][1,4,9]benzodioxazacyclopentadecine |
| 18 | 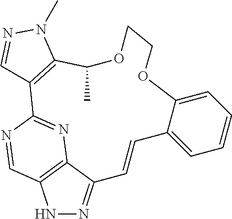 [SMILES: C[C@H]1OCCOc2ccccc2/C=C/c2n[nH]c3cnc(nc23)-c2cnn(C)c21] | (9R,18E)-8,9-dimethyl-8,9,11,12-tetrahydro- 2H-5,3-(azenometheno)dipyrazolo[3,4-f:4′,3′- j][1,4,9]benzodioxazacyclopentadecine |
| 19 | 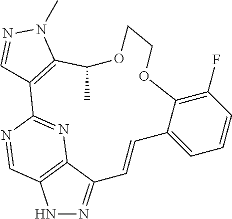 [SMILES: C[C@H]1OCCOc2c(F)cccc2/C=C/c2n[nH]c3cnc(nc23)-c2cnn(C)c21] | (9R,18E)-14-fluoro-8,9-dimethyl-8,9,11,12- tetrahydro-2H-5,3- (azenometheno)dipyrazolo[3,4-f:4′,3′- j][1,4,9]benzodioxazacyclopentadecine |
| 20 | 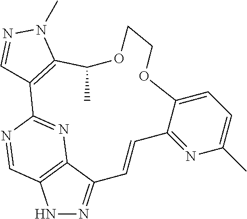 [SMILES: Cc1ccc2c(n1)/C=C/c1n[nH]c3cnc(nc13)-c1cnn(C)c1[C@@H](C)OCCO2] | (9R,18E)-8,9,16-trimethyl-8,9,11,12-tetrahydro- 2H-5,3-(azenometheno)dipyrazolo[3,4-f:4′,3′- j]pyrido[2,3-n][1,4,9]dioxazacyclopentadecine |
| 21 | 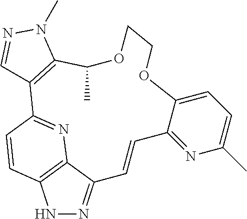 [SMILES: Cc1ccc2c(n1)/C=C/c1n[nH]c3ccc(nc13)-c1cnn(C)c1[C@@H](C)OCCO2] | (9R,18E)-8,9,16-trimethyl-8,9,11,12-tetrahydro- 2H-3,5-ethenodipyrazolo[3,4-f:4′,3′- j]pyrido[2,3-n][1,4,9]dioxazacyclopentadecine |
| 22 | 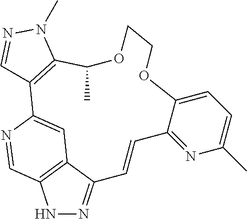 [SMILES: Cc1ccc2c(n1)/C=C/c1n[nH]c3cnc(cc13)-c1cnn(C)c1[C@@H](C)OCCO2] | (9R,18E)-8,9,16-trimethyl-8,9,11,12-tetrahydro- 2H-5,3- (azenometheno)dipyrazolo[3′,4′:9,10;4″,3″:13,1 4][1,4]dioxacyclopentadecino[6,5-b]pyridine |
| 23 | 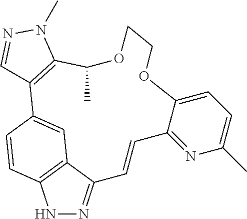 [SMILES: Cc1ccc2c(n1)/C=C/c1n[nH]c3ccc(cc13)-c1cnn(C)c1[C@@H](C)OCCO2] | (9R,18E)-8,9,16-trimethyl- 8,9,11,12-tetrahydro-2H-3,5- ethenodipyrazolo[3′,4′:9,10;4″,3″:13,14][1,4] dioxacyclopentadecino[6,5-b]pyridine |
| 24 | 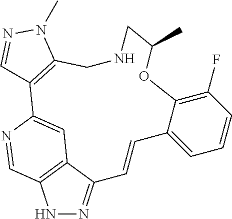 [SMILES: C[C@@H]1CNCc2c(cnn2C)-c2cc3c(n[nH]c3cn2)/C=C/c2cccc(F)c2O1] | (12R,18E)-14-fluoro-8,12-dimethyl- 2,8,9,10,11,12-hexahydro-5,3- (azenometheno)dipyrazolo[3,4-f:4′,3′- j][1,4]benzoxazacyclopentadecine |
| 26 | 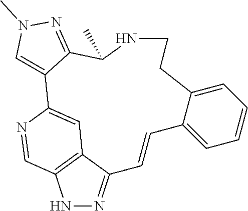 [SMILES: C[C@H]1NCCc2ccccc2/C=C/c2n[nH]c3cnc(cc23)-c2cn(C)nc21] | (9R,17E)-7,9-dimethyl-2,7,9,10,11,12- hexahydro-5,3-(azenometheno)dipyrazolo[3,4- e:4′,3′-i][3]benzazacyclotetradecine |
| 27 | 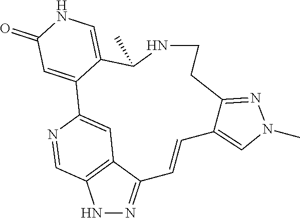 [SMILES: C[C@H]1NCCc2nn(C)cc2/C=C/c2n[nH]c3cnc(cc23)-c2cc(=O)[nH]cc21] | (4E,15R)-2,15-dimethyl-2,13,15,16,17,18- hexahydro-10,8-(azenometheno)dipyrazolo[4,3- g:4′,3′-k]pyrido[3,4-c]azacyclotetradecin- 12(7H)-one |
| 28 | 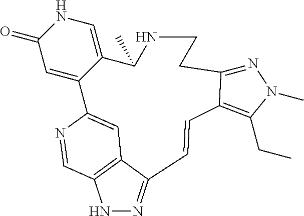 [SMILES: CCc1c2c(nn1C)CCN[C@H](C)c1c[nH]c(=O)cc1-c1cc3c(n[nH]c3cn1)/C=C/2] | (4E,15R)-3-ethyl-2,15-dimethyl- 2,13,15,16,17,18-hexahydro-10,8- (azenometheno)dipyrazolo[4,3-g:4′,3′- k]pyrido[3,4-c]azacyclotetradecin-12(7H)-one |
| 29 | 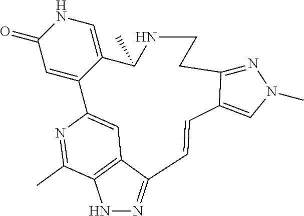 [SMILES: Cc1nc2cc3c(n[nH]c13)/C=C/c1cn(C)nc1CCN[C@H](C)c1c[nH]c(=O)cc1-2] | (4E,15R)-2,15,20-trimethyl-2,13,15,16,17,18- hexahydro-10,8-(azenometheno)dipyrazolo[4,3- g:4′,3′-k]pyrido[3,4-c]azacyclotetradecin- 12(7H)-one |
| 30 | 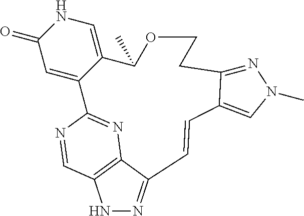 [SMILES: C[C@H]1OCCc2nn(C)cc2/C=C/c2n[nH]c3cnc(nc23)-c2cc(=O)[nH]cc21] | (10R,17E)-10,15-dimethyl-2,8,10,12,13,15- hexahydro-7H-5,3- (azenometheno)dipyrazolo[4,3-g:4′,3′- k]pyrido[3,4-c][1,6]oxazacyclotetradecin-7-one |
| 31 | 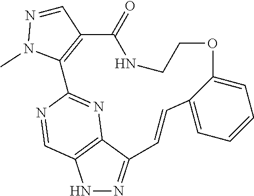 [SMILES: Cn1ncc2c1-c1ncc3[nH]nc(c3n1)/C=C/c1ccccc1OCCNC2=O] | (18E)-6-methyl-2,6,11,12-tetrahydro-5,3- (azenometheno)dipyrazolo[4,3-f:4′,3′- j][1,4,9]benzoxadiazacyclopentadecin-9(10H)- one |
| 32 | 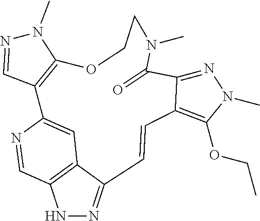 [SMILES: CCOc1c2c(nn1C)C(=O)N(C)CCOc1c(cnn1C)-c1cc3c(n[nH]c3cn1)/C=C/2] | (17E)-16-ethoxy-8,12,15-trimethyl-2,11,12,15- tetrahydro-8H-5,3- (azenometheno)tripyrazolo[3,4-f:3′,4′-j:4″,3″- n][1,4]oxazacyclopentadecin-13(10H)-one |
| 33 | 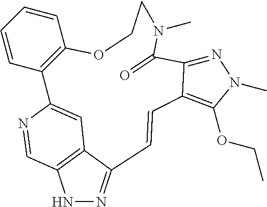 [SMILES: CCOc1c2c(nn1C)C(=O)N(C)CCOc1ccccc1-c1cc3c(n[nH]c3cn1)/C=C/2] | (18E)-17-ethoxy-13,16-dimethyl-2,12,13,16- tetrahydro-5,3-(azenometheno)dipyrazolo[3,4- f:3′,4′-j][1,4]benzoxazacyclopentadecin- 14(11H)-one |
| 34 | 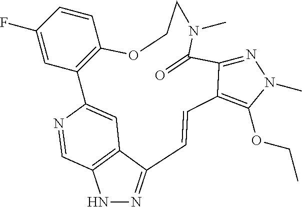 [SMILES: CCOc1c2c(nn1C)C(=O)N(C)CCOc1ccc(F)cc1-c1cc3c(n[nH]c3cn1)/C=C/2] | (18E)-17-ethoxy-7-fluoro-13,16-dimethyl- 2,12,13,16-tetrahydro-5,3- (azenometheno)dipyrazolo[3,4-f:3′,4′- j][1,4]benzoxazacyclopentadecin-14(11H)-one |
| 35 | 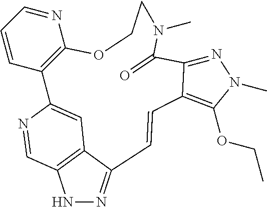 [SMILES: CCOc1c2c(nn1C)C(=O)N(C)CCOc1ncccc1-c1cc3c(n[nH]c3cn1)/C=C/2] | (18E)-17-ethoxy-13,16-dimethyl-2,12,13,16- tetrahydro-5,3-(azenometheno)dipyrazolo[3,4- f:3′,4′-j]pyrido[3,2- n][1,4]oxazacyclopentadecin-14(11H)-one |
| 36 | 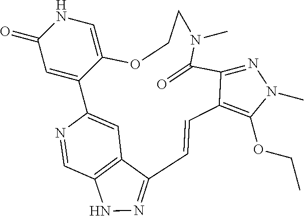 [SMILES: CCOc1c2c(nn1C)C(=O)N(C)CCOc1c[nH]c(=O)cc1-c1cc3c(n[nH]c3cn1)/C=C/2] | (18E)-17-ethoxy-13,16-dimethyl-2,12,13,16- tetrahydro-5,3-(azenometheno)dipyrazolo[3,4- f:3′,4′-j]pyrido[4,3- n][1,4]oxazacyclopentadecine-7,14(8H,11H)- dione |
| 37 | 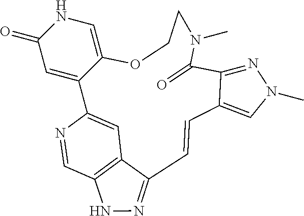 [SMILES: CN1CCOc2c[nH]c(=O)cc2-c2cc3c(n[nH]c3cn2)/C=C/c2cn(C)nc2C1=O] | (18E)-13,16-dimethyl-2,12,13,16-tetrahydro- 5,3-(azenometheno)dipyrazolo[3,4-f:3′,4′- j]pyrido[4,3-n][1,4]oxazacyclopentadecine- 7,14(8H,11H)-dione |
| 38 | 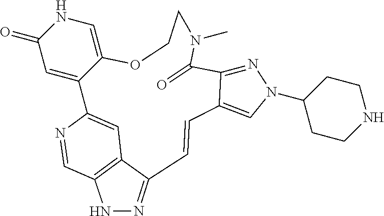 [SMILES: CN1CCOc2c[nH]c(=O)cc2-c2cc3c(n[nH]c3cn2)/C=C/c2cn(C3CCNCC3)nc2C1=O] | (18E)-13-methyl-16-(piperidin-4-yl)- 2,12,13,16-tetrahydro-5,3- (azenometheno)dipyrazolo[3,4-f:3′,4′- j]pyrido[4,3-n][1,4]oxazacyclopentadecine- 7,14(8H,11H)-dione |
| 39 | 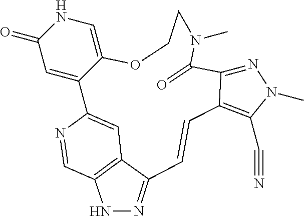 [SMILES: CN1CCOc2c[nH]c(=O)cc2-c2cc3c(n[nH]c3cn2)/C=C/c2c(nn(C)c2C#N)C1=O] | (18E)-13,16-dimethyl-7,14-dioxo- 2,7,8,11,12,13,14,16-octahydro-5,3- (azenometheno)dipyrazolo[3,4-f:3′,4′- j]pyrido[4,3-n][1,4]oxazacyclopentadecine-17- carbonitrile |
| 40 | 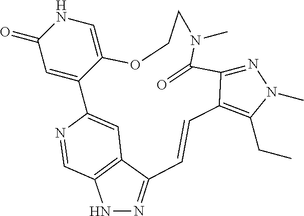 [SMILES: CCc1c2c(nn1C)C(=O)N(C)CCOc1c[nH]c(=O)cc1-c1cc3c(n[nH]c3cn1)/C=C/2] | (18E)-17-ethyl-13,16-dimethyl-2,12,13,16- tetrahydro-5,3-(azenometheno)dipyrazolo[3,4- f:3′,4′-j]pyrido[4,3- n][1,4]oxazacyclopentadecine-7,14(8H,11H)- dione |
| 41 | 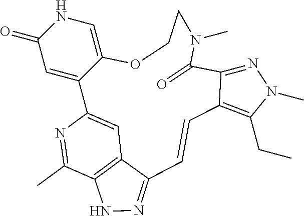 [SMILES: CCc1c2c(nn1C)C(=O)N(C)CCOc1c[nH]c(=O)cc1-c1cc3c(n[nH]c3c(C)n1)/C=C/2] | (18E)-17-ethyl-13,16,21-trimethyl-2,12,13,16- tetrahydro-5,3-(azenometheno)dipyrazolo[3,4- f:3′,4′-j]pyrido[4,3- n][1,4]oxazacyclopentadecine-7,14(8H,11H)- dione |
| 42 | 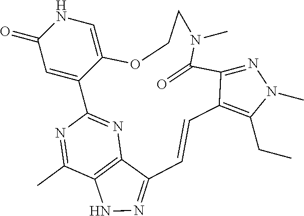 [SMILES: CCc1c2c(nn1C)C(=O)N(C)CCOc1c[nH]c(=O)cc1-c1nc(C)c3[nH]nc(c3n1)/C=C/2] | (18E)-17-ethyl-13,16,21-trimethyl-10,11,12,13- tetrahydro-2H-5,3- (azenometheno)dipyrazolo[3,4-j:3′,4′- n]pyrido[4,3-c][1,8]diazacyclopentadecine- 7,14(8H,16H)-dione |
| 43 | 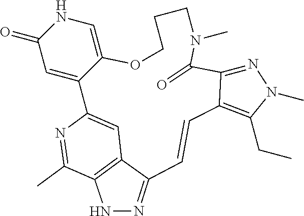 [SMILES: CCc1c2c(nn1C)C(=O)N(C)CCCOc1c[nH]c(=O)cc1-c1cc3c(n[nH]c3c(C)n1)/C=C/2] | (19E)-18-ethyl-14,17,22-trimethyl-11,12,13,14- tetrahydro-2H-5,3- (azenometheno)dipyrazolo[3,4-g:3′,4′- k]pyrido[4,3-0][1,5]oxazacyclohexadecine- 7,15(8H,17H)-dione |
| 44 | 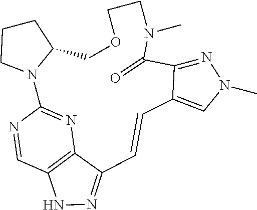 [SMILES: CN1CCOC[C@H]2CCCN2c2ncc3[nH]nc(c3n2)/C=C/c2cn(C)nc2C1=O] | (9aR,19E)-14,17-dimethyl- 2,8,9,9a, 10,13,14,17-octahydro-7H-5,3- (azenometheno)dipyrazolo[4,3-g:4′,3′- k]pyrrolo[2,1- c][1,4,6,14]oxatriazacyclohexadecin-15(12H)- one |
| 45 | 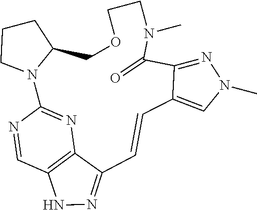 [SMILES: CN1CCOC[C@@H]2CCCN2c2ncc3[nH]nc(c3n2)/C=C/c2cn(C)nc2C1=O] | (9aS,19E)-14,17-dimethyl-2,8,9,9a,10,13,14,17- octahydro-7H-5,3- (azenometheno)dipyrazolo[4,3-g:4′,3′- k]pyrrolo[2,1- c][1,4,6,14]oxatriazacyclohexadecin-15(12H)- one |
| 46 | 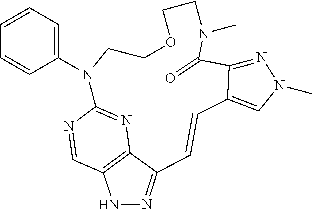 [SMILES: CN1CCOCCN(c2ccccc2)c2ncc3[nH]nc(c3n2)/C=C/c2cn(C)nc2C1=O] | (17E)-12,15-dimethyl-6-phenyl-2,7,8,11,12,15- hexahydro-6H-5,3- (azenometheno)dipyrazolo[4,3-g:4′,3′- k][1,4,6, 14]oxatriazacyclohexadecin-13(10H)- one |
| 47 | 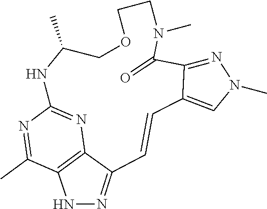 | (7R,17E)-7,12,15,20-tetramethyl- 2,7,8,11,12,15-hexahydro-6H-5,3- (azenometheno)dipyrazolo[4,3-g:4′,3′- k][1,4,6, 14]oxatriazacyclohexadecin-13(10H)- one |
| 48 | 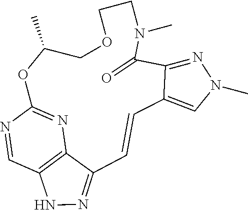 [SMILES: C[C@@H]1COCCN(C)C(=O)c2nn(C)cc2/C=C/c2n[nH]c3cnc(nc23)O1] | (7R,17E)-7,12,15-trimethyl-2,7,8,11,12,15- hexahydro-5,3-(azenometheno)dipyrazolo[4,3- d:4′,3′-h][1,14,3,11]dioxadiazacyclohexadecin- 13(10H)-one |
| 49 | 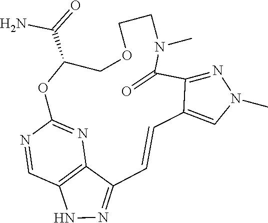 [SMILES: CN1CCOC[C@@H](C(N)=O)Oc2ncc3[nH]nc(c3n2)/C=C/c2cn(C)nc2C1=O] | (7S,17E)-12,15-dimethyl-13-oxo- 2,7,8,10,11,12,13,15-octahydro-5,3- (azenometheno)dipyrazolo[4,3-d:4′,3′- h][1,14,3, 11]dioxadiazacyclohexadecine-7- carboxamide |
| 50 | 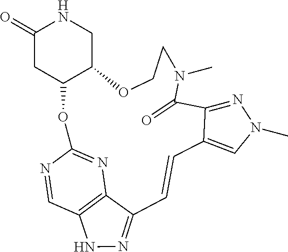 | (6aR,10aS,19E)-14,17-dimethyl- 2,6a,9,10,10a,13,14,17-octahydro-5,3- (azenometheno)dipyrazolo[4,3-d:4′,3′- h]pyrido[3,4- o][1,14,3,11]dioxadiazacyclohexadecine- 8,15(7H,12H)-dione |
| 51 | 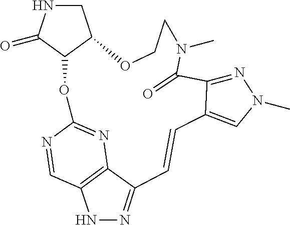 | (6aS,9aS,18E)-13,16-dimethyl-2,9,9a,12,13,16- hexahydro-6aH-5,3- (azenometheno)dipyrazolo[4,3-d:4′,3′- h]pyrrolo[3,4- o][1,14,3,11]dioxadiazacyclohexadecine- 7,14(8H,11H)-dione |
| 52 | 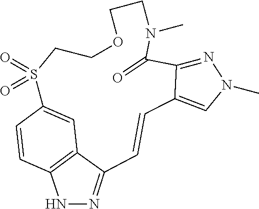 [SMILES: CN1CCOCCS(=O)(=O)c2ccc3[nH]nc(c3c2)/C=C/c2cn(C)nc2C1=O] | (17E)-12,15-dimethyl-2,7,8,11,12,15- hexahydro-6H-3,5-etheno-626-dipyrazolo[4,3- g:4′,3′-k][1,4,14]oxathiazacyclohexadecine- 6,6,13(10H)-trione |
| 53 | 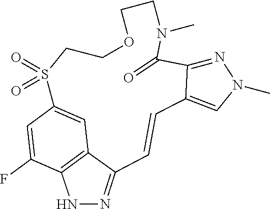 [SMILES: CN1CCOCCS(=O)(=O)c2cc(F)c3[nH]nc(c3c2)/C=C/c2cn(C)nc2C1=O] | (17E)-20-fluoro-12,15-dimethyl-2,7,8,11,12,15- hexahydro-6H-3,5-etheno-626-dipyrazolo[4,3- g:4′,3′-k][1,4,14]oxathiazacyclohexadecine- 6,6,13(10H)-trione |
| 54 | 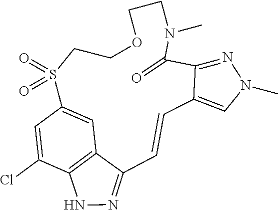 [SMILES: CN1CCOCCS(=O)(=O)c2cc(Cl)c3[nH]nc(c3c2)/C=C/c2cn(C)nc2C1=O] | (17E)-20-chloro-12,15-dimethyl- 2,7,8,11,12,15-hexahydro-6H-3,5-etheno-62.6- dipyrazolo[4,3-g:4′,3′- k][1,4,14]oxathiazacyclohexadecine- 6,6,13(10H)-trione |
| 55 | 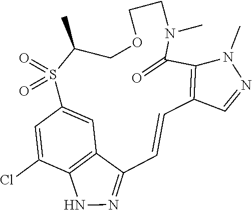 [SMILES: C[C@H]1COCCN(C)C(=O)c2c(cnn2C)/C=C/c2n[nH]c3c(Cl)cc(cc23)S1(=O)=O] | (7S,17E)-20-chloro-7,12,14-trimethyl- 2,7,8,11,12,14-hexahydro-6H-3,5-etheno-6).6- dipyrazolo[4,3-g:4′,3′- k][1,4,14]oxathiazacyclohexadecine- 6,6,13(10H)-trione |
| 56 | 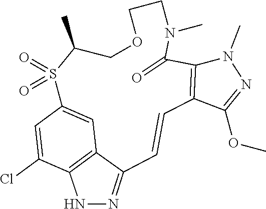 [SMILES: COc1nn(C)c2c1/C=C/c1n[nH]c3c(Cl)cc(cc13)S(=O)(=O)[C@@H](C)COCCN(C)C2=O] | (7S,17E)-20-chloro-16-methoxy-7,12,14- trimethyl-2,7,8,11,12,14-hexahydro-6H-3,5- etheno-626-dipyrazolo[4,3-g:4′,3′- k][1,4,14]oxathiazacyclohexadecine- 6,6,13(10H)-trione |
| 57 | 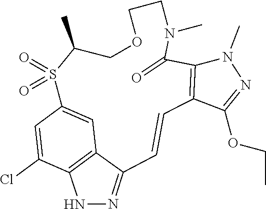 [SMILES: CCOc1nn(C)c2c1/C=C/c1n[nH]c3c(Cl)cc(cc13)S(=O)(=O)[C@@H](C)COCCN(C)C2=O] | (7S,17E)-20-chloro-16-ethoxy-7,12,14- trimethyl-2,7,8,11,12,14-hexahydro-6H-3,5- etheno-626-dipyrazolo[4,3-g:4′,3′- k][1,4,14]oxathiazacyclohexadecine- 6,6,13(10H)-trione |
| 58 | 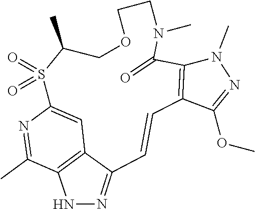 | (7S,17E)-16-methoxy-7,12,14,20-tetramethyl- 2,7,8,11,12,14-hexahydro-6H-5,3- (azenometheno)-626-dipyrazolo[4,3-g:4′,3′- k][1,4,14]oxathiazacyclohexadecine- 6,6,13(10H)-trione |
| 59 | 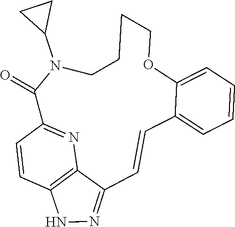 [SMILES: O=C1c2ccc3[nH]nc(c3n2)/C=C/c2ccccc2OCCCCN1C1CC1] | (17E)-7-cyclopropyl-8,9,10,11-tetrahydro-2H- 3,5-ethenopyrazolo[4,3- j][1,6,9]benzoxadiazacyclopentadecin-6(7H)- one |
| 60 | 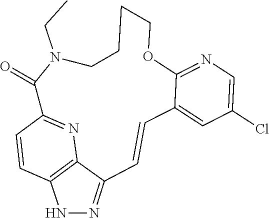 [SMILES: CCN1CCCCOc2ncc(Cl)cc2/C=C/c2n[nH]c3ccc(nc23)C1=O] | (17E)-15-chloro-7-ethyl-8,9,10,11-tetrahydro- 2H-3,5-ethenopyrazolo[4,3-j]pyrido[3,2- n][1,6,9]oxadiazacyclopentadecin-6(7H)-one |
| 61 | 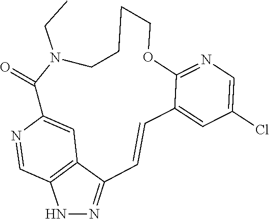 [SMILES: CCN1CCCCOc2ncc(Cl)cc2/C=C/c2n[nH]c3cnc(cc23)C1=O] | (17E)-15-chloro-7-ethyl-8,9,10,11-tetrahydro- 2H-5,3-(azenometheno)pyrazolo[4,3- j]pyrido[3,2-n][1,6]oxazacyclopentadecin- 6(7H)-one |
| 62 | 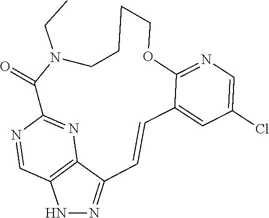 [SMILES: CCN1CCCCOc2ncc(Cl)cc2/C=C/c2n[nH]c3cnc(nc23)C1=O] | (17E)-15-chloro-7-ethyl-8,9,10,11-tetrahydro- 2H-5,3-(azenometheno)pyrazolo[4,3- j]pyrido[3,2-n][1,6,9]oxadiazacyclopentadecin- 6(7H)-one |
| 63 | 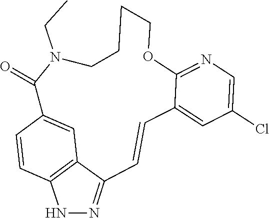 [SMILES: CCN1CCCCOc2ncc(Cl)cc2/C=C/c2n[nH]c3ccc(cc23)C1=O] | (17E)-15-chloro-7-ethyl-8,9,10,11-tetrahydro- 2H-3,5-ethenopyrazolo[4,3-j]pyrido[3,2- n][1,6]oxazacyclopentadecin-6(7H)-one |
| 64 | 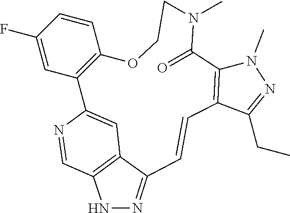 [SMILES: CCc1nn(C)c2c1/C=C/c1n[nH]c3cnc(cc13)-c1cc(F)ccc1OCCN(C)C2=O] | (18E)-17-ethyl-7-fluoro-13,15-dimethyl- 2,12,13,15-tetrahydro-5,3- (azenometheno)dipyrazolo[3,4-f:3′,4′- j][1,4]benzoxazacyclopentadecin-14(11H)-one |
| 65 | 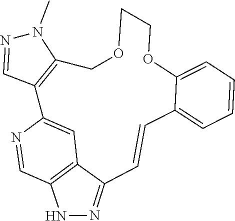 [SMILES: Cn1ncc2c1COCCOc1ccccc1/C=C/c1n[nH]c3cnc-2cc13] | (11E)-1-methyl-1,18,19,21-tetrahydro-8H- 10,7,4-(ethan[1]yl[1,2]diylidene)pyrazolo[3,4- f][1,4,9,12,13]benzodioxatriazacyclooctadecine |
| 66 | 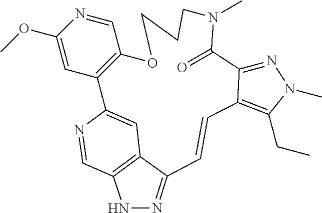 | (19E)-18-ethyl-7-methoxy-14,17-dimethyl- 2,11,12,13,14,17-hexahydro-15H-5,3- (azenometheno)dipyrazolo[3,4-g:3′,4′- k]pyrido[4,3-0][1,5]oxazacyclohexadecin-15- one |
| 67 | 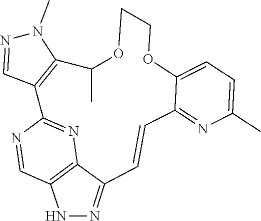 [SMILES: Cc1ccc2c(n1)/C=C/c1n[nH]c3cnc(nc13)-c1cnn(C)c1C(C)OCCO2] | (18E)-8,9,16-trimethyl-8,9,11,12-tetrahydro- 2H-5,3-(azenometheno)dipyrazolo[3,4-f:4′,3′- j]pyrido[2,3-n][1,4,9]dioxazacyclopentadecine |
| 68 | 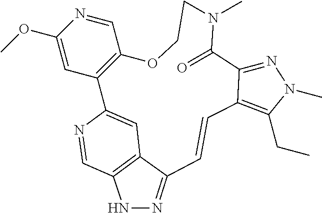 | (18E)-17-ethyl-7-methoxy-13,16-dimethyl- 2,12,13,16-tetrahydro-5,3- (azenometheno)dipyrazolo[3,4-f:3′,4′- j]pyrido[4,3-n][1,4]oxazacyclopentadecin- 14(11H)-one |
| 69 | 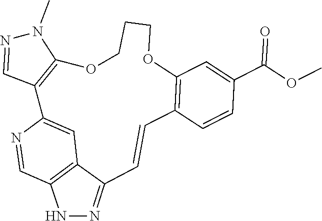 [SMILES: COC(=O)c1ccc2c(c1)OCCCOc1c(cnn1C)-c1cc3c(n[nH]c3cn1)/C=C/2] | methyl (11E)-1-methyl-19,20-dihydro- 1H,8H,18H-10,7,4- (ethan[1]yl[1,2]diylidene)pyrazolo[3,4- f][1,5,9,12,13]benzodioxatriazacyclooctadecine- 15-carboxylate |
| 70 | 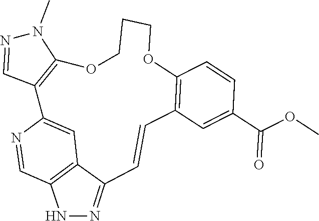 [SMILES: COC(=O)c1ccc2c(c1)/C=C/c1n[nH]c3cnc(cc13)-c1cnn(C)c1OCCCO2] | methyl (11E)-1-methyl-19,20-dihydro- 1H,8H,18H-10,7,4- (ethan[1]yl[1,2]diylidene)pyrazolo[3,4- f][1,5,9,12,13]benzodioxatriazacyclooctadecine- 14-carboxylate |
| 71 | 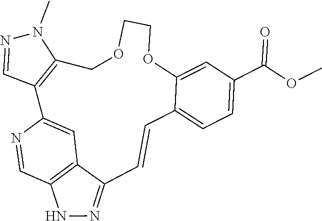 [SMILES: COC(=O)c1ccc2c(c1)OCCOCc1c(cnn1C)-c1cc3c(n[nH]c3cn1)/C=C/2] | methyl (11E)-1-methyl-1,18,19,21-tetrahydro- 8H-10,7,4- (ethan[1]yl[1,2]diylidene)pyrazolo[3,4- f][1,4,9,12,13]benzodioxatriazacyclooctadecine- 15-carboxylate |
| 72 | 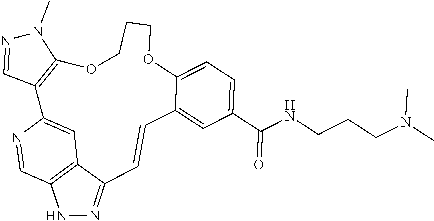 [SMILES: CN(C)CCCNC(=O)c1ccc2c(c1)/C=C/c1n[nH]c3cnc(cc13)-c1cnn(C)c1OCCCO2] | (11E)-N-[3-(dimethylamino)propyl]-1-methyl- 19,20-dihydro-1H,8H,18H-10,7,4- (ethan[1]yl[1,2]diylidene)pyrazolo[3,4- f][1,5,9,12,13]benzodioxatriazacyclooctadecine- 14-carboxamide |
| 73 | 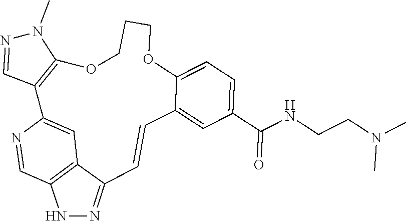 [SMILES: CN(C)CCNC(=O)c1ccc2c(c1)/C=C/c1n[nH]c3cnc(cc13)-c1cnn(C)c1OCCCO2] | (11E)-N-[2-(dimethylamino)ethyl]-1-methyl- 19,20-dihydro-1H,8H,18H-10,7,4- (ethan[1]yl[1,2]diylidene)pyrazolo[3,4- f][1,5,9,12,13]benzodioxatriazacyclooctadecine- 14-carboxamide |
| 74 | 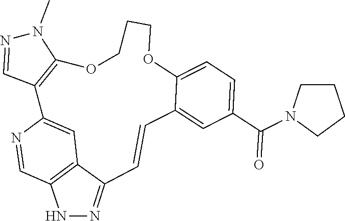 [SMILES: Cn1ncc2c1OCCCOc1ccc(C(=O)N3CCCC3)cc1/C=C/c1n[nH]c3cnc-2cc13] | [(11E)-1-methyl-19,20-dihydro-1H,8H,18H- 10,7,4-(ethan[1]yl[1,2]diylidene)pyrazolo[3,4- f][1,5,9,12,13]benzodioxatriazacyclooctadecin- 14-y1](pyrrolidin-1-yl)methanone |
| 75 | 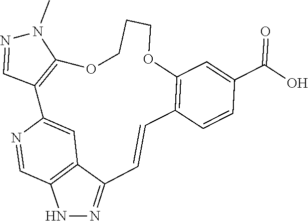 [SMILES: Cn1ncc2c1OCCCOc1cc(C(=O)O)ccc1/C=C/c1n[nH]c3cnc-2cc13] | (11E)-1-methyl-19,20-dihydro-1H,8H,18H- 10,7,4-(ethan[1]yl[1,2]diylidene)pyrazolo[3,4- f][1,5,9,12,13]benzodioxatriazacyclooctadecine- 15-carboxylic acid |
| 76 | 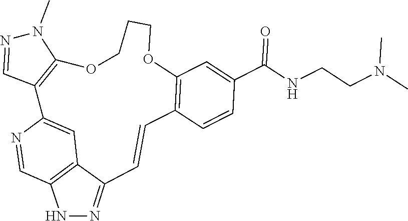 [SMILES: CN(C)CCNC(=O)c1ccc2c(c1)OCCCOc1c(cnn1C)-c1cc3c(n[nH]c3cn1)/C=C/2] | (11E)-N-[2-(dimethylamino)ethyl]-1-methyl- 19,20-dihydro-1H,8H,18H-10,7,4- (ethan[1]yl[1,2]diylidene)pyrazolo[3,4- f][1,5,9,12,13]benzodioxatriazacyclooctadecine- 15-carboxamide |
| 77 | 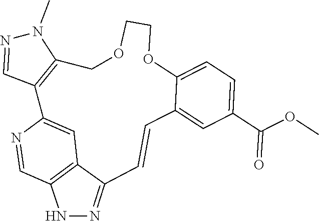 [SMILES: COC(=O)c1ccc2c(c1)/C=C/c1n[nH]c3cnc(cc13)-c1cnn(C)c1COCCO2] | methyl (11E)-1-methyl-1,18,19,21-tetrahydro- 8H-10,7,4- (ethan[1]yl[1,2]diylidene)pyrazolo[3,4- f][1,4,9,12,13]benzodioxatriazacyclooctadecine- 14-carboxylate |
| 78 | 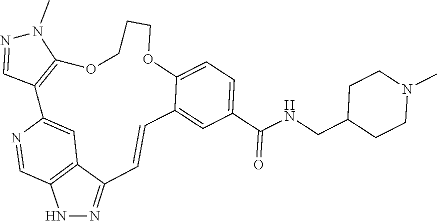 [SMILES: CN1CCC(CNC(=O)c2ccc3c(c2)/C=C/c2n[nH]c4cnc(cc24)-c2cnn(C)c2OCCCO3)CC1] | (11E)-1-methyl-N-[(1-methylpiperidin-4- yl)methyl]-19,20-dihydro-1H,8H,18H-10,7,4- (ethan[1]y][1,2]diylidene)pyrazolo[3,4- f][1,5,9,12,13]benzodioxatriazacyclooctadecine- 14-carboxamide |
| 79 | 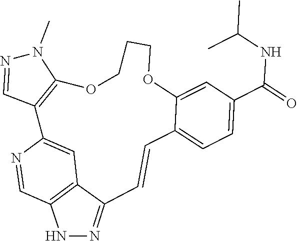 [SMILES: CC(C)NC(=O)c1ccc2c(c1)OCCCOc1c(cnn1C)-c1cc3c(n[nH]c3cn1)/C=C/2] | (11E)-1-methyl-N-(propan-2-yl)-19,20-dihydro- 1H,8H,18H-10,7,4- (ethan[1]yl[1,2]diylidene)pyrazolo[3,4- f][1,5,9,12,13]benzodioxatriazacyclooctadecine- 15-carboxamide |
| 80 | 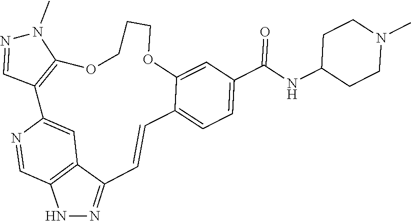 [SMILES: CN1CCC(NC(=O)c2ccc3c(c2)OCCCOc2c(cnn2C)-c2cc4c(n[nH]c4cn2)/C=C/3)CC1] | (11E)-1-methyl-N-(1-methylpiperidin-4-yl)- 19,20-dihydro-1H,8H,18H-10,7,4- (ethan[1]yl[1,2]diylidene)pyrazolo[3,4- f][1,5,9,12,13]benzodioxatriazacyclooctadecine- 15-carboxamide |
| 81 | 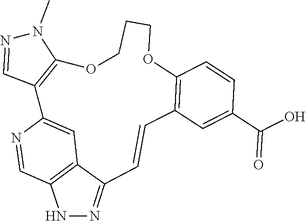 [SMILES: Cn1ncc2c1OCCCOc1ccc(C(=O)O)cc1/C=C/c1n[nH]c3cnc-2cc13] | (11E)-1-methyl-19,20-dihydro-1H,8H,18H- 10,7,4-(ethan[1]yl[1,2]diylidene)pyrazolo[3,4- f][1,5,9,12,13]benzodioxatriazacyclooctadecine- 14-carboxylic acid |
| 82 | 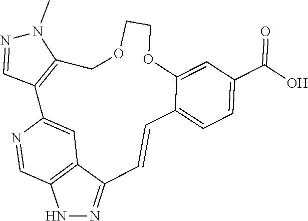 [SMILES: Cn1ncc2c1COCCOc1cc(C(=O)O)ccc1/C=C/c1n[nH]c3cnc-2cc13] | (11E)-1-methyl-1,18,19,21-tetrahydro-8H- 10,7,4-(ethan[1]yl[1,2]diylidene)pyrazolo[3,4- f][1,4,9,12,13]benzodioxatriazacyclooctadecine- 15-carboxylic acid |
| 83 | 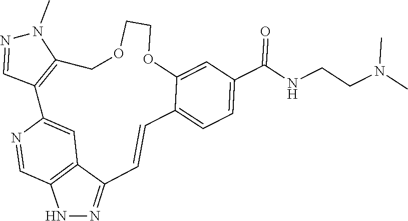 [SMILES: CN(C)CCNC(=O)c1ccc2c(c1)OCCOCc1c(cnn1C)-c1cc3c(n[nH]c3cn1)/C=C/2] | (11E)-N-[2-(dimethylamino)ethyl]-1-methyl- 1,18,19,21-tetrahydro-8H-10,7,4- (ethan[1]yl[1,2]diylidene)pyrazolo[3,4- f][1,4,9,12,13]benzodioxatriazacyclooctadecine- 15-carboxamide |
| 84 | 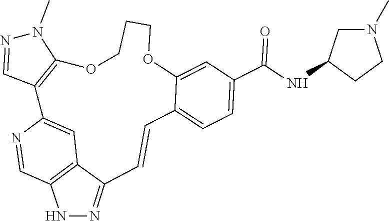 [SMILES: CN1CC[C@@H](NC(=O)c2ccc3c(c2)OCCCOc2c(cnn2C)-c2cc4c(n[nH]c4cn2)/C=C/3)C1] | (11E)-1-methyl-N-[(3R)-1-methylpyrrolidin-3- yl]-19,20-dihydro-1H,8H,18H-10,7,4- (ethan[1]yl[1,2]diylidene)pyrazolo[3,4- f][1,5,9,12,13]benzodioxatriazacyclooctadecine- 15-carboxamide |
| 85 | 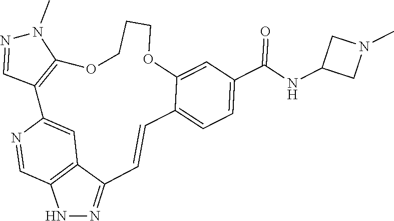 [SMILES: CN1CC(NC(=O)c2ccc3c(c2)OCCCOc2c(cnn2C)-c2cc4c(n[nH]c4cn2)/C=C/3)C1] | (11E)-1-methyl-N-(1-methylazetidin-3-yl)- 19,20-dihydro-1H,8H,18H-10,7,4- (ethan[1]yl[1,2]diylidene)pyrazolo[3,4- f][1,5,9,12,13]benzodioxatriazacyclooctadecine- 15-carboxamide |
| 86 | 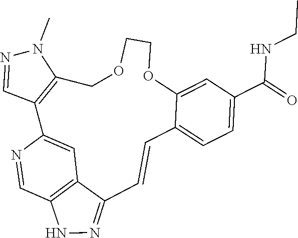 [SMILES: CCNC(=O)c1ccc2c(c1)OCCOCc1c(cnn1C)-c1cc3c(n[nH]c3cn1)/C=C/2] | (11E)-N-ethyl-1-methyl-1,18,19,21-tetrahydro- 8H-10,7,4- (ethan[1]yl[1,2]diylidene)pyrazolo[3,4- f][1,4,9,12,13]benzodioxatriazacyclooctadecine- 15-carboxamide |
| 87 | 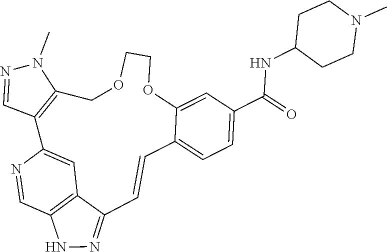 [SMILES: CN1CCC(NC(=O)c2ccc3c(c2)OCCOCc2c(cnn2C)-c2cc4c(n[nH]c4cn2)/C=C/3)CC1] | (11E)-1-methyl-N-(1-methylpiperidin-4-yl)- 1,18,19,21-tetrahydro-8H-10,7,4- (ethan[1]yl[1,2]diylidene)pyrazolo[3,4- f][1,4,9,12,13]benzodioxatriazacyclooctadecine- 15-carboxamide |
| 88 | 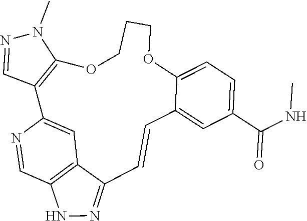 [SMILES: CNC(=O)c1ccc2c(c1)/C=C/c1n[nH]c3cnc(cc13)-c1cnn(C)c1OCCCO2] | (11E)-N,1-dimethyl-19,20-dihydro-1H,8H,18H- 10,7,4-(ethan[1]yl[1,2]diylidene)pyrazolo[3,4- f][1,5,9,12,13]benzodioxatriazacyclooctadecine- 14-carboxamide |
| 89 | 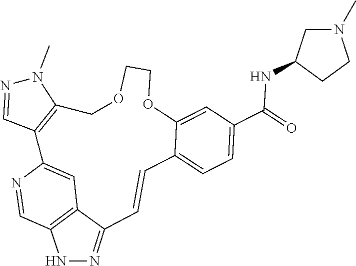 [SMILES: CN1CC[C@@H](NC(=O)c2ccc3c(c2)OCCOCc2c(cnn2C)-c2cc4c(n[nH]c4cn2)/C=C/3)C1] | (11E)-1-methyl-N-[(3R)-1-methylpyrrolidin-3- yl]-1,18,19,21-tetrahydro-8H-10,7,4- (ethan[1]yl[1,2]diylidene)pyrazolo[3,4- f[1,4,9,12,13]benzodioxatriazacyclooctadecine- 15-carboxamide |
| 90 | 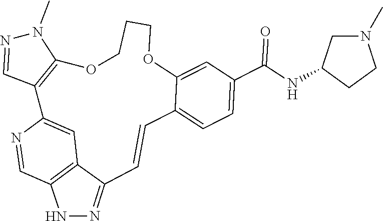 [SMILES: CN1CC[C@H](NC(=O)c2ccc3c(c2)OCCCOc2c(cnn2C)-c2cc4c(n[nH]c4cn2)/C=C/3)C1] | (11E)-1-methyl-N-[(3S)-1-methylpyrrolidin-3- yl]-19,20-dihydro-1H,8H,18H-10,7,4- (ethan[1]yl[1,2]diylidene)pyrazolo[3,4- f[1,5,9,12,13]benzodioxatriazacyclooctadecine 15-carboxamide |
| 91 | 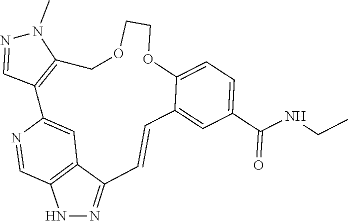 [SMILES: CCNC(=O)c1ccc2c(c1)/C=C/c1n[nH]c3cnc(cc13)-c1cnn(C)c1COCCO2] | (11E)-N-ethyl-1-methyl-1,18,19,21-tetrahydro- 8H-10,7,4- (ethan[1]yl[1,2]diylidene)pyrazolo[3,4- f][1,4,9,12,13]benzodioxatriazacyclooctadecine- 14-carboxamide |
| 92 | 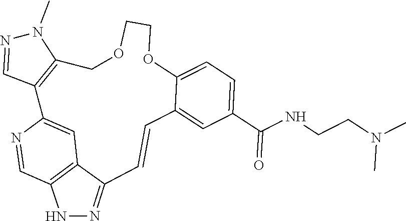 [SMILES: CN(C)CCNC(=O)c1ccc2c(c1)/C=C/c1n[nH]c3cnc(cc13)-c1cnn(C)c1COCCO2] | (11E)-N-[2-(dimethylamino)ethyl]-1-methyl- 1,18,19,21-tetrahydro-8H-10,7,4- (ethan[1]yl[1,2]diylidene)pyrazolo[3,4- f][1,4,9,12,13]benzodioxatriazacyclooctadecine- 14-carboxamide |
| 93 | 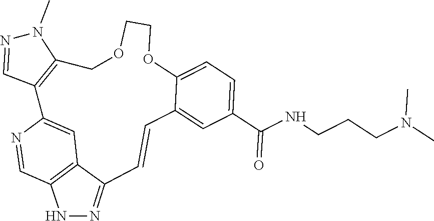 [SMILES: CN(C)CCCNC(=O)c1ccc2c(c1)/C=C/c1n[nH]c3cnc(cc13)-c1cnn(C)c1COCCO2] | (11E)-N-[3-(dimethylamino)propyl]-1-methyl- 1,18,19,21-tetrahydro-8H-10,7,4- (ethan[1]yl[1,2]diylidene)pyrazolo[3,4- f][1,4,9,12,13]benzodioxatriazacyclooctadecine- 14-carboxamide |
| 94 | 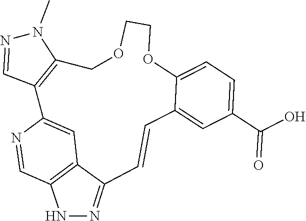 [SMILES: Cn1ncc2c1COCCOc1ccc(C(=O)O)cc1/C=C/c1n[nH]c3cnc-2cc13] | (11E)-1-methyl-1,18,19,21-tetrahydro-8H- 10,7,4-(ethan[1]yl[1,2]diylidene)pyrazolo[3,4- f][1,4,9,12,13]benzodioxatriazacyclooctadecine- 14-carboxylic acid |
| 95 | 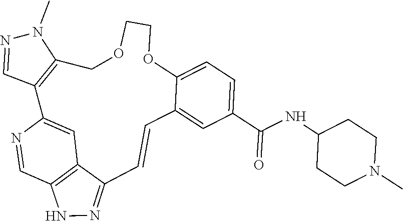 [SMILES: CN1CCC(NC(=O)c2ccc3c(c2)/C=C/c2n[nH]c4cnc(cc24)-c2cnn(C)c2COCCO3)CC1] | (11E)-1-methyl-N-(1-methylpiperidin-4-yl)- 1,18,19,21-tetrahydro-8H-10,7,4- (ethan[1]yl[1,2]diylidene)pyrazolo[3,4- f][1,4,9,12,13]benzodioxatriazacyclooctadecine- 14-carboxamide |
| 96 | 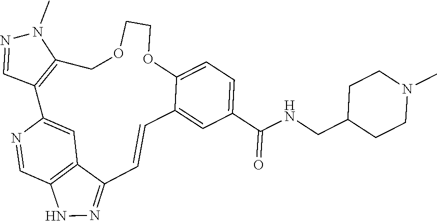 [SMILES: CN1CCC(CNC(=O)c2ccc3c(c2)/C=C/c2n[nH]c4cnc(cc24)-c2cnn(C)c2COCCO3)CC1] | (11E)-1-methyl-N-[(1-methylpiperidin-4- yl)methyl]-1,18,19,21-tetrahydro-8H-10,7,4- (ethan[1]yl[1,2]diylidene)pyrazolo[3,4- f][1,4,9,12,13]benzodioxatriazacyclooctadecine- 14-carboxamide |
| 97 | 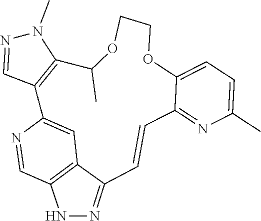 [SMILES: Cc1ccc2c(n1)/C=C/c1n[nH]c3cnc(cc13)-c1cnn(C)c1C(C)OCCO2] | (18E)-8,9,16-trimethyl-8,9,11,12-tetrahydro- 2H-5,3- (azenometheno)dipyrazolo[3′,4′:9,10;4″,3″:13,1 4][1,4]dioxacyclopentadecino[6,5-b]pyridine |
| 98 | 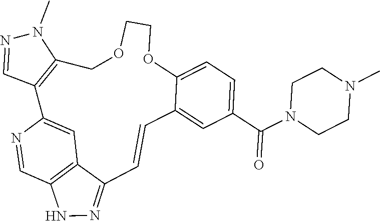 [SMILES: CN1CCN(C(=O)c2ccc3c(c2)/C=C/c2n[nH]c4cnc(cc24)-c2cnn(C)c2COCCO3)CC1] | (4-methylpiperazin-1-yl)[(11E)-1-methyl- 1,18,19,21-tetrahydro-8H-10,7,4- (ethan[1]yl[1,2]diylidene)pyrazolo[3,4- f][1,4,9,12,13]benzodioxatriazacyclooctadecin- 14-yl]methanone |
| 99 | 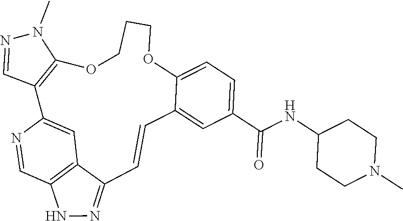 [SMILES: CN1CCC(NC(=O)c2ccc3c(c2)/C=C/c2n[nH]c4cnc(cc24)-c2cnn(C)c2OCCCO3)CC1] | (11E)-1-methyl-N-(1-methylpiperidin-4-yl)- 19,20-dihydro-1H,8H,18H-10,7,4- (ethan[1]yl[1,2]diylidene)pyrazolo[3,4- f][1,5,9,12,13]benzodioxatriazacyclooctadecine- 14-carboxamide |
| 100 | 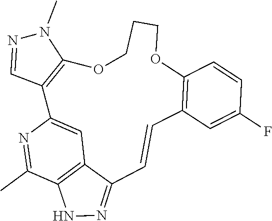 [SMILES: Cc1nc2cc3c(n[nH]c13)/C=C/c1cc(F)ccc1OCCCOc1c-2cnn1C] | (11E)-14-fluoro-1,6-dimethyl-19,20-dihydro- 1H,8H,18H-10,7,4- (ethan[1]yl[1,2]diylidene)pyrazolo[3,4- f[1,5,9,12,13]benzodioxatriazacyclooctadecine |
| 101 | 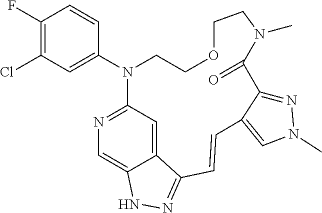 [SMILES: CN1CCOCCN(c2ccc(F)c(Cl)c2)c2cc3c(n[nH]c3cn2)/C=C/c2cn(C)nc2C1=O] | (17E)-6-(3-chloro-4-fluorophenyl)-12,15- dimethyl-2,7,8,11,12,15-hexahydro-6H-5,3- (azenometheno)dipyrazolo[3,4-f:3′,4′- j][1,4,14]oxadiazacyclohexadecin-13(10H)-one |
| 102 | 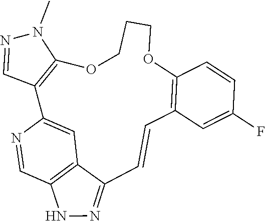 [SMILES: Cn1ncc2c1OCCCOc1ccc(F)cc1/C=C/c1n[nH]c3cnc-2cc13] | (11E)-14-fluoro-1-methyl-19,20-dihydro- 1H,8H,18H-10,7,4- (ethan[1]yl[1,2]diylidene)pyrazolo[3,4- f][1,5,9,12,13]benzodioxatriazacyclooctadecine |

and pharmaceutically acceptable salts thereof.

Those skilled in the art will recognize that the species listed or illustrated herein are not exhaustive, and that additional species within the scope of these defined terms may also be selected.

### Uses and Pharmaceutical Compositions

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the disclosure are sterile compositions. Pharmaceutical compositions may be prepared using compounding techniques known or that become available to those skilled in the art.

Sterile compositions are also contemplated by the disclosure, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the disclosure may be administered by a suitable route of delivery, such as oral, parenteral, rectal, nasal, topical, or ocular routes, or by inhalation. Preferably, the compositions are formulated for intravenous or oral administration.

For oral administration, the compounds the disclosure may be provided in a solid form, such as a tablet or capsule, or as a solution, emulsion, or suspension. To prepare the oral compositions, the compounds of the disclosure may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the pharmaceutical compositions may be administered using, for example, a spray formulation also containing a suitable carrier. The inventive compositions may be formulated for rectal administration as a suppository.

For topical applications, the compounds of the present disclosure are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the disclosure may utilize a patch formulation to effect transdermal delivery.

In some embodiments, the compounds and compositions described herein can be used to treat or used in methods from the treatment of disease, such as cancer. As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

Exemplary diseases include cancer, pain, neurological diseases, autoimmune diseases, and inflammation. As used herein, the term “cancer” includes, but is not limited to, ALCL, NSCLC, neuroblastoma, inflammatory myofibroblastic tumor, adult renal cell carcinoma, pediatric renal cell carcinoma, breast cancer, ER⁺ breast cancer, colonic adenocarcinoma, glioblastoma, glioblastoma multiforme, anaplastic thyroid cancer, cholangiocarcinoma, ovarian cancer, gastric adenocarcinoma, colorectal cancer, inflammatory myofibroblastic tumor, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cutaneous melanoma, head and neck squamous cell carcinoma, pediatric glioma CML, prostate cancer, lung squamous carcinoma, ovarian serous cystadenocarcinoma, skin cutaneous melanoma, castrate-resistant prostate cancer, Hodgkin lymphoma, and serous and clear cell endometrial cancer. In some embodiments, cancer includes, lung cancer, colon cancer, breast cancer, prostate cancer, hepatocellular carcinoma, renal cell carcinoma, gastric and esophago-gastric cancers, glioblastoma, head and neck cancers, inflammatory myofibroblastic tumors, and anaplastic large cell lymphoma. Pain includes, for example, pain from any source or etiology, including cancer pain, pain from chemotherapeutic treatment, nerve pain, pain from injury, or other sources. Autoimmune diseases include, for example, rheumatoid arthritis, Sjogren syndrome, Type I diabetes, and lupus. Exemplary neurological diseases include Alzheimer's Disease, Parkinson's Disease, Amyotrophic lateral sclerosis, and Huntington's disease. Exemplary inflammatory diseases include atherosclerosis, allergy, and inflammation from infection or injury.

In one aspect, the compounds and pharmaceutical compositions of the disclosure specifically target tyrosine receptor kinases, in particular EGFR. In some embodiments, the compounds and compositions described herein target particular EGFR mutations, such as L858R, Del19, Δ746-750, Δ746-750/T790M, Δ746-750/C979S, L858R/T790M, Del19/T790M, L858R/C979S, Del19/C979S, L858R/T790M/C979S, and A746-750/T790M/C979S. Thus, the compounds and pharmaceutical compositions described herein can be used to prevent, reverse, slow, or inhibit the activity of one or more kinases, or one or more mutations in the EGFR kinase. In preferred embodiments, methods of treatment target cancer. In other embodiments, methods are for treating lung cancer, such as non-small cell lung cancer.

As used herein, the term “EGFR mutation” refers to the EGFR protein (epidermal growth factor receptor) that is a tyrosine kinase receptor belonging to the ErbB family, and is encoded by the EGFR gene. The terms EGFR gene and ErbB family will be known and understood by one of skill in the art. It will be appreciated that an EGFR mutation describes a protein sequence mutation, such as L858R where a leucine to arginine mutation occurs at position 858 of the EGFR protein (a.k.a. EGFR L858R). It will further be appreciated that the production of an EGFR L858R protein is a gene product of the EGFR L858R gene that can be the result of a coding sequence mutation, e.g. thymine to guanine substitution, at position 2573 (T2573G), occurring in Exon 21 of the coding sequence. It will be appreciated that other EGFR mutations referred to herein can be described in a similar manner by reference to one or more mutations in the EGFR protein sequence and/or one or more mutations in the EGFR gene coding sequence. The descriptions of other EGFR mutations referenced herein will be well understood by a person having ordinary skill in the art. Furthermore, it will be appreciated that more than one mutation can occur in an EGFR sequence and can be gene product resulting from transcription and translation of an EGFR coding sequence having more than one mutation. Exemplary EGFR mutations include but are not limited to L858R, Del19, Δ746-750, Δ746-750/T790M, Δ746-750/C979S, L858R/T790M, Del19/T790M, L858R/C979S, Del19/C979S, L858R/T790M/C979S, and Δ746-750/T790M/C979S.

In the inhibitory methods of the disclosure, an “effective amount” means an amount sufficient to inhibit the target protein. Measuring such target modulation may be performed by routine analytical methods such as those described below. Such modulation is useful in a variety of settings, including in vitro assays. In such methods, the cell is preferably a cancer cell with abnormal signaling due to upregulation of EGFR, such as a cell expressing an EGFR protein having one or more EGFR mutations, such as L858R, Del19, Δ746-750, A746-750/T790M, Δ746-750/C979S, L858R/T790M, Del19/T790M, L858R/C979S, Del19/C979S, L858R/T790M/C979S, and Δ746-750/T790M/C979S.

In treatment methods according to the disclosure, an “effective amount” means an amount or dose sufficient to generally bring about the desired therapeutic benefit in subjects needing such treatment. Effective amounts or doses of the compounds of the disclosure may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

In some embodiments, the disclosure provides a method of treating disease, such as cancer, in a subject comprising, administering a therapeutically effective amount of a compound as described herein, or a pharmaceutical composition as described herein. In some embodiments, the disclosure provides a compound as described herein or a pharmaceutical composition as described herein, for use in a method of treating disease, such as cancer, in a subject. In some embodiments, the disclosure provides for the use of a compound as described herein in the manufacture of a medicament for the treatment of disease, such as cancer in a subject. In some embodiments, the methods, compositions, uses, compounds and medicaments described herein can be used in connection with disease, such as cancers described herein, including those that are mediated or driven by EGFR mutations, such as the exemplary mutations, L858R, Del19, Δ746-750, Δ746-750/T790M, Δ746-750/C979S, L858R/T790M, Del19/T790M, L858R/C979S, Del19/C979S, L858R/T790M/C979S, and A746-750/T790M/C979S.

### Drug Combinations

The inventive compounds described herein may be used in pharmaceutical compositions or methods in combination with one or more additional active ingredients in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present disclosure or may be included with a compound of the present disclosure in a single pharmaceutical composition. The additional active ingredients may be administered simultaneously with, prior to, or after administration of a compound of the present disclosure.

Combination agents include additional active ingredients are those that are known or discovered to be effective in treating the diseases and disorders described herein, including those active against another target associated with the disease. For example, compositions and formulations of the disclosure, as well as methods of treatment, can further comprise other drugs or pharmaceuticals, e.g., other active agents useful for treating or palliative for the target diseases or related symptoms or conditions. For cancer indications, additional such agents include, but are not limited to, kinase inhibitors, such as ALK inhibitors (e.g., crizotinib), Raf inhibitors (e.g., vemurafenib), VEGFR inhibitors (e.g., sunitinib), standard chemotherapy agents such as alkylating agents, antimetabolites, anti-tumor antibiotics, topoisomerase inhibitors, platinum drugs, mitotic inhibitors, antibodies, hormone therapies, or corticosteroids. For pain indications, suitable combination agents include anti-inflammatories such as NSAIDs. The pharmaceutical compositions of the disclosure may additional comprise one or more of such active agents, and methods of treatment may additionally comprise administering an effective amount of one or more of such active agents.

### Chemical Synthesis Methods

The following examples are offered to illustrate but not to limit the disclosure. One of skill in the art will recognize that the following synthetic reactions and schemes may be modified by choice of suitable starting materials and reagents in order to access other compounds of Formula (Ia)-(IXa) or (I)-(IX).

In some embodiments, the disclosure provides compounds of the formula (X)

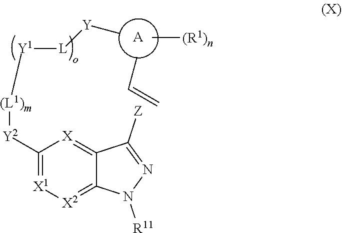

wherein A, L, L¹, X, X¹, X², Y, Y¹, Y², Z, R¹, R¹¹, m, n, and o are as defined herein.

In some embodiments, the disclosure provides compounds of the formula (XI)

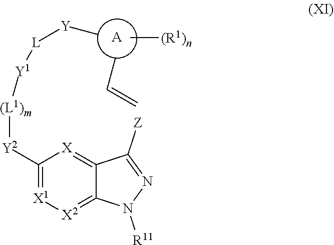

wherein A, L, L¹, X, X¹, X², Y, Y¹, Y², Z, R¹, R¹¹, m, and n are as defined herein.

In some embodiments, the disclosure provides compounds of the formula (XII)

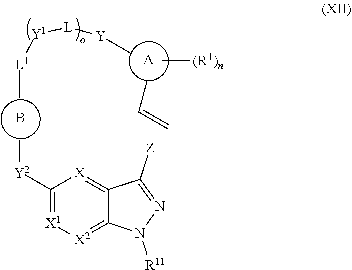

wherein A, B, L, L¹, X, X¹, X², Y, Y¹, Y², Z, R¹, R¹¹, n, and o are as described herein.

In some embodiments, the disclosure provides compounds of the formula (XIII)

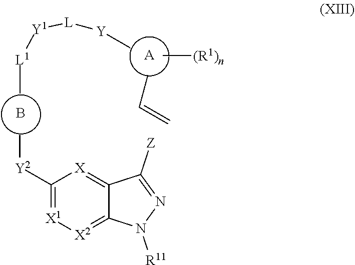

wherein A, B, L, L¹, X, X¹, X², Y, Y¹, Y², Z, R¹, R¹¹, and n are as described herein.

In some embodiments, the disclosure provides compounds of the formula (XIV)

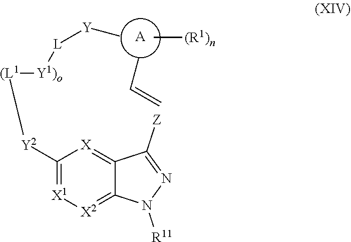

wherein A, L, L¹, X, X¹, X², Y, Y¹, Y², Z, R¹, R¹¹, n, and o are as described herein.

In some embodiments, the disclosure provides compounds of the formula (XV)

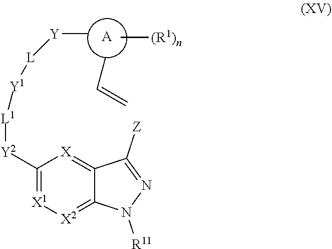

wherein A, L, L¹, X, X¹, X², Y, Y¹, Y², Z, R¹, R¹¹, and n are as described herein.

In some embodiments, Z is H, halogen, —OTf, —OMs, COOH, and the like. It will be appreciated that Z can be a variety of groups that are useful in coupling reactions, depending on the reaction conditions that are known to one of skill in the art for ring closing reactions. In some embodiments, Z is Cl, Br or I.

In some embodiments, the disclosure provides compounds of the formula (XVI)

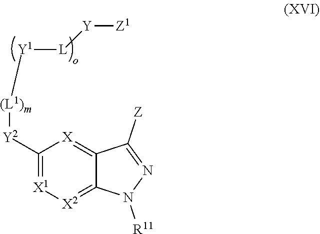

wherein L, L¹, X, X¹, X², Y, Y¹, Y², Z, Z¹, R¹¹, m, and o are as described herein.

In some embodiments, the disclosure provides compounds of the formula (XVII)

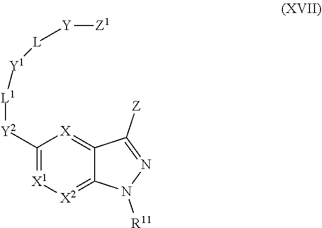

wherein L, L¹, X, X¹, X², Y, Y¹, Y², Z, Z¹, R¹¹, and m are as described herein.

In some embodiments, the disclosure provides compounds of the formula (XVIII)

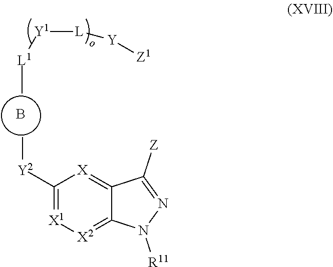

wherein B, L, L¹, X, X¹, X², Y, Y¹, Y², Z, Z¹, and R¹¹, and o are as described herein.

In some embodiments, the disclosure provides compounds of the formula (XIX)

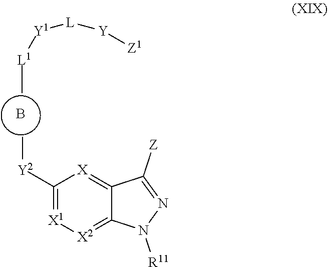

wherein B, L, L¹, X, X¹, X², Y, Y¹, Y², Z, Z¹, and R¹¹ are as described herein.

In some embodiments, the disclosure provides compounds of the formula (XX)

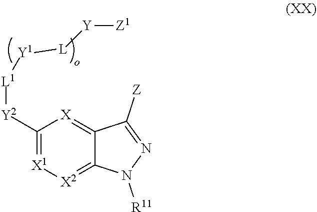

wherein L, L¹, X, X¹, X², Y, Y¹, Y², Z, Z¹, and R¹¹, and o are as described herein.

In some embodiments, the disclosure provides compounds of the formula (XXI)

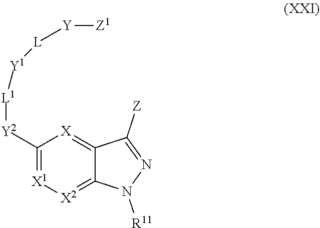

wherein L, L¹, X, X¹, X², Y, Y¹, Y², Z, Z¹, and R¹¹ are as described herein.

In some embodiments, Z is halogen. In some embodiments, Z is Br. In some embodiments, Z¹ is a leaving group or a protecting group. In some embodiments, Z¹ is a leaving group. In some embodiments, Z¹ is protecting group.

Abbreviations: The examples described herein use materials, including but not limited to, those described by the following abbreviations known to those skilled in the art:

| | |
|---|---|
| g | grams |
| eq | equivalents |
| mmol | millimoles |
| mL | milliliters |
| EtOAc | ethyl acetate |
| MHz | megahertz |
| ppm | parts per million |
| δ | chemical shift |
| s | singlet |
| d | doublet |
| t | triplet |
| q | quartet |
| quin | quintet |
| br | broad |
| m | multiplet |
| Hz | hertz |
| THF | tetrahydrofuran |
| ° C. | degrees Celsius |
| PE | petroleum ether |
| EA | ethyl acetate |
| R_{f} | retardation factor |
| N | normal |
| J | coupling constant |
| DMSO-d₆ | deuterated dimethyl sulfoxide |
| n-BuOH | n-butanol |
| DIEA | n,n-diisopropylethylamine |
| TMSCl | trimethylsilyl chloride |
| min | minutes |
| hr | hours |
| Me | methyl |
| Et | ethyl |
| i-Pr | isopropyl |
| TLC | thin layer chromatography |
| M | molar |
| Compd# | compound number |
| MS | mass spectrum |
| m/z | mass-to-charge ratio |
| Ms | methanesulfonyl |
| FDPP | pentafluorophenyl diphenylphosphinate |
| Boc | tert-butyloxycarbonyl |
| TFA | trifluoroacetic acid |
| Tos | toluenesulfonyl |
| DMAP | 4-(dimethylamino)pyridine |
| μM | micromolar |
| ATP | adenosine triphosphate |
| IC₅₀ | half maximal inhibitory concentration |
| U/mL | units of activity per milliliter |
| KHMDS | potassium bis(trimethylsilyl)amide |
| DIAD | diisopropyl azodicarboxylate |
| MeTHF | 2-methyltetrahydrofuran |
| MOM | methoxymethyl |
| DCM | dichloromethane |
| DMF | N,N-dimethylformamide |
| DPPA | diphenyl phosphoryl azide |
| DBU | 1,8-diazabicyclo[5.4.0]undec-7-ene |
| DIPEA | N,N-diisopropylethylamine |
| SEM | [2-(Trimethylsilyl)ethoxy]methyl acetal |
| Hex | hexanes |
| Pd(dppf)Cl₂ | [1,1′- |
| | Bis(diphenylphosphino)ferrocene]dichloropalladium(II) |
| MeCN (ACN) | Acetonitrile |
| Pd₂(dba)₃ | Tris(dibenzylideneacetone)dipalladium(0) |
| Hunig's Base | N,N-diisopropylethylamine |
| TBAF | Tert butyl ammonium fluoride |
| PPh₃ | Triphenyl phosphine |
| RT | Room Temperature |
| p-TSA | Para-Tolylsulfonic acid |
| t-BuOH | Tert-Butanol |
| Pd(amphos)Cl₂ | Dichlorobis[di-tert-butyl(4- |
| | dimethylaminophenyl)phosphine]palladium(II) |
| mCPBA | Meta-Chloroperoxy benzoic acid |
| AcOH | Acetic Acid |
| DMAc | N,N-Dimethylformamide |
| BPD | 4,4,5,5-tetramethyl-2-(4,4,5,5-tetramethyl- |
| | 1,3,2-dioxaborolan-2-yl)-1,3,2-dioxaborolane |
| MTBE | Methy tert-Butyl Ether |
| TBD | 3,4,6,7,8,9-hexahydro-2H- |
| | pyrimido[1,2-a]pyrimidine |
| DHP | 3,4-dihydro-2H-pyran |

General Method A

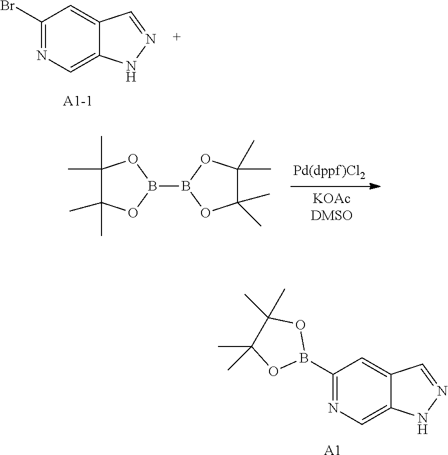

A1-1 (1.00 eq.), bis(pinacolato)diboron (1.05 eq.), potassium acetate (3.00 eq.) and anhydrous DMSO (0.26 M) are charged into a round bottom flask. After degassing the resulting reaction mixture with nitrogen for 15 minutes, 1,1-[Bis(diphenylphosphino)ferrocene]-dichloropalladium(II) (Pd(dppf)Cl₂, 0.05 eq.) is added. The reaction is then heated to 86° C. under nitrogen. After stirring for 20 hours, the reaction mixture is cooled to room temperature and slowly poured into 1.2 L of diethyl ether. The resulting mixture is transferred to a 2 L separation funnel, and the lower layer is discarded. The upper layer is washed with 1.0 M magnesium sulfate twice and brine solution, dried over sodium sulfate, and concentrated to dryness. The residue is purified on a silica gel column chromatography eluting with hexane-EtOAc (4:1) to afford the desired compound A1.

A1-A7 are prepared using General Method A as shown in the table below:

| Starting Material | bis(pinacolato)diboron | A |
|---|---|---|
| 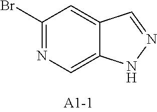 | 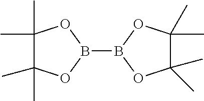 | 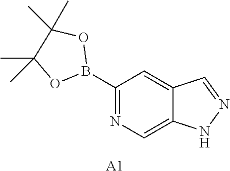 |
| 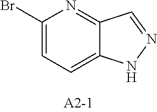 | 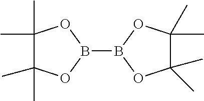 | 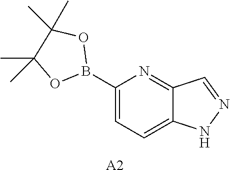 |
| 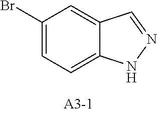 | 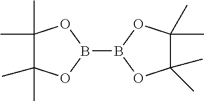 | 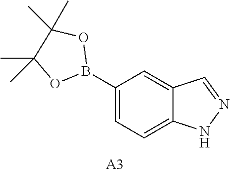 |
| 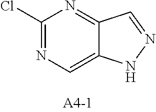 | 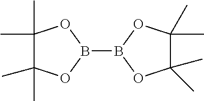 | 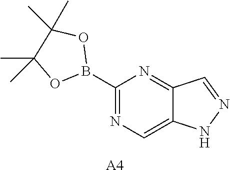 |
| 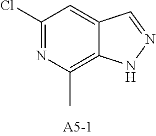 | 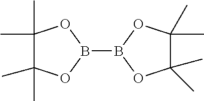 | 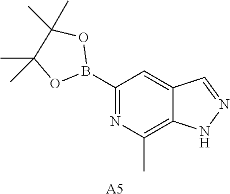 |
| 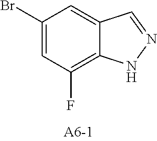 [SMILES: Fc1cc(Br)cc2cn[nH]c12] | 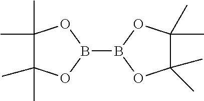 | 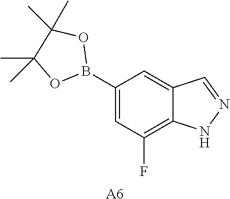 |
| 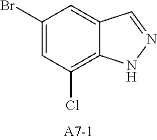 [SMILES: Clc1cc(Br)cc2cn[nH]c12] | 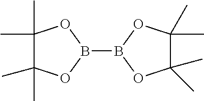 | 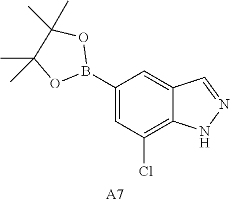 |
| 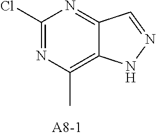 | 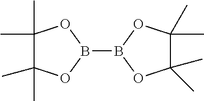 | 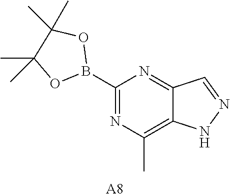 |

General Method B

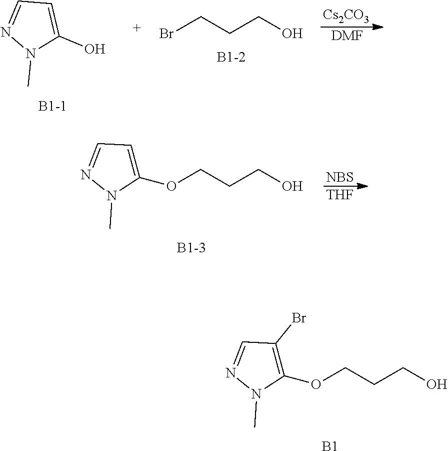

Step 1: A suspension of B1-1 (1.0 eq.), CS₂CO₃ or K₂CO₃ (1.5 eq.) and alkyl halide B1-2 (1.2 eq.) in anhydrous DMF (0.2 M) is stirred under N₂ at 60° C. until completion. The reaction mixture is then diluted with EtOAc and washed twice with HCl (aq) (1 M) and then brine, dried over MgSO₄, concentrated under vacuum, and purified on a silica gel column to provide B1-3.

Step 2. To a solution of B1-3 (1.0 eq.) in dry acetonitrile (0.25 M) is added N-bromosuccinimide (1.05 eq.) and the solution is stirred at ambient temperature until the reaction is completed. The reaction is quenched with aqueous sodium thiosulfate (0.1N) and the acetonitrile is then removed under vacuum. The residue is dissolved in water and extracted with ethyl acetate. The combined extracts are washed with water and brine, and then dried over magnesium sulfate. After filtration and condensation, the crude product is purified on a silica gel column to provide pure product B1.

B1-B14 are prepared using the General Method B or only Step 1 in General Method B:

| Starting material | Alkyl halide | B |
|---|---|---|
| 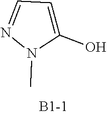 | 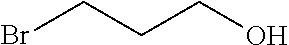 | 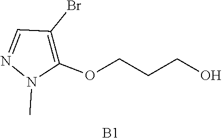 |
| 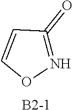 | 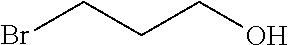 | 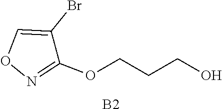 |
| 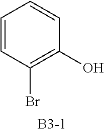 | 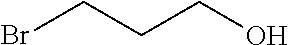 | 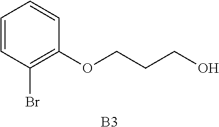 |
| 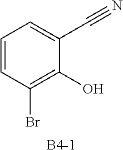 | 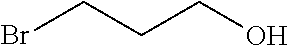 | 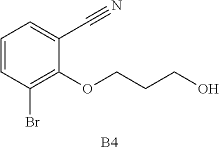 |
| 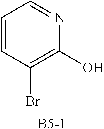 | 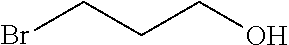 | 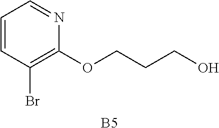 |
| 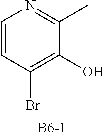 | 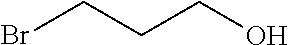 | 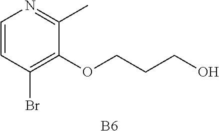 |
| 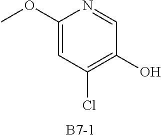 | 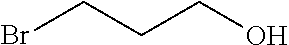 | 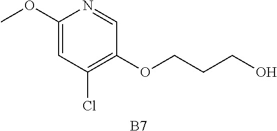 |
| 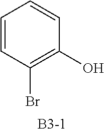 | 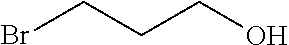 | 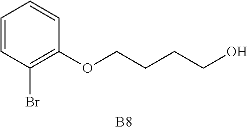 |
| 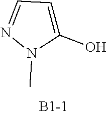 | 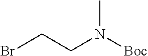 | 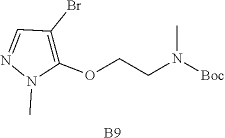 |
| 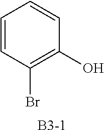 | 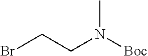 | 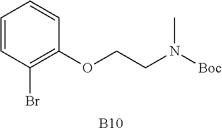 |
| 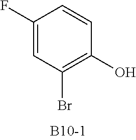 | 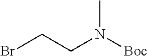 | 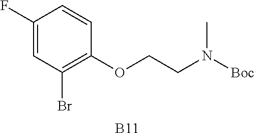 |
| 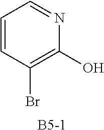 | 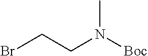 | 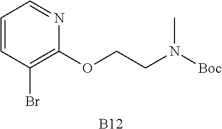 |
| 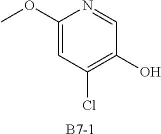 | 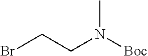 | 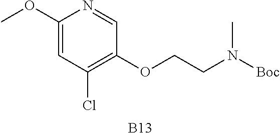 |
| 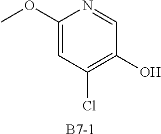 | 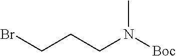 | 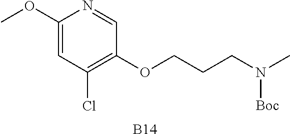 |

General Method C

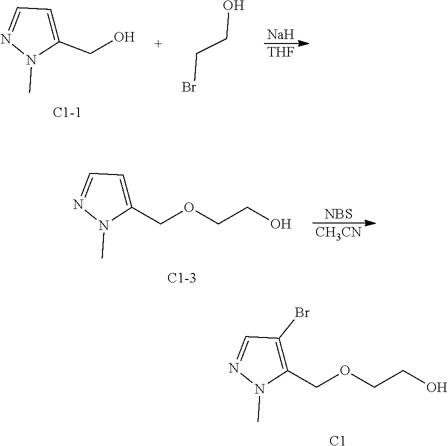

Step 1. C1-1 (1.0 eq.) is added to a suspension of NaH (60% in mineral oil, 1.1 eq.) in THF (0.5 M) at ambient temperature. After 30 min, to above suspension is added alkyl halide C1-2 (1.0 eq). After the reaction is complete, the reaction is quenched with saturated aqueous ammonium chloride solution and extracted with EtOAc for three times. The combined extracts are washed with brine, dried over Na₂SO₄, filtered, concentrated, and purified on a silica gel column to provide C1-3.

Step 2: To a solution of C1-3 (1.0 eq.) in dry acetonitrile (0.25 M) is added N-bromosuccinimide (1.05 eq.) and the solution is stirred at ambient temperature until the reaction is completed. The reaction is quenched with aqueous sodium thiosulfate (0.1N) and the acetonitrile is then removed under vacuum. The residue is dissolved in water and extracted with ethyl acetate. The combined extracts are washed with water and brine, and then dried over magnesium sulfate. After filtration and condensation, the crude product is purified on a silica gel column to provide pure product C1.

C1 and C2 are prepared using General Method C:

| Starting material 1 | Starting Material 2 | C |
|---|---|---|
| 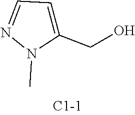 | 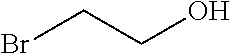 | 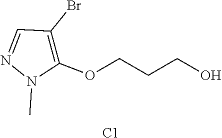 |
| 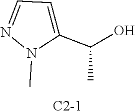 | 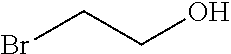 | 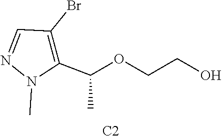 |

### Synthesis of tert-butyl [(4-bromo-1-methyl-1H-pyrazol-5-yl)methyl][(2S)-2-hydroxypropyl]carbamate (C3)

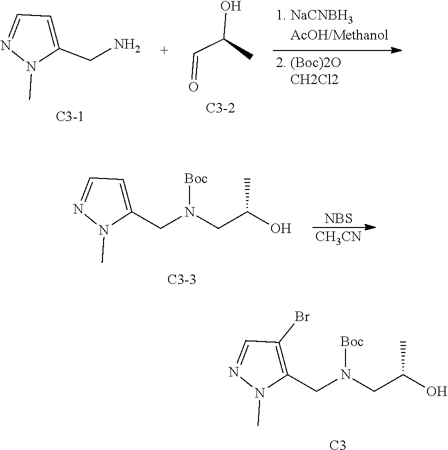

Step 1. To a solution of C3-1 (1 eq.) in methanol (0.2 M) and acetic acid (1.5 eq.) are added C3-2 (1 eq.) and NaCNBH₃ (2 eq.) at ambient temperature. The mixture is stirred for 1 hour and partitioned between water and ethyl acetate. The organic phase layer is separated, washed sequentially with saturated NaHCO₃ and brine, concentrated and dried under vacuum. The residue is dissolved in CH₂Cl₂ (0.2 M) and the solution is cooled to 0° C. To the solution is added di(tert-butyl) dicarbonate (1.2 eq) portionwise. The ice bath is removed, and the mixture is stirred for overnight at ambient temperature. The reaction solution is diluted with dichloromethane, washed with water, and dried over magnesium sulfate. After filtration and condensation, the residue is purified on a silica gel column to provide C₃₋₃.

Step 2. C3-3 is converted to C3 using the step 2 procedure in General Method C.

### Synthesis of 5-bromo-N-(2-hydroxyethyl)-1-methyl-1H-pyrazole-4-carboxamide (C₄)

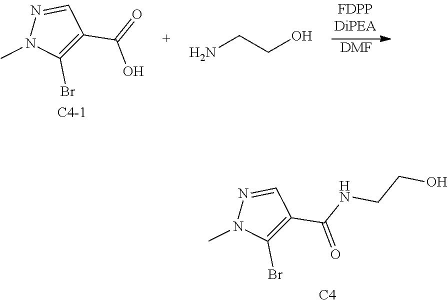

To a solution of C4-1 (1 eq.) and hydroxyethyl amine (1.1 eq.) in DMF (0.2 M) are added DIPEA (3 eq.) and pentafluorophenyl diphenylphosphinate (FDPP) (1.1 eq). The solution is stirred at ambient temperature until the amide formation is completed. The mixture is diluted with water and extracted with EtOAc for three times. The combined extracts are washed with water for three times, aqueous HCl (1N), saturated aqueous Na₂CO₃ and brine, dried over Na₂SO₄, and concentrated. The resulting residue is purified by a silica gel column to afford compound C4.

### Synthesis of tert-butyl [3-(4-chloro-6-methoxypyridin-3-yl)propyl]methylcarbamate (C5)

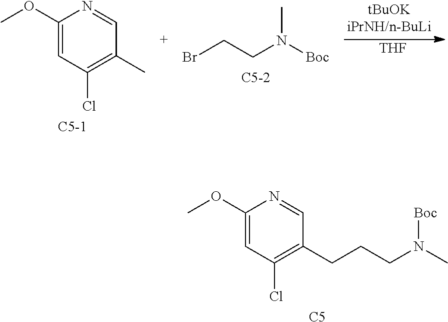

A solution of potassium tert-butoxide (3.75 eq.) and diisopropylamine (3.75 eq.) in anhydrous THF (0.8 M) is cooled to −78° C. under argon, n-butyllithium in hexane (1.6 M, 3.00 eq.) is added to the solution over 25 minutes. After 15 minutes of stirring at −78° C., a solution of compound C5-1 (1.00 eq.) in anhydrous THF (1.0 M) is added to the mixture over 15 minutes at −78° C. The mixture is stirred an additional 20 minutes at −78° C. Alkyl halide C₅-2 (1.2 eq.) is added in one portion at −78° C. After 10 minutes of stirring at −78° C., the mixture is poured into saturated aqueous NH₄Cl. THF is removed from the mixture in vacuo, extracted with ethyl acetate for three times. The combined organic layers are washed with saturated brine and dried over anhydrous Na₂SO₄. Filtration, evaporation of the solvent and purification on a silica gel column provide C5.

General Method D

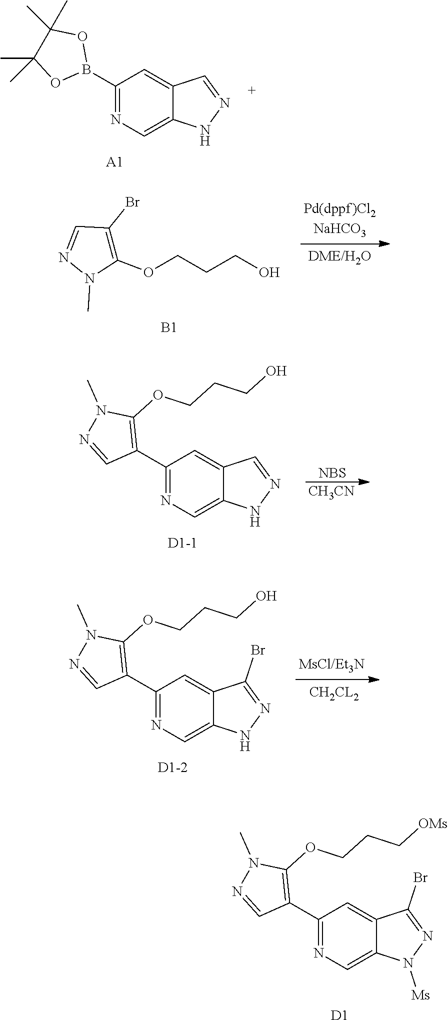

Step 1. To a solution of A1 (1.0 eq.) and B1 (1.2 eq.) and Cs₂CO₃ (3 eq.) in DME/H₂O (5:1, 0.2 M) under N₂, is added Pd(dppf)Cl₂ (0.05 eq.). The mixture is stirred at 85° C. overnight, cooled to ambient temperature, and quenched with H₂O. The resulting mixture is extracted with EtOAc for three times. The combined extracts are washed with brine and dried over anhydrous Na₂SO₄. After filtration and condensation, the resulting residue is purified by silica gel chromatography to afford the desired product D1-1.

Step 2. To a solution of D1-1 (1.0 eq.) in dry acetonitrile (0.25 M) is added N-bromosuccinimide (1.05 eq.) and the solution is stirred at ambient temperature until the reaction is completed. The reaction is quenched with aqueous sodium thiosulfate (0.1N) and the acetonitrile is then removed under vacuum. The residue is dissolved in water and extracted with ethyl acetate. The combined extracts are washed with water and brine, and then dried over magnesium sulfate. After filtration and condensation, the crude product was purified on a silica gel column to provide pure product D1-2.

Step 3. To a solution of D1-2 (1.0 eq.) and triethylamine (2.2 eq.) in dichloromethane (0.25 M) is added methanesulfonyl chloride (MsCl, 2.1 eq.) and the solution is stirred at ambient temperature until the reaction is completed. The reaction is quenched with water and extracted with ethyl acetate. The combined extracts are washed with water and brine, and then dried over magnesium sulfate. After filtration and condensation, the crude product is purified on a silica gel column to provide pure product D1.

D1-D29 are prepared via the General Method D using the corresponding two starting materials A and B or C or other commercially available starting materials as shown in the table below:

| Starting Material 1 | Starting Material 2 | D |
|---|---|---|
| 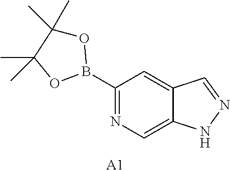 | 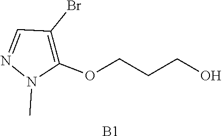 | 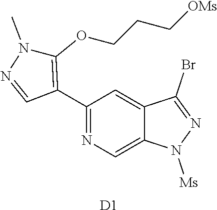 |
| 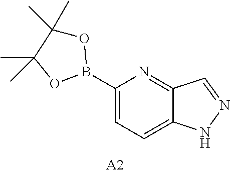 | 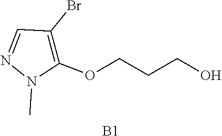 | 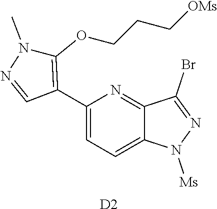 |
| 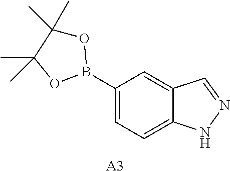 | 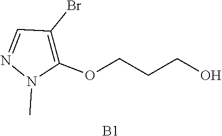 | 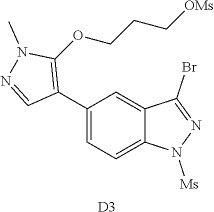 |
| 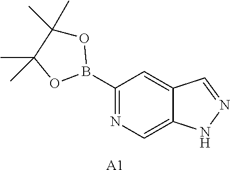 | 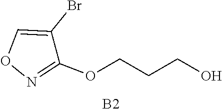 | 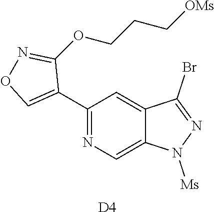 |
| 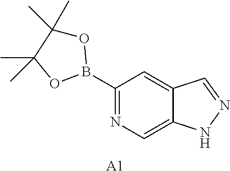 | 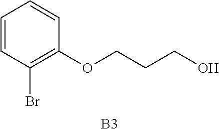 [SMILES: OCCCOc1ccccc1Br ; "B3"] | 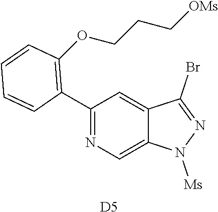 |
| 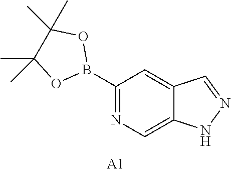 | 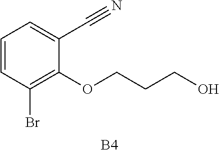 | 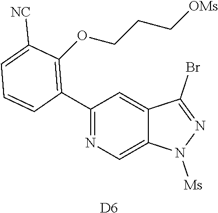 |
| 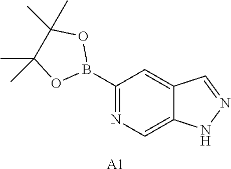 | 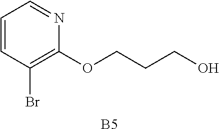 | 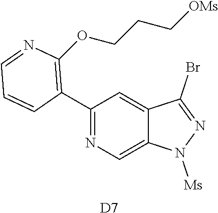 |
| 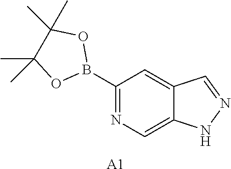 | 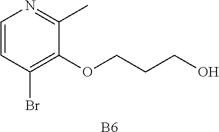 | 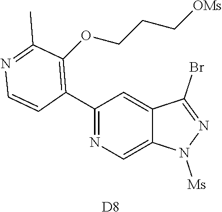 |
| 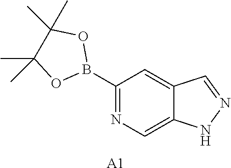 | 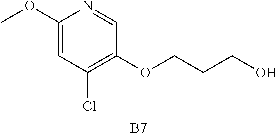 | 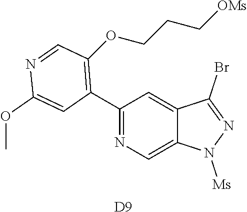 |
| 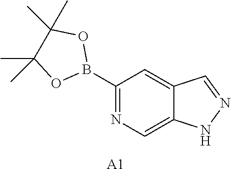 | 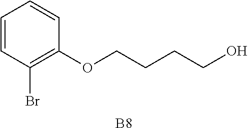 | 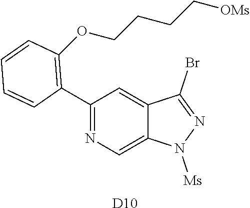 |
| 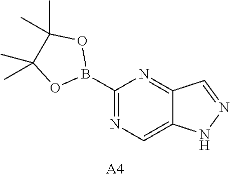 | 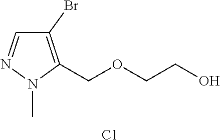 | 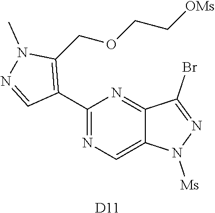 |
| 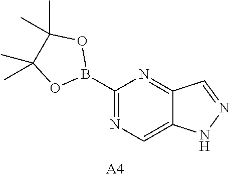 | 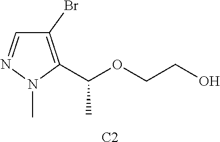 | 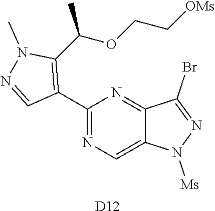 |
| 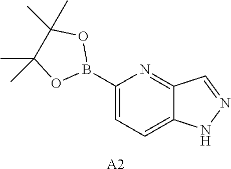 | 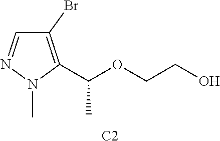 | 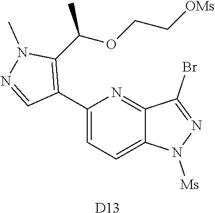 |
| 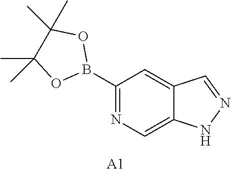 | 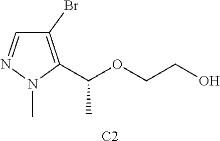 | 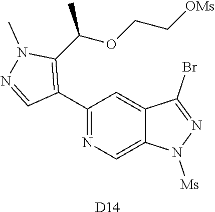 |
| 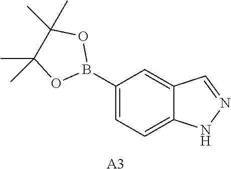 | 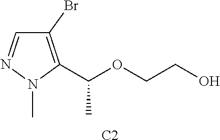 | 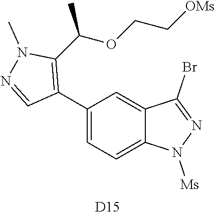 |
| 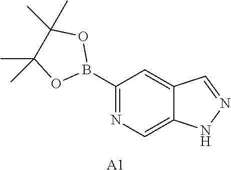 | 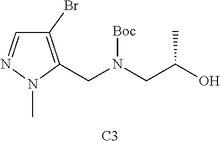 | 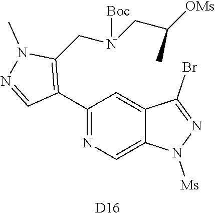 |
| 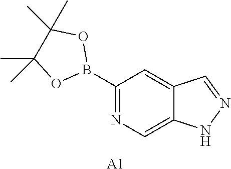 | 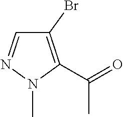 | 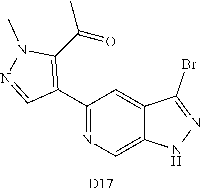 |
| 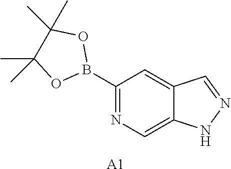 | 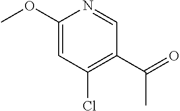 | 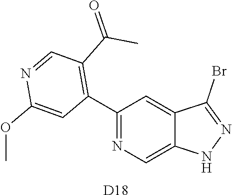 |
| 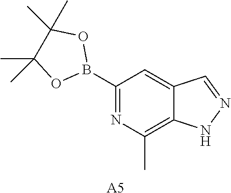 | 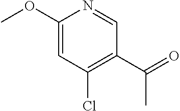 | 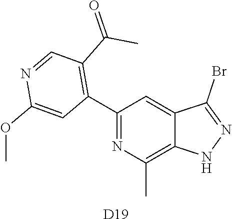 |
| 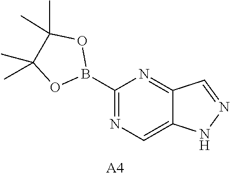 | 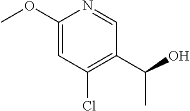 | 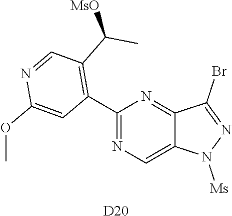 |
| 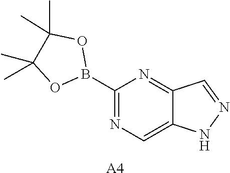 | 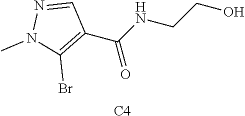 | 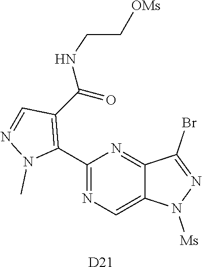 |
| 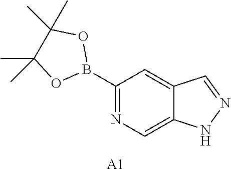 | 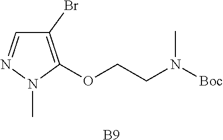 | 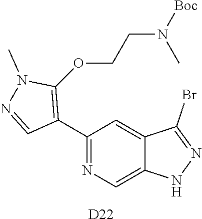 |
| 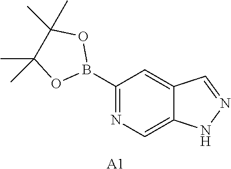 | 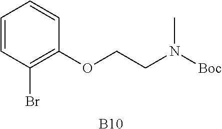 | 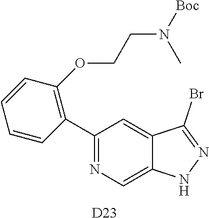 |
| 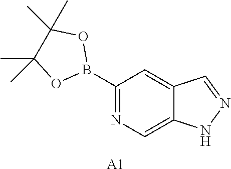 | 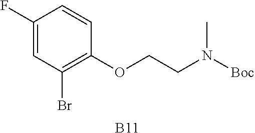 | 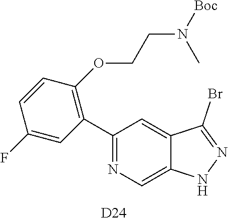 |
| 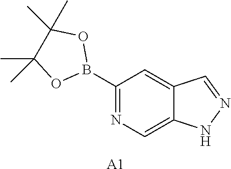 | 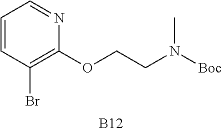 | 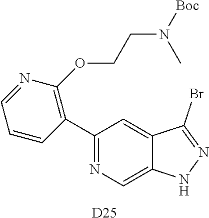 |
| 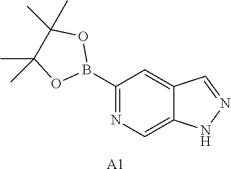 | 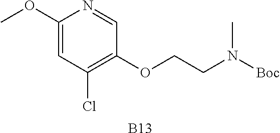 | 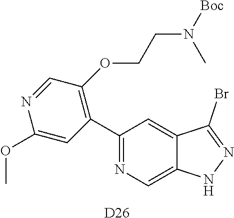 |
| 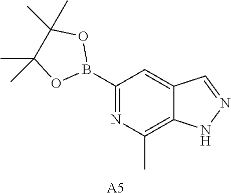 | 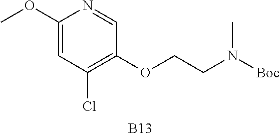 | 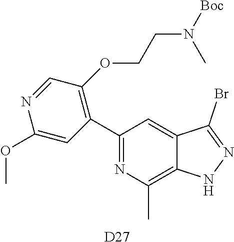 |
| 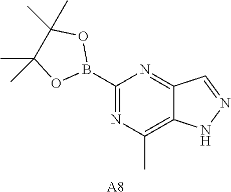 | 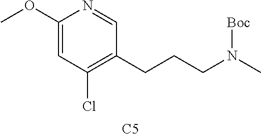 | 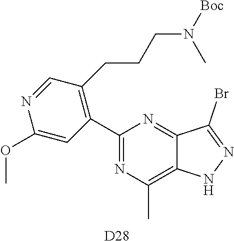 |
| 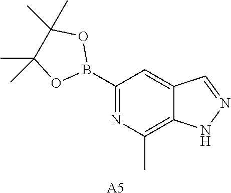 | 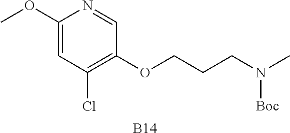 | 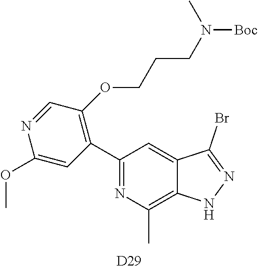 |

General Method E

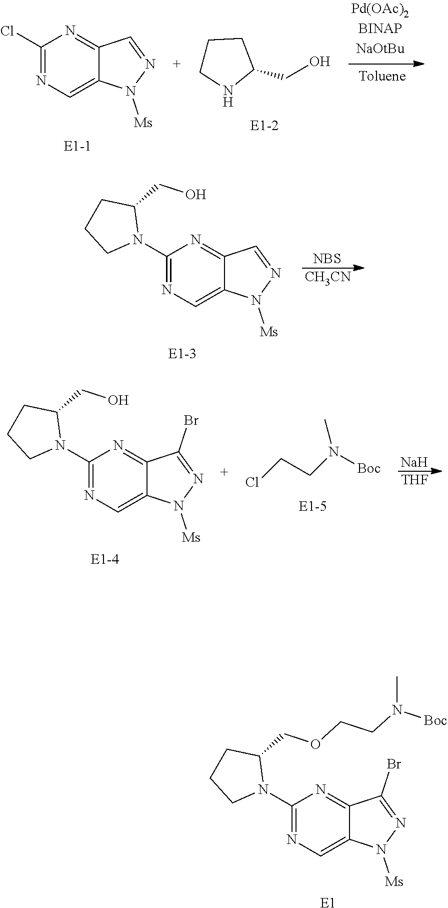

Step 1. To a stirring solution of E1-1 (1.0 eq.) in toluene (0.2 M) are added E1-2 (1.5 eq.) and sodium tert-butoxide (3 eq.), BINAP (0.05 eq.) and Pd(OAc)₂ (0.05) under nitrogen. The mixture is heated at 85° C. for 20 h and cooled to ambient temperature. The reaction is quenched with sat. aqueous ammonium chloride and extracted with EtOAc. The combined extracts are washed with brine and dried over Na₂SO₄. After filtration and concentration, the residue is purified on a silica gel column to provide E1-3.

Step 2. To a solution of E1-3 (1.0 eq.) in dry acetonitrile (0.25 M) is added N-bromosuccinimide (1.05 eq.) and the solution is stirred at ambient temperature until the reaction is completed. The reaction is quenched with aqueous sodium thiosulfate (0.1N) and the acetonitrile is then removed under vacuum. The residue is dissolved in water and extracted with ethyl acetate. The combined extracts are washed with water and brine, and then dried over magnesium sulfate. After filtration and condensation, the crude product was purified on a silica gel column to provide pure product E1-4.

Step 3. E1-4 (1.0 eq.) is added to a suspension of NaH (60% in mineral oil, 1.1 eq.) in THF (0.5 M) at ambient temperature. After 30 min, to above suspension is added E1-5 (1.0 eq). After the reaction is complete, the reaction is quenched with saturated aqueous ammonium chloride solution and extracted with EtOAc for three times. The combined extracts are washed with brine, dried over Na₂SO₄, filtered, concentrated, and purified on a silica gel column to provide E1.

E1-E4 are prepared via General Method E as shown below:

| Starting Material 1 | Starting Material 2 | E |
|---|---|---|
| 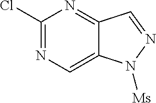 [SMILES: CS(=O)(=O)n1ncc2nc(Cl)ncc21] | 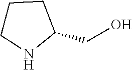 | 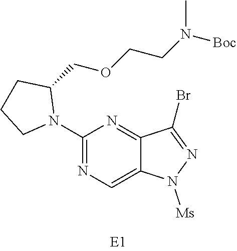 |
| 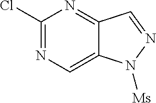 [SMILES: CS(=O)(=O)n1ncc2nc(Cl)ncc21] | 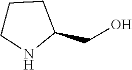 | 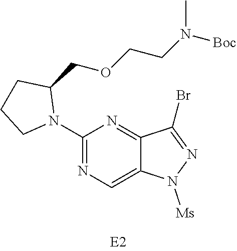 |
| 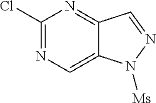 [SMILES: CS(=O)(=O)n1ncc2nc(Cl)ncc21] | 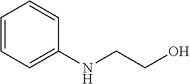 | 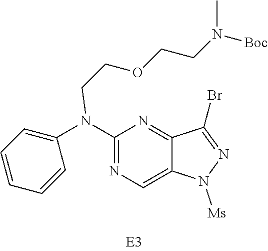 |
| 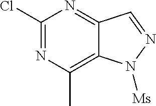 | 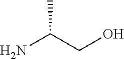 | 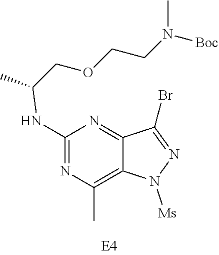 |

General Method F

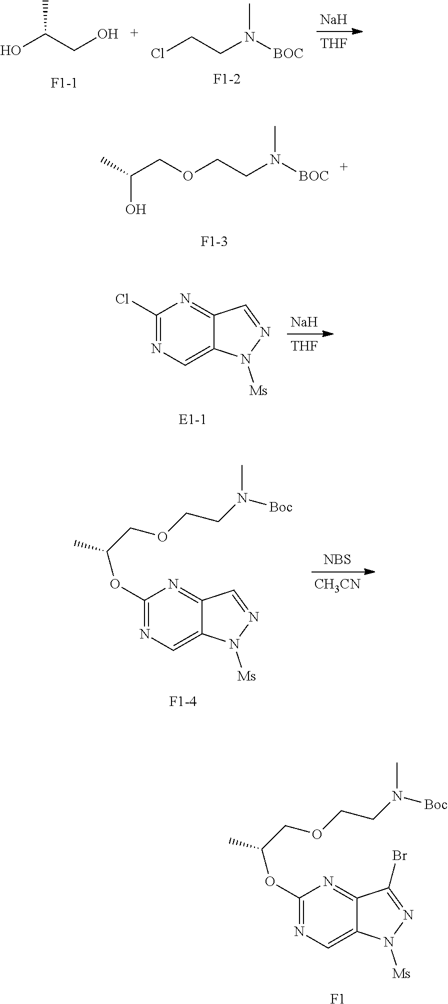

Step 1. F1-1 (1.0 eq.) is added to a suspension of NaH (60% in mineral oil, 1.0 eq.) in THF (0.5 M) at ambient temperature. After 30 min, to above suspension is added F1-2 (1.0 eq). After the reaction is complete, the reaction is quenched with saturated aqueous ammonium chloride solution and extracted with EtOAc for three times. The combined extracts are washed with brine, dried over Na₂SO₄, filtered, concentrated, and purified on a silica gel column to provide F1-3.

Step 2. F1-3 (1.0 eq.) is added to a suspension of NaH (60% in mineral oil, 1.1 eq.) in THF (0.5 M) at ambient temperature. After 30 min, to above suspension is added E1-1 (1.0 eq). The reaction is heated at 60° C. under nitrogen. After the reaction is complete, the reaction is cooled down and quenched with saturated aqueous ammonium chloride solution and extracted with EtOAc for three times. The combined extracts are washed with brine, dried over Na₂SO₄, filtered, concentrated, and purified on a silica gel column to provide F1-4.

Step 3. To a solution of F1-4 (1.0 eq.) in dry acetonitrile (0.25 M) is added N-bromosuccinimide (1.05 eq.) and the solution is stirred at ambient temperature until the reaction is completed. The reaction is quenched with aqueous sodium thiosulfate (0.1N) and the acetonitrile is then removed under vacuum. The residue is dissolved in water and extracted with ethyl acetate. The combined extracts are washed with water and brine, and then dried over magnesium sulfate. After filtration and condensation, the crude product was purified on a silica gel column to provide pure product F1.

F1-F4 are prepared using General Method F as shown below:

| Starting Material 1 | Starting Material 2 | F |
|---|---|---|
| 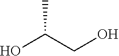 | 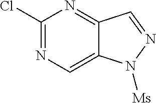 [SMILES: CS(=O)(=O)n1ncc2nc(Cl)ncc21] | 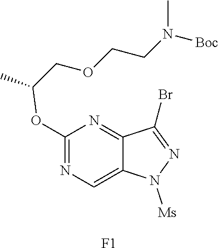 |
| 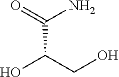 | 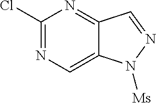 [SMILES: CS(=O)(=O)n1ncc2nc(Cl)ncc21] | 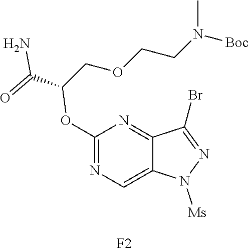 |
| 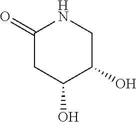 | 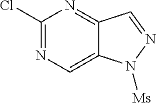 [SMILES: CS(=O)(=O)n1ncc2nc(Cl)ncc21] | 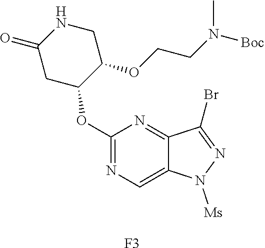 |
| 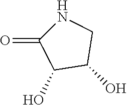 | 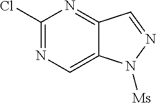 [SMILES: CS(=O)(=O)n1ncc2nc(Cl)ncc21] | 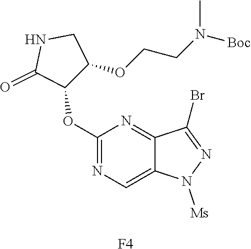 |

General Method H

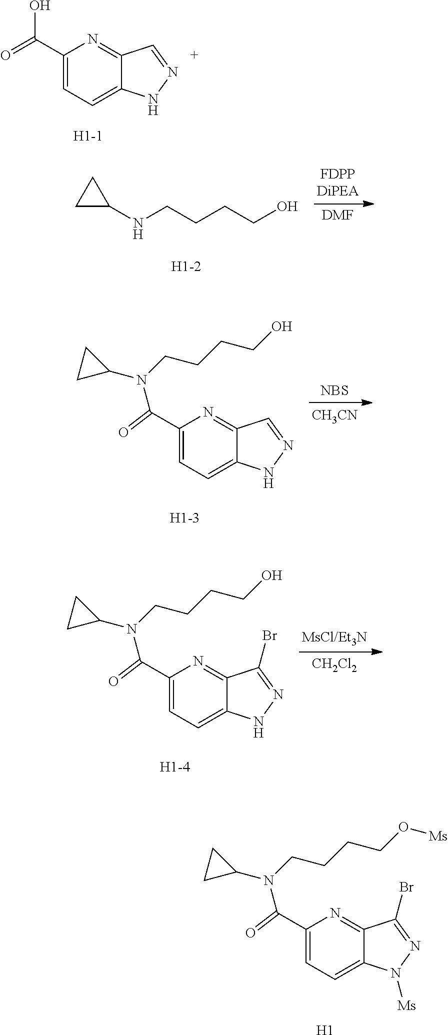

Step 1. To a solution of H1-1 (1.0 eq.) and H1-2 (1.0 eq.) in DMF (0.2 M) are added diisopropylethylamine (DiPEA, 3 eq.) and pentafluorophenyl diphenylphosphinate (FDPP) (1.1 eq). The solution is stirred at ambient temperature until the amide formation is completed. The mixture is diluted with water and extracted with EtOAc for three times. The combined extracts are washed with water for three times, aqueous HCl (1N), saturated aqueous Na₂CO₃ and brine, dried over Na₂SO₄, and concentrated. The resulting residue is purified by a silica gel column to afford H1-3.

Step 2. To a solution of H1-3 (1.0 eq.) in dry acetonitrile (0.25 M) is added N-bromosuccinimide (1.05 eq.) and the solution is stirred at ambient temperature until the reaction is completed. The reaction is quenched with aqueous sodium thiosulfate (0.1N) and the acetonitrile is then removed under vacuum. The residue is dissolved in water and extracted with ethyl acetate. The combined extracts are washed with water and brine, and then dried over magnesium sulfate. After filtration and condensation, the crude product was purified on a silica gel column to provide pure product H1-4.

Step 3. To a solution of H1-4 (1.0 eq.) and triethylamine (2.2 eq.) in dichloromethane (0.25 M) is added methanesulfonyl chloride (MsCl, 2.1 eq.) and the solution is stirred at ambient temperature until the reaction is completed. The reaction is quenched with water and extracted with ethyl acetate. The combined extracts are washed with water and brine, and then dried over magnesium sulfate. After filtration and condensation, the crude product is purified on a silica gel column to provide pure product H1.

H1-H5 are prepared using General Method H.

| Starting Material 1 | Starting Material 2 | H |
|---|---|---|
| 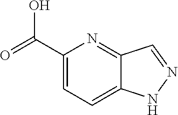 [SMILES: O=C(O)c1ccc2[nH]ncc2n1] | 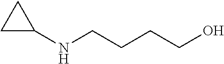 | 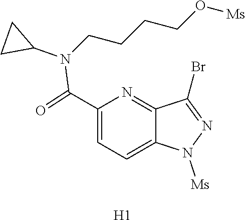 |
| 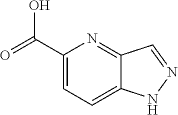 [SMILES: O=C(O)c1ccc2[nH]ncc2n1] | 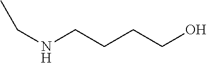 | 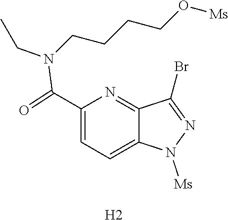 |
| 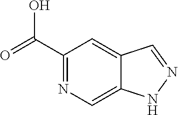 [SMILES: O=C(O)c1cc2cn[nH]c2cn1] | 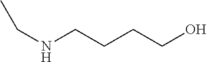 | 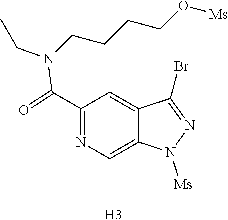 |
| 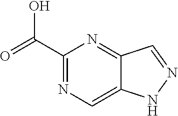 [SMILES: O=C(O)c1ncc2[nH]ncc2n1] | 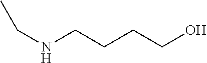 | 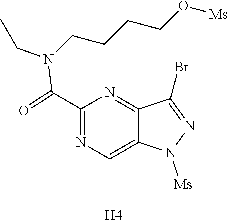 |
| 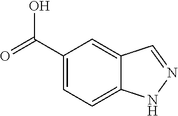 [SMILES: O=C(O)c1ccc2[nH]ncc2c1] | 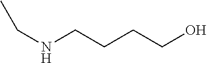 | 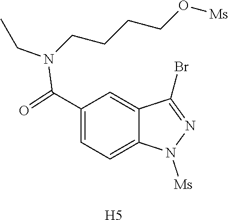 |

General Method I

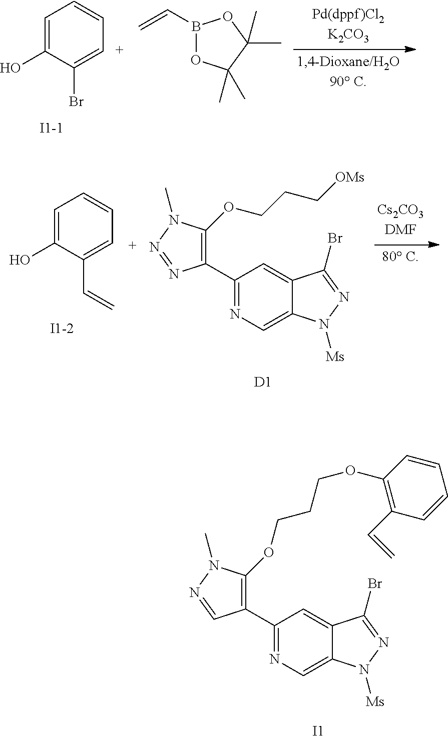

Step 1. To a solution of I1-1 (1.00 eq.) and vinylboronic acid pinacol ester (1.2 eq.) in 1,4-dioxane (0.5 M), is added potassium carbonate (2.00 eq.) in water (2 M) and the nitrogen gas is bubbled through the solution for 15 minutes. Pd(dppf}Cl₂-DCM (0.05) is then added to the solution. The reaction is stirred at 100° C. under nitrogen for 18 h. The solution is cooled, diluted with water, and extracted with ethyl acetate three times. The combined organic solution is then washed with 1 N aqueous NaOH solution and brine, and dried over anhydrous magnesium sulfate. The organic solution is then concentrated in vacuo. The crude product is purified on silica gel column to provide I1-2.

Step 2. To a solution of I1-2 (1.0 eq.) in DMF (0.2 M) are added cesium carbonate (3.0 eq.) and D1 (1.00 eq.). The reaction mixture is heated to reflux at 80° C. for 18 h. The solution is concentrated in vacuo, re-dissolved in ethyl acetate, and washed with water and brine. The organic layer is then dried over anhydrous magnesium sulfate and concentrated in vacuo. The crude product is purified on silica gel column to afford I1.

I1-I30 are prepared using General Method I:

| Starting Material 1 | Starting Material 2 | I |
|---|---|---|
| 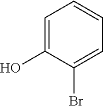 | 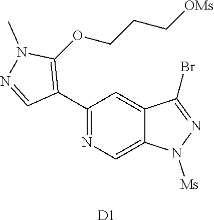 [SMILES: Cn1ncc(-c2cc3c(Br)nn(S(C)(=O)=O)c3cn2)c1OCCCOS(C)(=O)=O ; "D1"] | 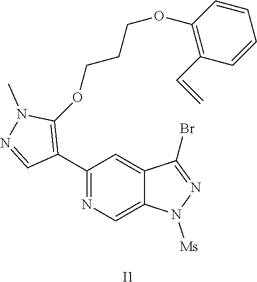 |
| 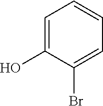 | 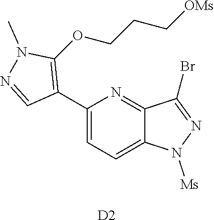 [SMILES: Cn1ncc(-c2ccc3c(n2)c(Br)nn3S(C)(=O)=O)c1OCCCOS(C)(=O)=O ; "D2"] | 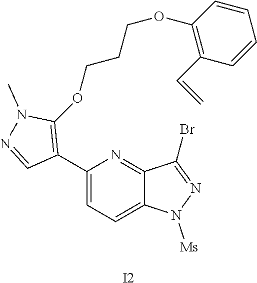 |
| 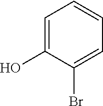 | 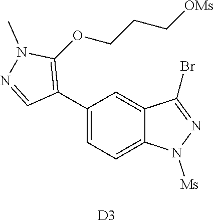 [SMILES: Cn1ncc(-c2ccc3c(c2)c(Br)nn3S(C)(=O)=O)c1OCCCOS(C)(=O)=O ; "D3"] | 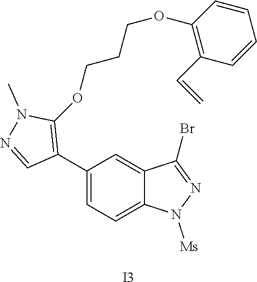 |
| 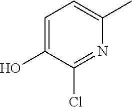 | 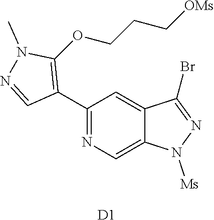 [SMILES: Cn1ncc(-c2cc3c(Br)nn(S(C)(=O)=O)c3cn2)c1OCCCOS(C)(=O)=O ; "D1"] | 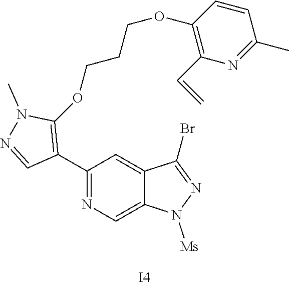 |
| 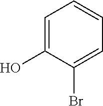 | 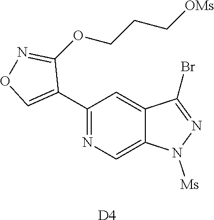 [SMILES: CS(=O)(=O)OCCCOc1nocc1-c1cc2c(Br)nn(S(C)(=O)=O)c2cn1 ; "D4"] | 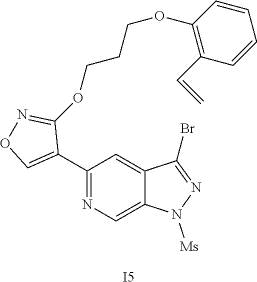 |
| 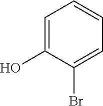 | 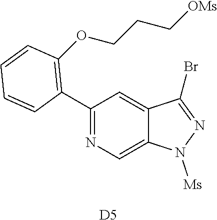 [SMILES: CS(=O)(=O)OCCCOc1ccccc1-c1cc2c(Br)nn(S(C)(=O)=O)c2cn1 ; "D5"] | 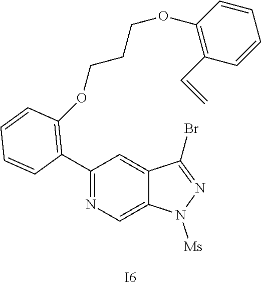 |
| 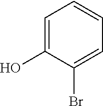 | 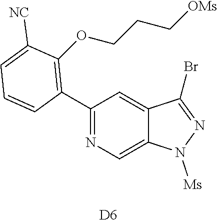 [SMILES: CS(=O)(=O)OCCCOc1c(C#N)cccc1-c1cc2c(Br)nn(S(C)(=O)=O)c2cn1 ; "D6"] | 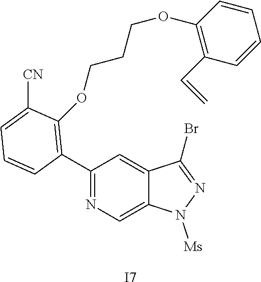 |
| 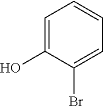 | 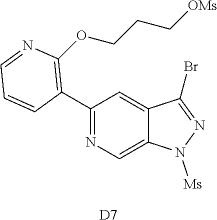 [SMILES: CS(=O)(=O)OCCCOc1ncccc1-c1cc2c(Br)nn(S(C)(=O)=O)c2cn1 ; "D7"] | 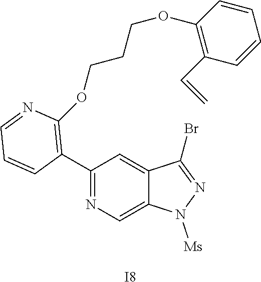 |
| 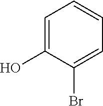 | 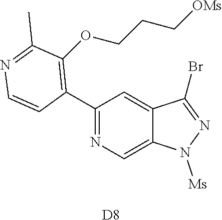 [SMILES: Cc1nccc(-c2cc3c(Br)nn(S(C)(=O)=O)c3cn2)c1OCCCOS(C)(=O)=O ; "D8"] | 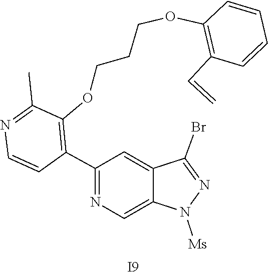 |
| 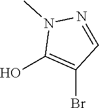 | 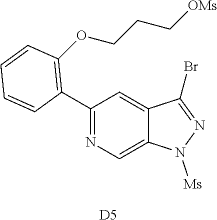 [SMILES: CS(=O)(=O)OCCCOc1ccccc1-c1cc2c(Br)nn(S(C)(=O)=O)c2cn1 ; "D5"] | 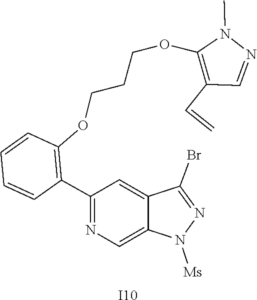 |
| 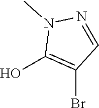 | 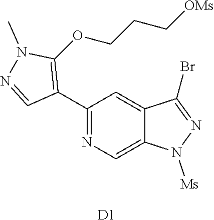 [SMILES: Cn1ncc(-c2cc3c(Br)nn(S(C)(=O)=O)c3cn2)c1OCCCOS(C)(=O)=O ; "D1"] | 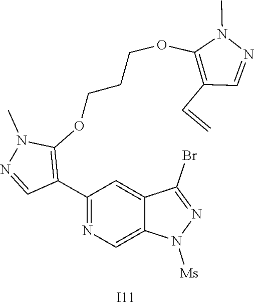 |
| 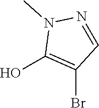 | 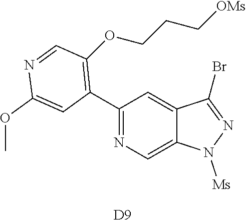 [SMILES: COc1cc(-c2cc3c(Br)nn(S(C)(=O)=O)c3cn2)c(OCCCOS(C)(=O)=O)cn1 ; "D9"] | 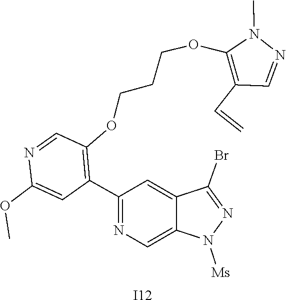 |
| 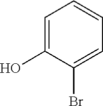 | 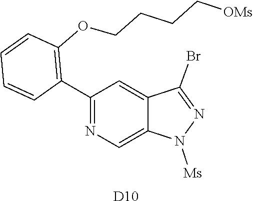 [SMILES: CS(=O)(=O)OCCCCOc1ccccc1-c1cc2c(Br)nn(S(C)(=O)=O)c2cn1 ; "D10"] | 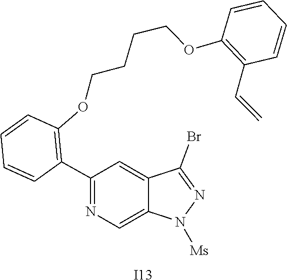 |
| 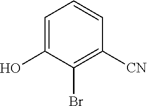 | 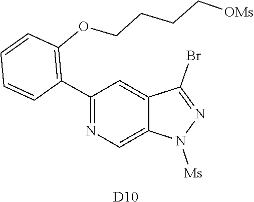 [SMILES: CS(=O)(=O)OCCCCOc1ccccc1-c1cc2c(Br)nn(S(C)(=O)=O)c2cn1 ; "D10"] | 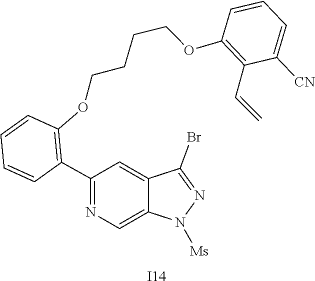 |
| 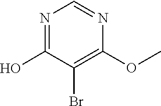 | 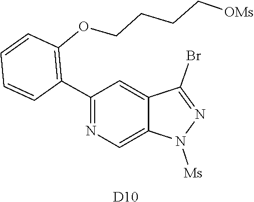 [SMILES: CS(=O)(=O)OCCCCOc1ccccc1-c1cc2c(Br)nn(S(C)(=O)=O)c2cn1 ; "D10"] | 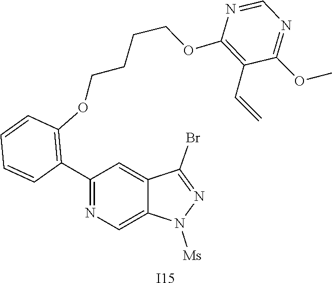 |
| 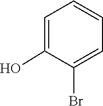 | 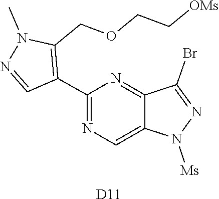 [SMILES: Cn1ncc(-c2ncc3c(n2)c(Br)nn3S(C)(=O)=O)c1COCCOS(C)(=O)=O ; "D11"] | 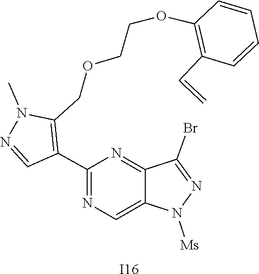 |
| 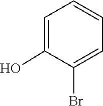 | 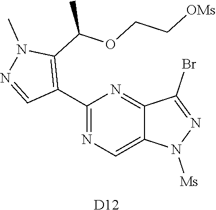 [SMILES: C[C@@H](OCCOS(C)(=O)=O)c1c(-c2ncc3c(n2)c(Br)nn3S(C)(=O)=O)cnn1C ; "D12"] | 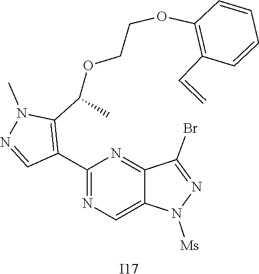 |
| 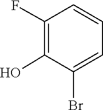 | 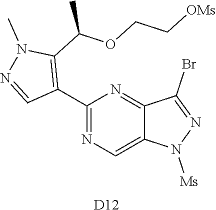 [SMILES: C[C@@H](OCCOS(C)(=O)=O)c1c(-c2ncc3c(n2)c(Br)nn3S(C)(=O)=O)cnn1C ; "D12"] | 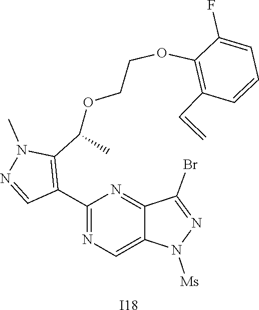 |
| 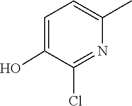 | 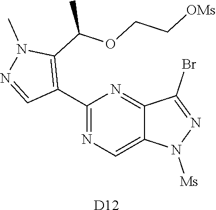 [SMILES: C[C@@H](OCCOS(C)(=O)=O)c1c(-c2ncc3c(n2)c(Br)nn3S(C)(=O)=O)cnn1C ; "D12"] | 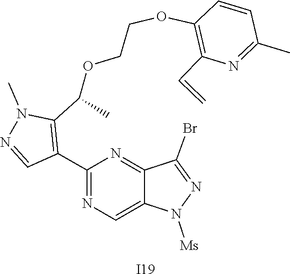 |
| 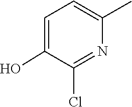 | 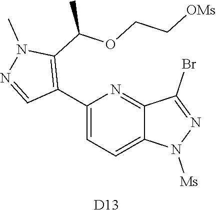 | 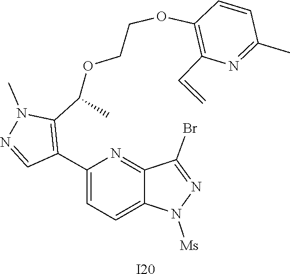 |
| 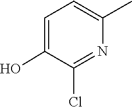 | 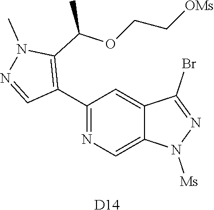 | 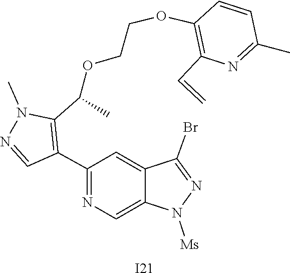 |
| 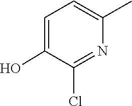 | 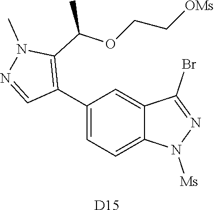 | 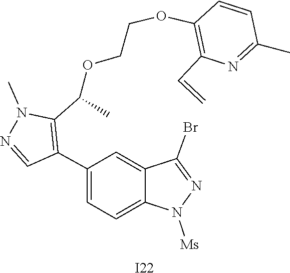 |
| 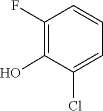 | 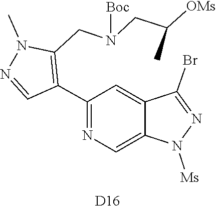 [SMILES: C[C@@H](CN(Cc1c(-c2cc3c(Br)nn(S(C)(=O)=O)c3cn2)cnn1C)C(=O)OC(C)(C)C)OS(C)(=O)=O ; "D16"] | 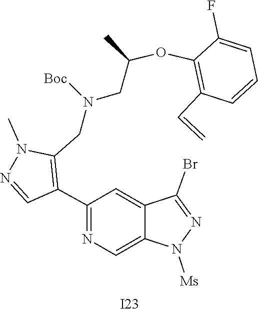 |
| | 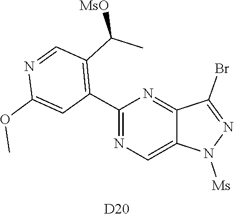 [SMILES: COc1cc(-c2ncc3c(n2)c(Br)nn3S(C)(=O)=O)c([C@H](C)OS(C)(=O)=O)cn1 ; "D20"] | 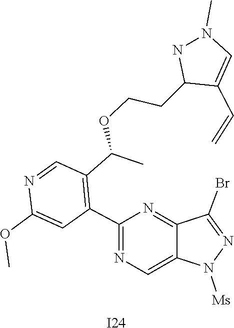 |
| 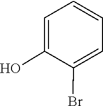 | 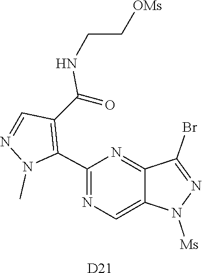 [SMILES: Cn1ncc(C(=O)NCCOS(C)(=O)=O)c1-c1ncc2c(n1)c(Br)nn2S(C)(=O)=O ; "D21"] | 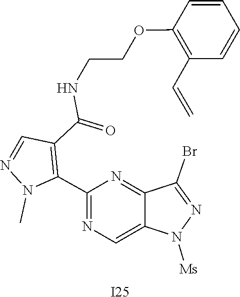 |
| 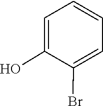 | 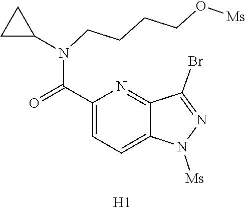 [SMILES: CS(=O)(=O)OCCCCN(C(=O)c1ccc2c(n1)c(Br)nn2S(C)(=O)=O)C1CC1 ; "H1"] | 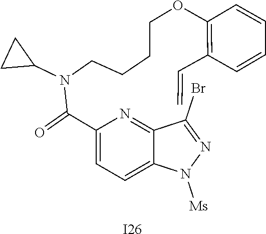 |
| 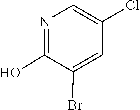 | 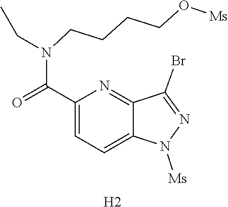 [SMILES: CCN(CCCCOS(C)(=O)=O)C(=O)c1ccc2c(n1)c(Br)nn2S(C)(=O)=O ; "H2"] | 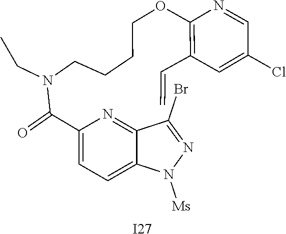 |
| 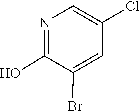 | 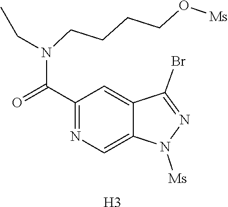 [SMILES: CCN(CCCCOS(C)(=O)=O)C(=O)c1cc2c(Br)nn(S(C)(=O)=O)c2cn1 ; "H3"] | 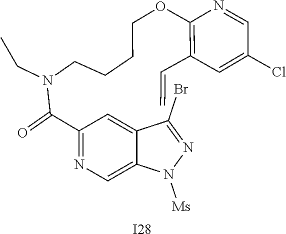 |
| 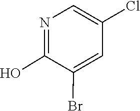 | 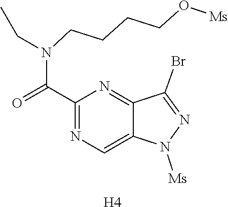 [SMILES: CCN(CCCCOS(C)(=O)=O)C(=O)c1ncc2c(n1)c(Br)nn2S(C)(=O)=O ; "H4"] | 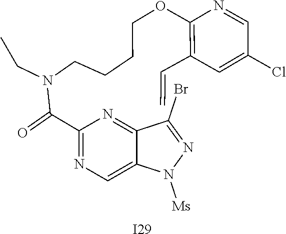 |
| 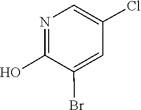 | 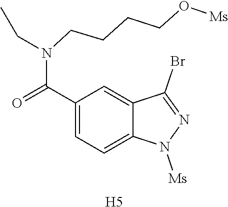 [SMILES: CCN(CCCCOS(C)(=O)=O)C(=O)c1ccc2c(c1)c(Br)nn2S(C)(=O)=O ; "H5"] | 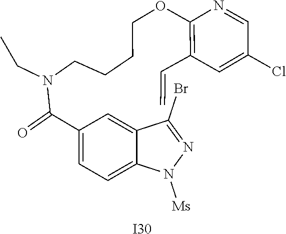 |

General Method J

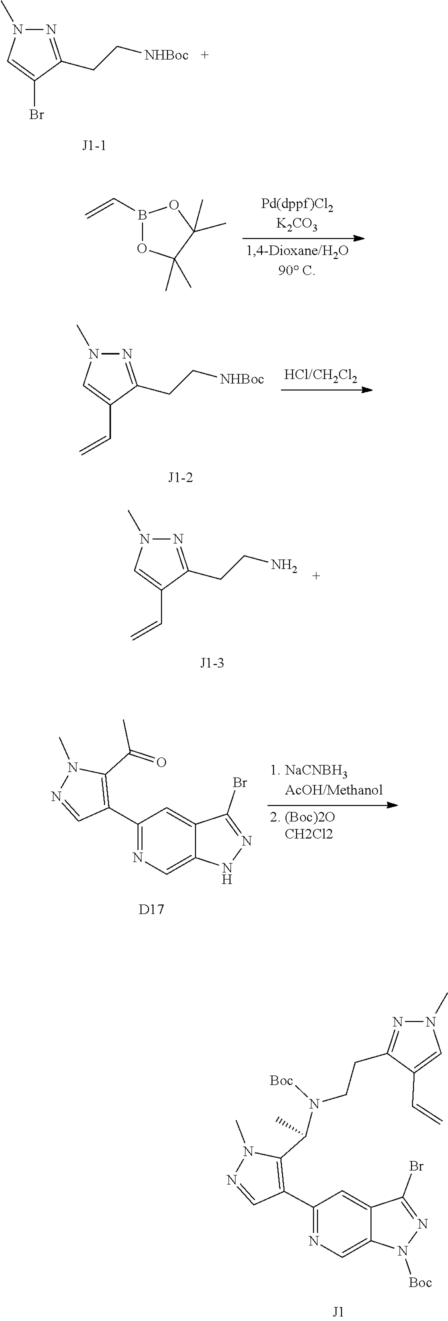

Step 1. To a solution of J1-1 (1.00 eq.) and vinylboronic acid pinacol ester (1.2 eq.) in 1,4-dioxane (0.5 M), is added potassium carbonate (2.00 eq.) in water (2 M) and the nitrogen gas is bubbled through the solution for 15 minutes. Pd(dppf}Cl2-DCM (0.05) is then added to the solution. The reaction is stirred at 100° C. under nitrogen for 18 h. The solution is cooled, diluted with water, and extracted with ethyl acetate three times. The combined organic solution is then washed with 1 N aqueous NaOH solution and brine, and dried over anhydrous magnesium sulfate. The organic solution is then concentrated in vacuo. The crude product is purified on silica gel column to provide J1-2.

Step 2. To a solution of J1-2 (1.00 eq.) in CH₂Cl₂(0.2 M) is added a solution of HCl in dioxane (4 eq HCl). The solution is stirred at 40° C. until the de-Boc is completed. The solvents are removed under rotavap. The residue is redissolved in ethyl acetate and washed with saturated aqueous Na₂CO₃ solution and dried over sodium sulfate. After filtration and condensation, J1-3 is obtained which is used for the next step without purification.

Step 3. To a solution of J1-3 (1 eq.) in methanol (0.2 M) and acetic acid (1.5 eq.) are added D17 (1 eq.) and NaCNBH₃ (2 eq.) at ambient temperature. The mixture is stirred for 1 hour and partitioned between water and ethyl acetate. The organic phase layer is separated, washed sequentially with saturated NaHCO₃ and brine, concentrated and dried under vacuum. The residue is dissolved in CH₂Cl₂ (0.2 M) and the solution is cooled to 0° C. To the solution is added di(tert-butyl) dicarbonate (2.2 eq) portionwise. The ice bath is removed, and the mixture is stirred for overnight at ambient temperature. The reaction solution is diluted with dichloromethane, washed with water, and dried over magnesium sulfate. After filtration and condensation, the residue is purified on a silica gel column to provide J1.

J1-J5 are prepared using General Method J.

| Starting Material 1 | Starting Material 2 | J |
|---|---|---|
| 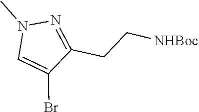 [SMILES: Cn1cc(Br)c(CCNC(=O)OC(C)(C)C)n1] | 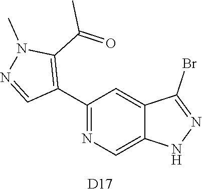 | 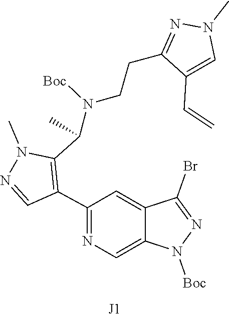 |
| 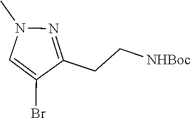 [SMILES: Cn1cc(Br)c(CCNC(=O)OC(C)(C)C)n1] | 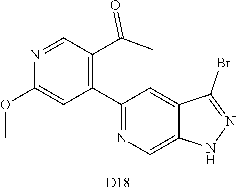 [SMILES: COc1cc(-c2cc3c(Br)n[nH]c3cn2)c(C(C)=O)cn1 ; "D18"] | 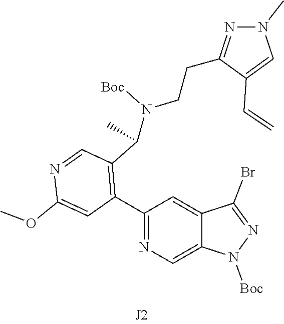 |
| 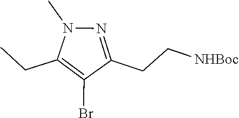 [SMILES: CCc1c(Br)c(CCNC(=O)OC(C)(C)C)nn1C] | 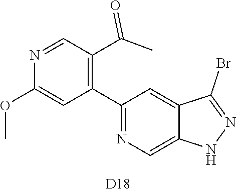 [SMILES: COc1cc(-c2cc3c(Br)n[nH]c3cn2)c(C(C)=O)cn1 ; "D18"] | 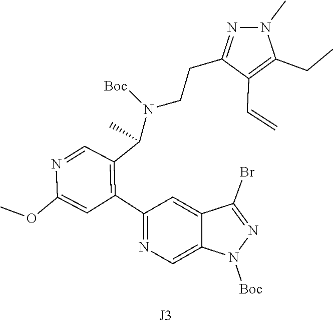 |
| 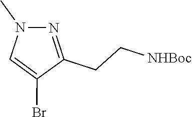 [SMILES: Cn1cc(Br)c(CCNC(=O)OC(C)(C)C)n1] | 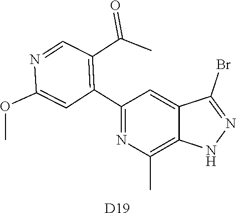 [SMILES: COc1cc(-c2cc3c(Br)n[nH]c3c(C)n2)c(C(C)=O)cn1 ; "D19"] | 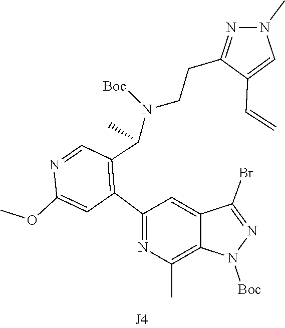 |

General Method K

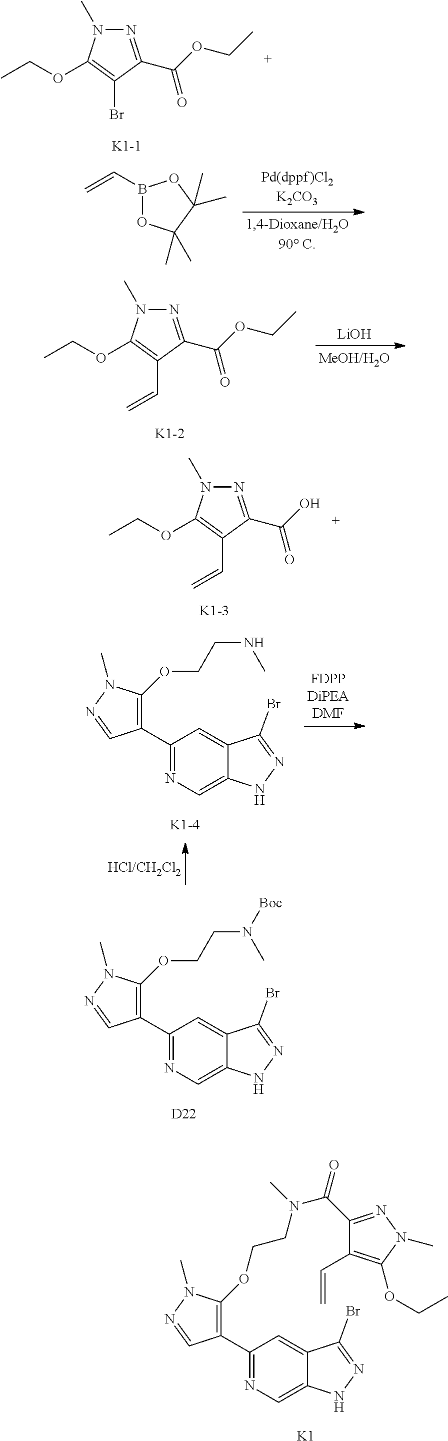

Step 1. To a solution of K1-1 (1.00 eq.) and vinylboronic acid pinacol ester (1.2 eq.) in 1,4-dioxane (0.5 M), is added potassium carbonate (2.00 eq.) in water (2 M) and the nitrogen gas is bubbled through the solution for 15 minutes. Pd(dppf}Cl2-DCM (0.05) is then added to the solution. The reaction is stirred at 100° C. under nitrogen for 18 h. The solution is cooled, diluted with water, and extracted with ethyl acetate three times. The combined organic solution is then washed with 1 N aqueous NaOH solution and brine, and dried over anhydrous magnesium sulfate. The organic solution is then concentrated in vacuo. The crude product is purified on silica gel column to provide K1-2.

Step 2. To a solution of K1-2 (1.0 eq.) in MeOH (0.2 M) is added LiOH (3 eq) in H₂O (1 M). The mixture is stirred at 60° C. until the hydrolysis reaction is completed. The solution is cooled to ambient temperature, concentrated to remove methanol, acidified by aqueous HCl (1 N) until pH˜4-5, and then extracted with CH₂Cl₂. The combined extracts are dried over Na₂SO₄, concentrated, and dried under vacuum to provide K1-3 which is used for the next step without purification.

Step 3. To a solution of D22 (1.00 eq.) in CH₂Cl₂(0.2 M) is added a solution of HCl in dioxane (4 eq HCl). The solution is stirred at 40° C. until the de-Boc is completed. The solvents are removed under rotavap and dried under vacuum to provide K1-4 which is used for the next step without purification.

Step 4. To a solution of K1-3 (1 eq.) and K1-4 (1.0 eq) in DMF (0.2 M) are added DIPEA (3 eq.) and pentafluorophenyl diphenylphosphinate (FDPP) (1.1 eq). The solution is stirred at ambient temperature until the amide formation is completed. The mixture is diluted with water and extracted with EtOAc for three times. The combined extracts are washed with water for three times, aqueous HCl (1N), saturated aqueous Na₂CO₃ and brine, dried over Na₂SO₄, and concentrated. The resulting residue is purified by a silica gel column to afford compound K1.

K1-K27 are prepared based on General Method K.

| | | |
|---|---|---|
| Starting Material 1 | Starting Material 2 | K |

| | | |
|---|---|---|
| 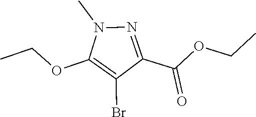 | 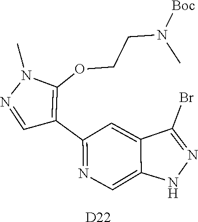 | 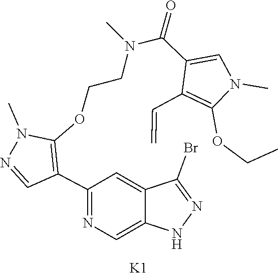 |
| 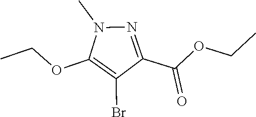 | 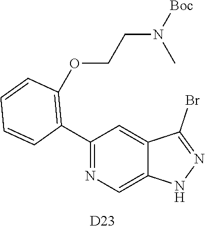 [SMILES: CN(CCOc1ccccc1-c1cc2c(Br)n[nH]c2cn1)C(=O)OC(C)(C)C ; "D23"] | 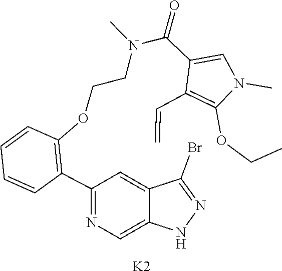 |
| 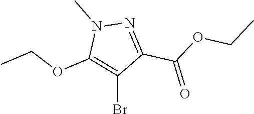 | 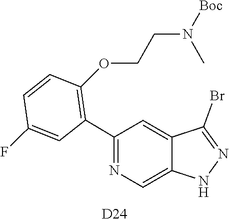 [SMILES: CN(CCOc1ccc(F)cc1-c1cc2c(Br)n[nH]c2cn1)C(=O)OC(C)(C)C ; "D24"] | 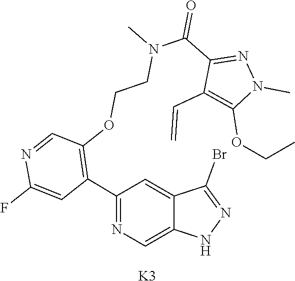 |
| 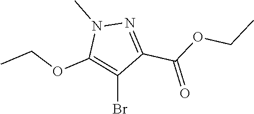 | 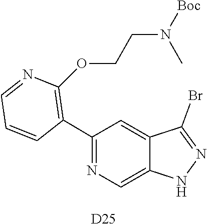 | 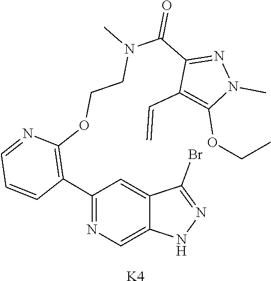 |
| 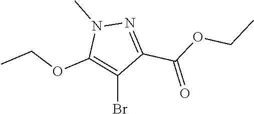 | 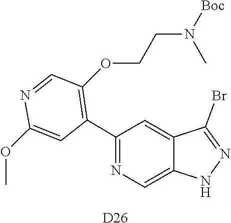 | 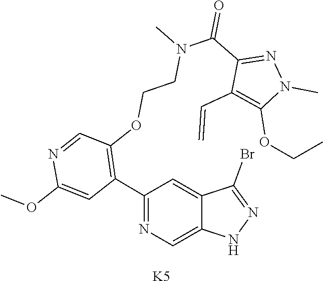 |
| 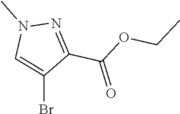 | 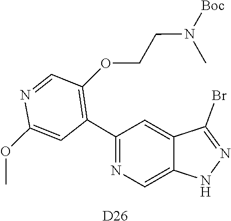 | 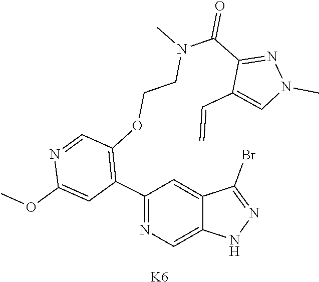 |
| 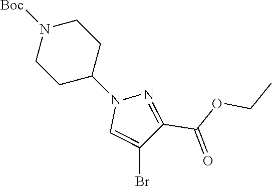 [SMILES: CCOC(=O)c1nn(C2CCN(C(=O)OC(C)(C)C)CC2)cc1Br] | 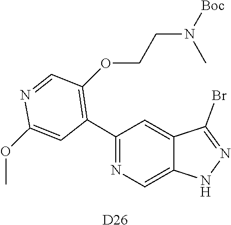 | 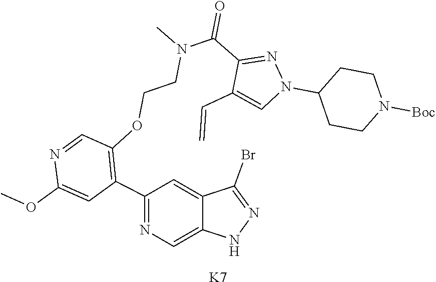 |
| 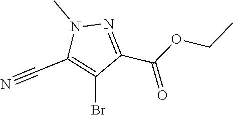 | 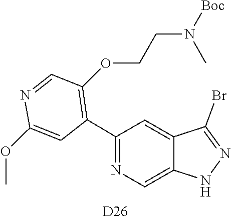 | 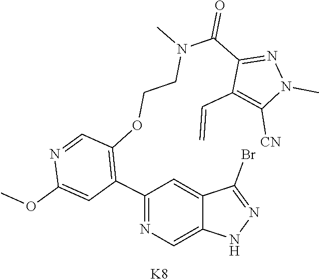 |
| 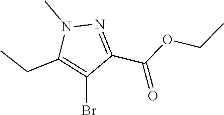 | 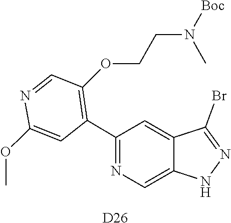 | 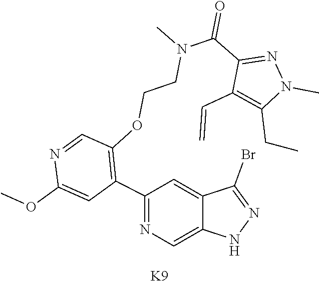 |
| 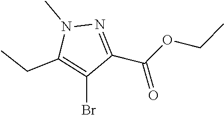 | 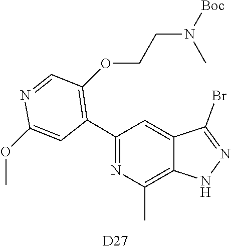 [SMILES: COc1cc(-c2cc3c(Br)n[nH]c3c(C)n2)c(OCCN(C)C(=O)OC(C)(C)C)cn1 ; "D27"] | 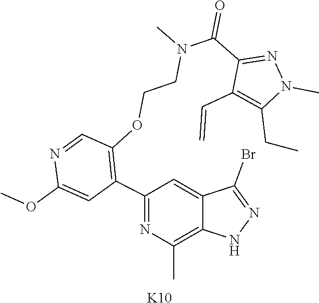 |
| 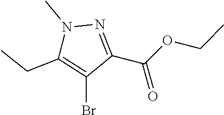 | 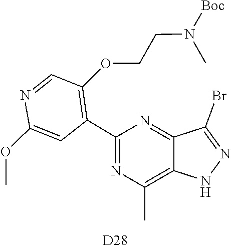 [SMILES: COc1cc(-c2nc(C)c3[nH]nc(Br)c3n2)c(OCCN(C)C(=O)OC(C)(C)C)cn1 ; "D28"] | 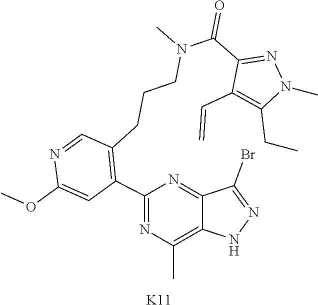 |
| 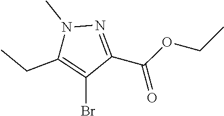 | 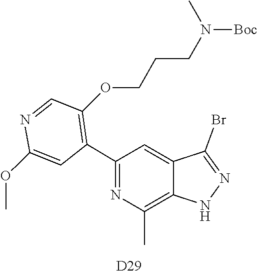 | 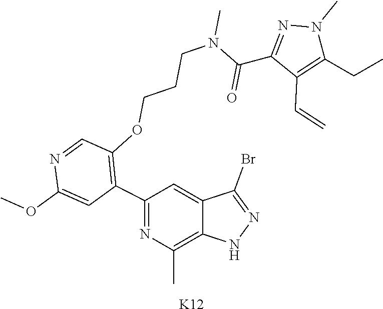 |
| 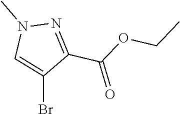 | 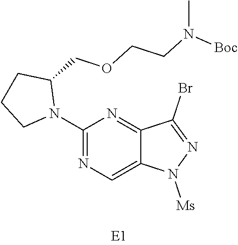 [SMILES: CN(CCOC[C@H]1CCCN1c1ncc2c(n1)c(Br)nn2S(C)(=O)=O)C(=O)OC(C)(C)C] | 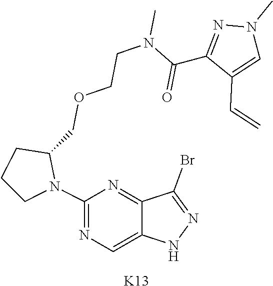 |
| 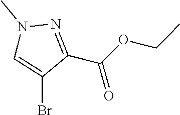 | 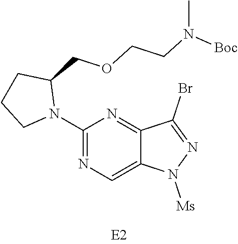 [SMILES: CN(CCOC[C@@H]1CCCN1c1ncc2c(n1)c(Br)nn2S(C)(=O)=O)C(=O)OC(C)(C)C] | 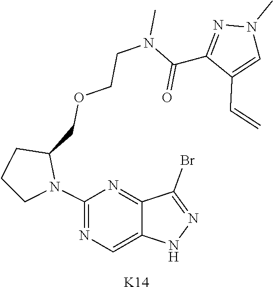 |
| 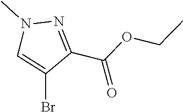 | 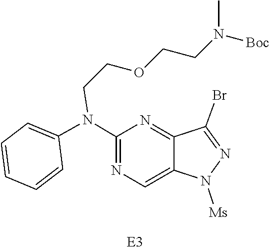 [SMILES: CN(CCOCCN(c1ccccc1)c1ncc2c(n1)c(Br)nn2S(C)(=O)=O)C(=O)OC(C)(C)C] | 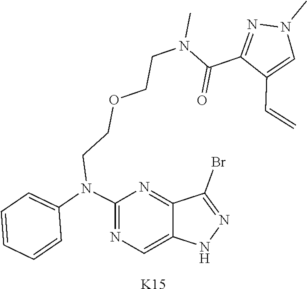 |
| 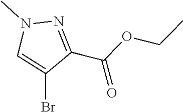 | 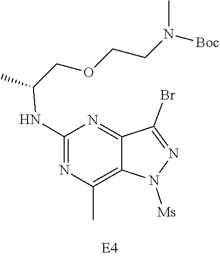 [SMILES: Cc1nc(N[C@H](C)COCCN(C)C(=O)OC(C)(C)C)nc2c(Br)nn(S(C)(=O)=O)c12] | 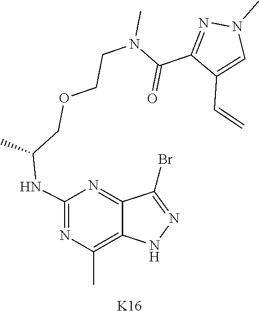 |
| 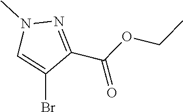 | 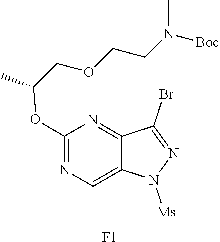 [SMILES: C[C@H](COCCN(C)C(=O)OC(C)(C)C)Oc1ncc2c(n1)c(Br)nn2S(C)(=O)=O ; "F1"] | 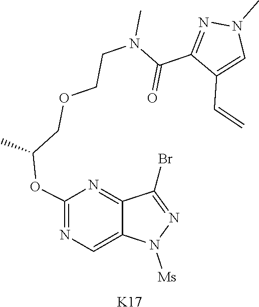 |
| 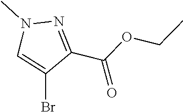 | 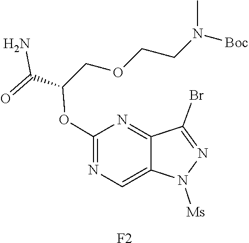 [SMILES: CN(CCOC[C@H](Oc1ncc2c(n1)c(Br)nn2S(C)(=O)=O)C(N)=O)C(=O)OC(C)(C)C ; "F2"] | 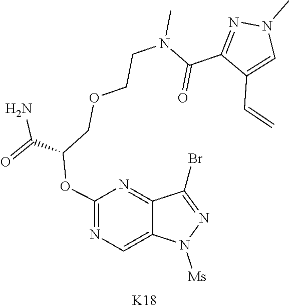 |
| 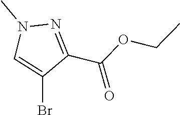 | 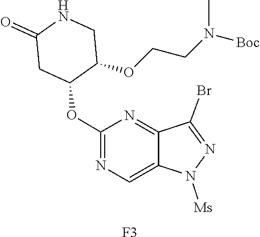 [SMILES: CN(CCO[C@H]1CNC(=O)C[C@H]1Oc1ncc2c(n1)c(Br)nn2S(C)(=O)=O)C(=O)OC(C)(C)C ; "F3"] | 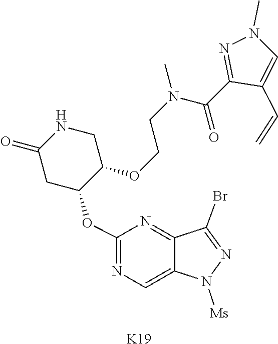 |
| 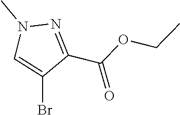 | 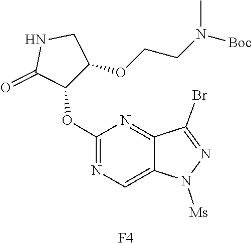 [SMILES: CN(CCO[C@H]1CNC(=O)[C@H]1Oc1ncc2c(n1)c(Br)nn2S(C)(=O)=O)C(=O)OC(C)(C)C ; "F4"] | 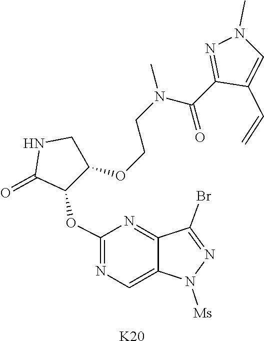 |
| 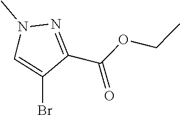 | 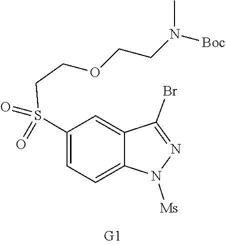 [SMILES: CN(CCOCCS(=O)(=O)c1ccc2c(c1)c(Br)nn2S(C)(=O)=O)C(=O)OC(C)(C)C] | 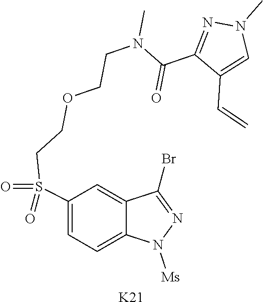 |
| 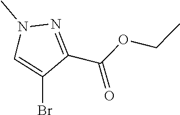 | 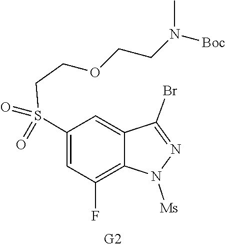 [SMILES: CN(CCOCCS(=O)(=O)c1cc(F)c2c(c1)c(Br)nn2S(C)(=O)=O)C(=O)OC(C)(C)C] | 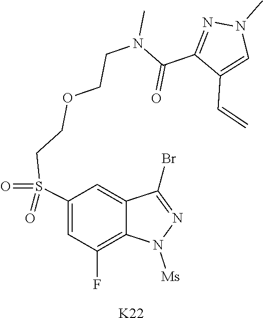 |
| 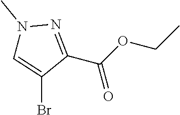 | 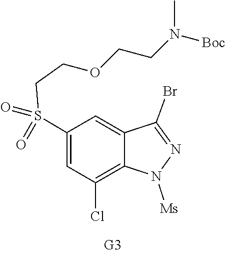 [SMILES: CN(CCOCCS(=O)(=O)c1cc(Cl)c2c(c1)c(Br)nn2S(C)(=O)=O)C(=O)OC(C)(C)C] | 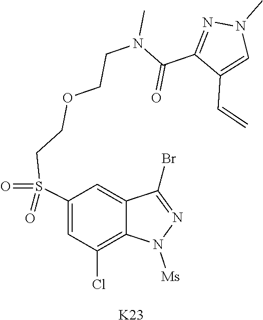 |
| 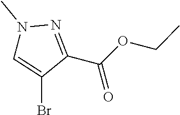 | 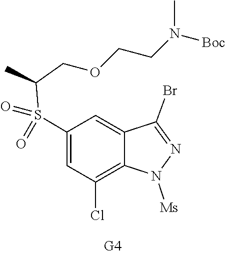 [SMILES: C[C@@H](COCCN(C)C(=O)OC(C)(C)C)S(=O)(=O)c1cc(Cl)c2c(c1)c(Br)nn2S(C)(=O)=O] | 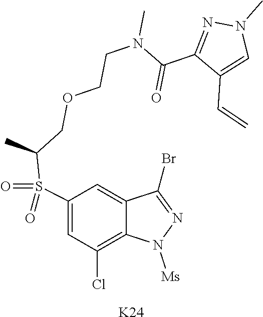 |
| 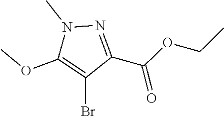 | 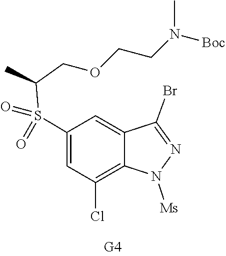 [SMILES: C[C@@H](COCCN(C)C(=O)OC(C)(C)C)S(=O)(=O)c1cc(Cl)c2c(c1)c(Br)nn2S(C)(=O)=O] | 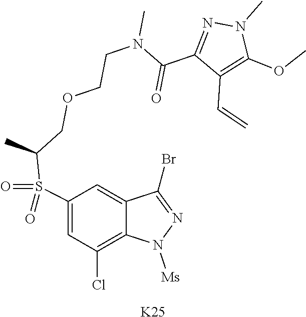 |
| 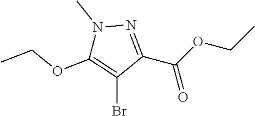 | 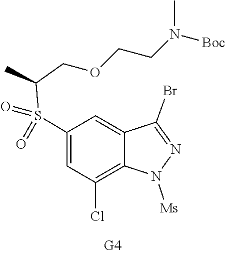 [SMILES: C[C@@H](COCCN(C)C(=O)OC(C)(C)C)S(=O)(=O)c1cc(Cl)c2c(c1)c(Br)nn2S(C)(=O)=O] | 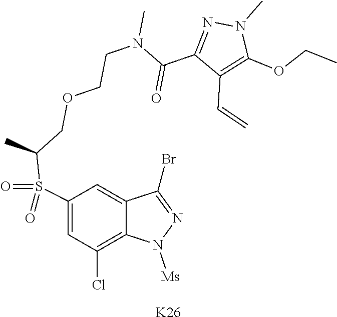 |
| 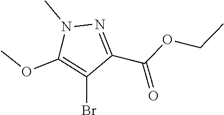 | 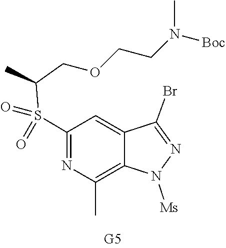 [SMILES: Cc1nc(S(=O)(=O)[C@@H](C)COCCN(C)C(=O)OC(C)(C)C)cc2c(Br)nn(S(C)(=O)=O)c12] | 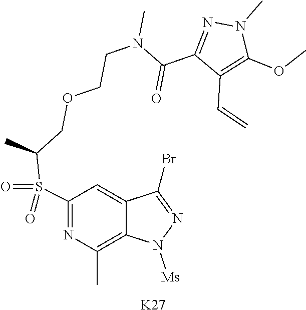 |

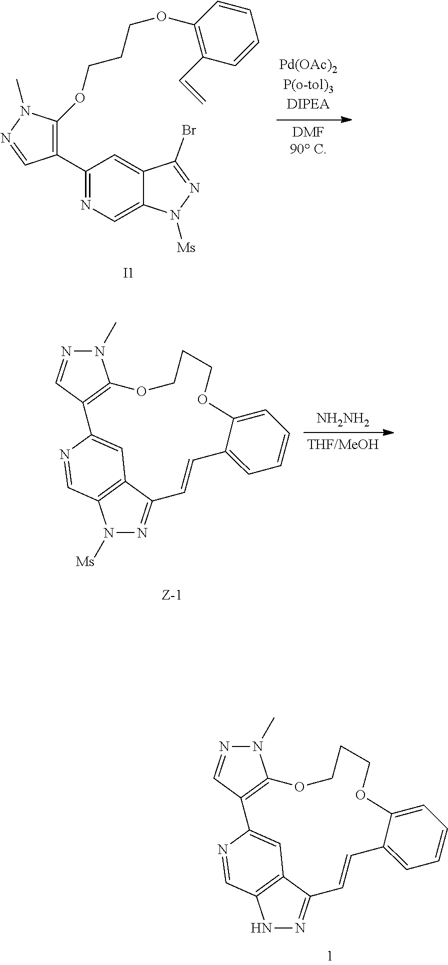

Step 1. To a solution of D1 (1.00 eq.), DIPEA (1.2 eq.) in DMF (0.1 M) are added Pd(OAc)₂ (0.05 eq.) and P(o-tol)₃ (0.07 eq). The mixture is heated at 90° C. until the reaction is complete. The reaction is cooled and concentrated. The residue is diluted with ethyl acetate and filtered via a celite pad. The filtration is washed with water and brine, and dried over anhydrous sodium sulfate. After filtration and concentration, the residue is purified by silica gel chromatography to provide Z-1.

Step 2. To a solution of Z-1 (1.0 eq.) in 1:1 THF/MeOH (0.2 M) is added hydrazine (10 eq.). The solution is stirred at room temperature for 16 h. The solvent is removed under reduced pressure and the residue is purified by silica gel chromatography to provide 1.

General Method Z2

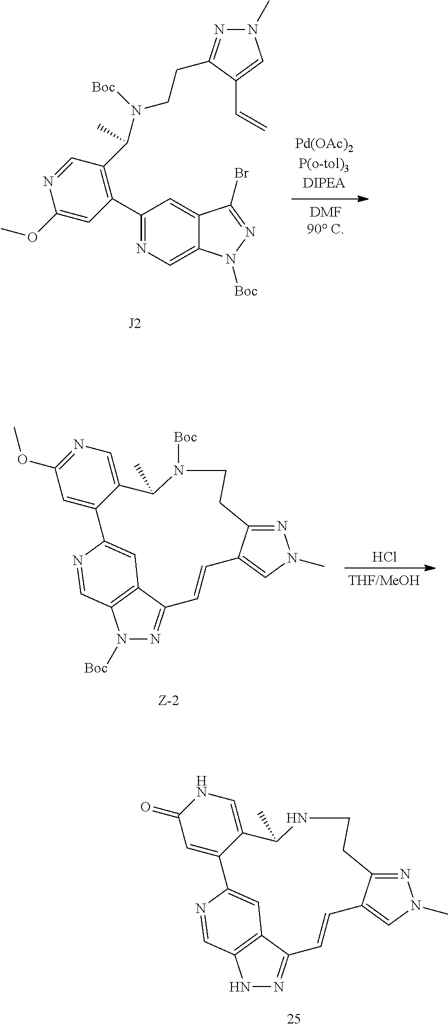

Step 1. Z-2 is obtained following the step 1 in General Method Z1.

Step 2. To a solution of Z-2 (1 eq.) in 1,4-dioxane (1 M) is added equal volume of Con. HCl. The solution is stirred at ambient temperature for 2 hours and then heated at 100° C. for 48 hours. The reaction is cooled and concentrated. The residue is dissolved in ethyl acetate, washed with saturated aqueous Na₂CO₃, and dried over sodium sulfate. After filtration and concentration, the residue is purified by a reversed phase chromatography to provide 25.

Compounds 1-61 can be prepared using either General Method Z1 or Z2 or part of procedures in General Method Z1 or Z2.

| Starting Material | Final Compound | Cpd. # |
|---|---|---|
| 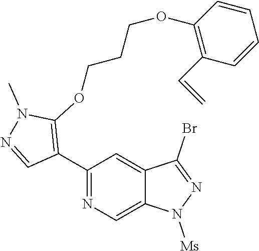 [SMILES: C=Cc1ccccc1OCCCOc1c(-c2cc3c(Br)nn(S(C)(=O)=O)c3cn2)cnn1C] | 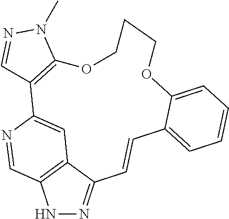 [SMILES: Cn1ncc2c1OCCCOc1ccccc1/C=C/c1n[nH]c3cnc-2cc13] | 1 |
| I1 | | |
| 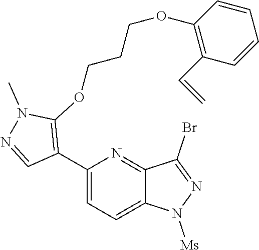 [SMILES: C=Cc1ccccc1OCCCOc1c(-c2ccc3c(n2)c(Br)nn3S(C)(=O)=O)cnn1C] | 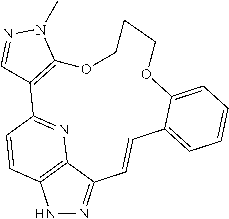 [SMILES: Cn1ncc2c1OCCCOc1ccccc1/C=C/c1n[nH]c3ccc-2nc13] | 2 |
| I2 | | |
| 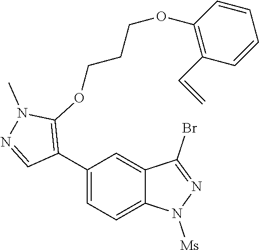 [SMILES: C=Cc1ccccc1OCCCOc1c(-c2ccc3c(c2)c(Br)nn3S(C)(=O)=O)cnn1C] | 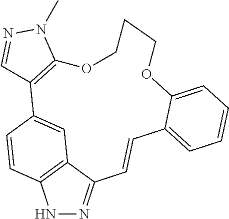 [SMILES: Cn1ncc2c1OCCCOc1ccccc1/C=C/c1n[nH]c3ccc-2cc13] | 3 |
| I3 | | |
| 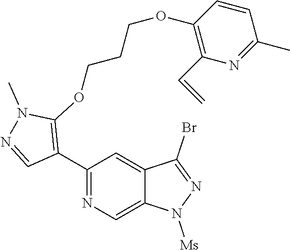 [SMILES: C=Cc1nc(C)ccc1OCCCOc1c(-c2cc3c(Br)nn(S(C)(=O)=O)c3cn2)cnn1C] | 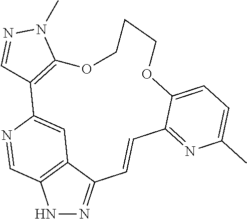 [SMILES: Cc1ccc2c(n1)/C=C/c1n[nH]c3cnc(cc13)-c1cnn(C)c1OCCCO2] | 4 |
| I4 | | |
| 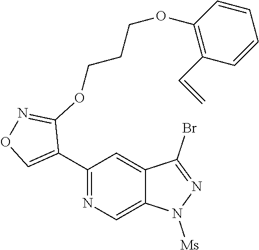 [SMILES: C=Cc1ccccc1OCCCOc1nocc1-c1cc2c(Br)nn(S(C)(=O)=O)c2cn1] | 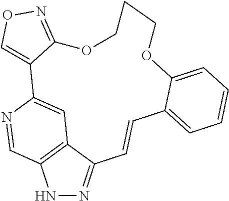 [SMILES: C1=C/c2n[nH]c3cnc(cc23)-c2conc2OCCCOc2ccccc2/1] | 5 |
| I5 | | |
| 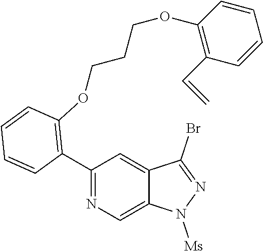 [SMILES: C=Cc1ccccc1OCCCOc1ccccc1-c1cc2c(Br)nn(S(C)(=O)=O)c2cn1] | 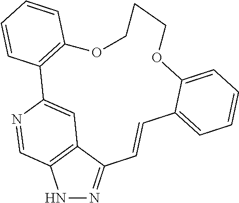 [SMILES: C1=C/c2n[nH]c3cnc(cc23)-c2ccccc2OCCCOc2ccccc2/1] | 6 |
| I6 | | |
| 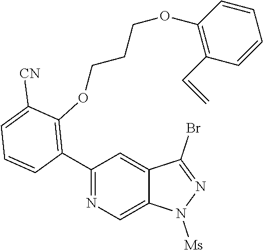 [SMILES: C=Cc1ccccc1OCCCOc1c(C#N)cccc1-c1cc2c(Br)nn(S(C)(=O)=O)c2cn1] | 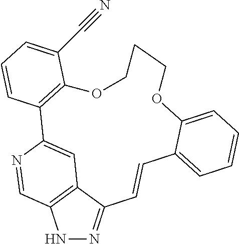 [SMILES: N#Cc1cccc2c1OCCCOc1ccccc1/C=C/c1n[nH]c3cnc-2cc13] | 7 |
| I7 | | |
| 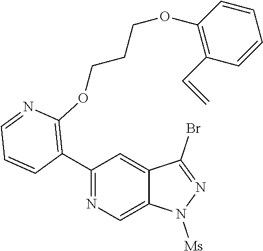 [SMILES: C=Cc1ccccc1OCCCOc1ncccc1-c1cc2c(Br)nn(S(C)(=O)=O)c2cn1] | 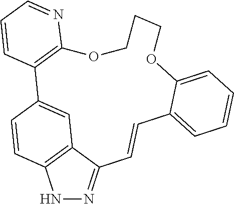 [SMILES: C1=C/c2n[nH]c3ccc(cc23)-c2cccnc2OCCCOc2ccccc2/1] | 8 |
| I8 | | |
| 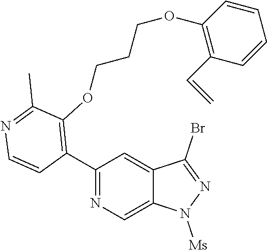 [SMILES: C=Cc1ccccc1OCCCOc1c(-c2cc3c(Br)nn(S(C)(=O)=O)c3cn2)ccnc1C] | 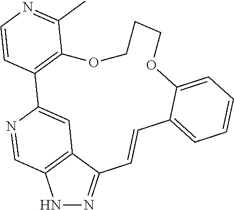 [SMILES: Cc1nccc2c1OCCCOc1ccccc1/C=C/c1n[nH]c3cnc-2cc13] | 9 |
| I9 | | |
| 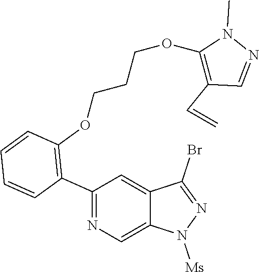 [SMILES: C=Cc1cnn(C)c1OCCCOc1ccccc1-c1cc2c(Br)nn(S(C)(=O)=O)c2cn1] | 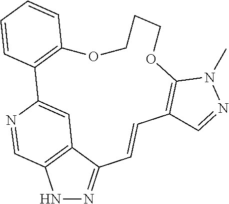 [SMILES: Cn1ncc2c1OCCCOc1ccccc1-c1cc3c(n[nH]c3cn1)/C=C/2] | 10 |
| I10 | | |
| 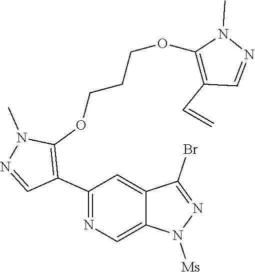 [SMILES: C=Cc1cnn(C)c1OCCCOc1c(-c2cc3c(Br)nn(S(C)(=O)=O)c3cn2)cnn1C] | 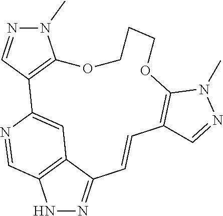 [SMILES: Cn1ncc2c1OCCCOc1c(cnn1C)-c1cc3c(n[nH]c3cn1)/C=C/2] | 11 |
| I11 | | |
| 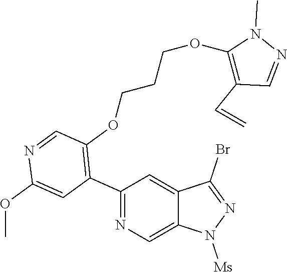 [SMILES: C=Cc1cnn(C)c1OCCCOc1cnc(OC)cc1-c1cc2c(Br)nn(S(C)(=O)=O)c2cn1] | 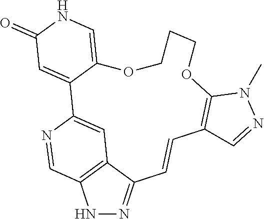 [SMILES: Cn1ncc2c1OCCCOc1c[nH]c(=O)cc1-c1cc3c(n[nH]c3cn1)/C=C/2] | 12 |
| I12 | | |
| 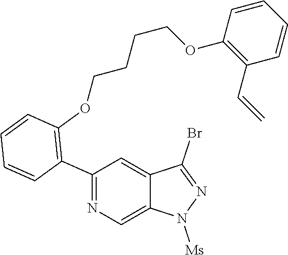 [SMILES: C=Cc1ccccc1OCCCCOc1ccccc1-c1cc2c(Br)nn(S(C)(=O)=O)c2cn1] | 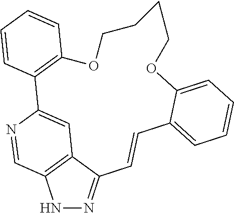 [SMILES: C1=C/c2n[nH]c3cnc(cc23)-c2ccccc2OCCCCOc2ccccc2/1] | 13 |
| I13 | | |
| 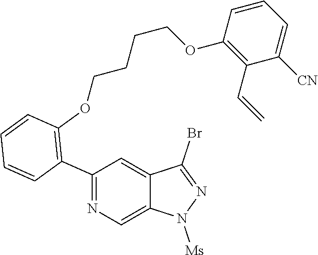 [SMILES: C=Cc1c(C#N)cccc1OCCCCOc1ccccc1-c1cc2c(Br)nn(S(C)(=O)=O)c2cn1] | 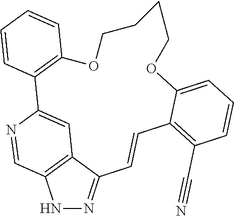 [SMILES: N#Cc1cccc2c1/C=C/c1n[nH]c3cnc(cc13)-c1ccccc1OCCCCO2] | 14 |
| I14 | | |
| 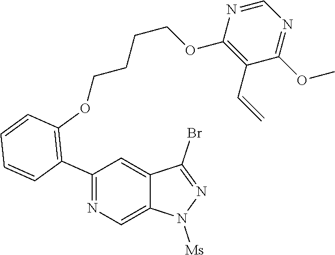 [SMILES: C=Cc1c(OC)ncnc1OCCCCOc1ccccc1-c1cc2c(Br)nn(S(C)(=O)=O)c2cn1] | 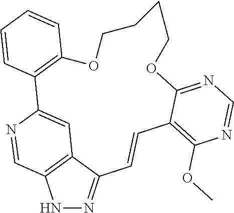 [SMILES: COc1ncnc2c1/C=C/c1n[nH]c3cnc(cc13)-c1ccccc1OCCCCO2] | 15 |
| I15 | | |
| 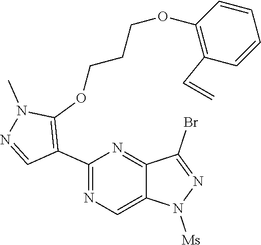 [SMILES: C=Cc1ccccc1OCCCOc1c(-c2ncc3c(n2)c(Br)nn3S(C)(=O)=O)cnn1C] | 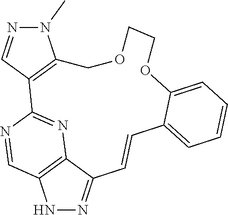 [SMILES: Cn1ncc2c1COCCOc1ccccc1/C=C/c1n[nH]c3cnc-2nc13] | 16 |
| I16 | | |
| 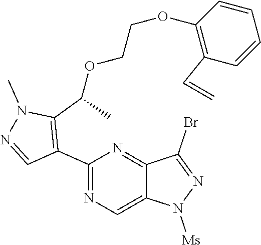 [SMILES: C=Cc1ccccc1OCCO[C@H](C)c1c(-c2ncc3c(n2)c(Br)nn3S(C)(=O)=O)cnn1C] | 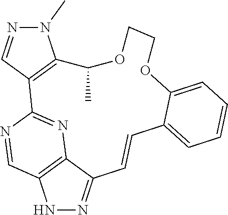 [SMILES: C[C@H]1OCCOc2ccccc2/C=C/c2n[nH]c3cnc(nc23)-c2cnn(C)c21] | 17 |
| I17 | | |
| 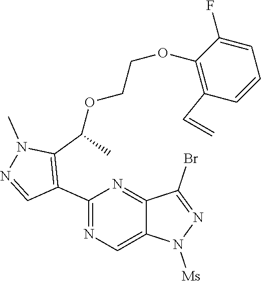 [SMILES: C=Cc1cccc(F)c1OCCO[C@H](C)c1c(-c2ncc3c(n2)c(Br)nn3S(C)(=O)=O)cnn1C] | 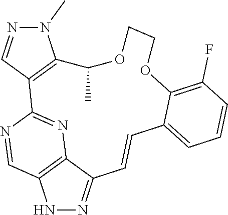 [SMILES: C[C@H]1OCCOc2c(F)cccc2/C=C/c2n[nH]c3cnc(nc23)-c2cnn(C)c21] | 18 |
| I18 | | |
| 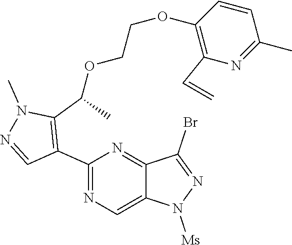 [SMILES: C=Cc1nc(C)ccc1OCCO[C@H](C)c1c(-c2ncc3c(n2)c(Br)nn3S(C)(=O)=O)cnn1C] | 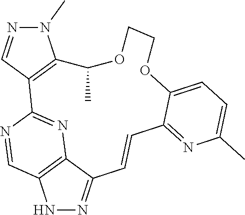 [SMILES: Cc1ccc2c(n1)/C=C/c1n[nH]c3cnc(nc13)-c1cnn(C)c1[C@@H](C)OCCO2] | 19 |
| I19 | | |
| 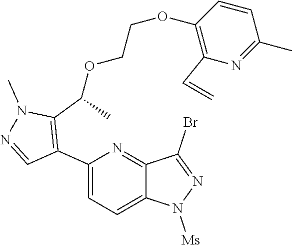 [SMILES: C=Cc1nc(C)ccc1OCCO[C@H](C)c1c(-c2ccc3c(n2)c(Br)nn3S(C)(=O)=O)cnn1C] | 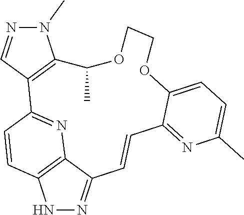 [SMILES: Cc1ccc2c(n1)/C=C/c1n[nH]c3ccc(nc13)-c1cnn(C)c1[C@@H](C)OCCO2] | 20 |
| I20 | | |
| 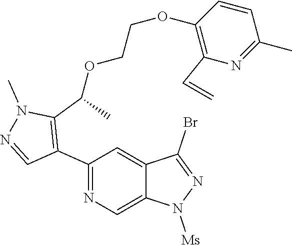 [SMILES: C=Cc1nc(C)ccc1OCCO[C@H](C)c1c(-c2cc3c(Br)nn(S(C)(=O)=O)c3cn2)cnn1C] | 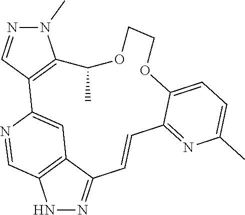 [SMILES: Cc1ccc2c(n1)/C=C/c1n[nH]c3cnc(cc13)-c1cnn(C)c1[C@@H](C)OCCO2] | 21 |
| I21 | | |
| 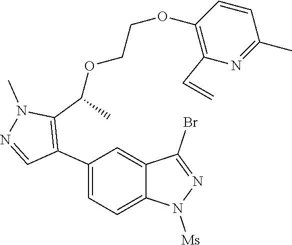 [SMILES: C=Cc1nc(C)ccc1OCCO[C@H](C)c1c(-c2ccc3c(c2)c(Br)nn3S(C)(=O)=O)cnn1C] | 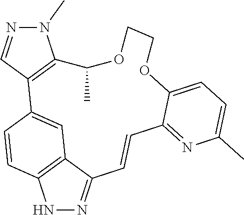 [SMILES: Cc1ccc2c(n1)/C=C/c1n[nH]c3ccc(cc13)-c1cnn(C)c1[C@@H](C)OCCO2] | 22 |
| I22 | | |
| 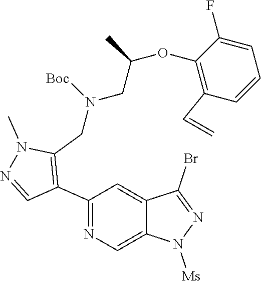 [SMILES: C=Cc1cccc(F)c1O[C@H](C)CN(Cc1c(-c2cc3c(Br)nn(S(C)(=O)=O)c3cn2)cnn1C)C(=O)OC(C)(C)C] | 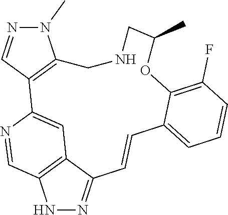 [SMILES: C[C@@H]1CNCc2c(cnn2C)-c2cc3c(n[nH]c3cn2)/C=C/c2cccc(F)c2O1] | 23 |
| I23 | | |
| 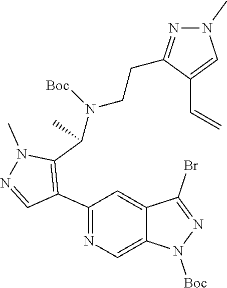 [SMILES: C=Cc1cn(C)nc1CCN(C(=O)OC(C)(C)C)[C@H](C)c1c(-c2cc3c(Br)nn(C(=O)OC(C)(C)C)c3cn2)cnn1C] | 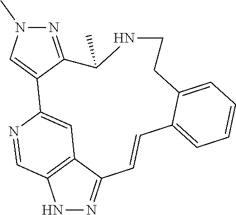 [SMILES: C[C@H]1NCCc2ccccc2/C=C/c2n[nH]c3cnc(cc23)-c2cn(C)nc21] | 24 |
| J1 | | |
| 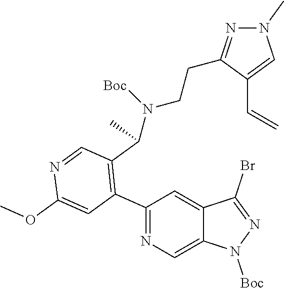 | 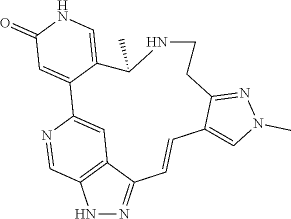 [SMILES: C[C@H]1NCCc2nn(C)cc2/C=C/c2n[nH]c3cnc(cc23)-c2cc(=O)[nH]cc21] | 25 |
| J2 | | |
| 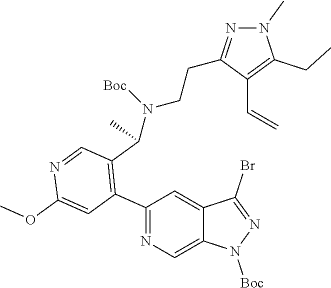 [SMILES: C=Cc1c(CCN(C(=O)OC(C)(C)C)[C@H](C)c2cnc(OC)cc2-c2cc3c(Br)nn(C(=O)OC(C)(C)C)c3cn2)nn(C)c1CC] | 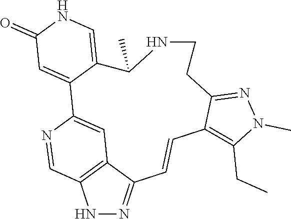 [SMILES: CCc1c2c(nn1C)CCN[C@H](C)c1c[nH]c(=O)cc1-c1cc3c(n[nH]c3cn1)/C=C/2] | 26 |
| J3 | | |
| 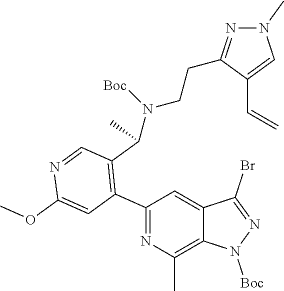 | 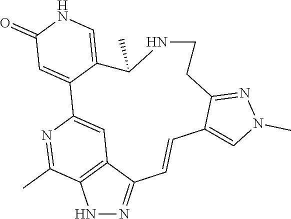 [SMILES: Cc1nc2cc3c(n[nH]c13)/C=C/c1cn(C)nc1CCN[C@H](C)c1c[nH]c(=O)cc1-2] | 27 |
| J4 | | |
| 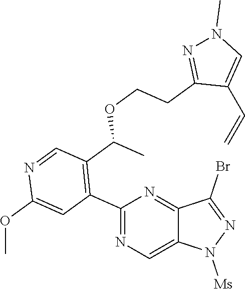 [SMILES: C=Cc1cn(C)nc1CCO[C@H](C)c1cnc(OC)cc1-c1ncc2c(n1)c(Br)nn2S(C)(=O)=O] | 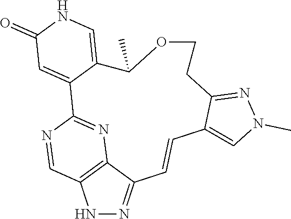 [SMILES: C[C@H]1OCCc2nn(C)cc2/C=C/c2n[nH]c3cnc(nc23)-c2cc(=O)[nH]cc21] | 28 |
| I24 | | |
| 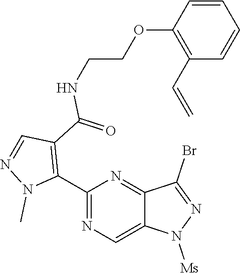 [SMILES: C=Cc1ccccc1OCCNC(=O)c1cnn(C)c1-c1ncc2c(n1)c(Br)nn2S(C)(=O)=O] | 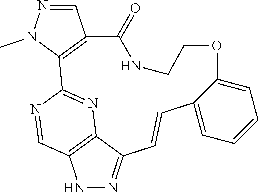 [SMILES: Cn1ncc2c1-c1ncc3[nH]nc(c3n1)/C=C/c1ccccc1OCCNC2=O] | 29 |
| I25 | | |
| 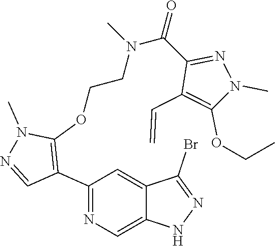 [SMILES: C=Cc1c(C(=O)N(C)CCOc2c(-c3cc4c(Br)n[nH]c4cn3)cnn2C)nn(C)c1OCC] | 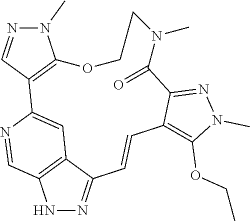 [SMILES: CCOc1c2c(nn1C)C(=O)N(C)CCOc1c(cnn1C)-c1cc3c(n[nH]c3cn1)/C=C/2] | 30 |
| K1 | | |
| 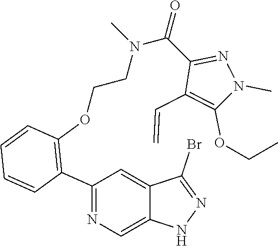 [SMILES: C=Cc1c(C(=O)N(C)CCOc2ccccc2-c2cc3c(Br)n[nH]c3cn2)nn(C)c1OCC] | 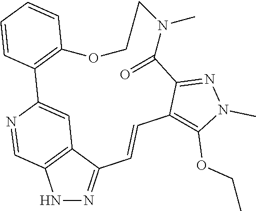 [SMILES: CCOc1c2c(nn1C)C(=O)N(C)CCOc1ccccc1-c1cc3c(n[nH]c3cn1)/C=C/2] | 31 |
| K2 | | |
| 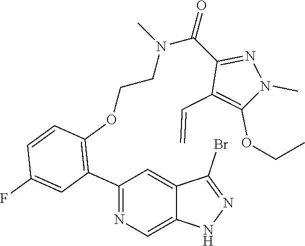 [SMILES: C=Cc1c(C(=O)N(C)CCOc2ccc(F)cc2-c2cc3c(Br)n[nH]c3cn2)nn(C)c1OCC] | 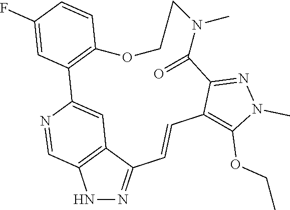 [SMILES: CCOc1c2c(nn1C)C(=O)N(C)CCOc1ccc(F)cc1-c1cc3c(n[nH]c3cn1)/C=C/2] | 32 |
| K3 | | |
| 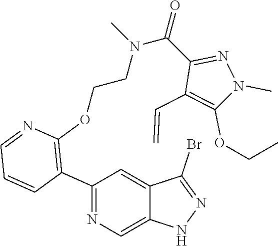 [SMILES: C=Cc1c(C(=O)N(C)CCOc2ncccc2-c2cc3c(Br)n[nH]c3cn2)nn(C)c1OCC] | 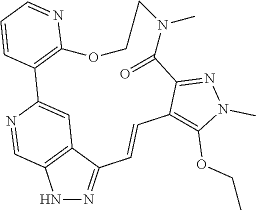 [SMILES: CCOc1c2c(nn1C)C(=O)N(C)CCOc1ncccc1-c1cc3c(n[nH]c3cn1)/C=C/2] | 33 |
| K4 | | |
| 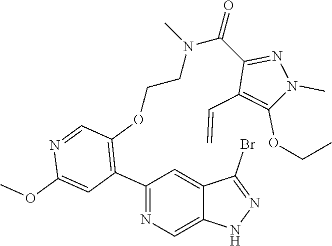 [SMILES: C=Cc1c(C(=O)N(C)CCOc2cnc(OC)cc2-c2cc3c(Br)n[nH]c3cn2)nn(C)c1OCC] | 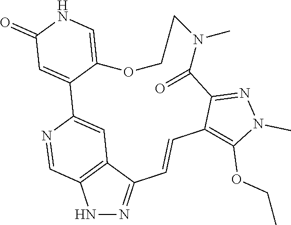 [SMILES: CCOc1c2c(nn1C)C(=O)N(C)CCOc1c[nH]c(=O)cc1-c1cc3c(n[nH]c3cn1)/C=C/2] | 34 |
| K5 | | |
| 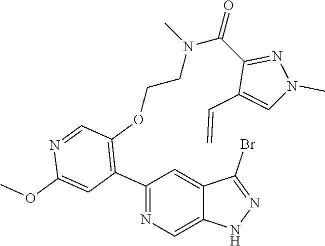 | 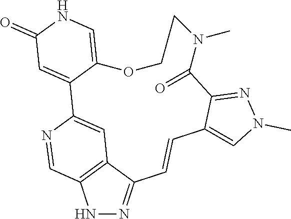 [SMILES: CN1CCOc2c[nH]c(=O)cc2-c2cc3c(n[nH]c3cn2)/C=C/c2cn(C)nc2C1=O] | 35 |
| K6 | | |
| 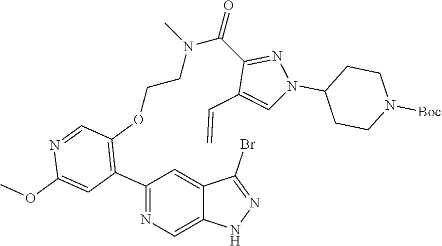 [SMILES: C=Cc1cn(C2CCN(C(=O)OC(C)(C)C)CC2)nc1C(=O)N(C)CCOc1cnc(OC)cc1-c1cc2c(Br)n[nH]c2cn1] | 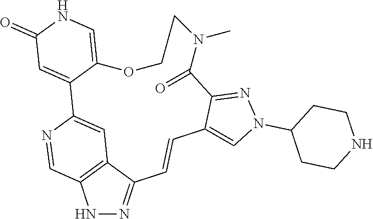 [SMILES: CN1CCOc2c[nH]c(=O)cc2-c2cc3c(n[nH]c3cn2)/C=C/c2cn(C3CCNCC3)nc2C1=O] | 36 |
| K7 | | |
| 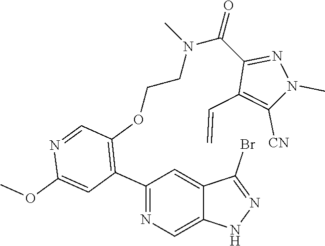 | 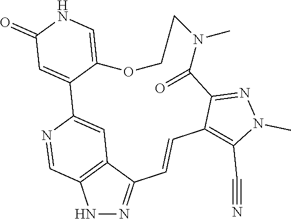 [SMILES: CN1CCOc2c[nH]c(=O)cc2-c2cc3c(n[nH]c3cn2)/C=C/c2c(nn(C)c2C#N)C1=O] | 37 |
| K8 | | |
| 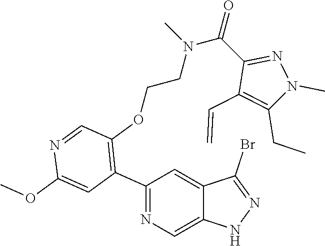 | 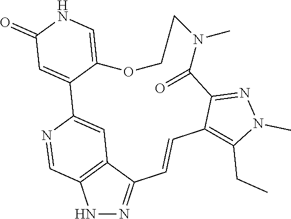 [SMILES: CCc1c2c(nn1C)C(=O)N(C)CCOc1c[nH]c(=O)cc1-c1cc3c(n[nH]c3cn1)/C=C/2] | 38 |
| K9 | | |
| 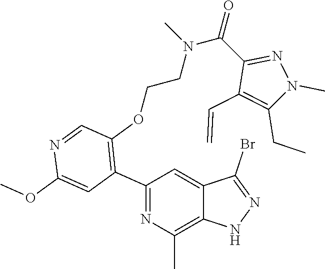 | 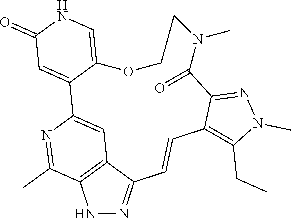 [SMILES: CCc1c2c(nn1C)C(=O)N(C)CCOc1c[nH]c(=O)cc1-c1cc3c(n[nH]c3c(C)n1)/C=C/2] | 39 |
| K10 | | |
| 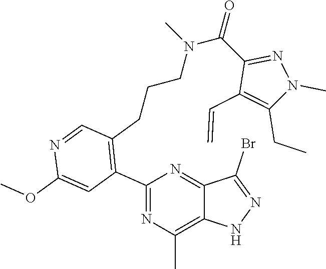 | 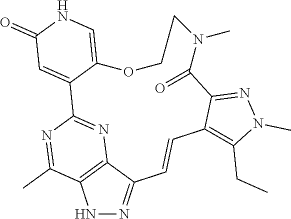 [SMILES: CCc1c2c(nn1C)C(=O)N(C)CCOc1c[nH]c(=O)cc1-c1nc(C)c3[nH]nc(c3n1)/C=C/2] | 40 |
| K11 | | |
| 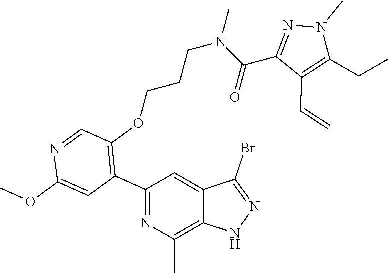 [SMILES: C=Cc1c(C(=O)N(C)CCCOc2cnc(OC)cc2-c2cc3c(Br)n[nH]c3c(C)n2)nn(C)c1CC] | 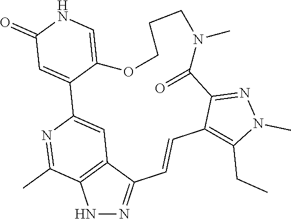 [SMILES: CCc1c2c(nn1C)C(=O)N(C)CCCOc1c[nH]c(=O)cc1-c1cc3c(n[nH]c3c(C)n1)/C=C/2] | 41 |
| K12 | | |
| 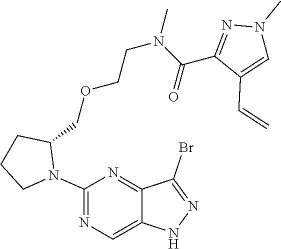 [SMILES: C=Cc1cn(C)nc1C(=O)N(C)CCOC[C@H]1CCCN1c1ncc2[nH]nc(Br)c2n1] | 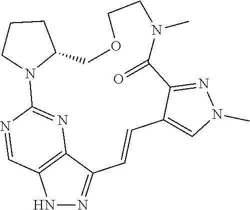 [SMILES: CN1CCOC[C@H]2CCCN2c2ncc3[nH]nc(c3n2)/C=C/c2cn(C)nc2C1=O] | 42 |
| K13 | | |
| 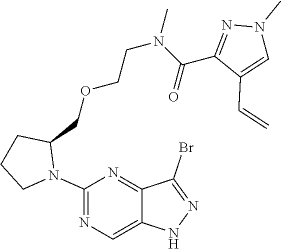 [SMILES: C=Cc1cn(C)nc1C(=O)N(C)CCOC[C@@H]1CCCN1c1ncc2[nH]nc(Br)c2n1] | 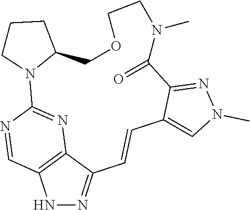 [SMILES: CN1CCOC[C@@H]2CCCN2c2ncc3[nH]nc(c3n2)/C=C/c2cn(C)nc2C1=O] | 43 |
| K14 | | |
| 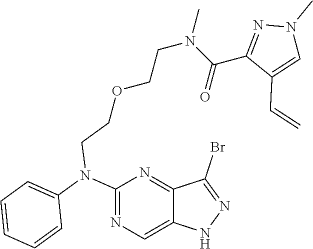 [SMILES: C=Cc1cn(C)nc1C(=O)N(C)CCOCCN(c1ccccc1)c1ncc2[nH]nc(Br)c2n1] | 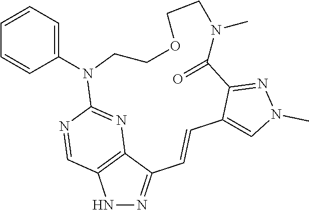 [SMILES: CN1CCOCCN(c2ccccc2)c2ncc3[nH]nc(c3n2)/C=C/c2cn(C)nc2C1=O] | 44 |
| K15 | | |
| 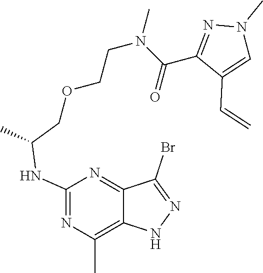 [SMILES: C=Cc1cn(C)nc1C(=O)N(C)CCOC[C@@H](C)Nc1nc(C)c2[nH]nc(Br)c2n1] | 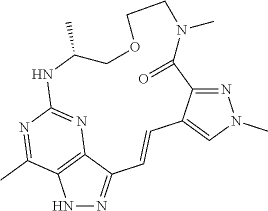 | 45 |
| K16 | | |
| 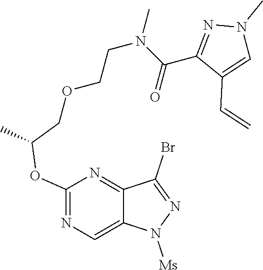 [SMILES: C=Cc1cn(C)nc1C(=O)N(C)CCOC[C@@H](C)Oc1ncc2c(n1)c(Br)nn2S(C)(=O)=O] | 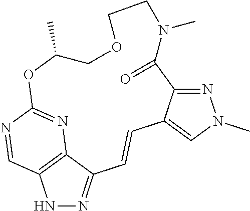 [SMILES: C[C@@H]1COCCN(C)C(=O)c2nn(C)cc2/C=C/c2n[nH]c3cnc(nc23)O1] | 46 |
| K17 | | |
| 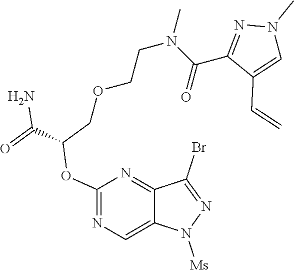 [SMILES: C=Cc1cn(C)nc1C(=O)N(C)CCOC[C@H](Oc1ncc2c(n1)c(Br)nn2S(C)(=O)=O)C(N)=O] | 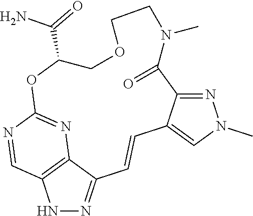 [SMILES: CN1CCOC[C@@H](C(N)=O)Oc2ncc3[nH]nc(c3n2)/C=C/c2cn(C)nc2C1=O] | 47 |
| K18 | | |
| 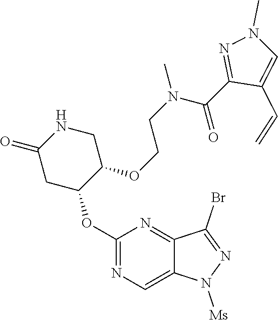 [SMILES: C=Cc1cn(C)nc1C(=O)N(C)CCO[C@H]1CNC(=O)C[C@H]1Oc1ncc2c(n1)c(Br)nn2S(C)(=O)=O] | 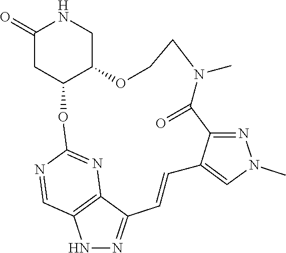 [SMILES: CN1CCO[C@H]2CNC(=O)C[C@H]2Oc2ncc3[nH]nc(c3n2)/C=C/c2cn(C)nc2C1=O] | 48 |
| K19 | | |
| 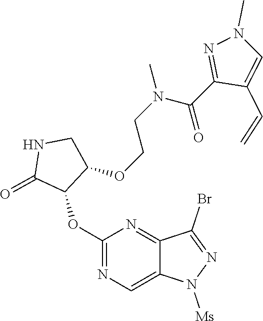 [SMILES: C=Cc1cn(C)nc1C(=O)N(C)CCO[C@H]1CNC(=O)[C@H]1Oc1ncc2c(n1)c(Br)nn2S(C)(=O)=O] | 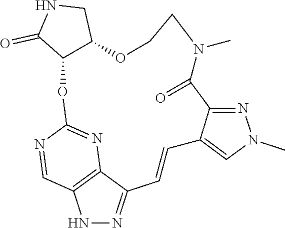 [SMILES: CN1CCO[C@H]2CNC(=O)[C@H]2Oc2ncc3[nH]nc(c3n2)/C=C/c2cn(C)nc2C1=O] | 49 |
| K20 | | |
| 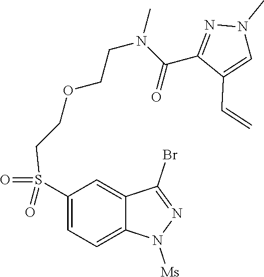 [SMILES: C=Cc1cn(C)nc1C(=O)N(C)CCOCCS(=O)(=O)c1ccc2c(c1)c(Br)nn2S(C)(=O)=O] | 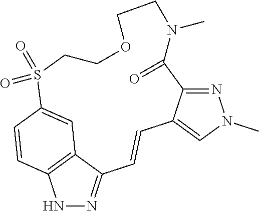 [SMILES: CN1CCOCCS(=O)(=O)c2ccc3[nH]nc(c3c2)/C=C/c2cn(C)nc2C1=O] | 50 |
| K21 | | |
| 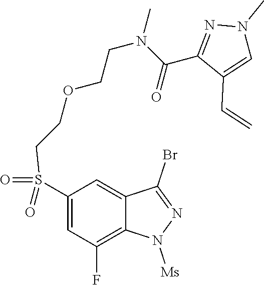 [SMILES: C=Cc1cn(C)nc1C(=O)N(C)CCOCCS(=O)(=O)c1cc(F)c2c(c1)c(Br)nn2S(C)(=O)=O] | 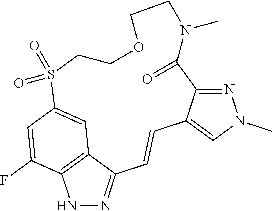 [SMILES: CN1CCOCCS(=O)(=O)c2cc(F)c3[nH]nc(c3c2)/C=C/c2cn(C)nc2C1=O] | 51 |
| K22 | | |
| 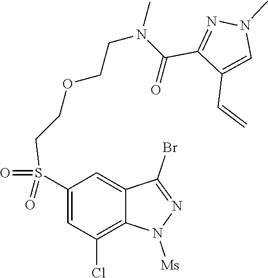 [SMILES: C=Cc1cn(C)nc1C(=O)N(C)CCOCCS(=O)(=O)c1cc(Cl)c2c(c1)c(Br)nn2S(C)(=O)=O] | 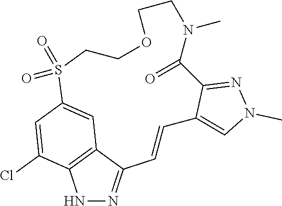 [SMILES: CN1CCOCCS(=O)(=O)c2cc(Cl)c3[nH]nc(c3c2)/C=C/c2cn(C)nc2C1=O] | 52 |
| K23 | | |
| 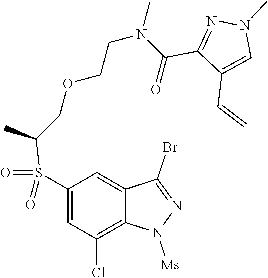 [SMILES: C=Cc1cn(C)nc1C(=O)N(C)CCOC[C@H](C)S(=O)(=O)c1cc(Cl)c2c(c1)c(Br)nn2S(C)(=O)=O] | 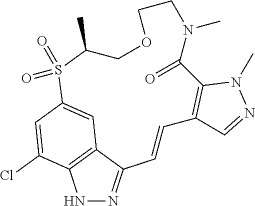 [SMILES: C[C@H]1COCCN(C)C(=O)c2c(cnn2C)/C=C/c2n[nH]c3c(Cl)cc(cc23)S1(=O)=O] | 53 |
| K24 | | |
| 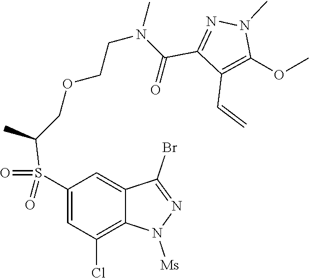 [SMILES: C=Cc1c(C(=O)N(C)CCOC[C@H](C)S(=O)(=O)c2cc(Cl)c3c(c2)c(Br)nn3S(C)(=O)=O)nn(C)c1OC] | 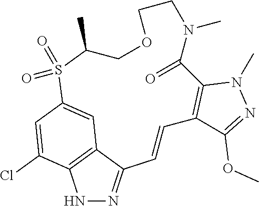 [SMILES: COc1nn(C)c2c1/C=C/c1n[nH]c3c(Cl)cc(cc13)S(=O)(=O)[C@@H](C)COCCN(C)C2=O] | 54 |
| K25 | | |
| 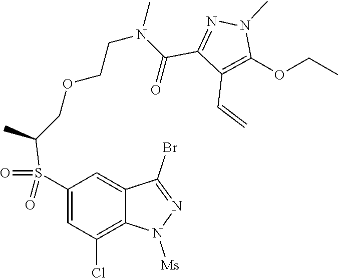 [SMILES: C=Cc1c(C(=O)N(C)CCOC[C@H](C)S(=O)(=O)c2cc(Cl)c3c(c2)c(Br)nn3S(C)(=O)=O)nn(C)c1OCC] | 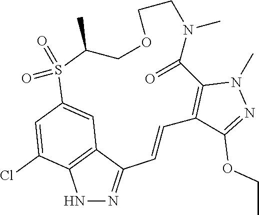 [SMILES: CCOc1nn(C)c2c1/C=C/c1n[nH]c3c(Cl)cc(cc13)S(=O)(=O)[C@@H](C)COCCN(C)C2=O] | 55 |
| K26 | | |
| 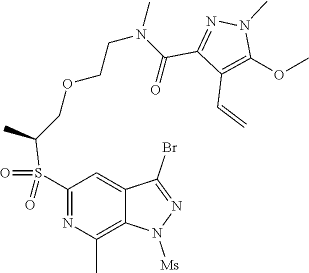 [SMILES: C=Cc1c(C(=O)N(C)CCOC[C@H](C)S(=O)(=O)c2cc3c(Br)nn(S(C)(=O)=O)c3c(C)n2)nn(C)c1OC] | 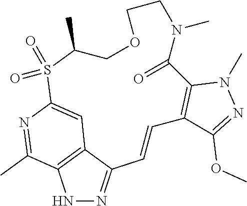 | 56 |
| K27 | | |
| 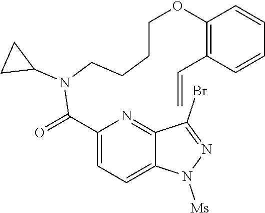 [SMILES: C=Cc1ccccc1OCCCCN(C(=O)c1ccc2c(n1)c(Br)nn2S(C)(=O)=O)C1CC1] | 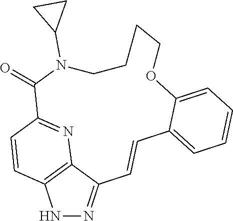 [SMILES: O=C1c2ccc3[nH]nc(c3n2)/C=C/c2ccccc2OCCCCN1C1CC1] | 57 |
| I26 | | |
| 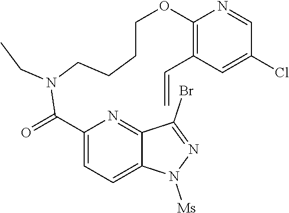 [SMILES: C=Cc1cc(Cl)cnc1OCCCCN(CC)C(=O)c1ccc2c(n1)c(Br)nn2S(C)(=O)=O] | 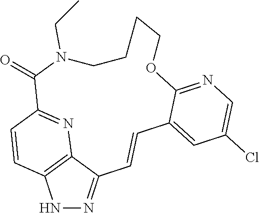 [SMILES: CCN1CCCCOc2ncc(Cl)cc2/C=C/c2n[nH]c3ccc(nc23)C1=O] | 58 |
| I27 | | |
| 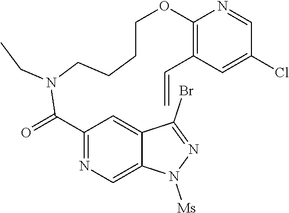 [SMILES: C=Cc1cc(Cl)cnc1OCCCCN(CC)C(=O)c1cc2c(Br)nn(S(C)(=O)=O)c2cn1] | 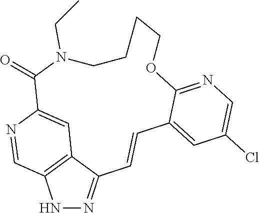 [SMILES: CCN1CCCCOc2ncc(Cl)cc2/C=C/c2n[nH]c3cnc(cc23)C1=O] | 59 |
| I28 | | |
| 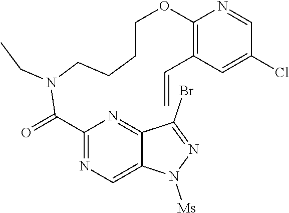 [SMILES: C=Cc1cc(Cl)cnc1OCCCCN(CC)C(=O)c1ncc2c(n1)c(Br)nn2S(C)(=O)=O] | 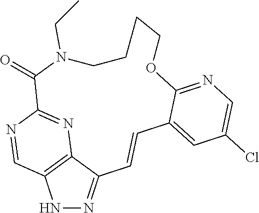 [SMILES: CCN1CCCCOc2ncc(Cl)cc2/C=C/c2n[nH]c3cnc(nc23)C1=O] | 60 |
| I29 | | |
| 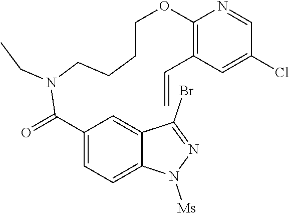 [SMILES: C=Cc1cc(Cl)cnc1OCCCCN(CC)C(=O)c1ccc2c(c1)c(Br)nn2S(C)(=O)=O] | 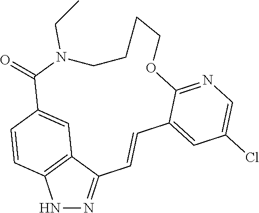 [SMILES: CCN1CCCCOc2ncc(Cl)cc2/C=C/c2n[nH]c3ccc(cc23)C1=O] | 61 |
| I30 | | |

### Example 1: Preparation of (11E)-1-methyl-19,20-dihydro-1H,8H,18H-10,7,4-(ethan[1]yl[1,2]diylidene)pyrazolo[3,4-f][1,5,9,12,13]benzodioxatriazacyclooctadecine (Cpd. 1)

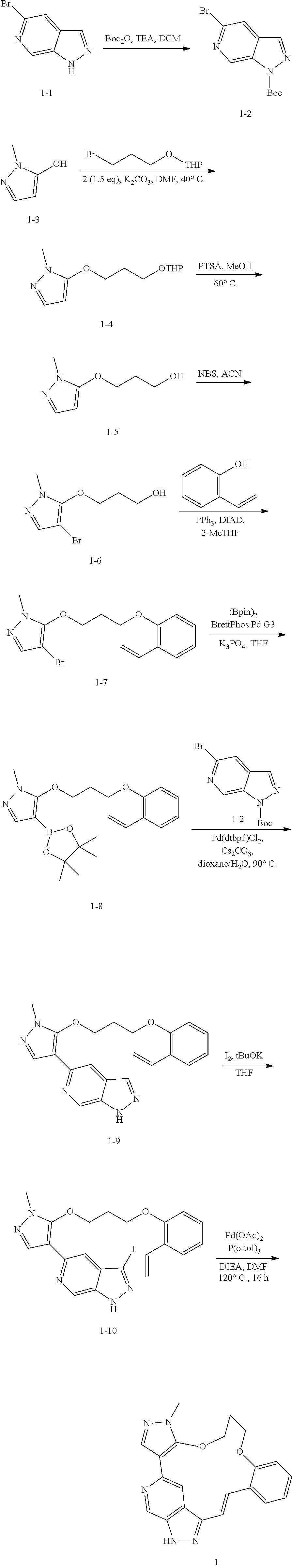

Step 1. To a solution of 5-bromo-1H-pyrazolo[3,4-c]pyridine (1 g, 5.05 mmol, 1 eq) in DCM (10 mL) was added TEA (1.53 g, 15.1 mmol, 2.11 mL, 3 eq) and tert-butoxycarbonyl tert-butyl carbonate (1.32 g, 6.06 mmol, 1.39 mL, 1.2 eq). The mixture was stirred at 25° C. for 2 hours. On completion, the reaction mixture was washed with 1M HCl (20 mL×3). The organic phase was washed with aq. NaHCO₃ (10 mL×3), dried over anhydrous sodium sulfate, filtered and concentrated to give a residue which was purified by column chromatography (SiO₂, Petroleum ether/Ethyl acetate=1:0 to 3:1) to give tert-butyl 5-bromopyrazolo[3,4-c]pyridine-1-carboxylate (1.4 g, 4.70 mmol, 92.99% yield) was obtained as yellow solid. ¹H NMR (400 MHz, DMSO-d₆) δ=9.17 (s, 1H), 8.52 (d, J=0.4 Hz, 1H), 8.21 (d, J=1.2 Hz, 1H), 1.67 (s, 9H).

Step 2. To a mixture of 2-methylpyrazol-3-ol (16.5 g, 168 mmol, 1 eq) and K₂CO₃ (69.5 g, 503 mmol, 3.00 eq) in DMF (700 mL) was added 2-(3-bromopropoxy)tetrahydropyran (56.1 g, 251 mmol, 1.5 eq), the resulting mixture was stirred at 40° C. for 12 hours. On completion, the mixture was added water (3 L) and extracted with ethyl acetate (500 ml×5). The combined organic phase was dried over anhydrous sodium sulfate, filtered and concentrated to give a residue. The residue was purified by column chromatography (SiO₂, Petroleum ether/Ethyl acetate=1:0 to 3:1) to give 1-methyl-5-(3-tetrahydropyran-2-yloxypropoxy)pyrazole (27.0 g, 112 mmol, 66.97% yield) as yellow oil. ¹H NMR (400 MHz, CDCl₃) δ=7.25 (d, J=2.0 Hz, 1H), 5.46 (d, J=2.0 Hz, 1H), 4.59-4.54 (m, 1H), 4.15-4.09 (m, 2H), 3.88 (td, J=6.4, 10.0 Hz, 1H), 3.80 (ddd, J=3.2, 8.0, 11.2 Hz, 1H), 3.60 (s, 3H), 3.56-3.47 (m, 2H), 2.05 (t, J=6.4 Hz, 2H), 1.83-1.74 (m, 1H), 1.72-1.65 (m, 1H), 1.57-1.47 (m, 4H).

Step 3. A mixture of 1-methyl-5-(3-tetrahydropyran-2-yloxypropoxy)pyrazole (27.0 g, 112 mmol, 1 eq), PTSA (3.87 g, 22.4 mmol, 0.2 eq) in MeOH (40 mL) was stirred at 60° C. for 16 hours. On completion, the mixture was concentrated in vacuum. It was added NaHCO₃ solution to adjust pH to the value of 7 and extracted with ethyl acetate (100 mL×4). The combined organic phase was dried over anhydrous sodium sulfate, filtered and concentrated to give 3-(2-methylpyrazol-3-yl)oxypropan-1-ol (11.9 g, 76.1 mmol, 67.81% yield) as yellow oil. ¹H NMR (400 MHz, DMSO-d₆) δ=7.19 (d, J=2.0 Hz, 1H), 5.61 (d, J=2.0 Hz, 1H), 4.58 (t, J=5.2 Hz, 1H), 4.10 (t, J=6.4 Hz, 2H), 3.58-3.53 (m, 2H), 3.52 (s, 3H), 1.86 (quin, J=6.4 Hz, 2H).

Step 4. A mixture of 3-(2-methylpyrazol-3-yl)oxypropan-1-ol (11.7 g, 74.9 mmol, 1 eq) in MeCN (250 mL) was added NBS (13.7 g, 77.1 mmol, 1.03 eq). The mixture was stirred at 25° C. for 1.5 hours. On completion, the mixture was concentrated in vacuum to give crude which was purified by prep-HPLC (FA condition) to give 3-(4-bromo-2-methyl-pyrazol-3-yl)oxypropan-1-ol (10.1 g, 42.9 mmol, 57.35% yield) as yellow oil. ¹H NMR (400 MHz, CDCl₃) δ=7.28 (s, 1H), 4.40 (t, J=6.0 Hz, 2H), 3.86 (t, J=6.0 Hz, 2H), 3.67 (s, 3H), 2.14 (s, 1H), 2.03 (quin, J=6.0 Hz, 2H).

Step 5. To a mixture of 3-(4-bromo-2-methyl-pyrazol-3-yl)oxypropan-1-ol (10 g, 42.5 mmol, 2 eq) and 2-vinylphenol (2.56 g, 21.2 mmol, 1 eq), PPh₃ (12.2 g, 46.7 mmol, 2.2 eq) in 2-MeTHF (450 mL) was stirred at 15° C. for 0.5 hours under N₂. Then the mixture was added DIAD (9.46 g, 46.7 mmol, 9.10 mL, 2.2 eq) at 0° C. and stirred at 15° C. for 16 hours. On completion, the mixture was concentrated to give a residue which was purified by column chromatography (SiO₂, Petroleum ether/Ethyl acetate=1:0 to 3:1) to give 4-bromo-1-methyl-5-[3-(2-vinylphenoxy)propoxy]pyrazole (5 g, 14.83 mmol, 69.71% yield) as yellow oil. ¹H NMR (400 MHz, DMSO-d₆) δ=7.51 (dd, J=1.2, 7.6 Hz, 1H), 7.39 (s, 1H), 7.30-7.22 (m, 1H), 7.05-7.01 (m, 1H), 7.00-6.91 (m, 2H), 5.78 (dd, J=1.6, 17.6 Hz, 1H), 5.24 (dd, J=1.6, 11.2 Hz, 1H), 4.40 (t, J=6.4 Hz, 2H), 4.19 (t, J=6.0 Hz, 2H), 3.61 (s, 3H), 2.21 (quin, J=6.0 Hz, 2H).

Step 6. To a mixture of 4-bromo-1-methyl-5-[3-(2-vinylphenoxy)propoxy]pyrazole (1 g, 2.97 mmol, 1 eq) and 4,4,5,5-tetramethyl-2-(4,4,5,5-tetramethyl-1,3,2-dioxaborolan-2-yl)-1,3,2-dioxaborolane (1.51 g, 5.93 mmol, 2 eq) in THF (35 mL) was added BrettPhos Pd G3 (268 mg, 296 μmol, 0.1 eq) and K₃PO₄ (1.89 g, 8.90 mmol, 3 eq). The mixture was stirred at 50° C. for 16 hours. On completion, the mixture was concentrated to give a residue which was purified by column chromatography (SiO₂, Petroleum ether/Ethyl acetate=1:0 to 3:1) to give 1-methyl-4-(4,4,5,5-tetramethyl-1,3,2-dioxaborolan-2-yl)-5-[3-(2-vinylphenoxy)propoxy]pyrazole (600 mg, 1.56 mmol, 52.65% yield) as yellow solid. ¹H NMR (400 MHz, DMSO-d₆) δ=7.51 (dd, J=1.6, 7.6 Hz, 1H), 7.35 (s, 1H), 7.28-7.22 (m, 1H), 7.03 (d, J=8.0 Hz, 1H), 7.00-6.90 (m, 2H), 5.78 (dd, J=1.6, 18.0 Hz, 1H), 5.22 (dd, J=1.2, 11.2 Hz, 1H), 4.48 (t, J=6.4 Hz, 2H), 4.16 (t, J=6.0 Hz, 2H), 3.54 (s, 3H), 2.24-2.14 (m, 2H), 1.21 (s, 12H).

Step 7. To a mixture of 1-methyl-4-(4,4,5,5-tetramethyl-1,3,2-dioxaborolan-2-yl)-5-[3-(2-vinylphenoxy)propoxy]pyrazole (553 mg, 1.44 mmol, 2 eq), tert-butyl 5-bromopyrazolo[3,4-c]pyridine-1-carboxylate (214 mg, 719 μmol, 1 eq) in H₂O (1 mL) and dioxane (10 mL) was added Cs₂CO₃ (703 mg, 2.16 mmol, 3 eq) and ditert-butyl(cyclopentyl)phosphane;dichloropalladium;iron (46.9 mg, 71.9 μmol, 0.1 eq). The mixture was degassed and purged with N₂ for 3 times, and then the mixture was stirred at 90° C. for 8 hours. On completion, the mixture was concentrated to give a residue which was purified by column chromatography (SiO₂, PE:THF=1:0 to 1:1) to give 5-[1-methyl-5-[3-(2-vinylphenoxy)propoxy]pyrazol-4-yl]-1H-pyrazolo[3,4-c]pyridine (140 mg, 372 μmol, 51.83% yield) as yellow oil. ¹H NMR (400 MHz, DMSO-d₆) δ=11.92-11.35 (m, 1H), 9.24 (s, 1H), 8.26 (s, 1H), 7.96 (s, 1H), 7.48 (dd, J=1.6, 7.6 Hz, 1H), 7.28-7.23 (m, 1H), 7.05 (d, J=8.0 Hz, 1H), 6.96-6.93 (m, 1H), 6.92-6.82 (m, 1H), 5.72 (dd, J=1.6, 18.0 Hz, 1H), 5.09 (dd, J=1.6, 11.2 Hz, 1H), 4.54 (t, J=6.4 Hz, 2H), 4.28-4.23 (m, 2H), 3.67 (s, 3H), 2.34-2.26 (m, 2H).

Step 8. To a solution of 5-[1-methyl-5-[3-(2-vinylphenoxy)propoxy]pyrazol-4-yl]-1H-pyrazolo[3,4-c]pyridine (113 mg, 301 μmol, 1 eq) in THF (2.2 mL) was added tBuOK (101 mg, 903 μmol, 3 eq). The resulting mixture was stirred at 0° C. for 6 minutes, after that a solution of 12 (91.6 mg, 361 μmol, 72.7 μL, 1.2 eq) in THF (3.3 mL) was added to the mixtures. After stirring for 2 hours, the mixture was filtered and concentrated to give a residue which was purified by column chromatography (SiO₂, PE:THF=1:0 to 1:1) to give 3-iodo-5-[1-methyl-5-[3-(2-vinylphenoxy)propoxy]pyrazol-4-yl]-1H-pyrazolo[3,4-c]pyridine (100 mg, 199 μmol, 66.27% yield) as white solid.

Step 9. To a solution of 3-iodo-5-[1-methyl-5-[3-(2-vinylphenoxy)propoxy]pyrazol-4-yl]-1H-pyrazolo[3,4-c]pyridine (79 mg, 157 μmol, 1 eq) in DMF (3 mL) was added tris-o-tolylphosphane (4.80 mg, 15.76 μmol, 0.1 eq), DIPEA (40.7 mg, 315 μmol, 54.9 μL, 2 eq) and Pd(OAc)₂ (1.77 mg, 7.88 μmol, 0.05 eq). The resulting mixture was stirred at 90° C. for 12 hours. On completion, the mixture was quenched with water (5 mL) and extracted with ethyl acetate (3 mL×3). The combined organic phase was dried over anhydrous sodium sulfate, filtered and concentrated to give a residue which was purified by prep-HPLC (column: Phenomenex Synergi Polar-RP 100×25 mm×4 um; mobile phase: [water(0.225% FA)-ACN]; B %: 30%-60%, 8 min) to give a crude which was further purified by prep-HPLC (column: Waters xbridge 150×25 mm×10 μm; mobile phase: [water(10 mM NH₄HCO₃)-ACN]; B %: 24%-54%, 11 min) to Cpd. 1 (4.99 mg, 13.3 μmol, 8.48% yield) as brown solid.

### Example 2: Preparation of (18E)-8-methyl-2,8,11,12-tetrahydro-10H-3,5-ethenodipyrazolo[3,4-f:4′,3′-j][1,5,9]benzodioxazacyclopentadecine (Cpd. 2)

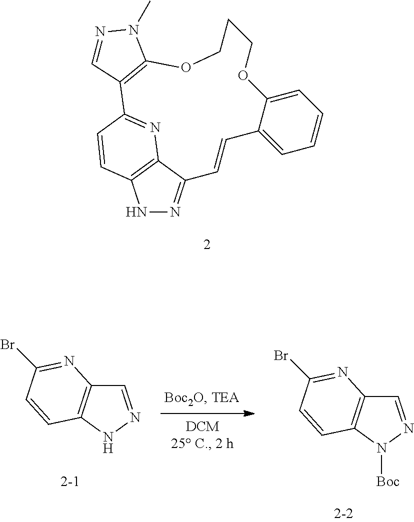 [SMILES: Cn1ncc2c1OCCCOc1ccccc1/C=C/c1n[nH]c3ccc-2nc13 ; Brc1ccc2[nH]ncc2n1 ; CC(C)(C)OC(=O)n1ncc2nc(Br)ccc21]

2-2 was prepared following a similar procedure as 1-2. Cpd. 2 was prepared following similar procedures as Cpd. 1 using 2-2 and 1-8 as starting materials.

### Example 3: Preparation of (11E)-1-methyl-19,20-dihydro-1H,8H,18H-10,7,4-(ethan[1]yl[1,2]diylidene)pyrazolo[3,4-f][1,5,12,13]benzodioxadiazacyclooctadecine (Cpd. 3)

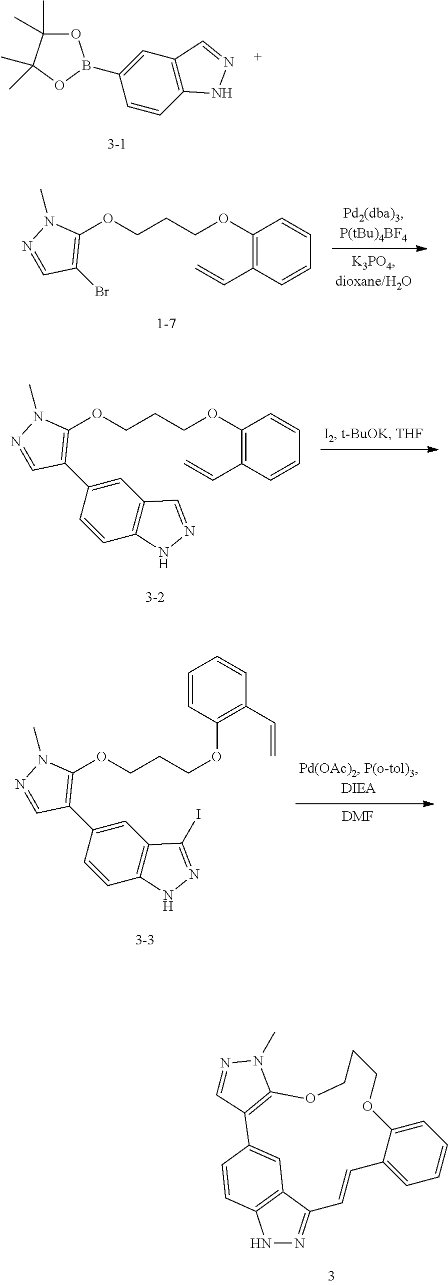

Step 1. To a mixture of 4-bromo-1-methyl-5-[3-(2-vinylphenoxy)propoxy]pyrazole (500 mg, 1.48 mmol, 1 eq.) and 5-(4,4,5,5-tetramethyl-1,3,2-dioxaborolan-2-yl)-1H-indazole (434 mg, 1.78 mmol, 1.2 eq.) in dioxane (10 mL) and H₂O (3.3 mL) was added K₃PO₄ (944 mg, 4.45 mmol, 3 eq.), tritert-butylphosphonium;tetrafluoroborate (43.0 mg, 148 μmol, 0.1 eq.) and Pd₂(dba)₃ (67.9 mg, 74.1 μmol, 0.05 eq.), the resulting mixture was stirred at 120° C. for 12 hours. On completion, the mixture was concentrated to give a residue. The residue was purified by prep-TLC (SiO₂, PE:EA, 1:1) to give 6-[1-methyl-5-[3-(2-vinylphenoxy) propoxy]pyrazol-4-yl]-1H-indazole (210 mg, 561 μmol, 37.8% yield) as a colorless oil. ¹H NMR (400 MHz, DMSO-d₆) δ=12.99 (s, 1H), 7.91 (s, 1H), 7.86 (s, 1H), 7.68 (s, 1H), 7.56-7.52 (m, 1H), 7.50-7.42 (m, 2H), 7.27-7.21 (m, 1H), 7.01-6.90 (m, 2H), 6.80 (dd, J=17.6, 11.2 Hz, 1H), 5.69 (dd, J=17.6, 1.2 Hz, 1H), 5.06 (dd, J=11.2, 1.2 Hz, 1H), 4.18-4.11 (m, 4H), 3.67 (s, 3H), 2.20 (q, J=6.0 Hz, 2H).

Step 2. To a solution of 6-[1-methyl-5-[3-(2-vinylphenoxy)propoxy]pyrazol-4-yl]-1H-indazole (210 mg, 561 μmol, 1 eq.) in THF (4 mL) was added t-BuOK (189 mg, 1.68 mmol, 3 eq.), the resulting mixture was stirred at 0° C. for 5 mins, after that a solution of I₂ (185 mg, 729 μmol, 1.3 eq.) in THF (6 mL) was added to the mixture, the mixture was stirred at 25° C. for another 2 hours. On completion, the mixture was filtered and concentrated to give a residue. The residue was purified by column chromatography (SiO₂, Petroleum ether/Ethyl acetate, from 1:0 to 1:1) to give 3-iodo-6-[1-methyl-5-[3-(2-vinylphenoxy)propoxy]pyrazol-4-yl]-1H-indazole (230 mg, 459 μmol, 81.9% yield) as a brown solid.

Step 3. To a solution of 3-iodo-6-[1-methyl-5-[3-(2-vinylphenoxy)propoxy]pyrazol-4-yl]-1H-indazole (60.0 mg, 119 μmol, 1 eq.) in DMF (6 mL) was added tris-o-tolylphosphane (3.65 mg, 11.9 μmol, 0.1 eq.), DIEA (31.0 mg, 239 μmol, 41.8 μL, 2 eq.) and Pd(OAc)₂ (1.35 mg, 6.00 μmol, 0.05 eq.), the resulting mixture was stirred at 120° C. for 16 hours. On completion, the mixture was quenched with water (15 mL) and extracted with ethyl acetate (8 mL×3), the combined organic phase was dried over anhydrous sodium sulfate, filtered and concentrated to give a residue. The crude product was purified by reversed-phase HPLC (column: Phenomenex Luna C18 150×25 mm×10 μm; mobile phase: [water(0.225% FA)-ACN]; B %: 44%-74%, 10 min) to give Cpd. 3 (6.20 mg, 16.6 μmol, 13.8% yield) as a yellow solid.

### Example 4: Preparation of (18E)-8-methyl-8,9,11,12-tetrahydro-2H-5,3-(azenometheno)dipyrazolo[3,4-f:4′,3′-j][1,4,9]benzodioxazacyclopentadecine (Cpd. 16)

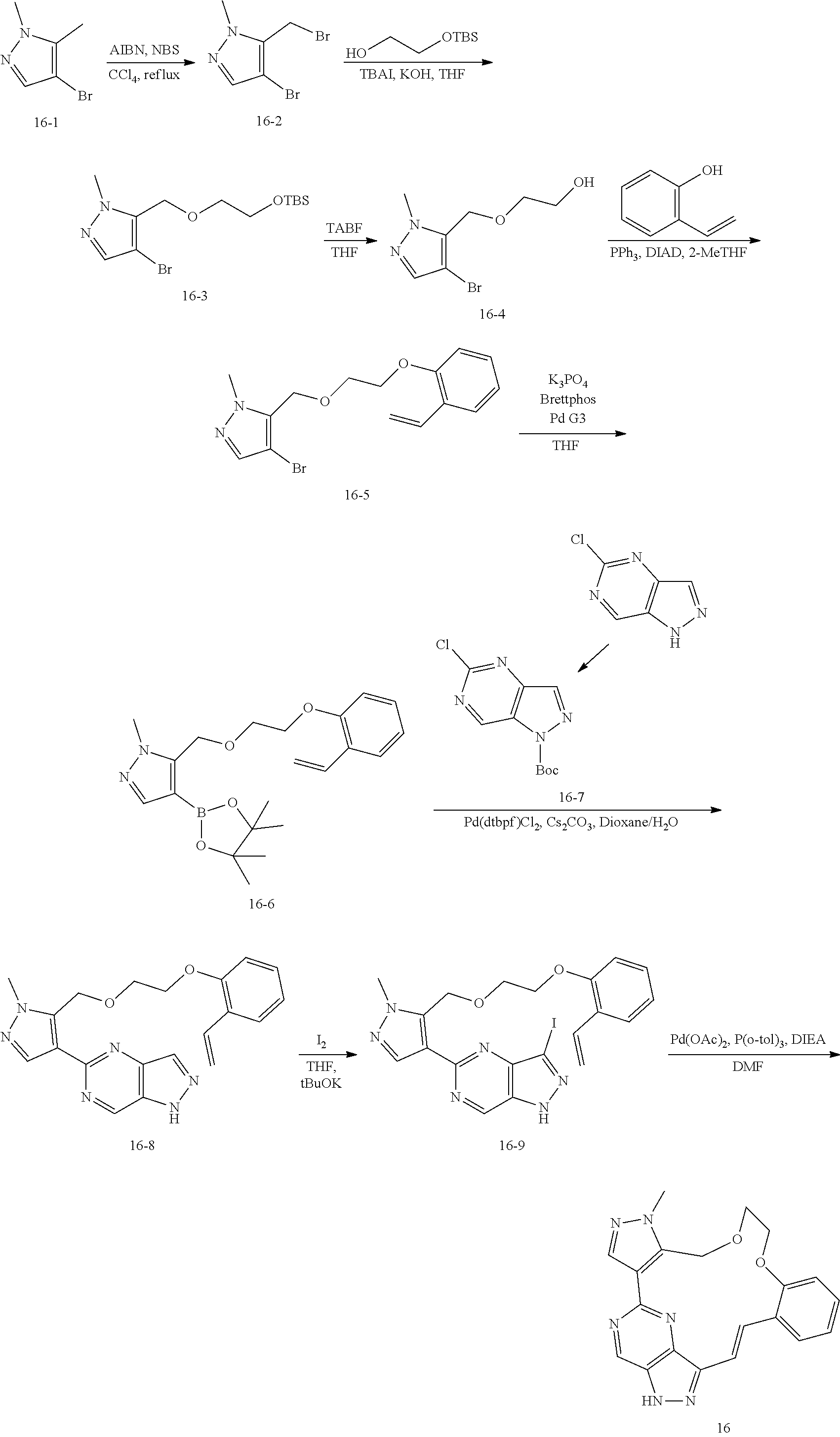

Step 1. To a solution of 4-bromo-1,5-dimethyl-pyrazole (15.0 g, 85.7 mmol, 1 eq) in CCl₄ (200 mL) was added AIBN (1.41 g, 8.57 mmol, 0.1 eq) and NBS (15.2 g, 85.7 mmol, 1 eq), the resulting mixture was stirred at 60° C. for 12 h. On completion, the mixture was concentrated to give a residue. The residue was purified by column chromatography (SiO₂, Petroleum ether/Ethyl acetate=10:1 to 5:1) to give 4-bromo-5-(bromomethyl)-1-methyl-pyrazole (20 g, 78.76 mmol, 91.91% yield) as a white solid. 1H NMR (400 MHz, DMSO-d₆) δ=7.56 (s, 1H), 4.75 (s, 2H), 3.87 (s, 3H).

Step 2. To a solution of 4-bromo-5-(bromomethyl)-1-methyl-pyrazole (20.0 g, 78.8 mmol, 1 eq) in THF (400 mL) was added 2-[tert-butyl(dimethyl)silyl]oxyethanol (20.8 g, 118 mmol, 1.5 eq), TBAI (2.91 g, 7.88 mmol, 0.1 eq) and KOH (13.3 g, 236 mmol, 3 eq), the resulting mixture was stirred at 25° C. for 12 h. On completion, the mixture was concentrated to give residue. The residue was purified by column chromatography (Petroleum ether/Ethyl acetate=10:1 to 5:1) to give 2-[(4-bromo-2-methyl-pyrazol-3-yl)methoxy]ethoxy-tert-butyl-dimethyl-silane (13.19 g, 37.76 mmol, 47.94% yield) as a brown oil.

Step 3. To a solution of 2-[(4-bromo-2-methyl-pyrazol-3-yl)methoxy]ethoxy-tert-butyl-dimethyl-silane (13.1 g, 37.5 mmol, 1 eq) in THF (132 mL) was added TBAF·3H₂O (17.8 g, 56.2 mmol, 1.5 eq), the resulting mixture was stirred at 25° C. for 12 h. On completion, the mixture was concentrated to give a residue. The residue was purified by column chromatography (Petroleum ether/Ethyl acetate=10:1 to 1:4) to give 2-[(4-bromo-2-methyl-pyrazol-3-yl)methoxy]ethanol (8.8 g, 37.43 mmol, 99.83% yield) as a colorless oil.

Step 4. The mixture of 2-[(4-bromo-2-methyl-pyrazol-3-yl)methoxy]ethanol (2.00 g, 8.51 mmol, 2 eq), 2-vinylphenol (511 mg, 4.25 mmol, 1 eq) and PPh₃ (2.45 g, 9.36 mmol, 2.2 eq) in 2-MeTHF (48 mL) was stirred at 25° C. for 30 min, then DIAD (1.89 g, 9.36 mmol, 2.2 eq) was added dropwise to the mixture at 0° C., the resulting mixture was stirred for another 24 h at 25° C. On completion, the mixture was concentrated to give a residue. The residue was purified by column chromatography (SiO₂, Petroleum ether/Ethyl acetate=10:1 to 3:1) to give 4-bromo-1-methyl-5-[2-(2-vinylphenoxy)ethoxymethyl]pyrazole (590 mg, 1.75 mmol, 41.13% yield) as a colorless oil. ¹H NMR (400 MHz, DMSO-d₆) δ=7.56-7.46 (m, 2H), 7.28-7.19 (m, 1H), 7.02-6.87 (m, 3H), 5.79 (dd, J=1.6, 17.9 Hz, 1H), 5.24 (dd, J=1.6, 11.2 Hz, 1H), 4.62 (s, 2H), 4.18-4.09 (m, 2H), 3.84 (s, 3H), 3.81-3.77 (m, 2H).

Step 5. To a mixture of 4-bromo-1-methyl-5-[2-(2-vinylphenoxy)ethoxymethyl]pyrazole (1.10 g, 3.26 mmol, 1 eq) and 4,4,5,5-tetramethyl-2-(4,4,5,5-tetramethyl-1,3,2-dioxaborolan-2-yl)-1,3,2-dioxaborolane (1.66 g, 6.52 mmol, 2 eq) in THF (22 mL) was added K₃PO₄ (2.08 g, 9.79 mmol, 3 eq) and BrettPhos Pd G3 (295 mg, 326 μmol, 0.1 eq), the resulting mixture was stirred at 50° C. for 12 h. On completion, the mixture was concentrated and purified by column chromatography (SiO₂, Petroleum ether/Ethyl acetate=10:1 to 3:1) to give 1-methyl-4-(4,4,5,5-tetramethyl-1,3,2-dioxaborolan-2-yl)-5-[2-(2-vinylphenoxy)ethoxymethyl]pyrazole (0.59 g, 1.54 mmol, 47.07% yield) as a yellow solid. ¹H NMR (400 MHz, DMSO-d₆) δ=7.52-7.46 (m, 2H), 7.27-7.19 (m, 1H), 7.01-6.87 (m, 3H), 5.79 (dd, J=1.5, 17.9 Hz, 1H), 5.22 (dd, J=1.5, 11.3 Hz, 1H), 4.78 (s, 2H), 4.15-4.09 (m, 2H), 3.93 (s, 1H), 3.81 (s, 3H), 3.76-3.71 (m, 2H), 1.23 (s, 9H).

Step 6a. To a mixture of 5-chloro-1H-pyrazolo[4,3-d]pyrimidine (100 mg, 647 μmol, 1 eq) and TEA (98.2 mg, 970 μmol, 1.5 eq) in DCM (2 mL) was added (Boc)₂O (169 mg, 776.41 μmol, 1.2 eq), the resulting mixture was stirred at 25° C. for 1 h. On completion, the mixture was concentrated to give a residue. The residue was purified by column chromatography (SiO₂, Petroleum ether/Ethyl acetate=5:1 to 3:1) to give tert-butyl 5-chloropyrazolo[4,3-d]pyrimidine-1-carboxylate (160 mg, 628.26 μmol, 97.10% yield) as a white solid.

Step 6. To a mixture of 1-methyl-4-(4,4,5,5-tetramethyl-1,3,2-dioxaborolan-2-yl)-5-[2-(2-vinylphenoxy)ethoxymethyl]pyrazole (93.0 mg, 242 μmol, 2 eq) and tert-butyl 5-chloropyrazolo[4,3-d]pyrimidine-1-carboxylate (30.8 mg, 121 μmol, 1 eq) in dioxane (2.25 mL) and H₂O (0.2 mL) was added Cs₂CO₃ (118 mg, 363 μmol, 3 eq), ditert-butyl(cyclopentyl)phosphane;dichloropalladium;iron (7.89 mg, 12.1 μmol, 0.1 eq), the resulting mixture was stirred at 90° C. for 16 h under N₂. On completion, the mixture was concentrated to give a residue. The residue was purified by column chromatography (SiO₂, Petroleum ether/Ethyl acetate=3:1 to 1:3) to give 5-[1-methyl-5-[2-(2-vinylphenoxy)ethoxymethyl]pyrazol-4-yl]-1H-pyrazolo[4,3-d]pyrimidine (20 mg, 53.1 μmol, 43.91% yield) as a yellow solid. 1H NMR (400 MHz, CDCl₃) δ=9.16 (m, 1H), 8.27 (d, J=2.5 Hz, 2H), 7.47 (dd, J=1.6, 7.7 Hz, 1H), 7.23-7.17 (m, 1H), 7.04 (dd, J=11.1, 17.8 Hz, 1H), 6.97-6.91 (m, 1H), 6.85 (d, J=8.3 Hz, 1H), 5.73 (dd, J=1.4, 17.8 Hz, 1H), 5.39 (s, 2H), 5.22 (dd, J=1.4, 11.2 Hz, 1H), 4.20-4.16 (m, 2H), 4.02 (s, 3H), 3.98-3.95 (m, 2H).

Step 7. To a solution of 5-[1-methyl-5-[2-(2-vinylphenoxy)ethoxymethyl]pyrazol-4-yl]-1H-pyrazolo[4,3-d]pyrimidine (138 mg, 366 μmol, 1 eq) in THF (2.5 mL) was added tBuOK (123.4 mg, 1.10 mmol, 3 eq), the resulting mixture was stirred at 0° C. for 5 min, then I₂ (93.0 mg, 366.6 μmol, 1 eq) in THF (0.7 mL) was added dropwise and stirred for another 1 h at 25° C. On completion, the mixture was filtered and concentrated to give a residue. The residue was purified by column chromatography (SiO₂, Petroleum ether/THF=10:1 to 1:1) to give 3-iodo-5-[1-methyl-5-[2-(2-vinylphenoxy)ethoxy methyl]pyrazol-4-yl]-1H-pyrazolo[4,3-d]pyrimidine (24 mg, 47.78 μmol, 13.03% yield) as a yellow oil.

Step 8. To a solution of 3-iodo-5-[1-methyl-5-[2-(2-vinylphenoxy)ethoxymethyl]pyrazol-4-yl]-1H-pyrazolo[4,3-d]pyrimidine (0.024 g, 47.78 μmol, 1 eq) in DMF (2.4 mL) was added tris-o-tolylphosphane (1.45 mg, 4.78 μmol, 0.1 eq), DIPEA (12.3 mg, 95.6 μmol, 2 eq) and Pd(OAc)2 (1.07 mg, 4.78 μmol, 0.1 eq), the resulting mixture was stirred at 120° C. for 12 h. On completion, the mixture was filtered and concentrated to give a residue. The residue was purified by prep-HPLC (column: Waters xbridge 150×25 mm×10 μm; mobile phase: [water(10 mM NH₄HCO₃)-ACN]; B %: 20%-50%, 11 min) to give Cpd. 17 (3.05 mg, 8.15 μmol, 17.05% yield) was obtained as a yellow solid.

### Example 5: Preparation of (18E)-17-ethyl-13,16-dimethyl-2,12,13,16-tetrahydro-5,3-(azenometheno)dipyrazolo[3,4-f:3′,4′-j]pyrido[4,3-n][1,4]oxazacyclopentadecine-7,14(8H,11H)-dione (Cpd. 38)

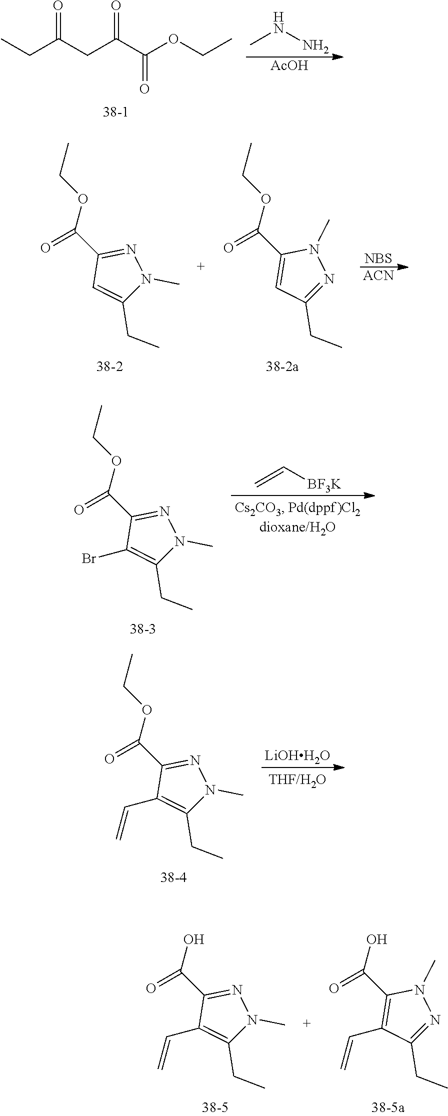

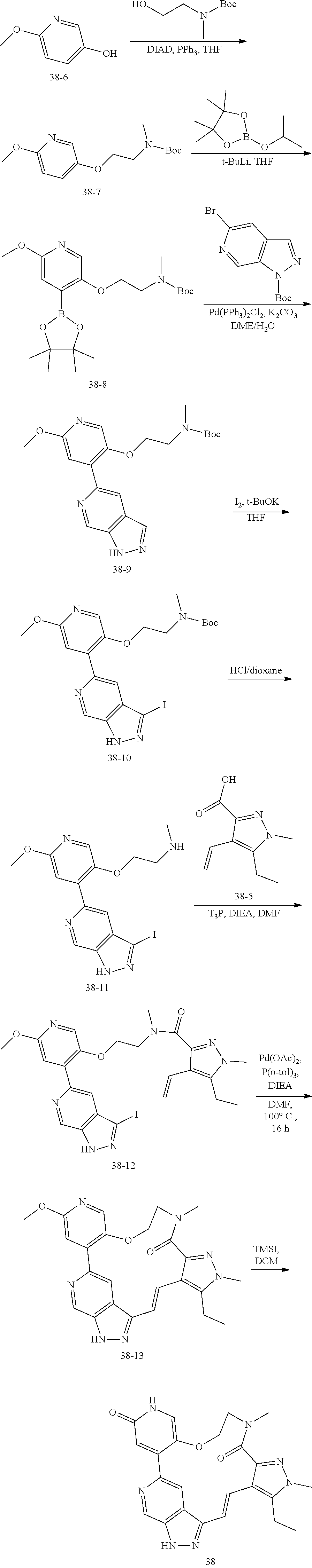

Step 1. To a solution of ethyl 2,4-dioxohexanoate (10.0 g, 58.1 mmol, 1 eq) in AcOH (65.7 g, 1.09 mol, 62.6 mL, 18.8 eq) was added methylhydrazine (7.45 g, 64.7 mmol, 8.51 mL, 40% purity, 1.11 eq) at 0° C. The mixture was stirred at 15° C. for 5 hours. LCMS showed desired MS in main peak. The mixture was concentrated in vacuum to give crude. The residue was purified by combi flash chromatography (120 g silica gel column, EtOAc in PE from 0% to 50%). Ethyl 5-ethyl-1-methyl-pyrazole-3-carboxylate (10.1 g, 55.5 mmol, 95.5% yield) was obtained as yellow oil. ¹H NMR (400 MHz, CDCl₃) δ=6.59 (s, 1H), 4.39 (q, J=14.4, 7.2 Hz, 2H), 3.85 (s, 3H), 2.62 (q, J=14.4, 7.2 Hz, 2H), 1.39 (t, J=7.2 Hz, 3H), 1.28 (t, J=7.6 Hz, 3H). Ethyl 5-ethyl-2-methyl-pyrazole-3-carboxylate (1.33 g, 7.30 mmol, 12.6% yield) was obtained as colorless oil. ¹H NMR (400 MHz, CDCl₃) δ=6.65 (s, 1H), 4.34 (q, J=7.2 Hz, 2H), 4.18-4.11 (m, 4H), 2.65 (q, J=15.2, 7.6 Hz, 2H), 1.38 (t, J=7.2 Hz, 3H), 1.25 (t, J=7.6 Hz, 3H)

Step 2. To a solution of ethyl 5-ethyl-1-methyl-pyrazole-3-carboxylate (10.0 g, 54.9 mmol, 1 eq) in MeCN (200 mL) was added NBS (10.7 g, 60.4 mmol, 1.1 eq). The mixture was stirred at 15° C. for 3 hours. TLC (Petroleum ether:Ethyl acetate/4:1, UV) showed starting material was consumed completely and another spot with lower polarity formed. The mixture was diluted with water (200 mL) and extracted with EtOAc (50 mL×3). The combined organic layer was dried over anhydrous Na₂SO₄, filtered and the filtrate was concentrated in vacuum to give crude (13.44 g). Ethyl 4-bromo-5-ethyl-1-methyl-pyrazole-3-carboxylate (13.4 g, 51.4 mmol, 93.8% yield) was obtained as yellow oil. ¹H NMR (400 MHz, CDCl₃) δ=4.41 (q, J=7.2 Hz, 2H), 3.91 (s, 3H), 2.78-2.64 (m, 2H), 1.40 (t, J=7.2 Hz, 3H), 1.18 (t, J=7.6 Hz, 3H)

Step 3. To a solution of ethyl 4-bromo-5-ethyl-1-methyl-pyrazole-3-carboxylate (13.4 g, 51.5 mmol, 1 eq), potassium hydride;trifluoro (vinyl) boron (13.8 g, 103 mmol, 2 eq), Cs₂CO₃ (50.3 g, 154 mmol, 3 eq), Pd(dppf)Cl₂ (3.77 g, 5.15 mmol, 0.1 eq) in dioxane (200 mL) and H₂O (40 mL) was stirred at 80° C. under N₂ for 3 hours. LCMS showed starting material remained and no desired MS detected. Then the mixture was stirred at 80° C. for 16 hours. TLC (Petroleum ether:Ethyl acetate/2:1, UV) showed starting material was consumed completely and another spot with lower polarity formed. The mixture was separated and organic layer was concentrated in vacuum to give crude (13.2 g). The residue was purified by combi flash chromatography (120 g silica gel column, EtOAc in PE from 0% to 60%). ethyl 5-ethyl-1-methyl-4-vinyl-pyrazole-3-carboxylate (7.62 g, 36.6 mmol, 71.1% yield) was obtained as brown oil. ¹H NMR (400 MHz, CDCl₃) δ=7.05 (dd, J=14.0, 11.6 Hz, 1H), 5.47-5.26 (m, 2H), 4.42 (J=7.2 Hz, 2H), 3.90 (s, 3H), 2.83-2.69 (m, 2H), 1.46-1.37 (m, 3H), 1.27-1.17 (m, 3H)

Step 4. To a solution of ethyl 5-ethyl-1-methyl-4-vinyl-pyrazole-3-carboxylate (1.00 g, 4.80 mmol, 1 eq) in THF (5 mL), MeOH (5 mL), H₂O (3 mL) was added LiOH·H₂O (604 mg, 14.4 mmol, 3 eq). The mixture was stirred at 15° C. for 5 hours. LCMS showed desired MS in main peak. The mixture was added 2 N HCl to just pH-5. The result solution was extracted with EtOAc (10 mL×4). The combined organic layer was dried over anhydrous Na₂SO₄, filtered and the filtrate was concentrated in vacuum to give crude. The residue was purified by Prep-HPLC (column: Phenomenex luna C18 150×25 mm×10 μm; mobile phase: [water (0.225% FA)-ACN]; B %: 40%-79%, 11 min). 5-ethyl-1-methyl-4-vinyl-pyrazole-3-carboxylic acid (746 mg, 4.14 mmol, 86.2% yield) was obtained as brown solid. ¹H NMR (400 MHz, MeOD-d₄) δ=7.03 (dd, J=18.0, 11.6 Hz, 1H), 5.44 (dd, J=18.0, 1.6 Hz, 1H), 5.27 (dd, J=11.8, 1.6 Hz, 1H), 3.87 (s, 3H), 2.84 (q, J=7.6 Hz, 2H) 1.23 (t, J=7.6 Hz, 3H)

Step 5. To a mixture of 6-methoxypyridin-3-ol (15.0 g, 120 mmol, 1 eq) and tert-butyl N-(2-hydroxyethyl)-N-methyl-carbamate (27.1 g, 155 mmol, 1.29 eq) in 2-MeTHF (100 mL) was added PPh₃ (47.2 g, 180 mmol, 1.5 eq) and DIAD (36.4 g, 180 mmol, 35.0 mL, 1.5 eq) at 0° C. under N₂. The mixture was stirred at 25° C. for 2 hours under N₂. Then the mixture was stirred at 50° C. for another 12 hours. The mixture was concentrated in vacuum. The residue was purified by silica gel chromatography (Petroleum ether/Ethyl acetate=100/1 to 30/1) to give a crude (25.0 g). The crude was purified by reverse-phase HPLC (0.1% NH₄HCO₃ condition) to give tert-butyl N-[2-[(6-methoxy-3-pyridyl)oxy]ethyl]-N-methyl-carbamate (8.00 g, 28.3 mmol, 23.6% yield) as a yellow oil which was confirmed by HNMR (EC1634-163-P1A).

Step 6. To a mixture of tert-butyl N-[2-[(6-methoxy-3-pyridyl)oxy]ethyl]-N-methyl-carbamate (3.00 g, 10.6 mmol, 1 eq) in THF (30 mL) was added t-BuLi (1.3 M, 16.4 mL, 2 eq) dropwise at −70° C. The mixture was stirred at −70° C. for 2 hours. Then 2-isopropoxy-4,4,5,5-tetramethyl-1,3,2-dioxaborolane (3.95 g, 21.3 mmol, 4.34 mL, 2 eq) was added to the mixture at −70° C. The mixture was warmed to 0° C. and stirred for 1 hour. To the mixture was added sat. NH₄Cl (5 mL) dropwise at 0° C., the mixture was extracted with EtOAc (100 mL×2). The combined organic phase was washed with brine (30 mL), dried with anhydrous Na₂SO₄, filtered and concentrated in vacuum to give tert-butyl N-[2-[[6-methoxy-4-(4,4,5,5-tetramethyl-1,3,2-dioxaborolan-2-yl)-3-pyridyl]oxy]ethyl]-N-methyl-carbamate (5 g, crude) which was used into the next step without further purification.

Step 7. A mixture of tert-butyl N-[2-[[6-methoxy-4-(4,4,5,5-tetramethyl-1,3,2-dioxaborolan-2-yl)-3-pyridyl]oxy]ethyl]-N-methyl-carbamate (5.00 g, 12.3 mmol, 1 eq), tert-butyl 5-bromopyrazolo[3,4-c]pyridine-1-carboxylate (750 mg, 2.52 mmol, 2.05e-1 eq), Pd(dppf)Cl₂·CH₂Cl₂ (250 mg, 306 μmol, 2.50e-2 eq) and K₂CO₃ (3.00 g, 21.7 mmol, 1.77 eq) in DME (60 mL) and H₂O (10 mL) was stirred at 100° C. for 2 hours under N₂. The mixture was concentrated in vacuum. The residue was purified by silica gel chromatography (Petroleum ether/Ethyl acetate=10/1 to 1/1) and then Prep-HPLC (column: Waters xbridge 150×25 mm 10 μm; mobile phase: [water(NH₄HCO₃)-ACN]; B %: 30%-60%, 11 min) to give tert-butyl N-[2-[[6-methoxy-4-(1H-pyrazolo[3,4-c]pyridin-5-yl)-3-pyridyl]oxy]ethyl]-N-methyl-carbamate (260 mg, 651 μmol, 5.32% yield) as a white solid.

Step 8. To a mixture of tert-butyl N-[2-[[6-methoxy-4-(1H-pyrazolo[3,4-c]pyridin-5-yl)-3-pyridyl]oxy]ethyl]-N-methyl-carbamate (260 mg, 651 μmol, 1 eq) in THF (5 mL) was added t-BuOK (220 mg, 1.96 mmol, 3.01 eq) and then I₂ (220 mg, 867 μmol, 175 μL, 1.33 eq) at 0° C. The mixture was stirred at 25° C. for 1 hour. To the mixture was added NaHSO₃ (aq. 5 mL). The mixture was stirred for 30 min at 20° C. The aqueous phase was extracted with ethyl acetate (30 mL×2). The combined organic phase was washed with brine (20 mL), dried with anhydrous Na₂SO₄, filtered and concentrated in vacuum. The mixture was triturated with (Petroleum ether:Ethyl acetate=3:1) to give tert-butyl N-[2-[[4-(3-iodo-1H-pyrazolo[3,4-c]pyridin-5-yl)-6-methoxy-3-pyridyl]oxy]ethyl]-N-methyl-carbamate (300 mg, 571 μmol, 87.7% yield) as a yellow solid.

Step 9. To a mixture of tert-butyl N-[2-[[4-(3-iodo-1H-pyrazolo[3,4-c]pyridin-5-yl)-6-methoxy-3-pyridyl]oxy]ethyl]-N-methyl-carbamate (300 mg, 571 μmol, 1 eq) in DCM (4 mL) was added HCl/dioxane (4 M, 2.00 mL, 14 eq) slowly at 0° C. The mixture was stirred at 25° C. for 1 hour. The mixture was concentrated in vacuum to give compound 2-[[4-(3-iodo-1H-pyrazolo[3,4-c]pyridin-5-yl)-6-methoxy-3-pyridyl]oxy]-N-methyl-ethanamine (260 mg, 563 μmol, 98.6% yield, HCl) as a yellow solid.

Step 10. To a mixture of 2-[[4-(3-iodo-1H-pyrazolo[3,4-c]pyridin-5-yl)-6-methoxy-3-pyridyl]oxy]-N-methyl-ethanamine (260 mg, 563 μmol, 1 eq, HCl) and 5-ethyl-1-methyl-4-vinyl-pyrazole-3-carboxylic acid (122 mg, 677 μmol, 1.2 eq) in DMF (5 mL) was added DIEA (364 mg, 2.82 mmol, 490 μL, 5 eq) and then T₃P (538 mg, 845 μmol, 502 μL, 50% purity, 1.5 eq). The mixture was stirred at 25° C. for 1 hour. The mixture was concentrated in vacuum. The residue was purified by prep-TLC (Dichloromethane:Methanol=10:1) to give compound 5-ethyl-N-[2-[[4-(3-iodo-1H-pyrazolo[3,4-c]pyridin-5-yl)-6-methoxy-3-pyridyl]oxy]ethyl]-N,1-dimethyl-4-vinyl-pyrazole-3-carboxamide (150 mg, 241 μmol, 42.9% yield, 94.5% purity) as a yellow solid.

Step 11. A mixture of 5-ethyl-N-[2-[[4-(3-iodo-1H-pyrazolo[3,4-c]pyridin-5-yl)-6-methoxy-3-pyridyl]oxy]ethyl]-N,1-dimethyl-4-vinyl-pyrazole-3-carboxamide (40.0 mg, 68.1 μmol, 1 eq), DIEA (26.4 mg, 204 μmol, 35.6 μL, 3 eq), tris-o-tolylphosphane (4.15 mg, 13.6 μmol, 0.2 eq) and diacetoxypalladium (1.53 mg, 6.81 μmol, 0.1 eq) in DMF (2 mL) was stirred at 120° C. for 16 hours under N₂. The mixture was concentrated in vacuum. The mixture was purified by prep-HPLC (column: Waters xbridge 150×25 mm×10 μm; mobile phase: [water(NH₄HCO₃)-ACN]; B %: 15%-45%, 11 min) to give compound 38-13 (5.63 mg, 12.1 μmol, 17.8% yield, 98.9% purity) as a white solid.

Step 12. To a solution of 38-13 (30.0 mg, 65.3 μmol, 1 eq) in DCM (3 mL) was added TMSI (131 mg, 653 μmol, 88.9 μL, 10 eq) dropwise at 0° C. The mixture was stirred at 50° C. for 2 hours. The mixture was concentrated in vacuum. The crude was purified by prep-HPLC (column: Phenomenex Luna C18 100×30 mm×5 μm; mobile phase: [water(FA)-ACN]; B %: 10%-40%, 8 min) to give Cpd. 40 (6.50 mg, 14.5 μmol, 22.2% yield, 99.2% purity) as an off-white solid.

### Example 6: Preparation of (18E)-17-ethyl-7-fluoro-13,15-dimethyl-2,12,13,15-tetrahydro-5,3-(azenometheno)dipyrazolo[3,4-f:3′,4′-j][1,4]benzoxazacyclopentadecin-14(11H)-one (Cpd. 62)

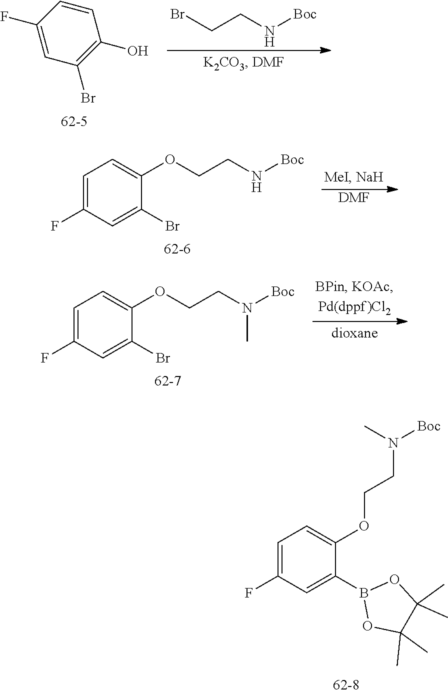

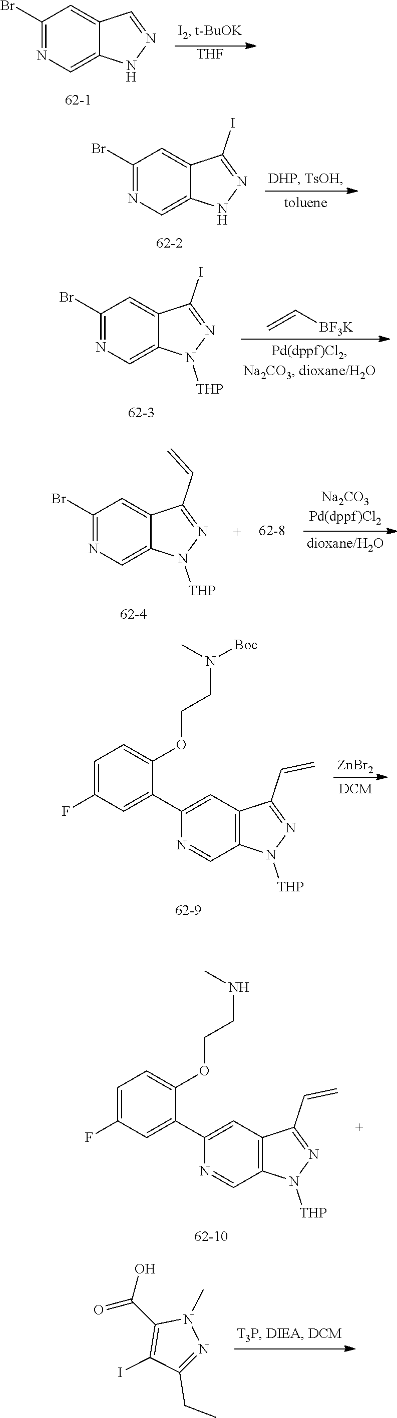

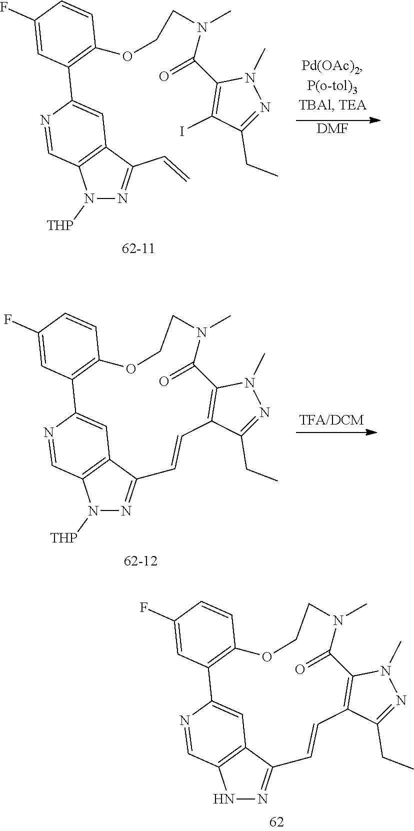

Step 1. To a solution of 5-bromo-1H-pyrazolo[3,4-c]pyridine (23.0 g, 116 mmol, 1 eq), t-BuOK (26.0 g, 232 mmol, 2 eq) in THF (300 mL) was added a solution of I₂ (32.4 g, 127 mmol, 1.1 eq) in THF (100 mL) dropwise at 0° C. The mixture was stirred at 0° C. for 3 hrs. On completion, the mixture was quenched with sat. NaHSO₃ (100 mL) and diluted with H₂O (300 mL), extracted with EA (3×300 mL), the organic layers were washed with brine (3×100 mL), dried over anhydrous Na₂SO₄, filtered and concentrated in vacuo to give 5-bromo-3-iodo-1H-pyrazolo[3,4-c]pyridine (37.5 g, 115 mmol, 99.67% yield) was obtained as yellow solid. ¹H NMR (400 MHz, DMSO-d₆) δ=8.80 (s, 1H), 7.55 (s, 1H).

Step 2. To a solution of 5-bromo-3-iodo-1H-pyrazolo[3,4-c]pyridine (25.0 g, 77.1 mmol, 1 eq) in toluene (250 mL) was added TsOH (2.66 g, 15.4 mmol, 0.2 eq) and 3,4-dihydro-2H-pyran (16.2 g, 192 mmol, 2.5 eq). The mixture was stirred at 90° C. for 2 hrs. On completion, the mixture was washed with NH₄Cl solution (2×100 mL), washed with brine (2×100 mL), dried with anhydrous Na₂SO₄, filtered and concentrated in vacuo. The residue was purified by column chromatography (SiO₂, PE/EA=100/8) to give 5-bromo-3-iodo-1-tetrahydropyran-2-yl-pyrazolo[3,4-c]pyridine (22.3 g, 54.6 mmol, 70.81% yield) was obtained as a yellow solid. ¹H NMR (400 MHz, DMSO-d₆) δ=9.08 (s, 1H), 7.72 (s, 1H), 6.00 (dd, J=1.6, 8.8 Hz, 1H), 3.92-3.84 (m, 1H), 3.80-3.72 (m, 1H), 2.35-2.26 (m, 1H), 2.06-1.96 (m, 2H), 1.79-1.66 (m, 1H), 1.63-1.55 (m, 2H).

Step 3. To a solution of 5-bromo-3-iodo-1-tetrahydropyran-2-yl-pyrazolo[3,4-c]pyridine (12.0 g, 29.4 mmol, 1 eq), potassium hydride; trifluoro (vinyl)boron (19.7 g, 147 mmol, 5 eq) in a mixture solvent of dioxane (120 mL) and H₂O (24 mL) was added Pd(dppf)Cl₂ (2.15 g, 2.94 mmol, 0.1 eq) and Na₂CO₃ (9.35 g, 88.2 mmol, 3 eq). The mixture was stirred at 40° C. for 72 hrs under N₂. On completion, the mixture was filtered and concentrated to give a residue. The residue was purified by column chromatography (SiO₂, PE/EA=50/1) to give 5-bromo-1-tetrahydropyran-2-yl-3-vinyl-pyrazolo[3,4-c] pyridine (8.6 g, 27.9 mmol, 94.89% yield) was obtained as a yellow solid. ¹H NMR (400 MHz, DMSO-d₆) δ=9.06 (s, 1H), 8.30 (s, 1H), 7.01 (dd, J=11.6, 18.0 Hz, 1H), 6.21 (d, J=18.0 Hz, 1H), 6.03-5.93 (m, 1H), 5.59 (d, J=11.6 Hz, 1H), 3.93-3.84 (m, 1H), 3.81-3.72 (m, 1H), 2.39-2.27 (m, 1H), 2.05-1.97 (m, 2H), 1.78-1.69 (m, 1H), 1.63-1.55 (m, 2H).

Step 4. To a solution of 2-bromo-4-fluoro-phenol (5.00 g, 26.2 mmol, 1 eq) in DMF (120 mL) was added K₂CO₃ (10.8 g, 78.5 mmol, 3 eq) and tert-butyl N-(2-bromoethyl) carbamate (7.04 g, 31.4 mmol, 1.2 eq). The mixture was stirred at 80° C. for 2 hours. LCMS showed starting material was consumed completely and desired MS in main peak. The mixture was diluted with water (300 mL) and extracted with EtOAc (50 mL×4). The combined organic layer was dried over anhydrous Na₂SO₄, filtered and the filtrate was concentrated in vacuum to give crude (9.45 g). Tert-butyl N-[2-(2-bromo-4-fluoro-phenoxy)ethyl] carbamate (9.45 g, crude) was obtained as light yellow oil. ¹H NMR (400 MHz, CDCl₃) δ=7.30 (dd, J=8.0, 3.2 Hz, 1H), 6.94-7.02 (m, 1H), 6.82-6.89 (m, 1H), 5.07 (s, 1H), 4.05 (t, J=4.8 Hz, 2H), 3.57 (q, J=10.4, 5.2 Hz, 2H), 1.46 (s, 11H)

Step 5. To a solution of tert-butyl N-[2-(2-bromo-4-fluoro-phenoxy)ethyl]carbamate (7.50 g, 22.4 mmol, 1 eq) in DMF (80 mL) was added NaH (1.35 g, 33.7 mmol, 60% purity, 1.5 eq) at 0° C. for 30 minutes. Then Mel (3.82 g, 26.9 mmol, 1.68 mL, 1.2 eq) was added to the mixture and stirred at 15° C. for 3 hours. TLC (Petroleum ether:Ethyl acetate/4:1, UV) showed starting material was consumed completely and another spot with smaller polarity formed. The mixture was quenched by water (150 mL) and extracted with EtOAc (50 mL×4). The combined organic layer was dried over anhydrous Na₂SO₄, filtered and the filtrate was concentrated in vacuum to give crude (7.8 g). It was used for next step without further purification. Tert-butyl N-[2-(2-bromo-4-fluoro-phenoxy)ethyl]-N-methyl-carbamate (7.80 g, 22.4 mmol, 99.8% yield) was obtained as yellow solid.

Step 6. To a solution of tert-butyl N-[2-(2-bromo-4-fluoro-phenoxy)ethyl]-N-methyl-carbamate (9.00 g, 25.6 mmol, 1 eq) and 4,4,5,5-tetramethyl-2-(4,4,5,5-tetramethyl-1,3,2-dioxaborolan-2-yl)-1,3,2-dioxaborolane (7.88 g, 31.0 mmol, 1.2 eq) in dioxane (200 mL) was added KOAc (7.61 g, 77.5 mmol, 3 eq) and Pd(dppf)Cl₂ (1.89 g, 2.58 mmol, 0.1 eq). The mixture was stirred at 100° C. under N₂ for 3 h. On completion, the mixture was concentrated in vacuum to give crude. The residue was purified by combi flash (120 g silica gel column, EtOAc in PE from 0% to 100%) to give tert-butyl N-[2-[4-fluoro-2-(4,4,5,5-tetramethyl-1,3,2-dioxaborolan-2-yl)phenoxy]ethyl]-N-methyl-carbamate (5.60 g, 14.2 mmol, 54.8% yield) was obtained as white solid.

Step 7. To a solution of tert-butyl N-[2-[4-fluoro-2-(4,4,5,5-tetramethyl-1,3,2-dioxaborolan-2-yl)phenoxy]ethyl]-N-methyl-carbamate (1.92 g, 4.87 mmol, 1.25 eq), 5-bromo-1-tetrahydropyran-2-yl-3-vinyl-pyrazolo[3,4-c]pyridine (1.20 g, 3.89 mmol, 1 eq), Cs₂CO₃ (3.81 g, 11.7 mmol, 3 eq) in dioxane (30 mL) and H₂O (6 mL) was added Pd(dppf)Cl₂·CH₂Cl₂ (317 mg, 389 μmol, 0.1 eq) at 25° C., the mixture was stirred at 90° C. for 12 hour under N₂. The reaction mixture was concentrated in vacuo. The residue was purified by column chromatography on silica gel (PE:EA=25:1-1:1) to give tert-butyl N-[2-[4-fluoro-2-(1-tetrahydropyran-2-yl-3-vinyl-pyrazolo[3,4-c]pyridin-5-yl)phenoxy]ethyl]-N-methyl-carbamate (1.7 g, 3.15 mmol, 80.9% yield, 92% purity) as a yellow oil. 1H NMR (400 MHz, CDCl₃) δ=9.17 (d, J=1.2 Hz, 1H), 8.35 (s, 1H), 7.58-7.56 (m, 1H), 7.10-6.94 (m, 3H), 6.14 (d, J=18.0 Hz, 1H), 5.84-5.81 (m, 1H), 5.61 (d, J=12.0 Hz, 1H), 4.16-4.05 (m, 3H), 3.86-3.69 (m, 1H), 3.54 (s, 2H), 2.82-2.61 (m, 3H), 2.20-2.11 (m, 2H), 1.82-1.72 (m, 2H), 1.65 (s, 2H), 1.41 (s, 9H).

Step 8. A mixture of tert-butyl N-[2-[4-fluoro-2-(1-tetrahydropyran-2-yl-3-vinyl-pyrazolo[3,4-c]pyridin-5-yl)phenoxy]ethyl]-N-methyl-carbamate (3.5 g, 7.05 mmol, 1 eq) in DCM (40 mL), then ZnBr₂ (7.94 g, 35.2 mmol, 1.76 mL, 5 eq) was added at 25° C. The mixture was stirred at 25° C. for 16 h. After cooled to 25° C., the mixture was diluted with water (300 mL), extracted with EA (3×100 mL). The combined organic layer was washed with brine (3×100 mL), dried over Na₂SO₄, filtered and concentrated in vacuo. The residue was purified by column chromatography on silica gel (DCM:MeOH=25:1-1:1) to give 2-[4-fluoro-2-(1-tetrahydropyran-2-yl-3-vinyl-pyrazolo[3,4-c]pyridin-5-yl)phenoxy]-N-methyl-ethanamine (2.5 g, 5.68 mmol, 80.5% yield, 90% purity) as yellow oil. ¹H NMR (400 MHz, DMSO-d₆) δ=9.33 (s, 1H), 8.51 (s, 1H), 7.60-7.59 (m, 1H), 7.33-7.20 (m, 2H), 7.11-7.10 (m, 1H), 6.24 (d, J=18.0 Hz, 1H), 6.08-6.02 (m, 1H), 5.62 (d, J=12 Hz, 1H), 4.39 (t, J=5.1 Hz, 2H), 4.13 (s, 1H), 3.27 (t, J=5.2 Hz, 2H), 3.16 (s, 3H), 2.54 (s, 2H), 2.42-2.35 (m, 1H), 2.04 (d, J=10.6 Hz, 2H), 1.81-1.73 (m, 1H), 1.67-1.58 (m, 2H).

Step 9. To a solution of 2-[4-fluoro-2-(1-tetrahydropyran-2-yl-3-vinyl-pyrazolo[3,4-c]pyridin-5-yl)phenoxy]-N-methyl-ethanamine (2.5 g, 6.31 mmol, 1 eq), 5-ethyl-4-iodo-2-methyl-pyrazole-3-carboxylic acid (2.12 g, 7.57 mmol, 1.2 eq), DIEA (4.07 g, 31.5 mmol, 5.49 mL, 5 eq) in DCM (40 mL) was added T₃P (6.02 g, 9.46 mmol, 5.63 mL, 50% purity, 1.5 eq) at 0° C. The mixture was stirred at 25° C. for 16 h. The mixture was diluted with water (100 mL) and extracted with EtOAc (50 mL×3). The combined organic layer was dried over anhydrous Na₂SO₄, filtered and the filtrate was concentrated in vacuum. The residue was purified by combi flash (4 g silica gel column, EtOAc in PE from 0% to 100%) to give 5-ethyl-N-[2-[4-fluoro-2-(1-tetrahydropyran-2-yl-3-vinyl-pyrazolo[3,4-c]pyridin-5-yl)phenoxy]ethyl]-4-iodo-N,2-dimethyl-pyrazole-3-carboxamide (2.5 g, 3.72 mmol, 58.9% yield, 97.9% purity) as a yellow oil. ¹H NMR (400 MHz, CDCl₃) δ=9.12 (d, J=1.0 Hz, 1H), 8.32 (d, J=1.2 Hz, 1H), 7.53-7.50 (m, 1H), 7.07-6.99 (m, 3H), 6.13-6.12 (m, 1H), 5.82-5.80 (m, 1H), 5.57-5.50 (m, 1H), 4.33 (t, J=5.2 Hz, 2H), 3.71-3.67 (m, 3H), 2.81 (s, 3H), 2.58-2.49 (m, 4H), 2.19-2.12 (m, 2H), 1.88-1.62 (m, 6H), 1.19 (t, J=7.6 Hz, 3H).

Step 10. To a solution of 5-ethyl-N-[2-[4-fluoro-2-(1-tetrahydropyran-2-yl-3-vinyl-pyrazolo[3,4-c]pyridin-5-yl)phenoxy]ethyl]-4-iodo-N,2-dimethyl-pyrazole-3-carboxamide (2.0 g, 3.04 mmol, 1 eq), TEA (1.54 g, 15.2 mmol, 2.11 mL, 5 eq), TBAI (336 mg, 911 μmol, 0.3 eq) and P(o-tolyl)3 (739 mg, 2.43 mmol, 0.8 eq) in DMF (200 mL), Pd(OAc)₂ (272 mg, 1.21 mmol, 0.4 eq) were added. The mixture was stirred at 100° C. for 16 h under nitrogen atmosphere. The mixture was added to water (400 mL) to quench, extracted with EA (3×100 mL). The combined organic layers were washed with brine (2×100 mL), dried over Na₂SO₄, filtered and concentrated in vacuo. The residue was purified by column chromatography on silica gel (PE:EA=25:1-0:1) to give compound 62-12 (1.3 g, 2.08 mmol, 34.3% yield, 85% purity) as a white solid. ¹H NMR (400 MHz, CDCl₃) δ=9.18 (d, J=12.4 Hz, 1H), 9.01 (s, 1H), 8.06-8.04 (m, 1H), 7.58-7.54 (m, 1H), 7.17-7.14 (m, 1H), 7.07-7.00 (m, 2H), 5.81 (d, J=9.6 Hz, 1H), 4.77-4.57 (m, 2H), 4.07-4.06 (m, 1H), 4.22-4.03 (m, 1H), 3.92 (s, 3H), 3.87-3.73 (m, 2H), 3.22 (s, 3H), 2.96-2.90 (m, 2H), 2.19-2.11 (m, 2H), 1.81 (t, J=9.8 Hz, 2H), 1.67 (s, 2H), 1.39 (t, J=7.6 Hz, 3H).

Step 11. To a mixture of 62-12 (1.3 g, 2.45 mmol, 1 eq) in DCM (10 mL) was added TFA (15.4 g, 135 mmol, 10 mL, 55.1 eq). The mixture was stirred at 25° C. for 1 hour. The reaction mixture was concentrated in vacuo. The crude product was purified by prep-HPLC (water(TFA)-ACN: 23%-53%) to give Cpd. 64 (702.01 mg, 1.54 mmol, 62.70% yield, 97.7% purity) as a yellow solid.

### Example 7: Preparation of (11E)-1-methyl-1,18,19,21-tetrahydro-8H-10,7,4-(ethan[1]yl[1,2]diylidene)pyrazolo[3,4-f][1,4,9,12,13]benzodioxatriazacyclooctadecine (Cpd. 63)

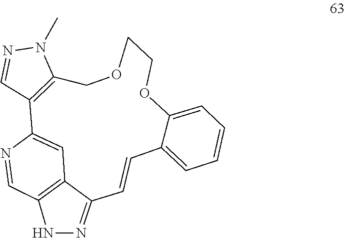 [SMILES: Cn1ncc2c1COCCOc1ccccc1/C=C/c1n[nH]c3cnc-2cc13]

Cpd. 63 was prepared following similar procedures as Cpd. 3 using 1-2 and 16-6 as starting materials.

### Example 8: Preparation of (19E)-18-ethyl-7-methoxy-14,17-dimethyl-2,11,12,13,14,17-hexahydro-15H-5,3-(azenometheno)dipyrazolo[3,4-g:3′,4′-k]pyrido[4,3-o][1,5]oxazacyclohexadecin-15-one (EX. 64)

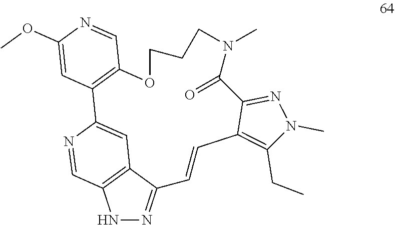

Cpd. 64 was prepared following similar procedures as 38-13 using tert-butyl N-(3-chloropropyl)-N-methyl-carbamate for alkylation reaction with 6-methoxypyridin-3-ol.

### Example 9: Preparation of (18E)-8,9,16-trimethyl-8,9,11,12-tetrahydro-2H-5,3-(azenometheno)dipyrazolo[3,4-f:4′,3′-j]pyrido[2,3-n][1,4,9]dioxazacyclopentadecine (Cpd. 65)

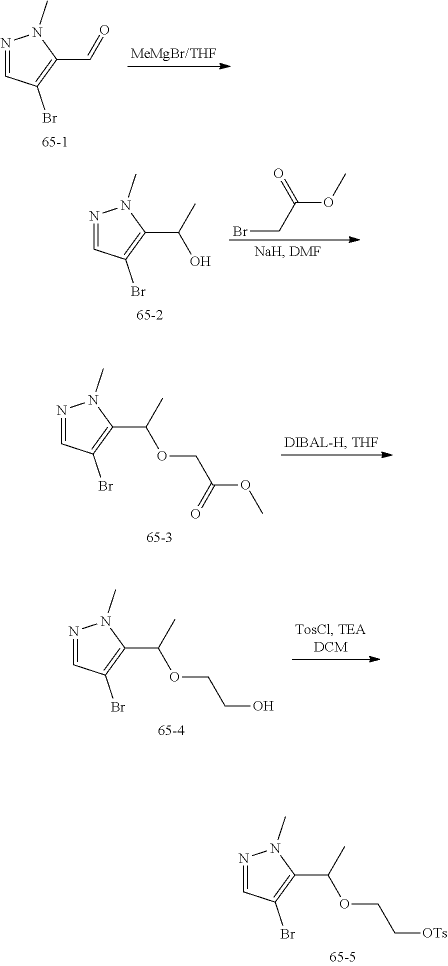

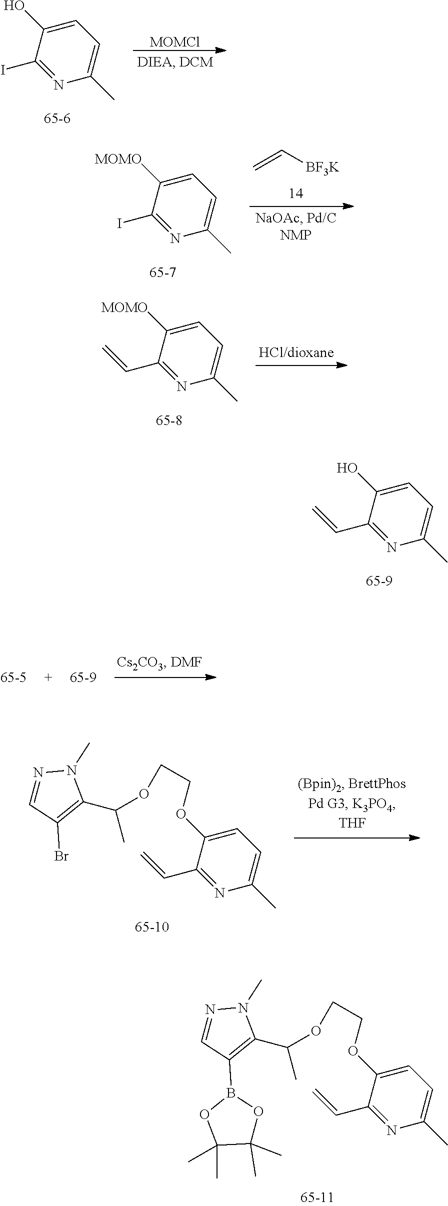

Step 1. To a solution of 4-bromo-2-methyl-pyrazole-3-carbaldehyde (4.80 g, 25.4 mmol, 1 eq) in THF (48 mL) was added MeMgBr (3 M, 9.31 mL, 1.1 eq) at 0° C. The mixture was stirred at 15° C. for 2 h. On completion, the mixture was quenched with water (100 mL) and extracted with ethyl acetate (50 mL×3), the combined organic phase was dried over anhydrous sodium sulfate, filtered and concentrated to give a residue. The residue was purified by column chromatography (SiO₂, Dichloromethane/Methanol=1:0 to 20:1) to give 1-(4-bromo-2-methyl-pyrazol-3-yl) ethanol (4.8 g, 92.18% yield) as colorless oil.

Step 2. To a solution of 1-(4-bromo-2-methyl-pyrazol-3-yl)ethanol (4.50 g, 21.9 mmol, 1 eq) in THF (75 mL) was added NaH (1.76 g, 43.9 mmol, 60% purity, 2 eq) at 0° C. The mixture was stirred at 0° C. for 0.5 h, after that a solution of methyl 2-bromoacetate (5.04 g, 32.9 mmol, 1.5 eq) in THF (30 mL) was added to the mixture, the mixture was stirred at 25° C. for 2 h. On completion, the mixture was quenched with water (150 mL) and extracted with ethyl acetate (100 mL×3), the combined organic phase was dried over anhydrous sodium sulfate, filtered and concentrated to give a residue. The residue was purified by column chromatography (SiO₂, Petroleum ether/Ethyl acetate=1:0 to 30:1) to give methyl 2-[1-(4-bromo-2-methyl-pyrazol-3-yl)ethoxy]acetate (2.9 g, 47.69% yield) as colorless oil.

Step 3. A solution of methyl 2-[1-(4-bromo-2-methyl-pyrazol-3-yl)ethoxy]acetate (2.50 g, 9.02 mmol, 1 eq) in THF (25 mL) was degassed and purged with N₂ for 3 times, and then DIBAL-H (1 M, 27.06 mL, 3 eq) was added dropwise at 0° C. The mixture was stirred at 25° C. for 1 h under N₂ atmosphere. On completion, the mixture was quenched with MeOH (10 mL), filtered and concentrated to give a residue. The residue was purified by column chromatography (SiO₂, Dichloromethane:Methanol=1:0 to 30:1) to give 2-[1-(4-bromo-2-methyl-pyrazol-3-yl)ethoxy]ethanol (1.7 g, 75.65% yield) as a white solid.

Step 4. To a solution of 2-[1-(4-bromo-2-methyl-pyrazol-3-yl)ethoxy]ethanol (1.46 g, 5.86 mmol, 1 eq) in DCM (15 mL) was added TEA (1.78 g, 17.5 mmol, 3 eq) and TosCl (1.68 g, 8.79 mmol, 1.5 eq). The mixture was stirred at 15° C. for 2 h. On completion, the mixture was quenched with water (20 mL) and extracted with ethyl acetate (15 mL×2), the combined organic phase was dried over anhydrous sodium sulfate, filtered and concentrated to give a residue. The residue was purified by column chromatography (SiO₂, Petroleum ether/Ethyl acetate=1:0 to 2:1) to give 2-[1-(4-bromo-2-methyl-pyrazol-3-yl)ethoxy]ethyl 4-methylbenzenesulfonate (2.35 g, 99.2% yield) as colorless oil.

Step 5. To a solution of 2-iodo-6-methyl-pyridin-3-ol (10.0 g, 42.5 mmol, 1 eq) in DCM (100 mL) was added DIEA (8.16 g, 63.1 mmol, 11.0 mL, 1.48 eq) and MOMCl (6.89 g, 85.5 mmol, 2.01 eq) at 0° C. The mixture was degassed and purged with N₂ for 3 times, and then the mixture was stirred at 15° C. for 16 h under N₂ atmosphere. On completion, the mixture was quenched with water (10 mL), the mixture was washed with 1 M HCl (40×2 mL), and then the organic phase was washed with aq. NaCl (50×2 mL), the organic phase was dried over anhydrous sodium sulfate, filtered and concentrated to give 2-iodo-3-(methoxymethoxy)-6-methyl-pyridine (11.7 g, 98.5% yield) as yellow oil.

Step 6. To a solution of 2-iodo-3-(methoxymethoxy)-6-methyl-pyridine (3.00 g, 10.7 mmol, 1 eq) in NMP (15 mL) was added potassium;trifluoro(vinyl)boranuide (1.58 g, 11.8 mmol, 1.1 eq), Pd/C (30 mg, 10% purity, 1 eq) and NaOAc (2.65 g, 32.2 mmol, 3 eq). The mixture was degassed and purged with N₂ for 3 times, and then the mixture was stirred at 100° C. for 16 hours under N₂ atmosphere. On completion, the mixture was quenched with water (20 mL) and extracted with ethyl acetate (10 mL×2), the combined organic phase was dried over anhydrous sodium sulfate, filtered and concentrated to give a residue. The residue was purified by column chromatography (SiO₂, Petroleum ether:Ethyl acetate=1:0 to 5:1) to give 3-(methoxymethoxy)-6-methyl-2-vinyl-pyridine (1 g, 51.91% yield) as a yellow oil.

Step 7. A solution of 3-(methoxymethoxy)-6-methyl-2-vinyl-pyridine (860 mg, 4.80 mmol, 1 eq) in HCl/dioxane (8 mL) was stirred at 0° C. for 2 h. On completion, the mixture was concentrated to give 6-methyl-2-vinyl-pyridin-3-ol (823 mg, 4.80 mmol, 99.9% yield, HCl) as white solid.

Step 8 To a mixture of 2-[1-(4-bromo-2-methyl-pyrazol-3-yl)ethoxy]ethyl 4-methylbenzenesulfonate (2.12 g, 5.26 mmol, 1.1 eq) and 6-methyl-2-vinyl-pyridin-3-ol (820 mg, 4.78 mmol, 1 eq, HCl) in DMF (21 mL) was added Cs₂CO₃ (4.67 g, 14.3 mmol, 3 eq). The mixture was stirred at 50° C. for 2 h. On completion, the mixture was quenched with water (40 mL) and extracted with ethyl acetate (30 mL×2), the combined organic phase was dried over anhydrous sodium sulfate, filtered and concentrated to give a residue. The residue was purified by column chromatography (SiO₂, Petroleum ether/Ethyl acetate=1:0 to 2:1) to give 3-[2-[1-(4-bromo-2-methyl-pyrazol-3-yl)ethoxy]ethoxy]-6-methyl-2-vinyl-pyridine (1.3 g, 74.3% yield) as colorless oil.

Step 9. To a solution of 3-[2-[1-(4-bromo-2-methyl-pyrazol-3-yl)ethoxy]ethoxy]-6-methyl-2-vinyl-pyridine (1.15 g, 3.14 mmol, 1 eq) in THF (23 mL) was added 4,4,5,5-tetramethyl-2-(4,4,5,5-tetramethyl-1,3,2-dioxaborolan-2-yl)-1,3,2-dioxaborolane (1.59 g, 6.28 mmol, 2 eq), BrettPhos Pd G3 (284 mg, 313 μmol, 0.1 eq) and K₃PO₄ (2.00 g, 9.42 mmol, 3 eq). The mixture was degassed and purged with N₂ for 3 times, and then the mixture was stirred at 50° C. for 16 h under N₂ atmosphere. On completion, the mixture was concentrated to give a residue. The residue was purified by column chromatography (SiO₂, Petroleum ether:Ethyl acetate=3:1) to give 6-methyl-3-[2-[1-[2-methyl-4-(4,4,5,5-tetramethyl-1,3,2-dioxaborolan-2-yl)pyrazol-3-yl]ethoxy]ethoxy]-2-vinyl-pyridine (300 mg, 23.1% yield) as colorless oil.

Cpd. 65 was prepared following similar procedures as Cpd. 16 using 65-11 and 16-7 as starting materials.

### Example 10: Preparation of (18E)-17-ethyl-7-methoxy-13,16-dimethyl-2,12,13,16-tetrahydro-5,3-(azenometheno)dipyrazolo[3,4-f:3′,4′-j]pyrido[4,3-n][1,4]oxazacyclopentadecin-14(11H)-one (Cpd. 66)

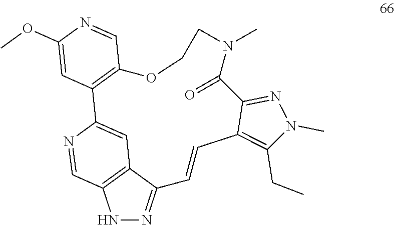

Cpd. 66 was prepared using similar procedures as 38-13.

### Example 11: Preparation of methyl (11E)-1-methyl-19,20-dihydro-1H,8H,18H-10,7,4-(ethan[1]yl[1,2]diylidene)pyrazolo[3,4-f][1,5,9,12,13]benzodioxatriazacyclooctadecine-15-carboxylate (Cpd. 67)

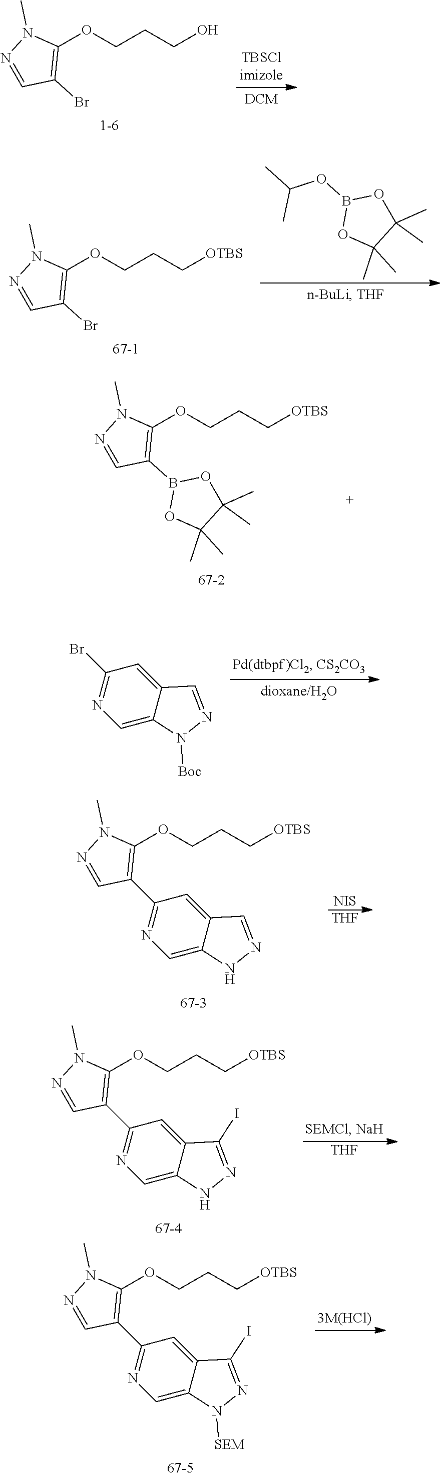

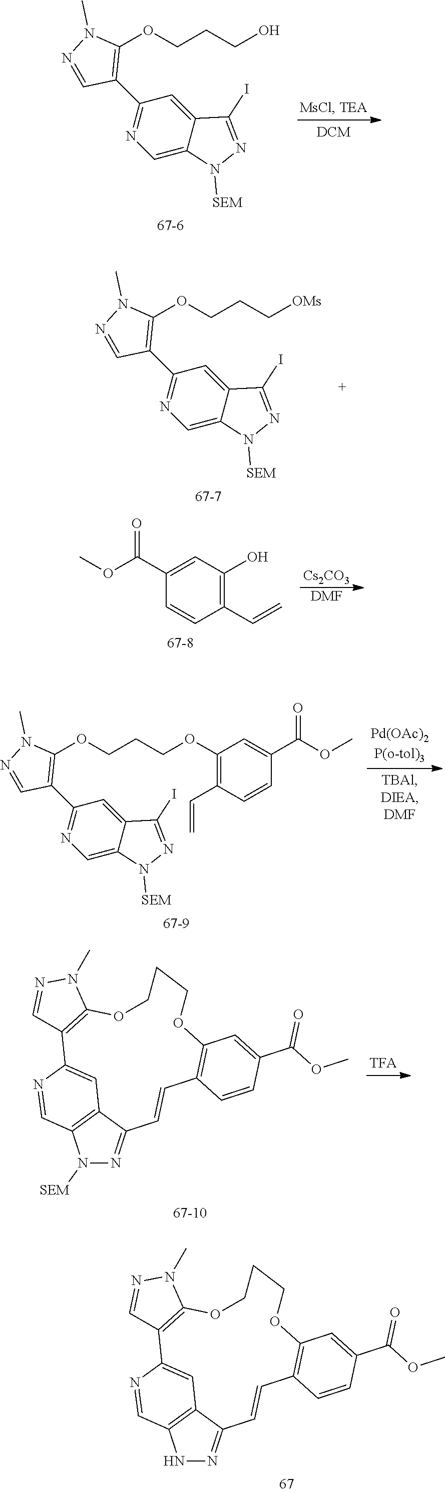

Step 1. To a solution of 3-(4-bromo-2-methyl-pyrazol-3-yl)oxypropan-1-ol (1.00 g, 4.25 mmol, 1.0 eq) in DCM (20 mL) was added imidazole (579 mg, 8.51 mmol, 2.0 eq). Then tert-butyl-chloro-dimethyl-silane (962 mg, 6.38 mmol, 782 μL, 1.5 eq) was added at 0° C. The reaction mixture was stirred at 25° C. for 1 hr. The mixture was added to water (30 mL), extracted with DCM (3×30 mL). The combined organic layers were washed with brine (2×20 mL), dried over Na₂SO₄, filtered and concentrated in vacuum to give 3-(4-bromo-2-methyl-pyrazol-3-yl)oxypropoxy-tert-butyl-dimethyl-silane (1.40 g, 3.89 mmol, 91.4% yield, 97.0% purity) as a yellow oil. ¹H NMR (400 MHz, DMSO-d₆) δ=7.38 (s, 1H), 4.28 (t, J=6.0 Hz, 2H), 3.77-3.73 (m, 2H), 3.62 (s, 3H), 1.89 (t, J=6.0 Hz, 2H), 0.86 (s, 9H), 0.04 (s, 6H).

Step 2. To a mixture of 3-(4-bromo-2-methyl-pyrazol-3-yl)oxypropoxy-tert-butyl-dimethyl-silane (1.30 g, 3.72 mmol, 1.0 eq) in 2-MeTHF (20 mL) was added n-BuLi (1 M, 7.44 mL, 2.0 eq) at −78° C. for 0.5 hr, then 2-isopropoxy-4,4,5,5-tetramethyl-1,3,2-dioxaborolane (2.08 g, 11.2 mmol, 2.28 mL, 3.0 eq) was added at −78° C. for 1 hr. The mixture was diluted with water (40 mL), extracted with EA (3×30 mL). The combined organic layer was washed with brine (30 mL), dried over Na₂SO₄, filtered and concentrated in vacuum to give tert-butyl-dimethyl-[3-[2-methyl-4-(4,4,5,5-tetramethyl-1,3,2-dioxaborolan-2-yl)pyrazol-3-yl]oxypropoxy]silane (1.40 g, 1.17 mmol, 31.3% yield, 33.0% purity) as a yellow oil.

Step 3. To a solution of tert-butyl-dimethyl-[3-[2-methyl-4-(4,4,5,5-tetramethyl-1,3,2-dioxaborolan-2-yl)pyrazol-3-yl]oxypropoxy]silane (1.40 g, 3.53 mmol, 1.0 eq), tert-butyl 5-bromopyrazolo[3,4-c]pyridine-1-carboxylate (1.26 g, 4.24 mmol, 1.2 eq), Cs₂CO₃ (3.45 g, 10.6 mmol, 3.0 eq) in dioxane (20 mL) and H₂O (4 mL) was added ditert-butyl(cyclopentyl)phosphane;dichloropalladium;iron (230 mg, 353 μmol, 0.1 eq) at 25° C., the mixture was stirred at 90° C. for 12 hours under N₂. The reaction mixture was concentrated in vacuo. The residue was purified by column chromatography on silica gel (DCM:MeOH=25:1-10:1) to give tert-butyl-dimethyl-[3-[2-methyl-4-(1H-pyrazolo[3,4-c]pyridin-5-yl)pyrazol-3-yl]oxypropoxy]silane (350 mg, 559 μmol, 15.8% yield, 62.0% purity) as a yellow oil. ¹H NMR (400 MHz, DMSO-d₆) δ=13.55 (s, 1H), 9.01 (s, 1H), 8.16 (s, 1H), 7.87 (s, 1H), 7.79 (s, 1H), 4.17 (t, J=6.4 Hz, 2H), 3.75 (t, J=6.4 Hz, 2H), 3.69 (s, 3H), 1.93 (t, J=6.4 Hz, 2H), 0.77 (s, 9H), −0.02-0.04 (m, 6H).

Step 4. To a solution of tert-butyl-dimethyl-[3-[2-methyl-4-(1H-pyrazolo[3,4-c]pyridin-5-yl)pyrazol-3-yl]oxypropoxy]silane (317 mg, 817 μmol, 1.0 eq) in THF (5 mL) at 0° C., potassium;2-methylpropan-2-olate (275 mg, 2.45 mmol, 3.0 eq) was added iodine (249 mg, 981 μmol, 197 μL, 1.2 eq) at 0° C. The mixture was stirred at 0° C. for 16 hr. The reaction mixture was quenched by water (10 mL) and extracted with EA (3×10 mL). The combined organic layers were washed with brine (2×5 mL), dried over Na₂SO₄, filtered and concentrated in vacuum. The residue was purified by column chromatography (SiO₂, Petroleum ether/Ethyl acetate=10/1 to 0/1) to give tert-butyl-[3-[4-(3-iodo-1H-pyrazolo[3,4-c]pyridin-5-yl)-2-methyl-pyrazol-3-yl]oxypropoxy]-dimethyl-silane (230 mg, 421 μmol, 51.5% yield, 94.0% purity) as yellow solid. ¹H NMR (400 MHz, DMSO-d₆) δ=13.96 (s, 1H), 9.01 (s, 1H), 8.16 (s, 1H), 7.84 (s, 1H), 7.52 (s, 1H), 4.17 (t, J=6.4 Hz, 2H), 3.75 (t, J=6.4 Hz, 2H), 3.69 (s, 3H), 1.97 (t, J=6.4 Hz, 2H), 0.77 (s, 9H), 0.01-0.004 (m, 6H).

Step 5. To a stirred solution of tert-butyl-[3-[4-(3-iodo-1H-pyrazolo[3,4-c]pyridin-5-yl)-2-methyl-pyrazol-3-yl]oxypropoxy]-dimethyl-silane (230 mg, 448 μmol, 1.0 eq) in DMF (2 mL) was added NaH (26.8 mg, 672 μmol, 60.0% purity, 1.5 eq) at 0° C. and the mixture was stirred at 0° C. for 0.5 h. Then 2-(chloromethoxy)ethyl-trimethyl-silane (89.6 mg, 537 μmol, 95.1 μL, 1.2 eq) was added dropped into the mixture and stirred at 0° C. for 1.5 hr. The mixture was added to water (10 mL) to quench, extracted with EA (3×10 mL). The combined organic layers were washed with brine (2×10 mL), dried over Na₂SO₄, filtered and concentrated in vacuum to give tert-butyl-[3-[4-[3-iodo-1-(2-trimethylsilylethoxymethyl)-pyrazolo[3,4-c]pyridin-5-yl]-2-methyl-pyrazol-3-yl]oxypropoxy]-dimethyl-silane (210 mg, 326 μmol, 72.8% yield) as a yellow solid.

Step 6. To a stirred solution of tert-butyl-[3-[4-[3-iodo-1-(2-trimethylsilylethoxymethyl)pyrazolo[3,4-c]pyridin-5-yl]-2-methyl-pyrazol-3-yl]oxypropoxy]-dimethyl-silane (330 mg, 512 μmol, 1.0 eq) in MeOH (20 mL) was added HCl (3 M, 11.0 mL, 64.3 eq). The mixture was stirred at 20° C. for 1 hr. The mixture was quenched with Sat. NaHCO₃ (10 mL), concentrated in vacuum to remove MeOH and the mixture was extracted with EA (30 mL). The organic layer was washed with brine (2×20 mL), concentrated in vacuum to afford crude. The crude was purified by silica gel column (PE:EA=100:30) to afford 3-[4-[3-iodo-1-(2-trimethylsilylethoxymethyl)pyrazolo[3,4-c]pyridin-5-yl]-2-methyl-pyrazol-3-yl]oxypropan-1-ol (250 mg, 472 μmol, 92.1% yield) as colorless solid.

Step 7. To a stirred solution of 3-[4-[3-iodo-1-(2-trimethylsilylethoxymethyl)pyrazolo[3,4-c]pyridin-5-yl]-2-methyl-pyrazol-3-yl]oxypropan-1-ol (250 mg, 472 μmol, 1.0 eq) and TEA (143 mg, 1.42 mmol, 197 μL, 3.0 eq) in DCM (20 mL) was added MsCl (81.14 mg, 708 μmol, 54.8 μL, 1.5 eq) at 0° C. and the mixture was stirred at 0° C. for 1 hr. On completion, the mixture was quenched with water (10 mL). Then, the organic layer was dried over sodium sulfate, washed with brine (2×20 mL), concentrated in vacuum to afford 3-[4-[3-iodo-1-(2-trimethylsilylethoxymethyl)pyrazolo[3,4-c]pyridin-5-yl]-2-methyl-pyrazol-3-yl]oxypropyl methanesulfonate (280 mg, 437 μmol, 92.7% yield, 95.0% purity) as off-white solid.

Step 8. The mixture of 3-[4-[3-iodo-1-(2-trimethylsilylethoxymethyl)pyrazolo[3,4-c]pyridin-5-yl]-2-methyl-pyrazol-3-yl]oxypropyl methanesulfonate (280 mg, 461 μmol, 1.0 eq), methyl 3-hydroxy-4-vinyl-benzoate (1.24 g, 691 μmol, 1.5 eq) and Cs₂CO₃ (450 mg, 1.38 mmol, 3.0 eq) in DMF (3 mL) was stirred at 60° C. for 16 hr. The mixture was diluted with EtOAc (50 mL), washed with brine (3×30 mL). The organic layer was dried over sodium sulfate, concentrated in vacuum to afford crude. The crude was purified by silica gel column (DCM:MeOH=100:4) to afford methyl 3-[3-[4-[3-iodo-1-(2-trimethylsilylethoxymethyl)pyrazolo[3,4-c]pyridin-5-yl]-2-methyl-pyrazol-3-yl]oxypropoxy]-4-vinyl-benzoate (120 mg, 156 μmol, 33.9% yield, 90.0% purity) as colorless solid.

Step 9. To a solution of methyl 3-[3-[4-[3-iodo-1-(2-trimethylsilylethoxymethyl)pyrazolo[3,4-c]pyridin-5-yl]-2-methyl-pyrazol-3-yl]oxypropoxy]-4-vinyl-benzoate (20.0 mg, 29.0 μmol, 1.0 eq), TEA (4.40 mg, 43.5 μmol, 6.06 μL, 1.5 eq) and TBAI (1.07 mg, 2.90 μmol, 0.1 eq) in DMF (2 mL) was added Pd(OAc)₂ (325 μg, 1.45 μmol, 0.05 eq) and P(o-tolyl)3 (883 μg, 2.90 μmol, 0.1 eq). The mixture was stirred at 60° C. for 2 hr under nitrogen atmosphere. The mixture was added to water (20 mL), extracted with EA (3×10 mL). The combined organic layers were washed with brine (2×10 mL), dried over Na₂SO₄, filtered and concentrated in vacuum. The residue was purified by column chromatography on silica gel (PE:EA=25:1-1:1) to give 67-10 (25.0 mg, 39.6 μmol, 34.1% yield, 89.0% purity) as a white solid.

Step 10. The mixture of 67-10 (10.0 mg, 17.8 μmol, 1.0 eq) in TFA (1.54 g, 13.5 mmol, 1 mL, 758 eq) was stirred at 25° C. for 12 hours. The reaction mixture was concentrated in vacuum. The crude product was purified by Pre-HPLC (water(TFA)-ACN: 30%-60%) to give Cpd. 67 (1.70 mg, 3.89 μmol, 21.8% yield, 98.8% purity) as yellow solid.

### Example 12: Preparation of methyl (11E)-1-methyl-19,20-dihydro-1H,8H,18H-10,7,4-(ethan[1]yl[1,2]diylidene)pyrazolo[3,4-f][1,5,9,12,13]benzodioxatriazacyclooctadecine-14-carboxylate (Cpd. 68)

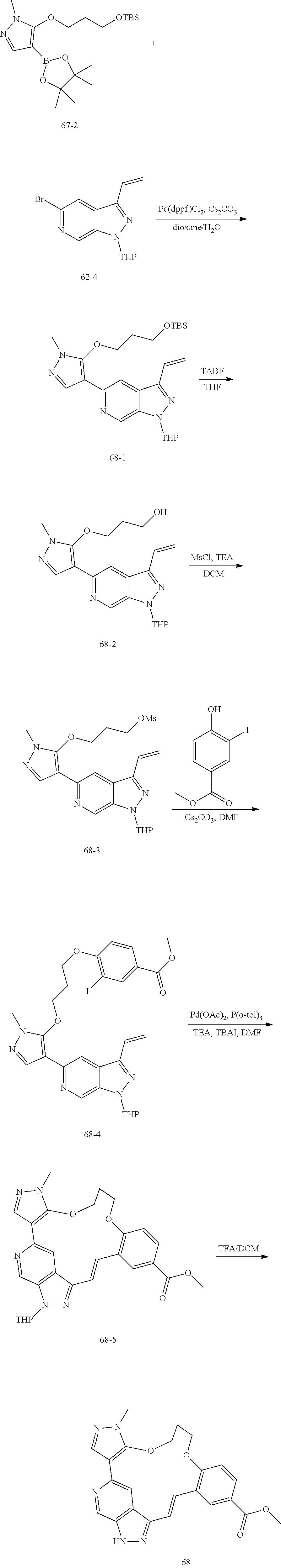

Step 1. To a mixture of tert-butyl-dimethyl-[3-[2-methyl-4-(4,4,5,5-tetramethyl-1,3,2-dioxaborolan-2-yl)pyrazol-3-yl]oxypropoxy]silane (771 mg, 1.95 mmol, 3 eq) and 5-bromo-1-tetrahydropyran-2-yl-3-vinyl-pyrazolo[3,4-c]pyridine (200 mg, 648 μmol, 1 eq) in dioxane (4 mL) and H2O (0.1 mL) was added ditert-butyl(cyclopentyl)phosphane;dichloropalladium;iron (42.3 mg, 64.9 μmol, 0.1 eq) and Cs2CO3 (634 mg, 1.95 mmol, 3 eq). The reaction mixture was stirred at 90° C. for 3 hour. On completion, the reaction mixture was diluted with water (30 mL) and extracted with EA (2×20 mL). The combined organic layers was dried over Na2SO4, filtered and concentrated in vacuo to give a residue. The residue was purified by column chromatography (SiO₂, PE:EA=50:1 to PE:EA=5:1,PE:EA=1:1, P1:Rf=0.40) to afford tert-butyl-dimethyl-[3-[2-methyl-4-(1-tetrahydropyran-2-yl-3-vinyl-pyrazolo[3,4-c]pyridin-5-yl)pyrazol-3-yl]oxypropoxy]silane (130 mg, 261 μmol, 40% yield) as light yellow oil.

Step 2. To a mixture of tert-butyl-dimethyl-[3-[2-methyl-4-(1-tetrahydropyran-2-yl-3-vinyl-pyrazolo[3,4-c]pyridin-5-yl)pyrazol-3-yl]oxypropoxy]silane (130 mg, 261 μmol, 1 eq) in THF (2 mL) was added TBAF (1 M, 313 μL, 1.2 eq) at 0° C. The reaction mixture was stirred at 0° C. for 6 hours. On completion, the reaction mixture was diluted with water (30 mL) and extracted with EA (2×30 mL). The combined organic layers was dried over Na₂SO₄, filtered and concentrated in vacuo to afford 3-[2-methyl-4-(1-tetrahydropyran-2-yl-3-vinyl-pyrazolo[3,4-c]pyridine-5-yl)pyrazol-3-yl]oxypropan-1-ol (100 mg, 260 μmol, 99.84% yield) as light yellow oil.

Step 3. To a mixture of 3-[2-methyl-4-(1-tetrahydropyran-2-yl-3-vinyl-pyrazolo[3,4-c]pyridin-5-yl) pyrazol-3-yl]oxypropan-1-ol (0.1 g, 260 μmol, 1 eq) and TEA (79.1 mg, 782 μmol, 108 μL, 3 eq) in DCM (5 mL) was added MsCl (59.7 mg, 521 μmol, 40.3 μL, 2 eq). The reaction mixture was stirred at 25° C. for 1 hour. On completion, the reaction mixture was diluted with water (30 mL) and extracted with DCM (2×30 mL). The combined organic layers was dried over Na₂SO₄, filtered and concentrated in vacuo to afford 3-[2-methyl-4-(1-tetrahydropyran-2-yl-3-vinyl-pyrazolo[3,4-c]pyridin-5-yl)pyrazol-3-yl]oxypropyl methanesulfonate (120 mg, 260 μmol, 99.70% yield) as light yellow oil.

Step 4. To a mixture of 3-[2-methyl-4-(1-tetrahydropyran-2-yl-3-vinyl-pyrazolo[3,4-c]pyridin-5-yl) pyrazol-3-yl]oxypropyl methanesulfonate (120 mg, 260 μmol, 1 eq) and methyl 4-hydroxy-3-iodo-benzoate (108 mg, 390 μmol, 1.5 eq) in DMF (3 mL) was added Cs₂CO₃ (254 mg, 780 μmol, 3 eq). The reaction mixture was stirred at 60° C. for 12 hour. On completion, The reaction mixture was diluted with water (10 mL) and extracted with EA (2×30 mL). The combined organic layers was dried over Na₂SO₄, filtered and concentrated in vacuo to give a residue. The residue was purified by column chromatography (SiO₂, PE:EA=50:1 to PE:EA=5:1,PE:EA=3:1, P1:Rf=0.43) to afford methyl 3-iodo-4-[3-[2-methyl-4-(1-tetrahydropyran-2-yl-3-vinyl-pyrazolo[3,4-c]pyridin-5-yl)pyrazol-3-yl]oxypropoxy]benzoate (110 mg, 170 μmol, 65.7% yield) as white solid. ¹H NMR (400 MHz, DMSO-d₆) δ=9.10 (d, J=1.2 Hz, 1H), 8.23 (d, J=2.0 Hz, 1H), 8.05 (d, J=1.2 Hz, 1H), 7.93 (dd, J=2.0, 8.8 Hz, 1H), 7.90 (s, 1H), 7.09 (d, J=8.8 Hz, 1H), 6.98 (dd, J=11.6, 18.1 Hz, 1H), 6.14 (d, J=18.0 Hz, 1H), 5.89 (dd, J=2.4, 9.6 Hz, 1H), 5.55 (d, J=12.4 Hz, 1H), 4.35 (td, J=6.0, 18.0 Hz, 4H), 3.90 (d, J=11.2 Hz, 1H), 3.82 (s, 3H), 3.80-3.72 (m, 1H), 3.69 (s, 3H), 2.40-2.30 (m, 1H), 2.26 (q, J=6.0 Hz, 2H), 2.05-1.98 (m, 2H), 1.81-1.69 (m, 1H), 1.65-1.56 (m, 2H).

Step 5. To a mixture of methyl 3-iodo-4-[3-[2-methyl-4-(1-tetrahydropyran-2-yl-3-vinyl-pyrazolo[3,4-c]pyridin-5-yl)pyrazol-3-yl]oxypropoxy]benzoate (90.0 mg, 139 μmol, 1 eq) in DMF (2 mL) was added Pd(OAc)₂ (3.14 mg, 13.9 μmol, 0.1 eq), TEA (21.2 mg, 209 μmol, 29.2 μL, 1.5 eq), TBAI (5.17 mg, 13.9 μmol, 0.1 eq) and P(o-tolyl)₃ (4.26 mg, 13.9 μmol, 0.1 eq). The reaction mixture was stirred at 60° C. for 12 hour. On completion, the reaction mixture was diluted with water (10 mL) and extracted with EA (2×20 mL). The combined organic layers was dried over Na₂SO₄, filtered and concentrated in vacuo to give a residue. The residue was purified by prep-TLC ((SiO₂,PE:EA=1:1, P1:Rf=0.3) to afford 68-5 (26 mg, 50.4 μmol, 36.0% yield) as white solid.

Step 6. To a mixture of 68-5 (24 mg, 46.5 μmol, 1 eq) in DCM (1.5 mL) was added TFA (2.31 g, 20.2 mmol, 1.5 mL, 435 eq). The reaction mixture was stirred at 25° C. for 1 hour. On completion, the reaction mixture was concentrated in vacuo. The residue was triturated with CAN, filtered and concentrated in vacuo to afford Cpd. 68 (1.9 mg, 3.38 μmol, 7.26% yield, 97% purity, TFA) as yellow solid.

### Example 13: Preparation of methyl (11E)-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-15-carboxylate (Cpd. 69)

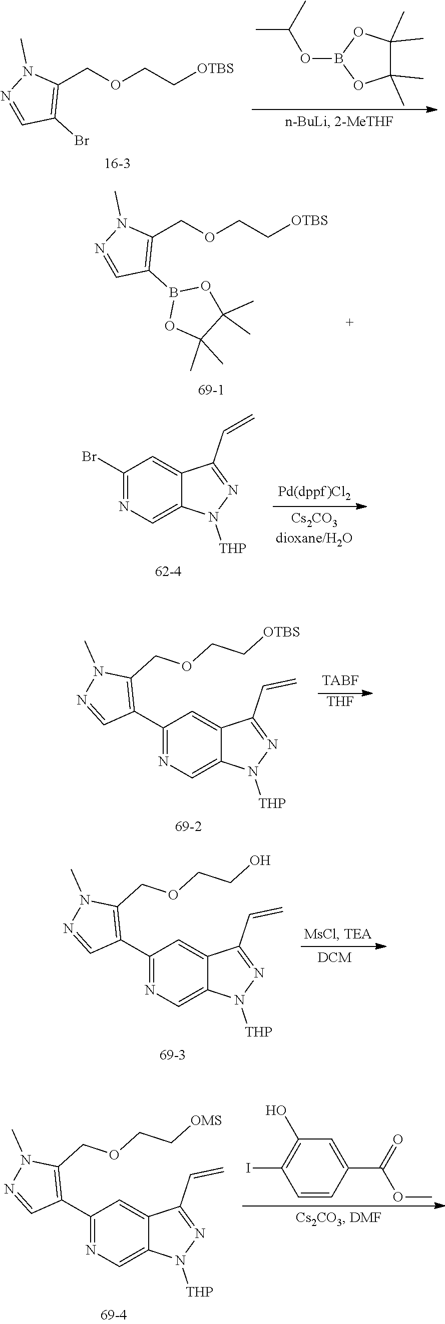

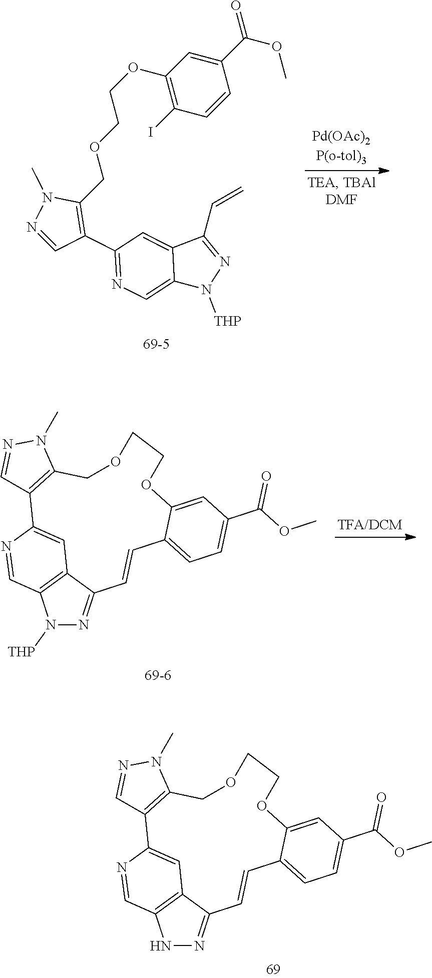

Step 1. To a solution of 2-[(4-bromo-2-methyl-pyrazol-3-yl)methoxy]ethoxy-tert-butyl-dimethyl-silane (2.0 g, 5.73 mmol, 1 eq) in 2-MeTHF (50 mL) was added n-BuLi (2.5 M, 3.44 mL, 1.5 eq) at −70° C. The mixture was stirred at −70° C. for 0.5 hr. Then 2-isopropoxy-4,4,5,5-tetramethyl-1,3,2-dioxaborolane (2.1 g, 11.4 mmol, 2 eq) was added and stirred at this temperature for 1.5 hrs. On completion, the mixture was added into NH₄Cl solution (60 mL), extracted with EA (3×100 mL), washed with brine (3×50 mL). The organic layer was dried over sodium sulfate, filtered and concentrated in vacuo to give a residue. The residue was purified by column chromatography (SiO₂, PE/EA=100/8) to give tert-butyl-dimethyl-[2-[[2-methyl-4-(4,4,5,5-tetramethyl-1,3,2-dioxaborolan-2-yl)pyrazol-3-yl] methoxy] ethoxy]silane (1.54 g, 2.02 mmol, 35.29% yield, 52% purity) was obtained as a yellow oil. ¹H NMR (400 MHz, CDCl₃) δ=7.69 (s, 1H), 4.58 (s, 2H), 3.92 (s, 3H), 3.79-3.77 (m, 2H), 3.75-3.72 (m, 2H), 1.31 (s, 12H), 0.89 (s, 9H), 0.07 (s, 6H).

Cpd. 69 was prepared following similar procedures as Cpd. 68 using 69-1 and 62-4 as starting materials. Methyl 3-hydroxy-4-iodo-benzoate was used in alkylation reaction (Step 4 in Cpd. 68).

### Example 14-16: Preparation of (11E)-N-[3-(dimethylamino)propyl]-1-methyl-19,20-dihydro-1H,8H,18H-10,7,4-(ethan[1]yl[1,2]diylidene)pyrazolo[3,4-f][1,5,9,12,13]benzodioxatriazacyclooctadecine-14-carboxamide (Cpd. 70), (11E)-N-[3-(dimethylamino)propyl]-1-methyl-19,20-dihydro-1H,8H,18H-10,7,4-(ethan[1]yl[1,2]diylidene)pyrazolo[3,4-f][1,5,9,12,13]benzodioxatriazacyclooctadecine-14-carboxamide (Cpd. 71) and [(11E)-1-methyl-19,20-dihydro-1H,8H,18H-10,7,4-(ethan[1]yl[1,2]diylidene)pyrazolo[3,4-f][1,5,9,12,13]benzodioxatriazacyclooctadecin-14-yl](pyrrolidin-1-yl)methanone (Cpd. 72)

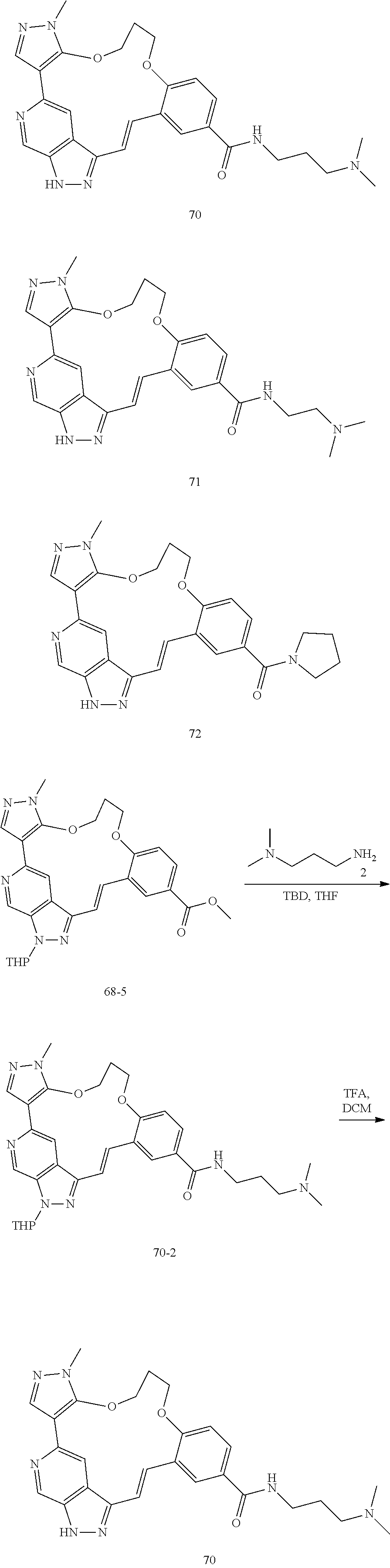

Step 1. To a mixture of 68-5 (100 mg, 193 μmol, 1 eq) and N′,N′-dimethylpropane-1,3-diamine (25.7 mg, 252 μmol, 31.5 μL, 1.3 eq) in THF (3 mL) was added 3,4,6,7,8,9-hexahydro-2H-pyrimido[1,2-a]pyrimidine (32.4 mg, 232 μmol, 1.2 eq). The reaction mixture was stirred at 70° C. for 1 hour. On completion, the reaction mixture was diluted with water (31 mL) and extracted with EA (2×20 mL). The combined organic layers was dried over Na₂SO₄, filtered and concentrated in vacuo to afford 70-2 (100 mg, 170 μmol, 88.0% yield) as light yellow solid.

Step 2. To a mixture of 70-2 (100 mg, 170 μmol, 1 eq) in DCM (2 mL) was added TFA (2 mL). The reaction mixture was stirred at 25° C. for 1 hour. On completion, the reaction mixture was concentrated in vacuo. The residue was purified by prep-HPLC (column: Welch Xtimate C18 150×25 mm×5 μm; mobile phase: [water(TFA)-ACN]; B %: 5%-35%, 10 min) to afford Cpd. 70 (35.16 mg, 57.1 μmol, 33.4% yield, 100% purity, TFA) as yellow solid.

Cpd. 71 and Cpd. 72 were prepared following similar procedures as Cpd. 70 using N′,N′-dimethylethane-1,2-diamine and pyrrolidine in Step 1 for amide formation, respectively.

### Example 17: Preparation of (11E)-1-methyl-19,20-dihydro-1H,8H,18H-10,7,4-(ethan[1]yl[1,2]diylidene)pyrazolo[3,4-f][1,5,9,12,13]benzodioxatriazacyclooctadecine-15-carboxylic acid (Cpd. 73)

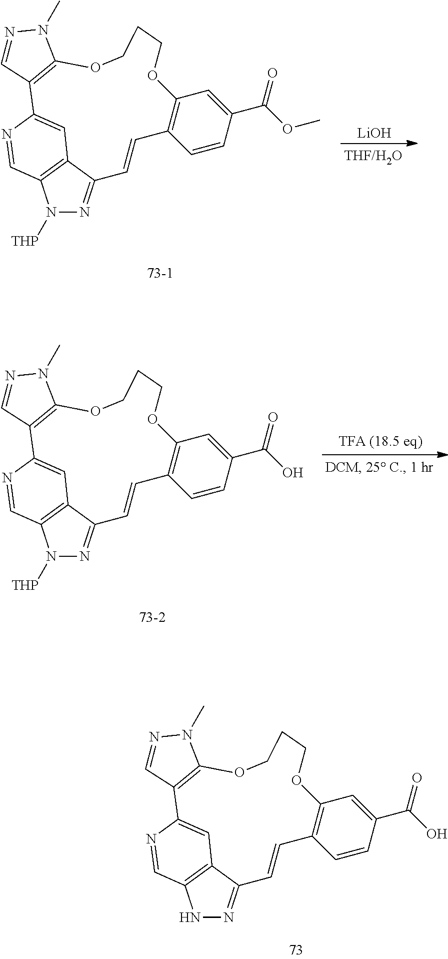

73-1 was prepared following similar procedures as 68-5. Methyl 3-hydroxy-4-iodo-benzoate was used in alkylation reaction (Step 4 in Cpd. 68).

Step 1. To a mixture of 73-1 (100 mg, 193 μmol, 1 eq) in THF (2 mL), H₂O (0.4 mL) and MeOH (2 mL) was added LiOH·H₂O (12.2 mg, 290 μmol, 1.5 eq). The reaction mixture was stirred at 50° C. for 12 hour. On completion, the reaction mixture was concentrated in vacuo. The residue was diluted with water (10 mL) and Acidified basified with TFA till pH=5-6, yellow solid was formed and filtered to afford 73-2 (96 mg, 191 μmol, 98% yield) as yellow solid. ¹H NMR (400 MHz, DMSO-d₆) δ=13.37-12.23 (m, 1H), 9.21 (s, 1H), 8.37 (s, 2H), 8.13 (d, J=17.2 Hz, 1H), 7.96-7.88 (m, 2H), 7.48 (d, J=17.2 Hz, 1H), 7.35 (d, J=8.8 Hz, 1H), 6.00 (d, J=8.8 Hz, 1H), 4.51 (s, 2H), 4.39 (t, J=4.8 Hz, 2H), 3.92 (d, J=11.6 Hz, 1H), 3.84-3.78 (m, 1H), 3.77 (s, 3H), 2.45-2.40 (m, 3H), 2.06 (d, J=10.4 Hz, 2H), 1.85-1.70 (m, 1H), 1.62 (s, 2H).

Step 2. To a solution of 73-2 (38.0 mg, 75.7 μmol, 1.0 eq) in DCM (5 mL) was added TFA (159 mg, 1.40 mmol, 104 μL, 18.5 eq) at 25° C., the mixture was stirred at 25° C. for 1 hr. The reaction mixture was concentrated in vacuum. The mixture was added water (10 mL), added TFA adjust pH to 4-5, filtered and concentrated to give a residue. The crude product was triturated with ACN at 25° C. for 10 mins to give Cpd. 73 (10.3 mg, 24.3 μmol, 32.1% yield, 97.9% purity) as a yellow solid.

### Example 18: Preparation of (11E)-N-[2-(dimethylamino)ethyl]-1-methyl-19,20-dihydro-1H,8H,18H-10,7,4-(ethan[1]yl[1,2]diylidene)pyrazolo[3,4-f][1,5,9,12,13]benzodioxatriazacyclooctadecine-15-carboxamide (Cpd. 74)

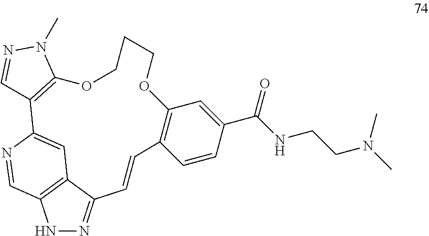 [SMILES: CN(C)CCNC(=O)c1ccc2c(c1)OCCCOc1c(cnn1C)-c1cc3c(n[nH]c3cn1)/C=C/2]

Cpd. 74 was prepared following similar procedures as Cpd. 70 using 73-1 and N′,N′-dimethylethane-1,2-diamine as starting materials.

### Example 19: Preparation of methyl (11E)-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-14-carboxylate (Cpd. 75)

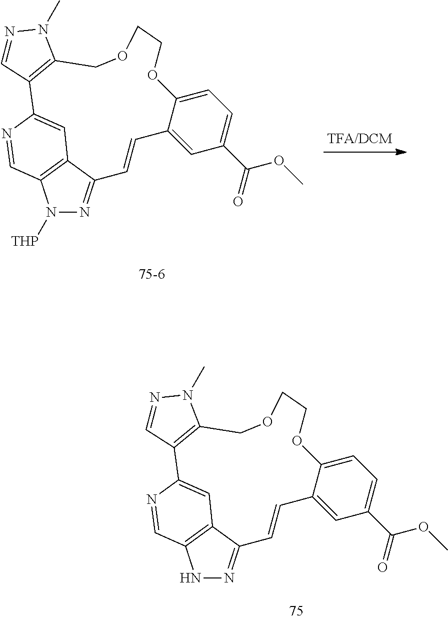

75-6 was prepared following similar procedures as 69-6 using 69-1 and 62-4 as starting materials and methyl 4-hydroxy-3-iodo-benzoate as alkylation reagent in Step 5 for Cpd. 69. 75-6 was further converted to Cpd. 75 as 69-6 to Cpd. 69.

### Example 20: Preparation of (11E)-1-methyl-N-[(1-methylpiperidin-4-yl)methyl]-19,20-dihydro-1H,8H,18H-10,7,4-(ethan[1]yl[1,2]diylidene)pyrazolo[3,4-f][1,5,9,12,13]benzodioxatriazacyclooctadecine-14-carboxamide (Cpd. 76)

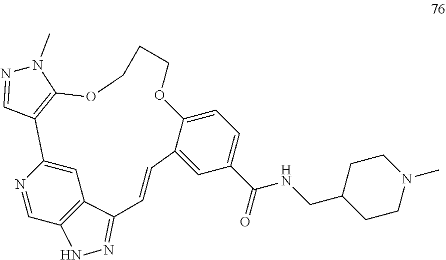 [SMILES: CN1CCC(CNC(=O)c2ccc3c(c2)/C=C/c2n[nH]c4cnc(cc24)-c2cnn(C)c2OCCCO3)CC1]

Cpd. 76 were prepared following similar procedures as Cpd. 70 using 1-methylpiperdin-4-yl)methanamine in Step 1 for amide formation.

### Example 21: Preparation of (11E)-1-methyl-N-(propan-2-yl)-19,20-dihydro-1H,8H,18H-10,7,4-(ethan[1]yl[1,2]diylidene)pyrazolo[3,4-f][1,5,9,12,13]benzodioxatriazacyclooctadecine-15-carboxamide (Cpd. 77)

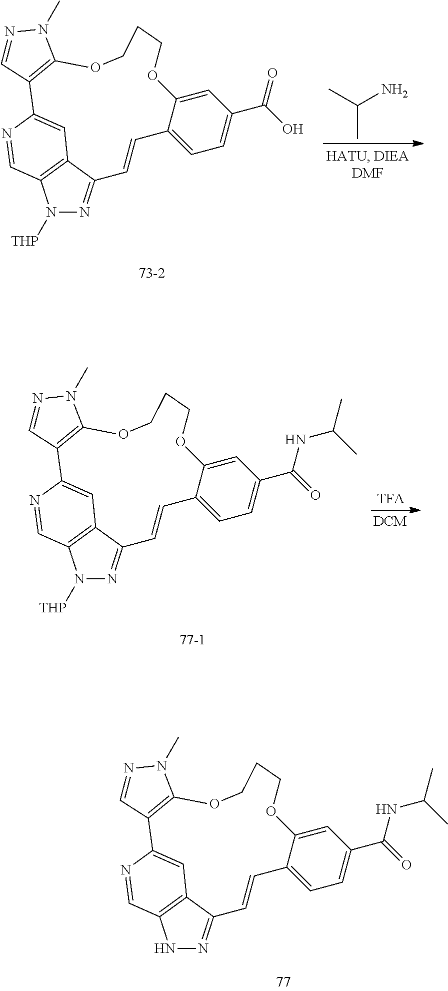

Step 1. To a solution of 73-2 (90.0 mg, 179 μmol, 1.0 eq) and HATU (88.7 mg, 233 μmol, 1.3 eq), DIEA (69.5 mg, 538 μmol, 93.7 μL, 3.0 eq) in DMF (4 mL) was added propan-2-amine (21.2 mg, 358 μmol, 30.8 μL, 2.0 eq) at 0° C. The mixture was stirred at 25° C. for 10 mins. The mixture was added to water (20 mL) to quench, extracted with EA (3×10 mL). The combined organic layers were washed with brine (2×10 mL), dried over Na₂SO₄, filtered and concentrated in vacuo. The crude product 77-1 was used directly without purification for the next step.

Step 2. To a mixture of 77-1 (80.0 mg, 147 μmol, 1.0 eq) in DCM (5 mL) was added TFA (7.70 g, 67.5 mmol, 5.00 mL, 458 eq) at 25° C. for 1 hour. The reaction mixture was concentrated in vacuo. The crude product was purified by prep-HPLC(water(TFA)-ACN: 17%-47%) to give Cpd. 77 (41.4 mg, 87.6 μmol, 59.4% yield, 97.0% purity) as a yellow solid.

### Example 22: Preparation of (11E)-1-methyl-N-(1-methylpiperidin-4-yl)-19,20-dihydro-1H,8H,18H-10,7,4-(ethan[1]yl[1,2]diylidene)pyrazolo[3,4-f][1,5,9,12,13]benzodioxatriazacyclooctadecine-15-carboxamide (Cpd. 78)

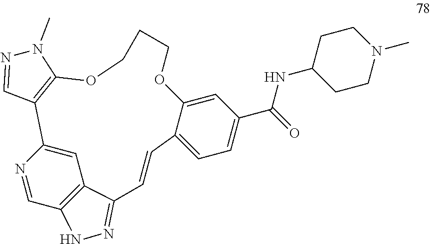 [SMILES: CN1CCC(NC(=O)c2ccc3c(c2)OCCCOc2c(cnn2C)-c2cc4c(n[nH]c4cn2)/C=C/3)CC1]

Cpd. 78 were prepared following similar procedures as Cpd. 77 using 1-methylpiperidin-4-amine in Step 1 for amide coupling.

### Example 23: Preparation of (11E)-1-methyl-19,20-dihydro-1H,8H,18H-10,7,4-(ethan[1]yl[1,2]diylidene)pyrazolo[3,4-f][1,5,9,12,13]benzodioxatriazacyclooctadecine-14-carboxylic acid (Cpd. 79)

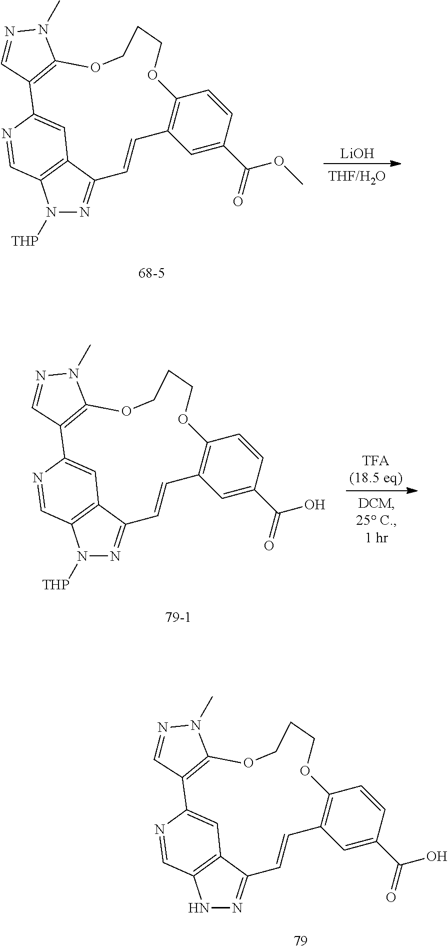

Cpd. 79 was obtained following similar procedures as Cpd. 73 using 68-5 as starting material.

### Example 24: Preparation of (11E)-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-15-carboxylic acid (Cpd. 80)

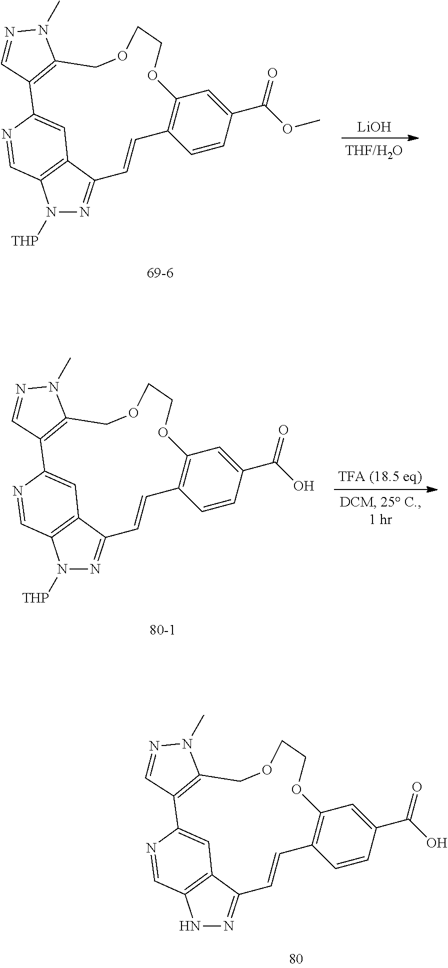

Cpd. 80 was obtained following similar procedures as Cpd. 73 using 69-6 as starting material.

### Example 25-27: Preparation of (11E)-N-[2-(dimethylamino)ethyl]-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-15-carboxamide (Cpd. 81), (11E)-1-methyl-N-[(3R)-1-methylpyrrolidin-3-yl]-19,20-dihydro-1H,8H,18H-10,7,4-(ethan[1]yl[1,2]diylidene)pyrazolo[3,4-f][1,5,9,12,13]benzodioxatriazacyclooctadecine-15-carboxamide (Cpd. 82), and (11E)-1-methyl-N-(1-methylazetidin-3-yl)-19,20-dihydro-1H,8H,18H-10,7,4-(ethan[1]yl[1,2]diylidene)pyrazolo[3,4-f][1,5,9,12,13]benzodioxatriazacyclooctadecine-15-carboxamide (Cpd. 83)

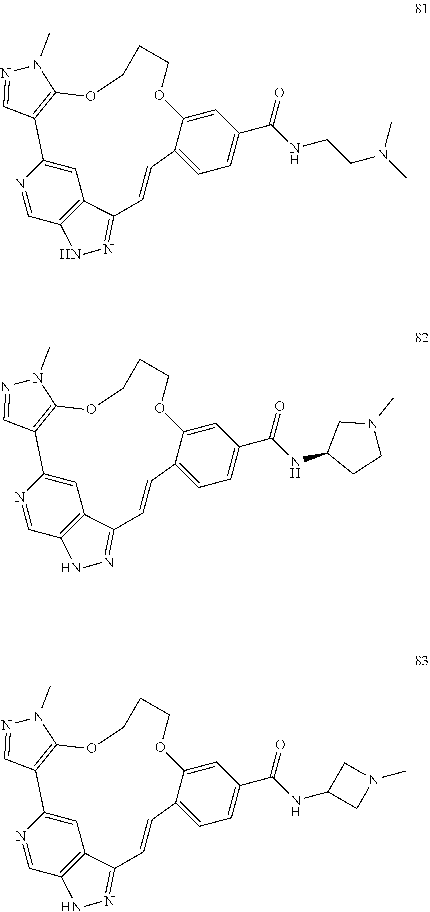 [SMILES: CN(C)CCNC(=O)c1ccc2c(c1)OCCCOc1c(cnn1C)-c1cc3c(n[nH]c3cn1)/C=C/2 ; CN1CC[C@@H](NC(=O)c2ccc3c(c2)OCCCOc2c(cnn2C)-c2cc4c(n[nH]c4cn2)/C=C/3)C1 ; CN1CC(NC(=O)c2ccc3c(c2)OCCCOc2c(cnn2C)-c2cc4c(n[nH]c4cn2)/C=C/3)C1]

Cpd. 81, Cpd. 82, and Cpd. 83 were prepared following similar procedures as Cpd. 77 using corresponding amines for amide coupling in step 1.

### Example 28 and 29: Preparation of (11E)-N-ethyl-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-15-carboxamide (Cpd. 84) and (11E)-1-methyl-N-(1-methylpiperidin-4-yl)-1,18,19,21-tetrahydro-8H-10,7,4-(ethan[1]yl[1,2]diylidene)pyrazolo[3,4-f][1,4,9,12,13]benzodioxatriazacyclooctadecine-15-carboxamide (Cpd. 85)

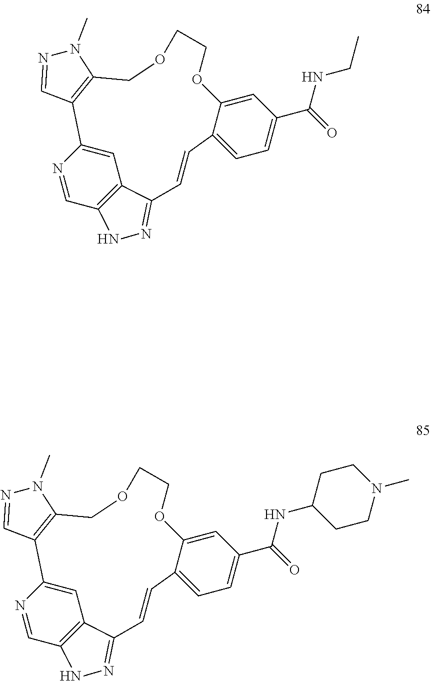 [SMILES: CCNC(=O)c1ccc2c(c1)OCCOCc1c(cnn1C)-c1cc3c(n[nH]c3cn1)/C=C/2 ; CN1CCC(NC(=O)c2ccc3c(c2)OCCOCc2c(cnn2C)-c2cc4c(n[nH]c4cn2)/C=C/3)CC1]

Cpd. 84 and Cpd. 85 were prepared following similar procedures as Cpd. 77 using 80-1 and corresponding amines for amide coupling in step 1.

### Example 30: Preparation of (11E)-N,1-dimethyl-19,20-dihydro-1H,8H,18H-10,7,4-(ethan[1]yl[1,2]diylidene)pyrazolo[3,4-f][1,5,9,12,13]benzodioxatriazacyclooctadecine-14-carboxamide (Cpd. 86)

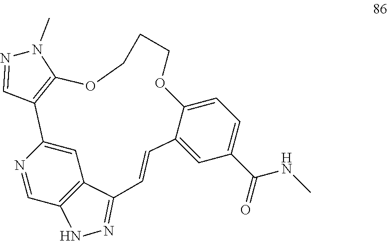 [SMILES: CNC(=O)c1ccc2c(c1)/C=C/c1n[nH]c3cnc(cc13)-c1cnn(C)c1OCCCO2]

Cpd. 86 was prepared following similar procedures as Cpd. 77 using 79-1 and methylamine for amide coupling in step 1.

### Example 31: Preparation of (11E)-1-methyl-N-[(3R)-1-methylpyrrolidin-3-yl]-1,18,19,21-tetrahydro-8H-10,7,4-(ethan[1]yl[1,2]diylidene)pyrazolo[3,4-f][1,4,9,12,13]benzodioxatriazacyclooctadecine-15-carboxamide (Cpd. 87)

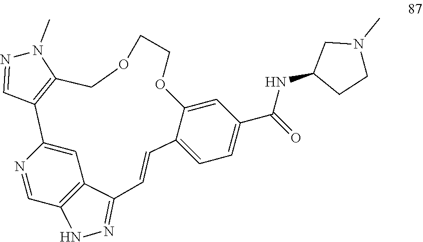 [SMILES: CN1CC[C@@H](NC(=O)c2ccc3c(c2)OCCOCc2c(cnn2C)-c2cc4c(n[nH]c4cn2)/C=C/3)C1]

Cpd. 87 were prepared following similar procedures as Cpd. 77 using 80-1 and the corresponding amine for amide coupling in step 1.

### Example 32: Preparation of (11E)-1-methyl-N-[(3S)-1-methylpyrrolidin-3-yl]-19,20-dihydro-1H,8H,18H-10,7,4-(ethan[1]yl[1,2]diylidene)pyrazolo[3,4-f][1,5,9,12,13]benzodioxatriazacyclooctadecine-15-carboxamide (Cpd. 88)

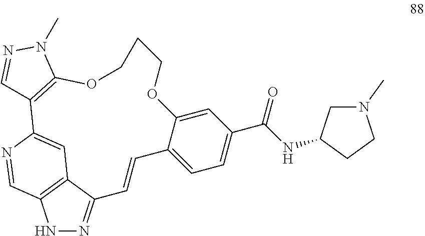 [SMILES: CN1CC[C@H](NC(=O)c2ccc3c(c2)OCCCOc2c(cnn2C)-c2cc4c(n[nH]c4cn2)/C=C/3)C1]

Cpd. 88 were prepared following similar procedures as Cpd. 77 using the corresponding amine for amide coupling in step 1.

### Example 33: Preparation of (11E)-N-ethyl-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-14-carboxamide (Cpd. 89)

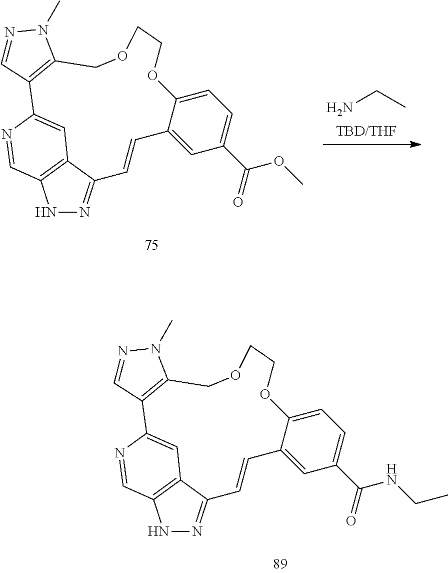

The mixture of Cpd. 75 (24.2 mg, 173 μmol, 1.5 eq) and ethanamine (104 mg, 2.32 mmol, 20.0 eq) in THF (2.0 mL) was added 3,4,6,7,8,9-hexahydro-2H-pyrimido[1,2-a]pyrimidine (32.4 mg, 232 μmol, 1.3 eq). The mixture was stirred at 50° C. for 16 hr. On completion, the mixture was concentrated in vacuum to afford crude. The crude was purified by reversed-phase HPLC (column: Welch Xtimate C18 150×25 mm×5 μm; mobile phase: [water (TFA)-ACN]; B %: 10%-40%, 10 mins) to give the residue. Then, the residue was triturated with MeCN (3.0 mL) at 20° C. for 20 mins, filtered and afford Cpd. 89 (30.0 mg, 65.4 μmol, 56.4% yield)

### Example 34 and 35: Preparation of (11E)-N-[2-(dimethylamino)ethyl]-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-14-carboxamide (Cpd. 90) and (11E)-N-[3-(dimethylamino)propyl]-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-14-carboxamide (Cpd. 91)

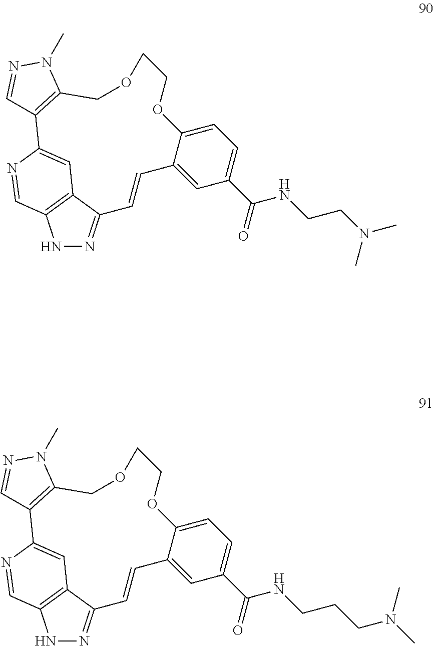 [SMILES: CN(C)CCNC(=O)c1ccc2c(c1)/C=C/c1n[nH]c3cnc(cc13)-c1cnn(C)c1COCCO2 ; CN(C)CCCNC(=O)c1ccc2c(c1)/C=C/c1n[nH]c3cnc(cc13)-c1cnn(C)c1COCCO2]

Cpd. 90 and Cpd. 91 were prepared following similar procedures as Cpd. 70 using 75-6 and the corresponding amines for amide formation in Step 1.

### Example 36: Preparation of (11E)-1-methyl-1,18,19,21-tetrahydro-8H-10,7,4-(ethan[1]yl[1,2]diylidene)pyrazolo[3,4-f][1,4,9,12,13]benzodioxatriazacyclooctadecine-14-carboxylic acid (Cpd. 92)

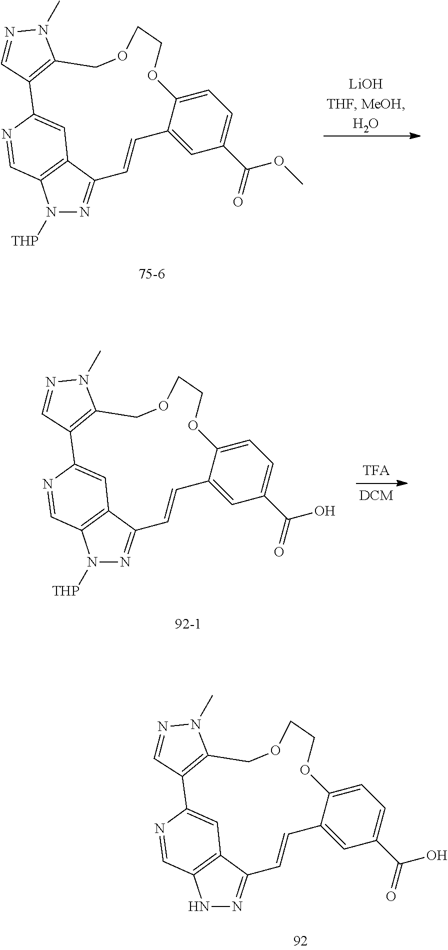

Cpd. 92 was prepared following similar procedures as Cpd. 80 using 75-6 as starting material.

### Example 37 and 38: Preparation of (11E)-1-methyl-N-(1-methylpiperidin-4-yl)-1,18,19,21-tetrahydro-8H-10,7,4-(ethan[1]yl[1,2]diylidene)pyrazolo[3,4-f][1,4,9,12,13]benzodioxatriazacyclooctadecine-14-carboxamide (Cpd. 93) and (11E)-1-methyl-N-[(1-methylpiperidin-4-yl)methyl]-1,18,19,21-tetrahydro-8H-10,7,4-(ethan[1]yl[1,2]diylidene)pyrazolo[3,4-f][1,4,9,12,13]benzodioxatriazacyclooctadecine-14-carboxamide (Cpd. 94)

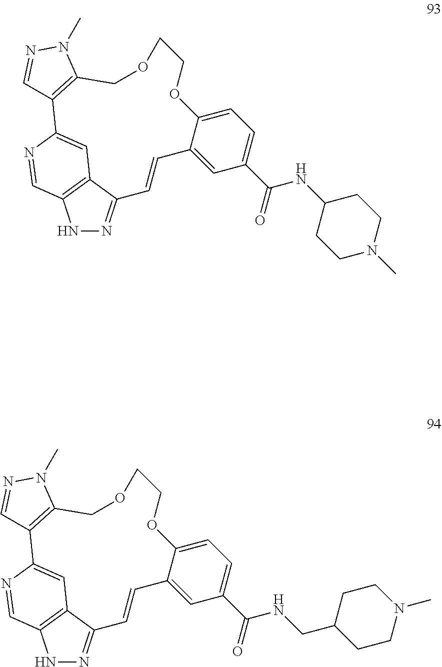 [SMILES: CN1CCC(NC(=O)c2ccc3c(c2)/C=C/c2n[nH]c4cnc(cc24)-c2cnn(C)c2COCCO3)CC1 ; CN1CCC(CNC(=O)c2ccc3c(c2)/C=C/c2n[nH]c4cnc(cc24)-c2cnn(C)c2COCCO3)CC1]

Cpd. 93 and Cpd. 94 were prepared following similar procedures as Cpd. 77 using 92-1 and corresponding amines for amide coupling in step 1.

### Example 39: Preparation of (18E)-8,9,16-trimethyl-8,9,11,12-tetrahydro-2H-5,3-(azenometheno)dipyrazolo[3′,4′:9,10;4″,3″:13,14][1,4]dioxacyclopentadecino[6,5-b]pyridine (Cpd. 95)

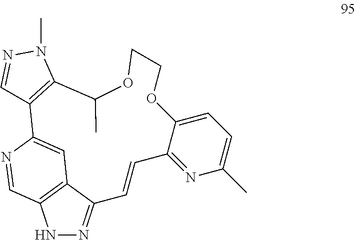 [SMILES: Cc1ccc2c(n1)/C=C/c1n[nH]c3cnc(cc13)-c1cnn(C)c1C(C)OCCO2]

Cpd. 95 was prepared following similar procedures as Cpd. 16 using 65-11 and 1-2 as starting materials.

### Example 40: Preparation of (4-methylpiperazin-1-yl)[(11E)-1-methyl-1,18,19,21-tetrahydro-8H-10,7,4-(ethan[1]yl[1,2]diylidene)pyrazolo[3,4-f][1,4,9,12,13]benzodioxatriazacyclooctadecin-14-yl]methanone (Cpd. 96)

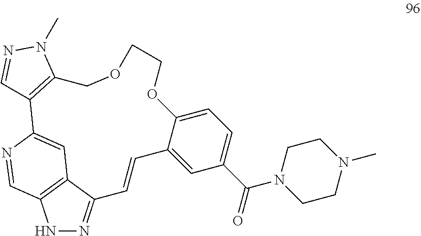 [SMILES: CN1CCN(C(=O)c2ccc3c(c2)/C=C/c2n[nH]c4cnc(cc24)-c2cnn(C)c2COCCO3)CC1]

Cpd. 96 was prepared following similar procedures as Cpd. 77 using 92-1 and corresponding amine for amide coupling in step 1.

### Example 41: Preparation of (11E)-1-methyl-N-(1-methylpiperidin-4-yl)-19,20-dihydro-1H,8H,18H-10,7,4-(ethan[1]yl[1,2]diylidene)pyrazolo[3,4-f][1,5,9,12,13]benzodioxatriazacyclooctadecine-14-carboxamide (Cpd. 97)

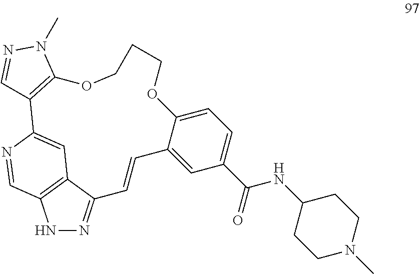 [SMILES: CN1CCC(NC(=O)c2ccc3c(c2)/C=C/c2n[nH]c4cnc(cc24)-c2cnn(C)c2OCCCO3)CC1]

Cpd. 97 was prepared following similar procedures as Cpd. 70 using the corresponding amine for amide formation.

### Example 42: Preparation of (11E)-14-fluoro-1,6-dimethyl-19,20-dihydro-1H,8H,18H-10,7,4-(ethan[1]yl[1,2]diylidene)pyrazolo[3,4-f][1,5,9,12,13]benzodioxatriazacyclooctadecine (Cpd. 98)

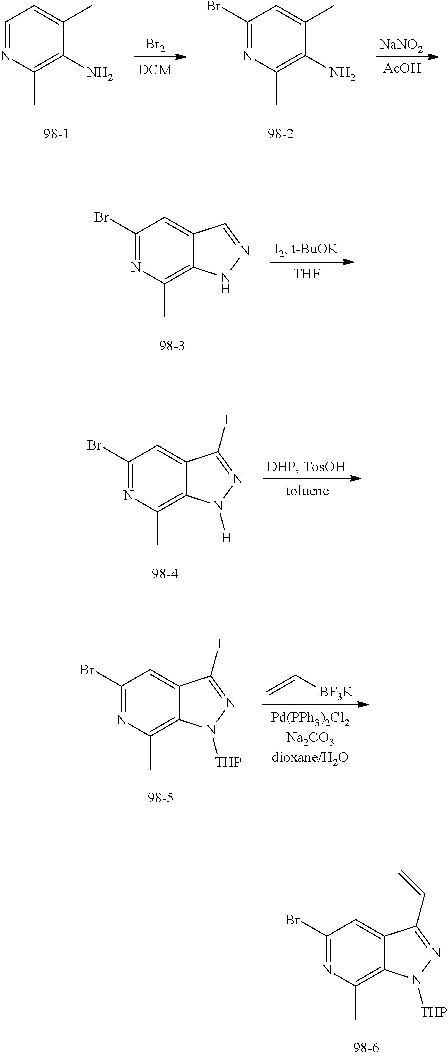

Step 1. To a mixture of 2,4-dimethylpyridin-3-amine (25.0 g, 204.64 mmol, 1 eq) in DCM (500 mL) was added a solution of Br₂ (163 g, 1.02 mol, 52.75 mL, 5 eq) in DCM (300 mL) dropwise at 0° C., the mixture was stirred at 25° C. for 12 hours. To the mixture was added aq. Na₂SO₃ (300 mL) and the mixture was stirred at 25° C. for 1 h. Then the mixture was adjusted to pH=7 by addition of NaHCO₃ slowly. The mixture was extracted with DCM (300 mL×3). The combine organic phase was washed with brine (50 mL), dried with Na₂SO₄, filtered and concentrated in vacuum. The residue was purified by column chromatography (SiO₂, Petroleum ether:Ethyl acetate=1:1) to afford 6-bromo-2,4-dimethyl-pyridin-3-amine (62.0 g, 308 mmol, 75.3% yield, 100% purity) as a yellow oil. ¹H NMR (400 MHz, CDCl₃) δ=7.02 (s, 1H), 3.45 (d, J=16.4 Hz, 2H), 2.37 (s, 3H), 2.13 (s, 3H).

Step 2. To a mixture of 6-bromo-2,4-dimethyl-pyridin-3-amine (30.0 g, 149 mmol, 1 eq) in AcOH (300 mL) was added NaNO₂ (11.3 g, 164 mmol, 1.1 eq) at 0° C., the mixture was stirred at 25° C. for 12 hours. The reaction mixture was concentrated in vacuum. The residue was diluted with H₂O (50 mL) and extracted with DCM (50 mL×3). The combined organic layers were washed with brine (30 mL×3), dried over Na₂SO₄, filtered and concentrated under reduced pressure to give a residue. The crude product was purified by reversed-phase HPLC (0.1% FA condition) to afford Compound 5-bromo-7-methyl-1H-pyrazolo[3,4-c]pyridine (20.0 g, 94.3 mmol, 63.2% yield) as a yellow solid. ¹H NMR (400 MHz, CDCl₃) δ=11.63-11.02 (m, 1H), 8.09 (s, 1H), 7.71 (s, 1H), 2.84 (s, 3H).

Step 3. To a solution of 5-bromo-7-methyl-1H-pyrazolo[3,4-c]pyridine (34.0 g, 160 mmol, 1 eq) in THF (450 mL) was added t-BuOK (54.0 g, 481 mmol, 3 eq), was added 1₂ (40.7 g, 160 mmol, 32.3 mL, 1 eq). The mixture was stirred at 25° C. for 3 hr. On completion, filtered and the filtrate was concentrated under reduced pressure to give a residue. The residue was purified by combi flash (12 g silica gel column, THF in PE 0-100%) to give 5-bromo-3-iodo-7-methyl-1H-pyrazolo[3,4-c] pyridine (51.0 g, 151 mmol, 94.1% yield) as yellow solid.

Step 4. To a solution of 5-bromo-3-iodo-7-methyl-1H-pyrazolo[3,4-c]pyridine (3.10 g, 9.17 mmol, 1 eq) in toluene (31 mL) was added TsOH (316 mg, 1.83 mmol, 0.2 eq) and 3,4-dihydro-2H-pyran (1.93 g, 22.9 mmol, 2.10 mL, 2.5 eq). The mixture was stirred at 90° C. for 16 hr, filtered and the filtrate was concentrated in vacuum. The residue was purified by combi flash (12 g silica gel column, EtOAc in PE 0-100%). 5-bromo-3-iodo-7-methyl-1-tetrahydropyran-2-yl-pyrazolo[3,4-c] pyridine (2.57 g, 6.09 mmol, 66.38% yield) was obtained as light yellow solid. ¹H NMR (400 MHz, DMSO-d₆) δ=7.53 (s, 1H), 6.12 (dd, J=9.2, 2.2 Hz, 1H), 3.88 (d, J=11.6 Hz, 1H), 3.73 (d, J=2.4 Hz, 1H), 2.90 (s, 3H), 2.45-2.37 (m, 1H), 2.12-2.03 (m, 2H), 1.81-1.66 (m, 2H), 1.47-1.43 (m, 1H).

Step 5. To a solution of potassium hydride;trifluoro(vinyl)boron (816 mg, 6.09 mmol, 1 eq), 5-bromo-3-iodo-7-methyl-1-tetrahydropyran-2-yl-pyrazolo[3,4-c]pyridine (2.57 g, 6.09 mmol, 1 eq) in a mixture solvent of dioxane (25 mL) and H₂O (5 mL) was added Pd(dppf)Cl₂ (445 mg, 609 μmol, 0.1 eq) and Na₂CO₃ (1.94 g, 18.3 mmol, 3 eq). The mixture was stirred at 80° C. for 16 hr under N₂. Dried with anhydrous Na₂SO₄, filtered and concentrated in vacuum. The residue was purified by combi flash (20 silica gel column, DCM 100%) to give 5-bromo-7-methyl-1-tetrahydropyran-2-yl-3-vinyl-pyrazolo[3,4-c] pyridine (1.00 g, 3.10 mmol, 50.9% yield) as black brown solid.

Cpd. 98 was prepared following similar procedures as Cpd. 68 using 67-2 coupled with 98-6. 4-Fluoro-2-iodo-phenol was used for the alkylation reaction in Step 4.

### Example 43: Preparation of (17E)-6-(3-chloro-4-fluorophenyl)-12,15-dimethyl-2,7,8,11,12,15-hexahydro-6H-5,3-(azenometheno)dipyrazolo[3,4-f:3′,4′-j][1,4,14]oxadiazacyclohexadecin-13(10H)-one (Cpd. 99)

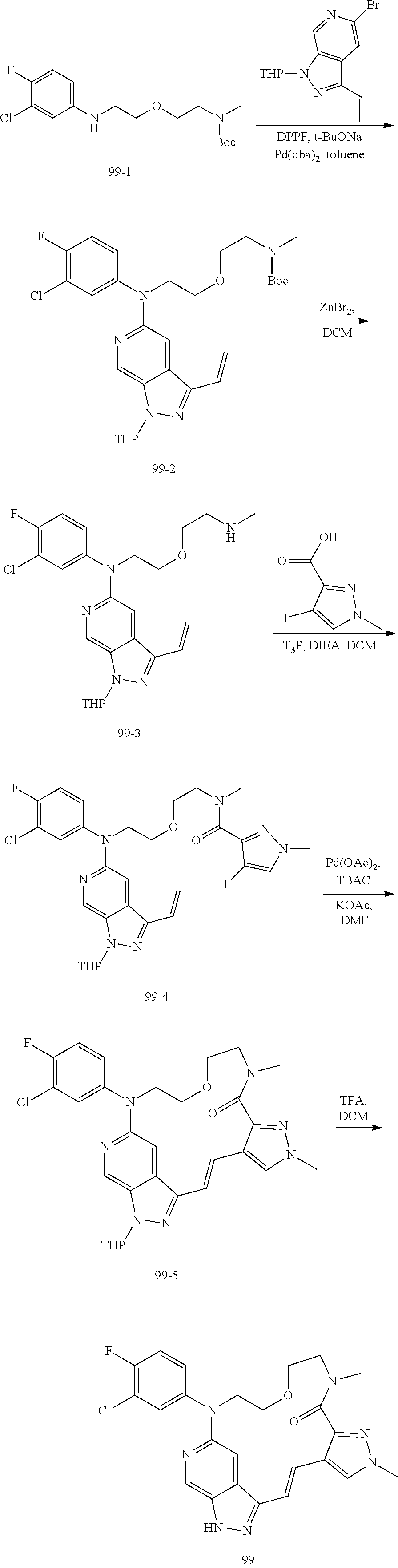

Step 1. The mixture of tert-butyl N-[2-[2-(3-chloro-4-fluoro-anilino)ethoxy]ethyl]-N-methyl-carbamate (200 mg, 577 μmol, 1 eq), 5-bromo-1-tetrahydropyran-2-yl-3-vinyl-pyrazolo[3,4-c]pyridine (196 mg, 634 μmol, 1.1 eq), DPPF (63.9 mg, 115 μmol, 0.2 eq), Pd(dba)₂ (33.2 mg, 57.7 μmol, 0.1 eq) and t-BuONa (83.1 mg, 865 μmol, 1.5 eq) in toluene (10 mL) was stirred at 110° C. for 12 h. On completion, the mixture was concentrated to give a residue. The residue was purified by column chromatography (SiO₂, PE/THF=4:1 to 3:1) to give tert-butyl N-[2-[2-(3-chloro-4-fluoro-N-(1-tetrahydropyran-2-yl-3-vinyl-pyrazolo[3,4-c]pyridin-5-yl)anilino)ethoxy]ethyl]-N-methyl-carbamate (274 mg, 477 μmol, 82.77% yield) as a green oil.

Step 2. To a solution of tert-butyl N-[2-[2-(3-chloro-4-fluoro-N-(1-tetrahydropyran-2-yl-3-vinyl-pyrazolo[3,4-c]pyridin-5-yl)anilino)ethoxy]ethyl]-N-methyl-carbamate (254 mg, 442 μmol, 1 eq) in DCM (3.5 mL) was added ZnBr₂ (498 mg, 2.21 mmol, 5 eq), the mixture was stirred at 25° C. for 2 h. On completion, the mixture was quenched with water (10 mL) and extracted with DCM (15 mL×3), the combined organic phase was dried over anhydrous sodium sulfate, filtered and concentrated to give a residue. The residue was concentrated to give N-(3-chloro-4-fluoro-phenyl)-N-[2-[2-(methylamino)ethoxy]ethyl]-1-tetrahydropyran-2-yl-3-vinyl-pyrazolo[3,4-c]pyridin-5-amine (177 mg, 373 μmol, 84.40% yield) as a yellow oil.

Step 3. To a solution of N-(3-chloro-4-fluoro-phenyl)-N-[2-[2-(methylamino)ethoxy]ethyl]-1-tetrahydropyran-2-yl-3-vinyl-pyrazolo[3,4-c]pyridin-5-amine (177 mg, 373 μmol, 1.05 eq), 4-iodo-1-methyl-pyrazole-3-carboxylic acid (89.6 mg, 356 μmol, 1 eq) DIEA (368 mg, 2.85 mmol, 8 eq) in DCM (3.5 mL) was added T₃P (340 mg, 534 μmol, 50% purity, 1.5 eq) at 0° C. The mixture was stirred at 25° C. for 15 h. The mixture was quenched with water (10 mL) and extracted with DCM (12 mL×3), the combined organic phase was dried over anhydrous sodium sulfate, filtered and concentrated to give a residue was purified by column chromatography (SiO₂, PE/THF=1:1) to give N-[2-[2-(3-chloro-4-fluoro-N-(1-tetrahydropyran-2-yl-3-vinyl-pyrazolo[3,4-c]pyridin-5-yl)anilino)ethoxy]ethyl]-4-iodo-N,1-dimethyl-pyrazole-3-carboxamide (131 mg, 185 μmol, 52.03% yield) as a green oil.

Step 4. To a solution of N-[2-[2-(3-chloro-4-fluoro-N-(1-tetrahydropyran-2-yl-3-vinyl-pyrazolo[3,4-c]pyridin-5-yl)anilino)ethoxy]ethyl]-4-iodo-N,1-dimethyl-pyrazole-3-carboxamide (100 mg, 141 μmol, 1 eq) in DMF (5 mL) was added KOAc (69.3 mg, 706 μmol, 5 eq), TBAC (78.5 mg, 283 μmol, 2 eq) and Pd(OAc)₂ (3.17 mg, 14.1 μmol, 0.1 eq), the resulting mixture was degassed and purged with N₂ for 3 times, and then the mixture was stirred at 80° C. for 12 h. The mixture was filtered and concentrated to give a residue. The reaction was purified by Prep-HPLC (column: Welch Xtimate C18 150×25 mm×5 μm; mobile phase: [water(TFA)-ACN]; B %: 35%-65%, 10 min) to give 99-5 (10.0 mg, 17.2 μmol, 12.21% yield) as a green solid.

Step 5. A mixture of 99-5 (10.0 mg, 17.2 μmol, 1 eq) and TFA (0.25 mL) in DCM (0.25 mL) was stirred at 25° C. of 2 h. The mixture was filtered and concentrated to give a residue. The reaction was purified by Prep-HPLC (column: Welch Xtimate C18 150×25 mm×5 μm; mobile phase: [water(TFA)-ACN]; B %: 20%-50%, 10 min) to give Cpd. 99 (2.60 mg, 5.24 μmol, 30.41% yield) as a yellow solid.

### Example 44: Preparation of (11E)-14-fluoro-1-methyl-19,20-dihydro-1H,8H,18H-10,7,4-(ethan[1]yl[1,2]diylidene)pyrazolo[3,4-f][1,5,9,12,13]benzodioxatriazacyclooctadecine (Cpd. 100)

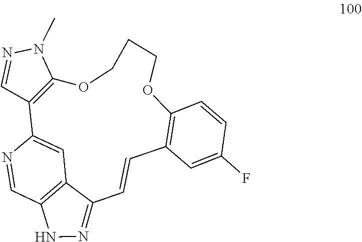 [SMILES: Cn1ncc2c1OCCCOc1ccc(F)cc1/C=C/c1n[nH]c3cnc-2cc13]

Cpd. 100 was prepared following similar procedures as Cpd. 68 using 4-fluoro-2-iodo-phenol for the alkylation reaction in Step 4.

| Cpd. | | MS m/z | |
|---|---|---|---|
| # | Structure | [M + H]⁺ | 1H NMR (400 MHz, DMSO-d₆) δ ppm |
| 1 | 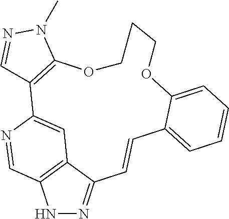 [SMILES: Cn1ncc2c1OCCCOc1ccccc1/C=C/c1n[nH]c3cnc-2cc13] | 374.1 | 13.70-13.27 (m, 1H), 9.01 (s, 1H), 8.44 (s, 1H), 8.13 (d, J = 17.2 Hz, 1H), 7.96-7.87 (m, 2H), 7.51 (d, J = 17.2 Hz, 1H), 7.37-7.23 (m, 2H), 7.10 (t, J = 7.2 Hz, 1H), 4.49 (t, J = 5.6 Hz, 2H), 4.31 (t, J = 5.2 Hz, 2H), 3.76 (s, 3H), 2.45-2.41 (m, 2H) |
| 2 | 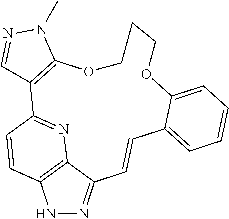 [SMILES: Cn1ncc2c1OCCCOc1ccccc1/C=C/c1n[nH]c3ccc-2nc13] | 374.1 | 13.18 (s, 1H), 8.86 (d, J = 16.8 Hz, 1H), 8.28 (s, 1H), 7.99-7.94 (m, 2H), 7.79 (d, J = 7.2 Hz, 1H), 7.70 (d, J = 8.8 Hz, 1H), 7.38 (d, J = 16.8 Hz, 1H), 7.32-7.26 (m, 1H), 7.23-7.19 (m, 1H), 7.08 (t, J = 7.2 Hz, 1H), 4.73 (t, J = 6.4 Hz, 2H), 4.28 (t, J = 5.2 Hz, 2H), 3.72 (s, 3H), 2.23 (quin, J = 5.6 Hz, 2H) |
| 3 | 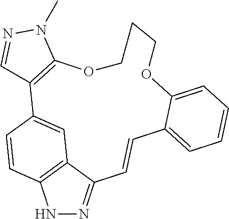 [SMILES: Cn1ncc2c1OCCCOc1ccccc1/C=C/c1n[nH]c3ccc-2cc13] | 373.1 | 13.08 (s, 1H), 8.55 (s, 1H), 8.10 (d, J = 17.2 Hz, 1H), 7.91-7.85 (m, 2H), 7.68 (d, J = 8.8 Hz, 1H), 7.54 (d, J = 8.8 Hz, 1H), 7.47 (d, J = 17.2 Hz, 1H), 7.34- 7.27 (m, 1H), 7.26-7.20 (m, 1H), 7.09 (t, J = 7.6 Hz, 1H), 4.48 (t, J = 6.0 Hz, 2H), 4.28 (t, J = 5.2 Hz, 2H), 3.74 (s, 3H), 2.40 (td, J = 5.2, 10.8 Hz, 2H) |
| 16 | 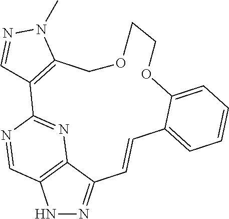 [SMILES: Cn1ncc2c1COCCOc1ccccc1/C=C/c1n[nH]c3cnc-2nc13] | 375.2 | 13.66 (s, 1H), 9.31 (s, 1H), 8.95 (d, J = 16.9 Hz, 1H), 8.09 (s, 1H), 7.94 (d, J = 6.4 Hz, 1H), 7.52 (d, J = 16.9 Hz, 1H), 7.32 (s, 1H), 7.20 (d, J = 8.0 Hz, 1H), 7.11-7.06 (m, 1H), 5.47 (s, 2H), 4.30- 4.26 (m, 2H), 4.09-4.05 (m, 2H), 3.97 (s, 3H) |
| 38 | 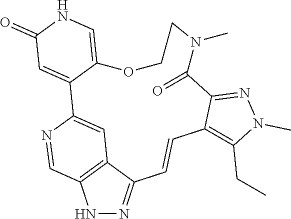 [SMILES: CCc1c2c(nn1C)C(=O)N(C)CCOc1c[nH]c(=O)cc1-c1cc3c(n[nH]c3cn1)/C=C/2] | 446.1 | 13.90-13.63 (m, 1H), 9.15 (s, 1H), 8.93 (s, 1H), 7.72-7.47 (m, 1H), 7.38- 7.25 (m, 2H), 7.09-7.00 (m, 1H), 4.72- 4.45 (m, 1H), 4.41-4.27 (m, 2H), 3.86 (s, 3H), 3.08-2.92 (m, 6H), 1.28 (t, J = 7.2 Hz, 3H) |
| 62 | 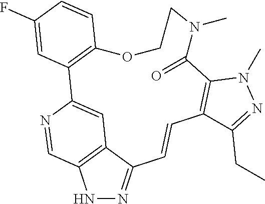 [SMILES: CCc1nn(C)c2c1/C=C/c1n[nH]c3cnc(cc13)-c1cc(F)ccc1OCCN(C)C2=O] | 447.3 | 9.16 (d, J = 0.8 Hz, 1H), 9.03 (s, 1H), 7.79-7.77 (m 1H), 7.49 (d, J = 17.4 Hz, 1H), 7.35-7.32 (m, 1H), 7.24- 7.15 (m, 2H), 4.66-4.62 (m, 1H), 4.61- 4.57 (m, 2H), 3.85 (s, 3H), 3.47-3.40 (m, 1H), 3.17 (s, 3H), 2.94 (q, J = 7.6 Hz, 2H), 1.38 (t, J = 7.6 Hz, 3H) |
| 63 | 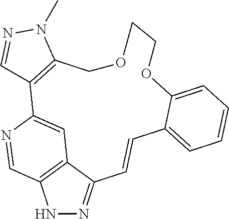 [SMILES: Cn1ncc2c1COCCOc1ccccc1/C=C/c1n[nH]c3cnc-2cc13] | 374.3 | 13.90-13.30 (m, 1H), 9.08 (s, 1H), 8.36 (s, 1H), 8.22 (d, J = 17.3 Hz, 1H), 7.85 (d, J = 7.8 Hz, 1H), 7.81 (s, 1H), 7.46 (d, J = 17.3 Hz, 1H), 7.34-7.27 (m, 1H), 7.14 (d, J = 8.1 Hz, 1H), 7.08- 7.01 (m, 1H), 4.69 (s, 2H), 4.35-4.31 (m, 2H), 4.03-3.99 (m, 2H), 3.95 (s, 3H |
| 64 | 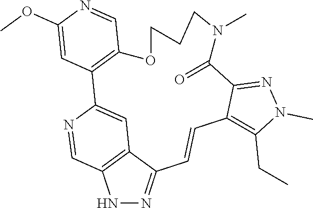 | 474.2 | 14.24-13.29 (m, 1H), 9.15 (s, 1H), 8.01 (s, 1H), 7.92 (s, 1H), 7.55 (s, 1H), 5.84 (d, J = 2.4 Hz, 1H), 5.54 (d, J = 2.4 Hz, 1H), 4.38-4.27 (m, 1H), 3.83 (s, 3H), 3.73 (s, 3H), 3.68-3.57 (m, 2H), 3.53-3.45 (m, 1H), 2.98 (s, 3H), 2.26-2.13 (m, 2H), 1.94-1.82 (m, 1H), 1.81-1.70 (m, 1H), 0.67 (t, J = 7.2 Hz, 3H) |
| 65 | 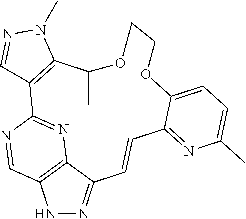 [SMILES: Cc1ccc2c(n1)/C=C/c1n[nH]c3cnc(nc13)-c1cnn(C)c1C(C)OCCO2] | 404.2 | 14.08-13.37 (m, 1H), 9.30 (t, J = 8.0 Hz, 2H), 8.07 (s, 1H), 7.67 (d, J = 16.4 Hz, 1H), 7.36 (d, J = 8.4 Hz, 1H), 7.17 (d, J = 8.4 Hz, 1H), 6.86 (q, J = 6.8 Hz, 1H), 4.41-4.32 (m, 1H), 4.17-4.08 (m, 2H), 4.06 (s, 3H), 4.03-3.90 (m, 2H), 2.47 (s, 3H), 1.39 (d, J = 6.8 Hz, 3H) |
| 66 | 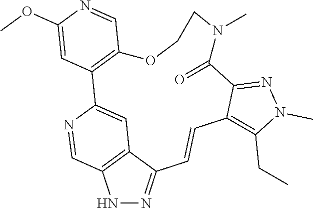 | 460.2 | 13.85-13.49 (m, 1H), 9.15 (s, 1H), 9.01 (s, 1H), 8.23 (s, 1H), 7.65 (s, 1H), 7.36 (d, J = 17.2 Hz, 1H), 7.05 (d, J = 17.2 Hz, 1H), 4.67-4.45 (m, 3H), 3.87 (s, 7H), 3.03 (s, 3H), 2.99 (q, J = 7.6 Hz, 2H), 1.27 (t, J = 7.6 Hz, 3H) |
| 67 | 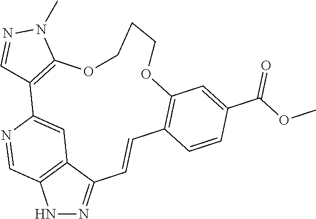 [SMILES: COC(=O)c1ccc2c(c1)OCCCOc1c(cnn1C)-c1cc3c(n[nH]c3cn1)/C=C/2] | 432.1 | 13.70 (m, 1H), 9.04 (s, 1H), 8.44 (s, 1H), 8.14 (d, J = 16.0 Hz, 1H), 8.07 (d, J = 8.0 Hz, 1H), 7.93 (s, 1H), 7.77 (d, J = 4.0 Hz, 1H), 7.71-7.68 (m, 1H), 7.68-7.65 (m, 1H), 4.51 (t, J = 5.2 Hz, 2H), 4.37 (t, J = 5.2 Hz, 2H), 3.88 (s, 3H), 3.77 (s, 3H), 2.44 (s, 2H) |
| 68 | 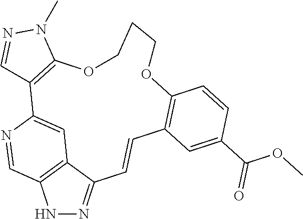 [SMILES: COC(=O)c1ccc2c(c1)/C=C/c1n[nH]c3cnc(cc13)-c1cnn(C)c1OCCCO2] | 432.1 | 13.83-13.45 (m, 1H), 9.04 (s, 1H), 8.41-8.36 (m, 2H), 8.11 (d, J = 17.2 Hz, 1H), 7.95-7.90 (m, 2H), 7.53 (d, J = 17.2 Hz, 1H), 7.37 (d, J = 8.8 Hz, 1H), 4.52 (t, J = 6.0 Hz, 2H), 4.40 (t, J = 5.6 Hz, 2H), 3.87 (s, 3H), 3.76 (s, 3H), 2.44 (d, J = 5.6 Hz, 2H) |
| 69 | 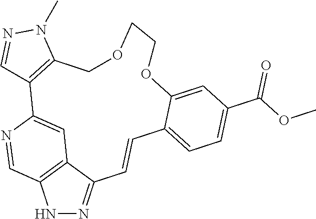 [SMILES: COC(=O)c1ccc2c(c1)OCCOCc1c(cnn1C)-c1cc3c(n[nH]c3cn1)/C=C/2] | 432.1 | 13.70 (s, 1H), 9.11 (s, 1H), 8.35 (s, 1H), 8.23 (d, J = 17.2 Hz, 1H), 8.02 (d, J = 8.4 Hz, 1H), 7.80 (s, 1H), 7.65 (d, J = 4.4 Hz, 1H), 7.63-7.60 (m, 2H), 4.70 (s, 2H), 4.43-4.38 (m, 2H), 4.05- 4.02 (m, 2H), 3.96 (s, 3H), 3.87 (s, 3H) |
| 70 | 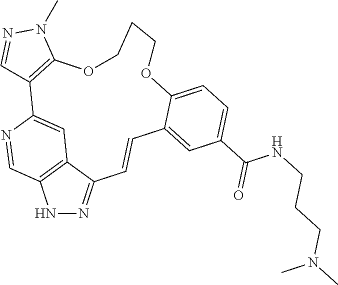 [SMILES: CN(C)CCCNC(=O)c1ccc2c(c1)/C=C/c1n[nH]c3cnc(cc13)-c1cnn(C)c1OCCCO2] | 502.2 | 13.87-13.31 (m, 1H), 9.05 (s, 1H), 8.66 (t, J = 5.6 Hz, 1H), 8.45-8.36 (m, 2H), 8.15 (d, J = 17.2 Hz, 1H), 7.93 (s, 1H), 7.85 (dd, J = 2.0, 8.8 Hz, 1H), 7.59 (d, J = 17.2 Hz, 1H), 7.35 (d, J = 8.8 Hz, 1H), 4.52 (t, J = 5.6 Hz, 2H), 4.37 (t, J = 5.2 Hz, 2H), 3.77 (s, 3H), 3.37 (q, J = 6.4 Hz, 2H), 3.16-3.09 (m, 2H), 2.81 (d, J = 4.8 Hz, 6H), 2.47- 2.41 (m, 2H), 1.97-1.86 (m, 2H) |
| 71 | 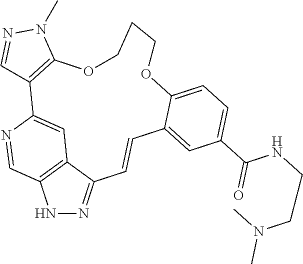 [SMILES: CN(C)CCNC(=O)c1ccc2c(c1)/C=C/c1n[nH]c3cnc(cc13)-c1cnn(C)c1OCCCO2] | 488.2 | 9.45 (s, 1H), 9.05 (s, 1H), 8.74 (t, J = 5.6 Hz, 1H), 8.46-8.38 (m, 2H), 8.15 (d, J = 17.2 Hz, 1H), 7.93 (s, 1H), 7.88- 7.83 (m, 1H), 7.57 (d, J = 17.2 Hz, 1H), 7.37 (d, J = 8.8 Hz, 1H), 4.52 (t, J = 5.6 Hz, 2H), 4.37 (t, J = 5.2 Hz, 2H), 3.77 (s, 3H), 3.68-3.63 (m, 2H), 3.35- 3.26 (m, 2H), 2.88 (d, J = 4.4 Hz, 6H), 2.48-2.41 (m, 2H) |
| 72 | 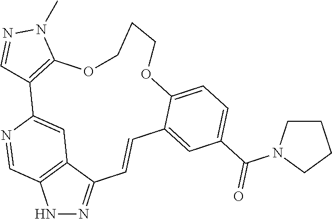 [SMILES: Cn1ncc2c1OCCCOc1ccc(C(=O)N3CCCC3)cc1/C=C/c1n[nH]c3cnc-2cc13] | 471.1 | 13.80-13.41 (m, 1H), 9.05 (s, 1H), 8.43 (s, 1H), 8.12 (d, J = 17.6 Hz, 1H), 8.04 (d, J = 2.0 Hz, 1H), 7.93 (s, 1H), 7.58 (d, J = 17.6 Hz, 1H), 7.47 (dd, J = 2.0, 8.4 Hz, 1H), 7.30 (d, J = 8.4 Hz, 1H), 4.50 (t, J = 5.6 Hz, 2H), 4.35 (t, J = 5.6 Hz, 2H), 3.76 (s, 3H), 3.58-3.54 (m, 4H), 2.46-2.42 (m, 2H), 1.92- 1.82 (m, 4H) |
| 73 | 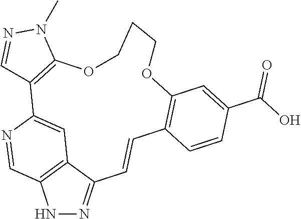 [SMILES: Cn1ncc2c1OCCCOc1cc(C(=O)O)ccc1/C=C/c1n[nH]c3cnc-2cc13] | 418.1 | 13.69 (s, 1H), 13.08 (s, 1H), 9.04 (s, 1H), 8.44 (s, 1H), 8.14 (d, J = 17.2 Hz, 1H), 8.04 (d, J = 8.0 Hz, 1H), 7.93 (s, 1H), 7.75 (d, J = 1.2 Hz, 1H), 7.69- 7.61 (m, 2H), 4.50 (t, J = 5.6 Hz, 2H), 4.36 (t, J = 5.6 Hz, 2H), 3.76 (s, 3H), 2.44-2.43 (m, 2H) |
| 74 | 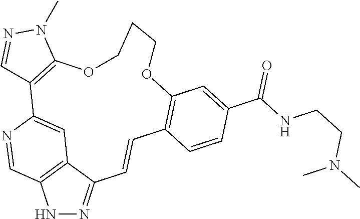 [SMILES: CN(C)CCNC(=O)c1ccc2c(c1)OCCCOc1c(cnn1C)-c1cc3c(n[nH]c3cn1)/C=C/2] | 488.2 | 13.62 (s, 1H), 9.46-9.31 (m, 1H), 9.04 (s, 1H), 8.75 ( t, J = 5.2 Hz, 1H), 8.45 (s, 1H), 8.15-8.05 (m, 2H), 7.94 (s, 1H), 7.74 (d, J = 1.2 Hz, 1H), 7.70- 7.60 (m, 2H), 4.54-4.50 (m, 2H), 4.36 (t, J = 5.2 Hz, 2H), 3.77 (s, 3H), 3.63 (d, J = 5.8 Hz, 2H), 3.31-3.27 (m, 2H), 2.87 (d, J = 4.6 Hz, 6H), 2.44 (s, 2H) |
| 75 | 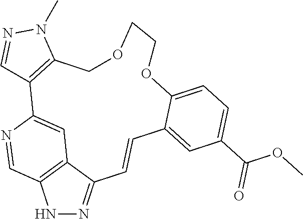 [SMILES: COC(=O)c1ccc2c(c1)/C=C/c1n[nH]c3cnc(cc13)-c1cnn(C)c1COCCO2] | 432.1 | 13.74 (s, 1H), 9.13 (s, 1H), 8.35 (d, J = 2.0 Hz, 1H), 8.32 (s, 1H), 8.18 (d, J = 17.2 Hz, 1H), 7.91 (dd, J = 8.4, 2.4 Hz, 1H), 7.80 (s, 1H), 7.51 (d, J = 17.2 Hz, 1H), 7.24 (d, J = 8.4 Hz, 1H), 4.70 (s, 2H), 4.43-4.37 (m, 2H), 4.08-4.02 (m, 2H), 3.96 (s, 3H), 3.86 (s, 3H) |
| 76 | 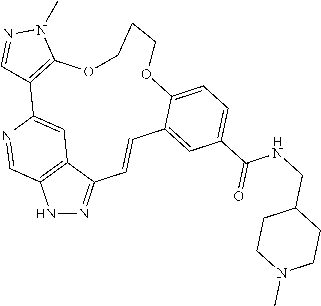 [SMILES: CN1CCC(CNC(=O)c2ccc3c(c2)/C=C/c2n[nH]c4cnc(cc24)-c2cnn(C)c2OCCCO3)CC1] | 528.2 | 13.86-13.49 (m, 1H), 9.32 (d, J = 4.0 Hz, 1H), 9.05 (s, 1H), 8.61 (t, J = 5.6 Hz, 1H), 8.43 (s, 2H), 8.14 (d, J = 17.2 Hz, 1H), 7.94 (s, 1H), 7.88-7.80 (m, 1H), 7.60 (d, J = 17.2 Hz, 1H), 7.34 (d, J = 8.4 Hz, 1H), 4.52 (t, J = 5.6 Hz, 2H), 4.36 (t, J = 5.2 Hz, 2H), 3.77 (s, 3H), 3.44-3.40 (m, 2H), 3.23 (t, J = 6.0 Hz, 2H), 2.98-2.85 (m, 2H), 2.74 (d, J = 4.8 Hz, 2H), 2.46-2.41 (m, 2H), 1.95-1.85 (m, 2H), 1.84-1.75 (m, 1H), 1.49-1.35 (m, 2H) |
| 77 | 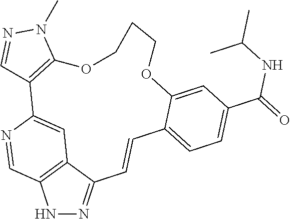 [SMILES: CC(C)NC(=O)c1ccc2c(c1)OCCCOc1c(cnn1C)-c1cc3c(n[nH]c3cn1)/C=C/2] | 459.1 | 13.56 (s, 1H), 9.04 (s, 1H), 8.46 (s, 1H), 8.13 (d, J = 16.0 Hz, 1H), 8.00 (d, J = 8.0 Hz, 1H), 7.94 (s, 1H), 7.73 (d, J = 1.6 Hz, 1H), 7.64 (s, 1H), 7.62-7.59 (m, 2H), 4.51 (t, J = 5.6 Hz, 2H), 4.36 (t, J = 5.4 Hz, 2H), 4.12-4.10 (m 1H), 3.77 (s, 3H), 2.47-2.44 (m, 2H), 1.19 (d, J = 6.8 Hz, 6H) |
| 78 | 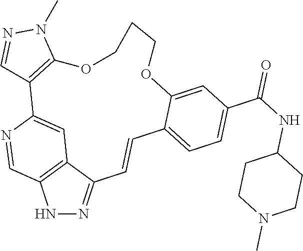 [SMILES: CN1CCC(NC(=O)c2ccc3c(c2)OCCCOc2c(cnn2C)-c2cc4c(n[nH]c4cn2)/C=C/3)CC1] | 514.2 | 13.59 (s, 1H), 9.41 (s, 1H), 9.05 (s, 1H), 8.50 (d, J = 7.6 Hz, 1H), 8.46 (s, 1H), 8.13 (d, J = 16.0 Hz, 1H), 8.03 (d, J = 8.0 Hz, 1H), 7.95 (s, 1H), 7.74 (d, J = 1.6 Hz, 1H), 7.67-7.62 (m, 2H), 4.52 (t, J = 5.6 Hz, 2H), 4.37 (t, J = 5.4 Hz, 2H), 4.07-4.02 (m, 1H), 3.77 (s, 3H), 3.49 (d, J = 12.0 Hz, 2H), 3.17- 3.08 (m, 2H), 2.79 (d, J = 4.6 Hz, 3H), 2.48-2.44 (m, 2H), 2.10-2.03 (m, 2H), 1.85-1.74 (m, 2H) |
| 79 | 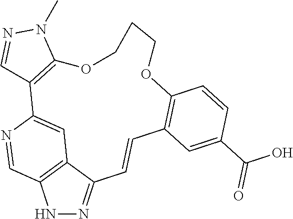 [SMILES: Cn1ncc2c1OCCCOc1ccc(C(=O)O)cc1/C=C/c1n[nH]c3cnc-2cc13] | 418.1 | 13.79-13.40 (m, 1H), 13.17-12.54 (m, 1H), 9.03 (s, 1H), 8.41-8.33 (m, 2H), 8.12 (d, J = 17.2 Hz, 1H), 7.94- 7.88 (m, 2H), 7.51 (d, J = 17.2 Hz, 1H), 7.35 (d, J = 8.8 Hz, 1H), 4.52 (t, J = 6.0 Hz, 2H), 4.39 (t, J = 5.6 Hz, 2H), 3.77 (s, 3H), 2.47-2.42 (m, 2H) |
| 80 | 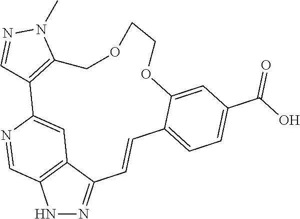 [SMILES: Cn1ncc2c1COCCOc1cc(C(=O)O)ccc1/C=C/c1n[nH]c3cnc-2cc13] | 418.1 | 14.38-13.54 (m, 1H), 13.43-12.12 (m, 1H), 9.18 (s, 1H), 8.40 (s, 1H), 8.23 (d, J = 17.2 Hz, 1H), 8.02-7.94 (m, 1H), 7.83 (s, 1H), 7.64-7.58 (m, 3H), 4.71 (s, 2H), 4.42-4.37 (m, 2H), 4.06- 4.01 (m, 2H), 3.97 (s, 3H) |
| 81 | 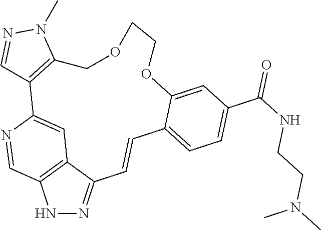 [SMILES: CN(C)CCNC(=O)c1ccc2c(c1)OCCOCc1c(cnn1C)-c1cc3c(n[nH]c3cn1)/C=C/2] | 488.3 | 14.12-13.41 (m, 1H), 9.39 (s, 1H), 9.14 (s, 1H), 8.74 (t, J = 5.6 Hz, 1H), 8.38 (s, 1H), 8.23 (d, J = 17.2 Hz, 1H), 8.01 (d, J = 8.0 Hz, 1H), 7.82 (s, 1H), 7.64-7.55 (m, 3H), 4.71 (s, 2H), 4.41- 4.38 (m, 2H), 4.06-4.03 (m, 2H), 3.97 (s, 3H), 3.63 (q, J = 5.6 Hz, 2H), 3.28 (q, J = 5.6 Hz, 2H), 2.87 (d, J = 4.8 Hz, 6H) |
| 82 | 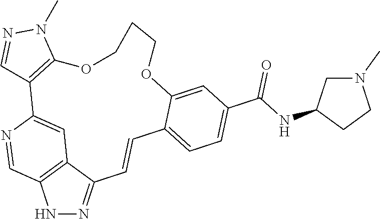 [SMILES: CN1CC[C@@H](NC(=O)c2ccc3c(c2)OCCCOc2c(cnn2C)-c2cc4c(n[nH]c4cn2)/C=C/3)C1] | 500.2 | 13.63 (s, 1H), 10.00-9.97 (m, 1H), 9.04 (d, J = 0.8 Hz, 1H), 8.44 (s, 1H), 8.12 (d, J = 16.0 Hz, 1H), 8.04 (d, J = 8.0 Hz, 1H), 7.94 (s, 1H), 7.72 (s, 1H), 7.67 (s, 1H), 7.64-7.60 (m, 2H), 4.57 (d, J = 8.8 Hz, 1H), 4.50 (t, J = 5.6 Hz, 2H), 4.35 (t, J = 5.2 Hz, 2H), 3.76 (s, 3H), 3.61-3.50 (m, 1H), 3.36-3.30 (m, 1H), 3.31-3.22 (m, 1H), 3.17- 3.00 (m, 1H), 2.90-2.85 (m, 2H), 2.46- 2.43 (m, 2H), 2.33-2.04 (m, 2H) |
| 83 | 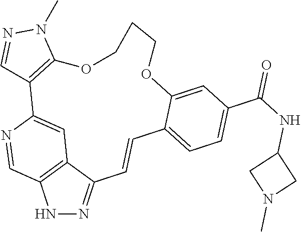 [SMILES: CN1CC(NC(=O)c2ccc3c(c2)OCCCOc2c(cnn2C)-c2cc4c(n[nH]c4cn2)/C=C/3)C1] | 486.2 | 13.63 (s, 1H), 9.93 (s, 1H), 9.14 (d, J = 0.8 Hz, 1H), 9.08 (s, 1H), 8.44 (s, 1H), 8.12 (d, J = 16.0 Hz, 1H), 8.04 (d, J = 8.0 Hz, 1H), 7.94 (s, 1H), 7.72 (s, 1H), 7.67 (s, 1H), 7.64-7.63 (d, J = 8.4 Hz, 1H), 4.75 (t, J = 5.2 Hz, 1H), 4.38 (t, J = 5.6 Hz, 2H), 4.36-4.35 (m, 2H), 4.10-4.00 (m, 2H), 4.08-3.77 (m, 2H), 3.75 (s, 3H), 2.93 (d, J = 8.8 Hz, 3H) 2.46-2.43 (m, 2H) |
| 84 | 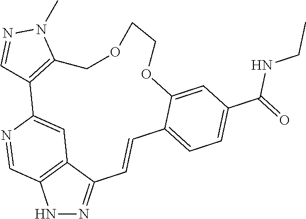 [SMILES: CCNC(=O)c1ccc2c(c1)OCCOCc1c(cnn1C)-c1cc3c(n[nH]c3cn1)/C=C/2] | 445.2 | 14.00-13.56 (m, 1H), 9.16 (s, 1H), 8.49 (t, J = 5.2 Hz, 1H), 8.40 (s, 1H), 8.22 (d, J = 17.2 Hz, 1H), 7.95 (d, J = 8.0 Hz, 1H), 7.83 (s, 1H), 7.61-7.52 (m, 3H), 4.71 (s, 2H), 4.42-4.38 (m, 2H), 4.05-4.02 (m, 2H), 3.97 (s, 3H), 3.35-3.27 (m, 2H), 1.16-1.12 (t, J = 7.2 Hz, 3H) |
| 85 | 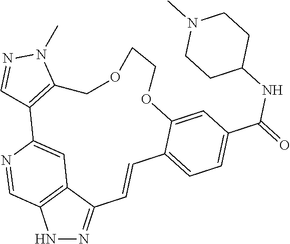 [SMILES: CN1CCC(NC(=O)c2ccc3c(c2)OCCOCc2c(cnn2C)-c2cc4c(n[nH]c4cn2)/C=C/3)CC1] | 514.2 | 14.07-13.31 (m, 1H), 9.44-9.31 (m, 1H), 9.11 (s, 1H), 8.47 (d, J = 7.6 Hz, 1H), 8.36 (s, 1H), 8.22 (d, J = 17.2 Hz, 1H), 7.98 (d, J = 7.6 Hz, 1H), 7.82 (s, 1H), 7.63-7.55 (m, 3H), 4.71 (s, 2H), 4.41 (s, 2H), 4.04 (s, 3H), 3.96 (s, 3H), 3.46 (s, 2H), 3.15-3.09 (m, 2H), 2.78 (s, 3H), 2.05 (d, J = 12.4 Hz, 2H), 1.84- 1.72 (m, 2H) |
| 86 | 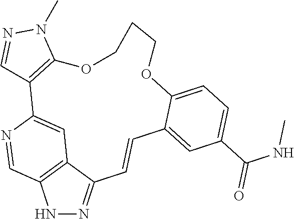 [SMILES: CNC(=O)c1ccc2c(c1)/C=C/c1n[nH]c3cnc(cc13)-c1cnn(C)c1OCCCO2] | 431.1 | 14.03-13.41 (m, 1H), 9.07 (s, 1H), 8.48-8.42 (m, 2H), 8.40 (d, J = 2.0 Hz, 1H), 8.12 (d, J = 17.2 Hz, 1H), 7.96-7.92 (m, 1H), 7.82 (dd, J = 2.0, 8.8 Hz, 1H), 7.57 (d, J = 17.2 Hz, 1H), 7.31 (d, J = 8.8 Hz, 1H), 4.51 (t, J = 5.6 Hz, 2H), 4.34 (t, J = 5.2 Hz, 2H), 3.76 (s, 3H), 2.82 (d, J = 4.4 Hz, 2H), 2.46- 2.37 (m, 3H) |
| 87 | 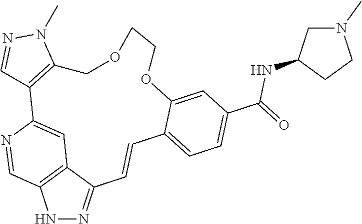 [SMILES: CN1CC[C@@H](NC(=O)c2ccc3c(c2)OCCOCc2c(cnn2C)-c2cc4c(n[nH]c4cn2)/C=C/3)C1] | 500.2 | 13.94-13.58 (m, 1H), 9.97-9.80 (m, 1H), 9.14 (s, 1H), 8.78-8.66 (m, 1H), 8.38 (s, 1H), 8.23 (d, J = 17.2 Hz, 1H), 8.01 (d, J = 8.0 Hz, 1H), 7.82 (s, 1H), 7.66-7.50 (m, 3H), 4.71 (s, 2H), 4.61- 4.53 (m, 1H), 4.40 (d, J = 3.6 Hz, 2H), 4.04 (s, 2H), 3.96 (s, 3H), 3.77-3.57 (m, 2H), 3.41-3.00 (m, 2H), 2.90 (dd, J = 4.4, 15.2 Hz, 3H), 2.33-2.07 (m, 2H) |
| 88 | 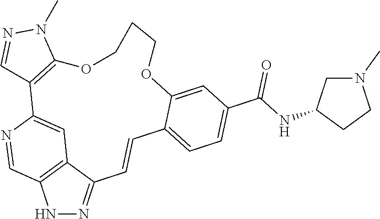 [SMILES: CN1CC[C@H](NC(=O)c2ccc3c(c2)OCCCOc2c(cnn2C)-c2cc4c(n[nH]c4cn2)/C=C/3)C1] | 500.2 | 13.79-13.57 (s, 1H), 13.85-13.55 (s, 1H), 10.00-9.77 (m, 1H), 9.04 (s, 1H), 8.69 (d, J = 6.0 Hz,1H), 8.46 (s, 1H), 8.16-8.10 (m, 1H), 8.08-8.04 (m, 1H), 7.94 (s, 1H), 7.73 (d, J = 2.4 Hz, 1H), 7.70-7.60 (m, 2H), 4.51 (t, J = 5.5 Hz, 2H), 4.38-4.35 (m, 2H), 3.93- 3.92 (s, 3H), 3.77 (s, 3H), 3.31-3.01 (m, 2H), 2.90-2.85 (m, 4H), 2.48- 2.43 (m, 2H) |
| 89 | 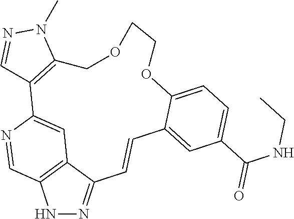 [SMILES: CCNC(=O)c1ccc2c(c1)/C=C/c1n[nH]c3cnc(cc13)-c1cnn(C)c1COCCO2] | 445.1 | 13.97-13.52 (s, 1H), 9.15 (s, 1H), 8.45 (t, J = 5.6 Hz, 1H), 8.38 (d, J = 2.0 Hz, 1H), 8.37 (s, 1H), 8.22 (d, J = 17.2 Hz, 1H), 7.85-7.82 (m, 1H), 7.82 (s, 1H), 7.57 (d, J = 17.2 Hz, 1H), 7.20 (d, J = 8.4 Hz, 1H), 4.71 (s, 2H), 4.41-4.35 (m, 2H), 4.07-4.01 (m, 2H), 3.96 (s, 3H), 3.32 (dd, J = 5.6, 7.2 Hz, 2H), 1.16 (t, J = 7.2 Hz, 3H) |
| 90 | 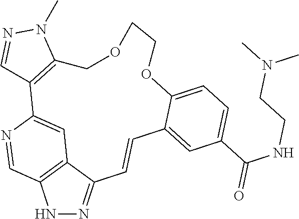 [SMILES: CN(C)CCNC(=O)c1ccc2c(c1)/C=C/c1n[nH]c3cnc(cc13)-c1cnn(C)c1COCCO2] | 488.1 | 13.76 (s, 1H), 9.43-9.30 (s, 1H), 9.14 (s, 1H), 8.70 (t, J = 5.6 Hz, 1H), 8.37 (d, J = 2.0 Hz, 1H), 8.35 (s, 1H), 8.23 (d, J = 17.2 Hz, 1H), 7.85 (dd, J = 2.0, 8.4 Hz, 1H), 7.81 (s, 1H), 7.53 (d, J = 17.2 Hz, 1H), 7.24 (d, J = 8.4 Hz, 1H), 4.71 (s, 2H), 4.42-4.37 (m, 2H), 4.07- 4.02 (m, 2H), 3.96 (s, 3H), 3.64 (q, J = 5.8 Hz, 2H), 3.29 (q, J = 5.8 Hz, 2H), 2.88 (s, 3H), 2.87 (s, 3H). |
| 91 | 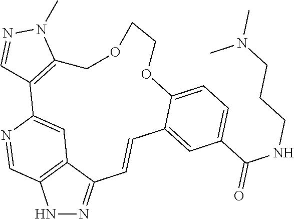 [SMILES: CN(C)CCCNC(=O)c1ccc2c(c1)/C=C/c1n[nH]c3cnc(cc13)-c1cnn(C)c1COCCO2] | 502.1 | 13.78 (s, 1H), 9.43-9.29 (s, 1H), 9.15 (s, 1H), 8.63 (t, J = 5.8 Hz, 1H), 8.38 (d, J = 2.0 Hz, 1H), 8.35 (s, 1H), 8.22 (d, J = 17.2 Hz, 1H), 7.84 (dd, J = 2.0, 8.8 Hz, 1H), 7.81 (s, 1H), 7.55 (d, J = 17.2 Hz, 1H), 7.22 (d, J = 8.8 Hz, 1H), 4.71 (s, 2H), 4.38 (d, J = 4.3 Hz, 2H), 4.07-4.01 (m, 2H), 3.96 (s, 3H), 3.36 (q, J = 6.4 Hz, 2H), 3.01 (m, 2H), 2.81 (s, 3H), 2.80 (s, 3H), 1.73 (m, 2H) |
| 92 | 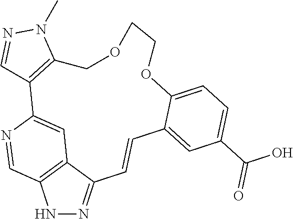 [SMILES: Cn1ncc2c1COCCOc1ccc(C(=O)O)cc1/C=C/c1n[nH]c3cnc-2cc13] | 418.0 | 13.65 (s, 1H), 12.78 (s, 1H), 9.10 (s, 1H), 8.32 (s, 2H), 8.20 (d, J = 17.2 Hz, 1H), 7.93-7.87 (m, 1H), 7.79 (s, 1H), 7.47 (d, J = 17.2 Hz, 1H), 7.22 (d, J = 8.4 Hz, 1H), 4.70 (s, 2H), 4.43-4.37 (m, 2H), 4.05 (m, 2H), 3.95 (s, 3H) |
| 93 | 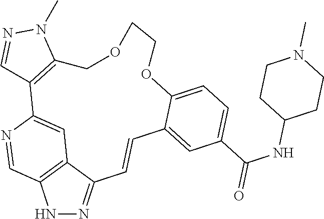 [SMILES: CN1CCC(NC(=O)c2ccc3c(c2)/C=C/c2n[nH]c4cnc(cc24)-c2cnn(C)c2COCCO3)CC1] | 514.2 | 13.77-13.63 (m, 1H), 9.62-9.48 (m, 1H), 9.12 (s, 1H), 8.42 (d, J = 7.2 Hz, 1H), 8.37 (d, J = 2.0 Hz, 1H), 8.34 (s, 1H), 8.22 (d, J = 17.2 Hz, 1H), 7.84 (dd, J = 2.0, 8.4 Hz, 1H), 7.80 (s, 1H), 7.56 (d, J = 17.2 Hz, 1H), 7.21 (d, J = 8.4 Hz, 1H), 4.71 (s, 2H), 4.38 (m, 2H), 4.04 ( m, 2H), 3.96 (s, 3H), 3.48 (m, 3H), 3.36-3.27 (m, 1H), 3.18-3.06 (m, 2H), 2.78 (m, 3H), 2.08 (m, 2H), 1.88-1.75 (m, 2H), |
| 94 | 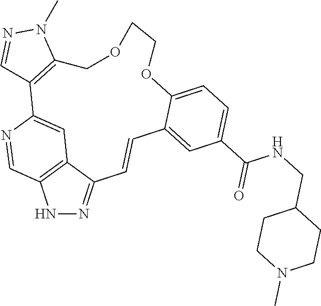 [SMILES: CN1CCC(CNC(=O)c2ccc3c(c2)/C=C/c2n[nH]c4cnc(cc24)-c2cnn(C)c2COCCO3)CC1] | 528.2 | 13.69 (s, 1H), 9.12 (s, 1H), 8.56 (t, J = 5.6 Hz, 1H), 8.38 (d, J = 2.0 Hz, 1H), 8.34 (s, 1H), 8.22 (d, J = 17.2 Hz, 1H), 7.83 (dd, J = 2.0, 8.4 Hz, 1H), 7.80 (s, 1H), 7.56 (d, J = 17.2 Hz, 1H), 7.21 (d, J = 8.4 Hz, 1H), 4.70 (s, 2H), 4.41- 4.36 (m, 2H), 4.06-4.02 (m, 2H), 3.96 (s, 3H), 3.46-3.41 (m, 2H), 3.22 ( t, J = 6.0 Hz, 2H), 2.97-2.86 (m, 2H), 2.75 (d, J = 4.4 Hz, 3H), 1.90 (d, J = 13.6 Hz, 2H), 1.85-1.77 (m, 1H), 1.47- 1.33 (m, 2H) |
| 95 | 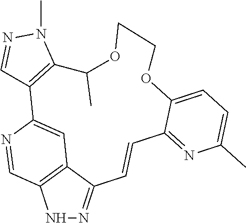 [SMILES: Cc1ccc2c(n1)/C=C/c1n[nH]c3cnc(cc13)-c1cnn(C)c1C(C)OCCO2] | 403.1 | 13.90-13.70 (m, 1H), 9.11 (s, 1H), 9.00-8.89 (m, 1H), 8.24 (d, J = 16.8 Hz, 1H), 7.84-7.75 (m, 2H), 7.68- 7.62 (m, 1H), 7.25 (d, J = 8.0 Hz, 1H), 5.15-5.05 (m, 1H), 4.35-4.19 (m, 2H), 4.10-4.02 (m, 2H), 3.99 (s, 3H), 3.47-3.46 (m, 3H), 1.41 (d, J = 6.8 Hz, 3H) |
| 96 | 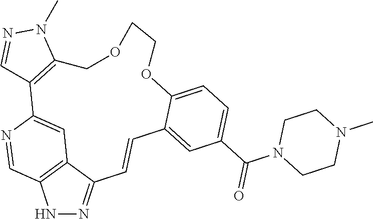 [SMILES: CN1CCN(C(=O)c2ccc3c(c2)/C=C/c2n[nH]c4cnc(cc24)-c2cnn(C)c2COCCO3)CC1] | 500.1 | 13.89 (s, 1H), 10.50-10.01 (m, 1H), 9.19 (s, 1H), 8.38 (s, 1H), 8.20 (d, J = 17.2 Hz, 1H), 7.94 (s, 1H), 7.83 (s, 1H), 7.55 (d, J = 17.2 Hz, 1H), 7.42 (d, J = 8.4 Hz, 1H), 7.22 (d, J = 8.4 Hz, 1H), 4.70 (s, 2H), 4.37 (m, 2H), 4.03 (m, 2H), 3.96 (s, 3H), 3.70-2.95 (m, 8H), 2.85 (s, 3H) |
| 97 | 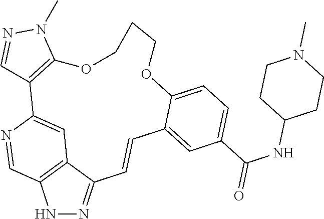 [SMILES: CN1CCC(NC(=O)c2ccc3c(c2)/C=C/c2n[nH]c4cnc(cc24)-c2cnn(C)c2OCCCO3)CC1] | 514.2 | 13.77-13.49 (m, 1H), 9.50-9.29 (m, 1H), 9.04 (s, 1H), 8.46-8.39 (m, 2H), 8.19-8.11 (m, 1H), 7.93 (s, 1H), 7.88- 7.83 (m, 1H), 7.64-7.56 (m, 1H), 7.34 (d, J = 8.8 Hz, 1H), 4.52 (t, J = 5.6 Hz, 2H), 4.37 (t, J = 5.2 Hz, 2H), 4.12- 3.99 (m, 1H), 3.77 (s, 3H), 3.19-3.08 (m, 2H), 2.84-2.77 (m, 3H), 2.63- 2.60 (m, 2H), 2.48-2.43 (m, 2H), 2.14- 2.04 (m, J = 13.6 Hz, 2H), 1.87-1.71 (m, 2H) |
| 98 | 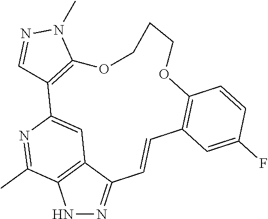 [SMILES: Cc1nc2cc3c(n[nH]c13)/C=C/c1cc(F)ccc1OCCCOc1c-2cnn1C] | 460.0 | 13.8 (s, 1H), 8.28 (s, 1H), 8.08-7.98 (d, J = 17.2 Hz, 1H), 7.92 (s, 1H), 7.78 (dd, J = 10.0, 6.8 Hz, 1H), 7.61 (d, J = 17.2 Hz, 1H), 7.30 (t, J = 4.8 Hz, 1H), 7.19-7.09 (m, 1H), 4.44 (t, J = 5.2 Hz, 2H), 4.29 (t, J = 5.2 Hz, 2H), 3.76 (s, 3H), 2.79 (s, 3H), 2.44-2.40 (m, 2H) |
| 99 | 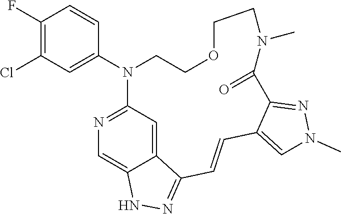 [SMILES: CN1CCOCCN(c2ccc(F)c(Cl)c2)c2cc3c(n[nH]c3cn2)/C=C/c2cn(C)nc2C1=O] | 496.2 | 13.49-13.25 (m, 1H), 8.67 (s, 1H), 8.35 (s, 1H), 7.75 (s, 1H), 7.32-7.22 (m, 3H), 7.09-6.98 (m, 2H), 4.06 (t, J = 5.2 Hz, 2H), 3.89 (s, 3H), 3.76-3.69 (m, 6H), 3.03 (s, 3H) |
| 100 | 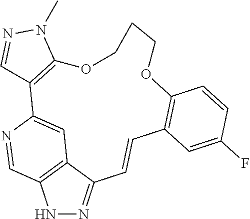 [SMILES: Cn1ncc2c1OCCCOc1ccc(F)cc1/C=C/c1n[nH]c3cnc-2cc13] | 392.1 | 13.64 (s, 1H), 9.02 (s, 1H), 8.44 (s, 1H), 8.05 (dd, J = 1.2, 17.2 Hz, 1H), 7.93 (s, 1H), 7.81 (dd, J = 2.8, 9.6 Hz, 1H), 7.63 (d, J = 17.2 Hz, 1H), 7.34- 7.30 (m, 1H), 7.17-7.09 (m, 1H), 4.47 (t, J = 5.2 Hz, 2H), 4.28 (t, J = 5.2 Hz, 2H), 3.76 (s, 3H), 2.45-2.40 (m, 2H) |

Screen Assays

Biochemical Assay

The inhibition activities of Compounds 1-63 against enzymatic kinases will be evaluated at Reaction Biology Corporation (See, www.reactionbiology.com) using HotSpot assay platform, a radiometric assay based on conventional filter-binding assays, that directly measures kinase catalytic activity toward a specific substrate (Anastassiadis T, et al. Comprehensive Assay of Kinase Catalytic Activity Reveals Features of Kinase Inhibitor Selectivity. Nat Biotechnol. 2011, 29:1039-45). Briefly, specific kinase/substrate pairs along with required cofactors are prepared in reaction buffer; 20 mM Hepes pH 7.5, 10 mM MgCl₂, 1 mM EGTA, 0.02% Brij35, 0.02 mg/ml BSA, 0.1 mM Na₃VO₄, 2 mM DTT, 1% DMSO. Compounds are delivered into the reaction, followed ˜20 minutes later by addition of a mixture of ATP (Sigma, St. Louis MO) and ³³P ATP (Perkin Elmer, Waltham MA) to a final concentration of 10 μM. Reactions are carried out at room temperature for 120 min, followed by spotting of the reactions onto P81 ion exchange filter paper (Whatman Inc., Piscataway, NJ). Unbound phosphate is removed by extensive washing of filters in 0.75% phosphoric acid. After subtraction of background derived from control reactions containing inactive enzyme, kinase activity data is expressed as the percent remaining kinase activity in test samples compared to vehicle (dimethyl sulfoxide) reactions. IC₅₀ values and curve fits are obtained using Prism (GraphPad Software).

Cellular Assay

The inhibition of cellular activity of wild-type and mutant EGFRs will be evaluated at ProQinase GmbH (See, www.proqinase.com) using ProQinase's cellular phosphorylation assays that have been designed to measure compound activity in a physiological environment on a physiological substrate. The cellular kinase assays include EGFR wild-type, EGFR L858R mutant, EGFR T790M mutant, EGFR G719S mutant, EGFR L861Q mutant, EGFR Δ752-759 mutant, EGFR L858R/T790M mutant, EGFR Δ746-750/T790M mutant, EGFR Δ746-750/C797S mutant, EGFR T790M/C797S/L858R mutant, EGFR Δ746-750/T790M/C797S mutant, and EGFR Δ747-749/A750P mutant. The detailed experimental protocols are available at ProQinase GmbH website.

Biochemical Assays for Inhibition of Wild-Type and Mutant EGFRs

The inhibitory activities against EGFR WT, EGFR Δ752-759 mutant, EGFR Δ746-750/T790M mutant, EGFR Δ746-750/C797S mutant, EGFR Δ746-750/T790M/C797S mutant. EGFR L858R mutant, EGFR L858R/T790M mutant, EGFR L858R/C797S mutant, and EGFR L858R/T790M/C797S mutant were evaluated at Reaction Biology Corporation (See, www.reactionbiology.com) using HotSpot assay platfrom, a radiometric assay based on conventional filter-binding assays, that directly measures kinase catalytic activity toward a specific substrate (Anastassiadis T, et al. Comprehensive Assay of Kinase Catalytic Activity Reveals Features of Kinase Inhibitor Selectivity. Nat Biotechnol. 2011, 29:1039-45). Briefly, specific kinase/substrate pairs along with required cofactors were prepared in reaction buffer; 20 mM Hepes pH 7.5, 10 mM MgCl₂, 1 mM EGTA, 0.02% Brij35, 0.02 mg/ml BSA, 0.1 mM Na₃VO₄, 2 mM DTT, 1% DMSO. Compounds were delivered into the reaction, followed ˜20 minutes later by addition of a mixture of ATP (Sigma, St. Louis MO) and ³³P ATP (Perkin Elmer, Waltham MA) to a final concentration of 10 μM. Reactions were carried out at room temperature for 120 min, followed by spotting of the reactions onto P81 ion exchange filter paper (Whatman Inc., Piscataway, NJ). Unbound phosphate was removed by extensive washing of filters in 0.75% phosphoric acid. After subtraction of background derived from control reactions containing inactive enzyme, kinase activity data was expressed as the percent remaining kinase activity in test samples compared to vehicle (dimethyl sulfoxide) reactions. IC₅₀ values and curve fits were obtained using Prism (GraphPad Software).

| | | | | | | EGFR | | EGFR | |
|---|---|---|---|---|---|---|---|---|---|
| | | | | EGFR | | (Δ746- | EGFR | (Δ746- | |
| | | EGFR | EGFR | (L858R/ | EGFR | 750/ | (Δ746- | 750/ | |
| | EGFR | (L858R/ | (L858R/ | T790M/ | (Δ746- | C797 | 750/ | T790M/ | EGFR |
| | (L858R) | C797S) | T790M)_ | C797S/)_ | 750) | S) | T790M) | C797S) | WT |
| Cpd | IC₅₀ | IC₅₀ | IC50, | IC50, | IC₅₀ | IC₅₀ | IC₅₀ | IC₅₀ | IC₅₀ |
| # | (nM) | (nM) | nM | nM | (nM) | (nM) | (nM) | (nM) | (nM) |
| 1 | 29.1 | 55.0 | 4.1 | 15.0 | 6.8 | 4.8 | 2.1 | 0.22 | 46.4 |
| 2 | 257.9 | 595.4 | 301.4 | 691.8 | 49.1 | 16.2 | 22.9 | 29.3 | 554.0 |
| 3 | 373.7 | 1071.0 | 138.3 | 278.5 | 79.0 | 24.4 | 13.2 | 10.2 | 1047.0 |
| 16 | 350.4 | 481.2 | 168 | 263.4 | 32.9 | 61.3 | 38.4 | 5.1 | 551.2 |
| 38 | 1144 | | | 1134 | 963.8 | | | | 1000 |
| 62 | 135.8 | 103.5 | 36.3 | 32.4 | 45.5 | 5.7 | 4.1 | 0.5 | 305.9 |
| 63 | 50.0 | 61.9 | 5.8 | 8.8 | 14.9 | 7.7 | 2.8 | 0.2 | 98.4 |
| 64 | >10,000 | | | | | | | | |
| 65 | 140.3 | 444.7 | 23.4 | 91.2 | 280.8 | 34.1 | 35.9 | 3.9 | 437.7 |
| 66 | >10,000 | | | | | | | | |
| 67 | 1720.0 | | | 111.8 | 1528.0 | | | | 1000.0 |
| 68 | 93.2 | | | 16.6 | 93.3 | | | | 159.3 |
| 69 | 1000.0 | | | 98.3 | 1000.0 | | | | 1000.0 |
| 70 | 2.2 | | | 5.3 | 2.9 | | | | 5.9 |
| 71 | 1.6 | | | 3.7 | 2.4 | | | | 3.9 |
| 72 | 23.7 | | | 64.7 | 18.3 | | | | 48.7 |
| 73 | 39.0 | | | 219.5 | 35.8 | | | | 88.6 |
| 74 | 2.8 | | | 8.3 | 4.0 | | | | 5.7 |
| 75 | 164.8 | | | 184.0 | 51.0 | | | | 937.9 |
| 76 | 1.2 | | | 3.1 | 2.2 | | | | 2.2 |
| 77 | 57.9 | | | 172.5 | 34.6 | | | | 174.0 |
| 78 | 2.9 | | | 10.6 | 5.3 | | | | 6.2 |
| 81 | 28.1 | | | 14.1 | 40.1 | | | | 94.6 |
| 82 | 5.4 | | | 12.0 | 28.3 | | | | 9.8 |
| 83 | 6.0 | | | 13.8 | 34.4 | | | | 11.2 |
| 84 | 432.9 | | | 159.6 | 240.3 | | | | 884.4 |
| 85 | 40.7 | | | 31.0 | 70.0 | | | | 140.4 |
| 86 | 4.9 | | | 8.8 | 15.6 | | | | 24.1 |
| 87 | 16.3 | | | 17.2 | 34.6 | | | | 80.1 |
| 88 | 4.1 | | | 10.3 | 24.6 | | | | 19.3 |
| 89 | 32.0 | | | 23.7 | 23.8 | | | | 158.5 |
| 90 | 22.2 | | | 8.6 | 16.8 | | | | 73.7 |
| 91 | 28.2 | | | 10.2 | 19.7 | | | | 97.2 |
| 92 | 66.4 | | | 55.5 | 47.1 | | | | 270.9 |
| 93 | 7.2 | | | 4.2 | 13.6 | | | | 55.8 |
| 94 | 9.2 | | | 9.9 | 17.6 | | | | 101.4 |
| 95 | 74.9 | | | 35.0 | 139.0 | | | | 91.6 |
| 96 | 470.0 | | | 343.0 | 740.0 | | | | 586.0 |
| 97 | 4.5 | | | 3.4 | 9.7 | | | | 3.1 |
| 98 | 1114.0 | | | 192.5 | 835.8 | | | | 1235.0 |
| 99 | 25.5 | 50.2 | >10,000 | >10,000 | 84.4 | 11.3 | 8531.0 | 3380.0 | 33.5 |
| 100 | 13.9 | | | 27.5 | 24.0 | | | | 176.2 |

What is claimed is:

1. A compound of the formula Ia 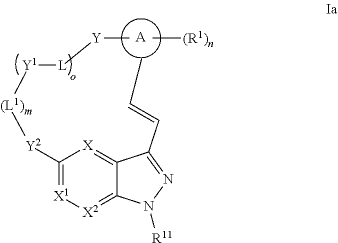
wherein
A is a ring selected from 5- to 10-membered heteroarylene or C₆-C₁₀ arylene;
X is C(R²) or N;
X¹ is C(R³) or N;
X² is C(R⁴) or N;
Y is O, N(R⁵)C(O), C(O)N(R⁵), N(R⁶), N(R⁵)S(O), S(O)N(R⁵), N(R⁵)S(O)₂, S(O)₂N(R⁵), S, S(O), S(O)₂, or Y is absent;
each Y¹ is independently O, C(O)N(R⁷), N(R⁷)C(O), N(R⁸), N(R⁷)S(O), S(O)N(R⁷), N(R⁷)S(O)₂, S(O)₂N(R⁷), S, S(O), S(O)₂, or absent;
Y² is O, C(O)N(R⁹), N(R⁹)C(O), N(R¹⁰), N(R⁹)S(O), S(O)N(R⁹), N(R⁹)S(O)₂, S(O)₂N(R⁹), S, S(O), S(O)₂, or Y² is absent;
each L is independently a C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or optionally two hydrogen atoms on one carbon atom in C₁-C₆ alkylene are optionally substituted by a C₂-C₅ alkylene to provide a C₃-C₆ cycloalkylene; or optionally two hydrogen atoms on two carbon atoms in C₁-C₆ alkylene are optionally substituted by a C₁-C₄ alkylene to provide a C₃-C₆ cycloalkylene; or optionally one hydrogen atom on one carbon atom in C₁-C₆ alkylene and one of R⁵, R⁶, R⁷, or R⁸ taken together with the atoms to which they are attached combine to form a 3- to 7-membered heterocycloalkylene;
each L¹, when present, is independently C₁-C₆ alkylene or a ring B selected from the group consisting of 5- to 10-membered heteroarylene, 3- to 10-membered heterocycloalkylene, 3- to 6-membered cycloalkylene, and C₆-C₁₀ arylene, wherein each hydrogen atom in 5- to 10-membered heteroarylene, 3- to 10-membered heterocycloalkylene, C₆-C₁₀ arylene, and C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R², R³, and R⁴, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, R⁸, R⁹, R¹⁰, and R¹¹, when present, is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{c} and R^{d} or R^{e} and R^{f}, taken together with the atom to which they are attached, form a a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m is 0, 1, 2, 3, or 4;
n is 0, 1, 2, 3, or 4; and
o is 0, 1, 2, or 3;
or a pharmaceutically acceptable salt thereof.

2. The compound of claim 1, or a pharmaceutically acceptable salt thereof, having the formula IIa 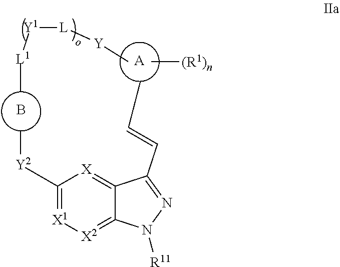
wherein
ring B is a 5- to 10-membered heteroarylene, 3- to 10-membered heterocycloalkylene, or C₆-C₁₀ arylene, wherein each hydrogen atom in 3- to 10-membered heteroarylene, 5- to 10-membered heterocycloalkylene, and C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂; and
L¹ is C₁-C₆ alkylene, wherein each hydrogen atom in C₁-C₆ alkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

3. The compound of claim 1, or a pharmaceutically acceptable salt thereof, having the formula IVa 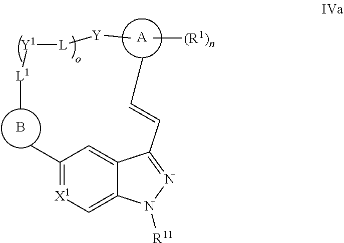
wherein
ring A is a ring selected from 5- to 10-membered heteroarylene or C₆-C₁₀ arylene;
X¹ is C(R³) or N;
Y is O, N(R⁵)C(O), S, S(O), or S(O)₂;
each Y¹ is independently O, S, S(O), or S(O)₂;
each L is independently a C₁-C₆ alkylene, wherein each hydrogen atom in C₁-C₆ alkylene is independently optionally substituted by deuterium;
L¹ is absent, or L¹ is C₁-C₆ alkylene, wherein each hydrogen atom in C₁-C₆ alkylene is independently optionally substituted by deuterium or C₁-C₆ alkyl;
ring B is a 5- to 10-membered heteroarylene or C₆-C₁₀ arylene, wherein each hydrogen atom in 3- to 10-membered heteroarylene or C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R¹ is independently deuterium, halogen, C₁-C₆ alkyl, C₂-C₆ alkenyl, C₂-C₆ alkynyl, C₃-C₆ cycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, and R¹¹, when present, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{c} and R^{d} or R^{e} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂; and
n is 0, 1, 2, 3, or 4.

4. The compound of claim 1, having the formula Va, V, Via, VI, VIIa, VII, VIIIa, VIII, IXa, or IX 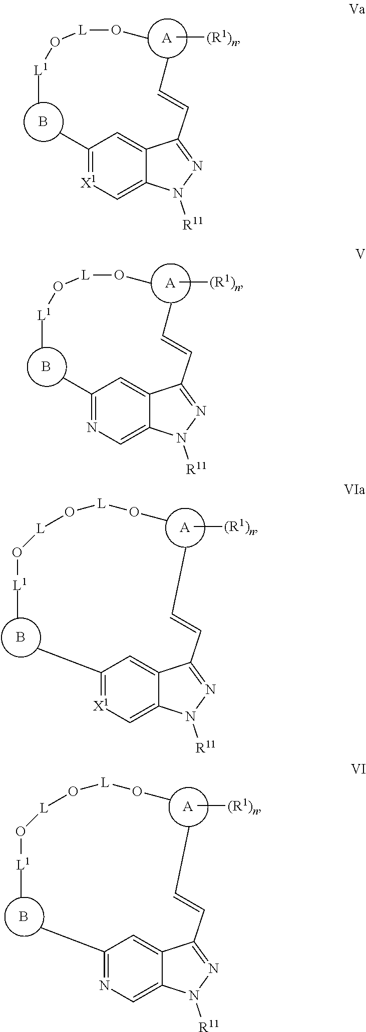 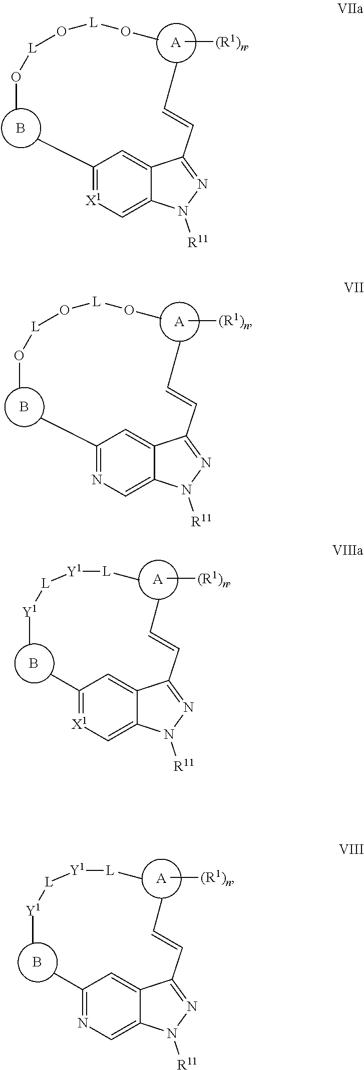 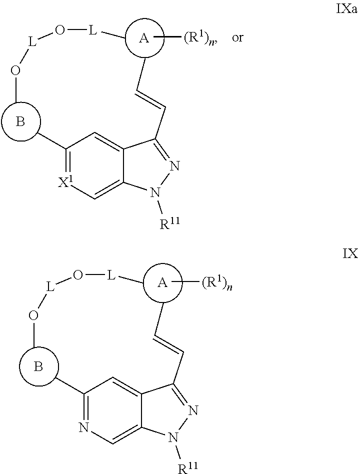
or a pharmaceutically acceptable salt thereof.

5. The compound of any one of the preceding claims, or a pharmaceutically acceptable salt thereof, wherein ring B is a 5- to 10-membered heteroarylene, wherein each hydrogen atom in 5- to 10-membered heteroarylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

6. The compound of any one of claims 1 to 4, or a pharmaceutically acceptable salt thereof, wherein ring B is a 5- to 10-membered heteroarylene selected from the group consisting of 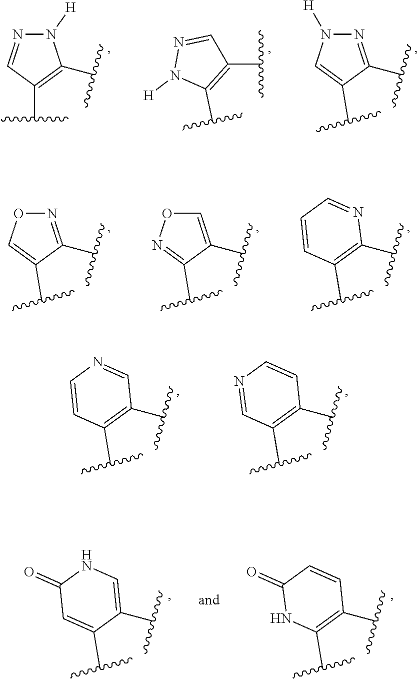
wherein each hydrogen atom in ring B is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

7. The compound of any one of claims 1 to 4, or a pharmaceutically acceptable salt thereof, wherein ring B is

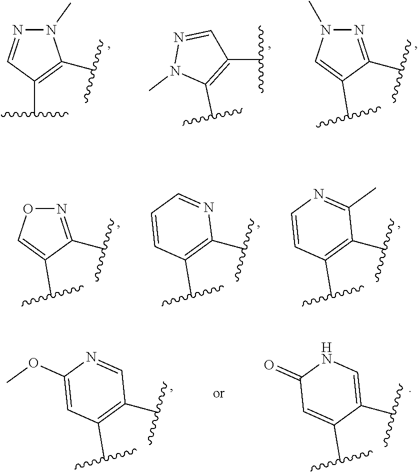

8. The compound of any one of claims 1 to 4, or a pharmaceutically acceptable salt thereof, wherein ring B is a 3- to 10-membered heterocycloalkylene, wherein each hydrogen atom in 3- to 10-membered heterocycloalkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

9. The compound of any one of claims 1 to 4, or a pharmaceutically acceptable salt thereof, wherein ring B is a 3- to 10-membered heterocycloalkylene selected from the group consisting of 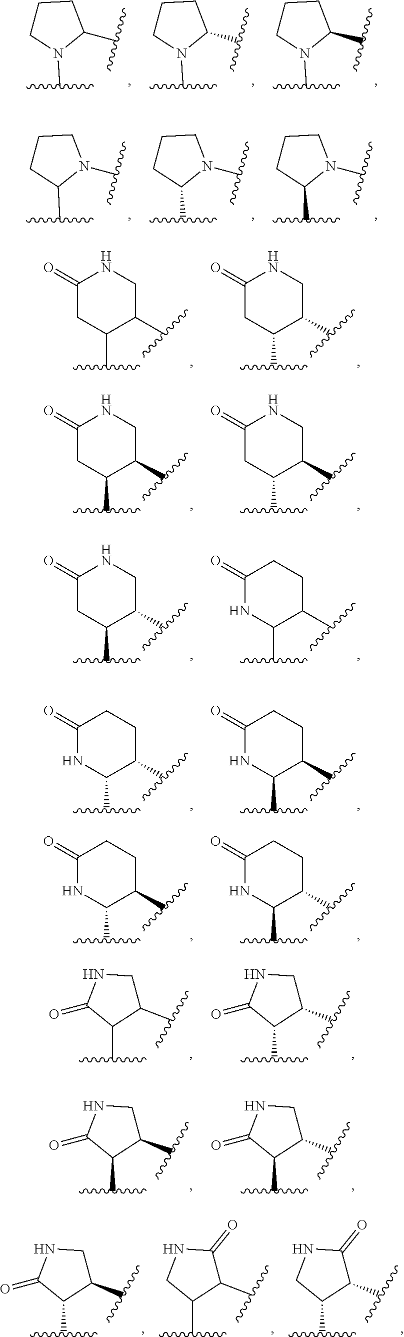 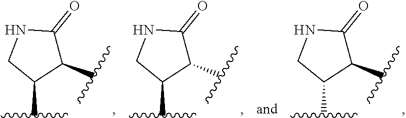
wherein each hydrogen atom in 3- to 10-membered heterocycloalkylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

10. The compound of any one of claims 1 to 4, or a pharmaceutically acceptable salt thereof, wherein ring B is a 3- to 10-membered heterocycloalkylene selected from the group consisting of

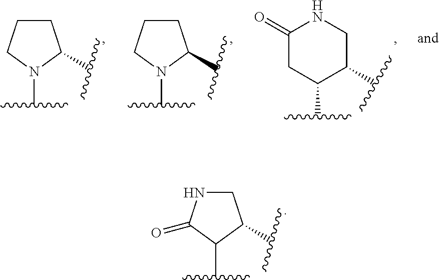 [SMILES: *[C@H]1CCCN1* ; *[C@@H]1CCCN1* ; *[C@@H]1CC(=O)NC[C@@H]1* ; *C1C(=O)NC[C@@H]1*]

11. The compound of any one of claims 1 to 4, or a pharmaceutically acceptable salt thereof, wherein ring B is a C₆-C₁₀ arylene, wherein each hydrogen atom in C₆-C₁₀ arylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

12. The compound of any one of claims 1 to 4, or a pharmaceutically acceptable salt thereof, wherein ring B is a phenylene optionally substituted with one or more deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

13. The compound of any one of claims 1 to 4, or a pharmaceutically acceptable salt thereof, wherein ring B is selected from the group consisting of

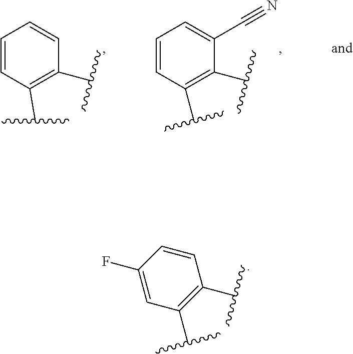 [SMILES: *c1ccccc1* ; *c1cccc(C#N)c1* ; *c1ccc(F)cc1*]

14. The compound of any one of claims 1 to 4, or a pharmaceutically acceptable salt thereof, wherein 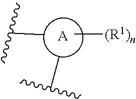
is a 5- to 10-membered heteroarylene, and n is 0, 1, 2, 3, or 4.

15. The compound of any one of claims 1 to 4, or a pharmaceutically acceptable salt thereof, wherein 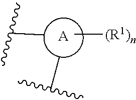
is a 5- to 10-membered heteroarylene selected from the group consisting of 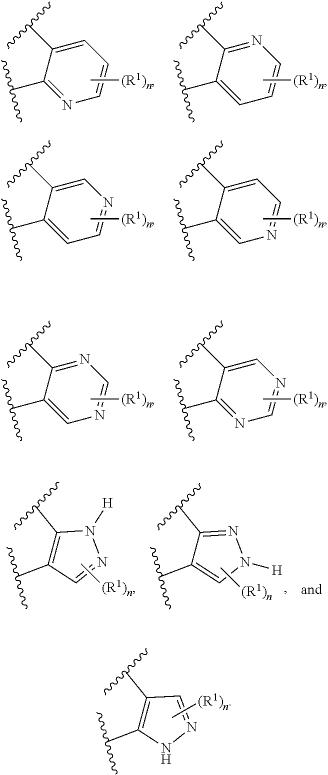
and n is 0, 1, or 2.

16. The compound of any one of the preceding claims, or a pharmaceutically acceptable salt thereof, and n is 0, 1, or 2.

17. The compound of any one of claims 1 to 4, or a pharmaceutically acceptable salt thereof, wherein each R¹, when present, is independently fluoro, chloro, methyl, ethyl, methoxy, ethoxy, —C(O)OR^{a}, —C(O)NR^{a}R^{b}, —CN, or 4-piperidinyl.

18. The compound of any one of claims 1 to 4, or a pharmaceutically acceptable salt thereof, wherein 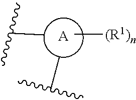
is a 5- to 10-membered heteroarylene selected from the group consisting of

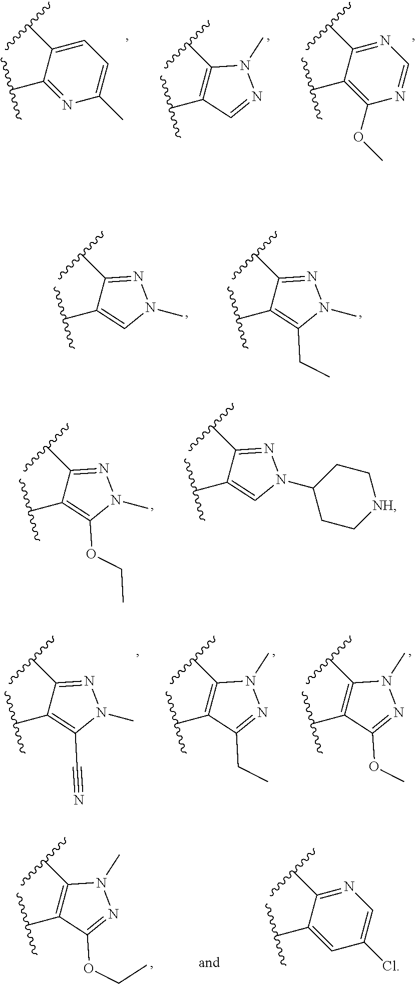

19. The compound of any one of claims 1 to 4, or a pharmaceutically acceptable salt thereof, wherein 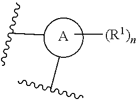
is a C₆-C₁₀ arylene, and n is 0, 1, 2, 3, or 4.

20. The compound of any one of claims 1 to 4, or a pharmaceutically acceptable salt thereof, wherein 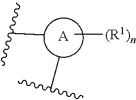
is a phenylene, and n is 0, 1, 2, 3, or 4.

21. The compound of any one of claims 1 to 4, or a pharmaceutically acceptable salt thereof, wherein n is 0, 1, or 2.

22. The compound of any one of the preceding claims, or a pharmaceutically acceptable salt thereof, wherein each R¹, when present, is independently fluoro, chloro, methyl, ethyl, methoxy, ethoxy, —C(O)OR^{a}, —C(O)NR^{a}R^{b}, —CN, or 4-piperidinyl.

23. The compound of any one of claims 1 to 4, or a pharmaceutically acceptable salt thereof, wherein 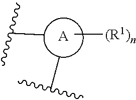
is selected from the group consisting of

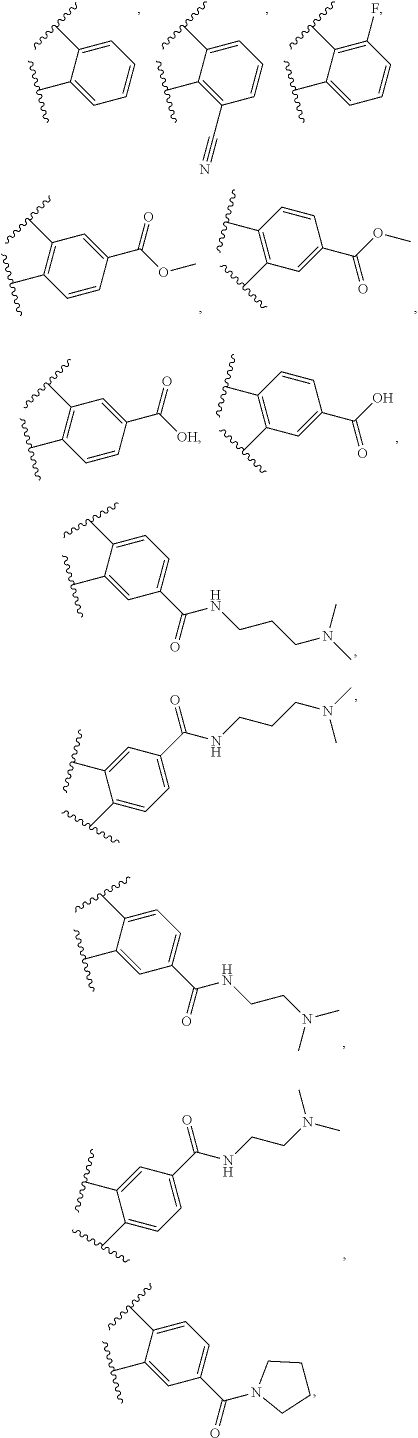

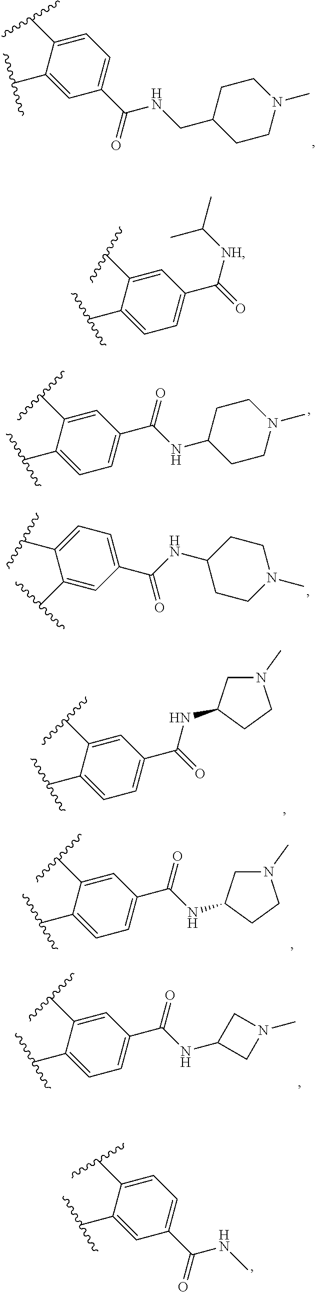

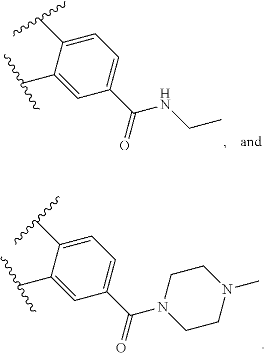 [SMILES: *c1ccc(C(=O)NCC)cc1* ; *c1ccc(C(=O)N2CCN(C)CC2)cc1*]

24. The compound of any one of claims 1 to 4, or a pharmaceutically acceptable salt thereof, wherein L is an ethylene, propylene, or butylene, wherein each hydrogen atom in ethylene, propylene, and butylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂.

25. The compound of any one of the preceding claims, or a pharmaceutically acceptable salt thereof, wherein L is an ethylene, propylene, or butylene, each of which is optionally substituted by a C₁-C₆ alkyl.

26. The compound of any one of the preceding claims, or a pharmaceutically acceptable salt thereof, wherein Y is O.

27. The compound of any one of claims 1 to 25, or a pharmaceutically acceptable salt thereof, wherein Y is N(R⁵)C(O).

28. The compound of any one of the preceding claims, or a pharmaceutically acceptable salt thereof, wherein R⁵, when present, is H or methyl.

29. The compound of any one of claims 1 to 25, or a pharmaceutically acceptable salt thereof, wherein Y is absent.

30. The compound of any one of the preceding claims, or a pharmaceutically acceptable salt thereof, wherein Y¹ is O.

31. The compound of any one of claims 1 to 29, or a pharmaceutically acceptable salt thereof, wherein Y¹ is C(O)N(R⁷).

32. The compound of any one of the preceding claims, or a pharmaceutically acceptable salt thereof, wherein R⁷, when present, is H or methyl.

33. The compound of any one of claims 1 to 29, or a pharmaceutically acceptable salt thereof, wherein Y¹ is N(R⁸).

34. The compound of any one of the preceding claims, or a pharmaceutically acceptable salt thereof, wherein R¹, when present, is H or methyl.

35. The compound of any one of claims 1 to 29, or a pharmaceutically acceptable salt thereof, wherein Y¹ is absent.

36. The compound of any one of the preceding claims, or a pharmaceutically acceptable salt thereof, wherein Y² is O.

37. The compound of any one of claims 1 to 35, or a pharmaceutically acceptable salt thereof, wherein Y² is C(O)N(R⁹).

38. The compound of claim 37, or a pharmaceutically acceptable salt thereof, wherein R⁹ is H, methyl, ethyl, or cyclopropyl.

39. The compound of any one of claims 1 to 35, or a pharmaceutically acceptable salt thereof, wherein Y² is N(R¹⁰).

40. The compound of claim 39, or a pharmaceutically acceptable salt thereof, wherein R¹⁰ is H, methyl, or phenyl.

41. The compound of any one of claims 1 to 35, or a pharmaceutically acceptable salt thereof, wherein Y² is S(O)₂.

42. The compound of any one of claims 1 to 35, or a pharmaceutically acceptable salt thereof, wherein Y² is absent.

43. The compound of any one of the preceding claims, or a pharmaceutically acceptable salt thereof, wherein m is 1.

44. The compound of any one of the preceding claims, or a pharmaceutically acceptable salt thereof, wherein m is 2.

45. The compound of any one of claims 1 to 4, or a pharmaceutically acceptable salt thereof, wherein at least one L¹ is methylene, ethylene, or propylene, wherein each hydrogen atom in methylene, ethylene, and propylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

46. The compound of any one of the preceding claims, or a pharmaceutically acceptable salt thereof, wherein at least one L¹ is methylene, ethylene, or propylene, each of which is substituted with a C₁-C₆ alkyl or a —C(O)NR^{c}R^{d}.

47. The compound of any one of the preceding claims, or a pharmaceutically acceptable salt thereof, wherein at least one L¹ is methylene, ethylene, or propylene, each of which is substituted with a methyl or a —C(O)NR^{c}R^{d}, wherein R^{c} and R^{d} are each H.

48. The compound of any one of claims 1 to 42, or a pharmaceutically acceptable salt thereof, wherein m is 0.

49. The compound of any one of the preceding claims, or a pharmaceutically acceptable salt thereof, wherein X is C(R²).

50. The compound of any one of the preceding claims, or a pharmaceutically acceptable salt thereof, wherein X¹ is CH or N.

51. The compound of any one of claims 1 to 49, or a pharmaceutically acceptable salt thereof, wherein X¹ is C(R³).

52. The compound of any one of the preceding claims, or a pharmaceutically acceptable salt thereof, wherein X² is C(R⁴).

53. The compound of any one of the claims 1 to 51, or a pharmaceutically acceptable salt thereof, wherein X² is N.

54. The compound of any one of claims 1 to 48, or a pharmaceutically acceptable salt thereof, wherein X is N.

55. The compound of any one of claims 1 to 48, or 54, or a pharmaceutically acceptable salt thereof, wherein X¹ is C(R³).

56. The compound of any one of claims 1 to 48, or 54, or a pharmaceutically acceptable salt thereof, wherein X¹ is N.

57. The compound of any one of claims 1 to 48, or 54 to 56, or a pharmaceutically acceptable salt thereof, wherein X² is C(R⁴).

58. The compound of any one of claims 1 to 48, or 54 to 56, or a pharmaceutically acceptable salt thereof, wherein X² is N.

59. The compound of any one of the preceding claims, or a pharmaceutically acceptable salt thereof, wherein R², when present, is H.

60. The compound of any one of the preceding claims, or a pharmaceutically acceptable salt thereof, wherein R³, when present, is H.

61. The compound of any one of the preceding claims, or a pharmaceutically acceptable salt thereof, wherein R⁴, when present, is H, fluoro, chloro, or methyl.

62. The compound of any one of the preceding claims, or a pharmaceutically acceptable salt thereof, wherein R¹¹ is H.

63. The compound of claim 1, selected from the group consisting of 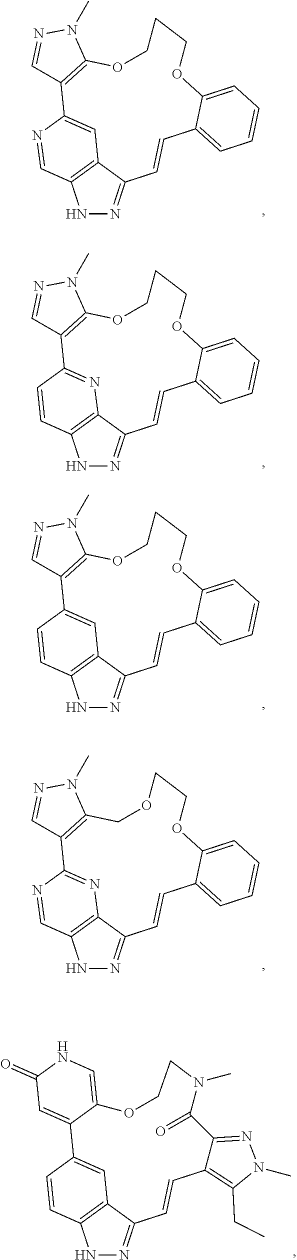 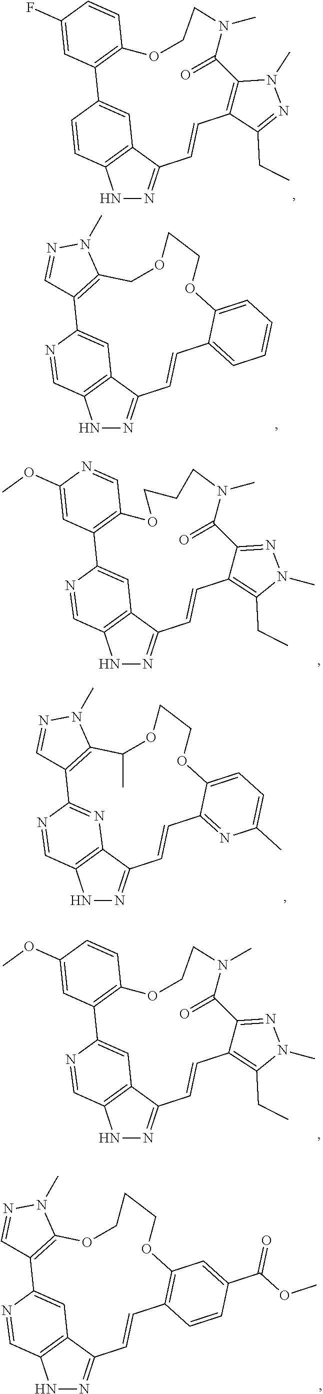 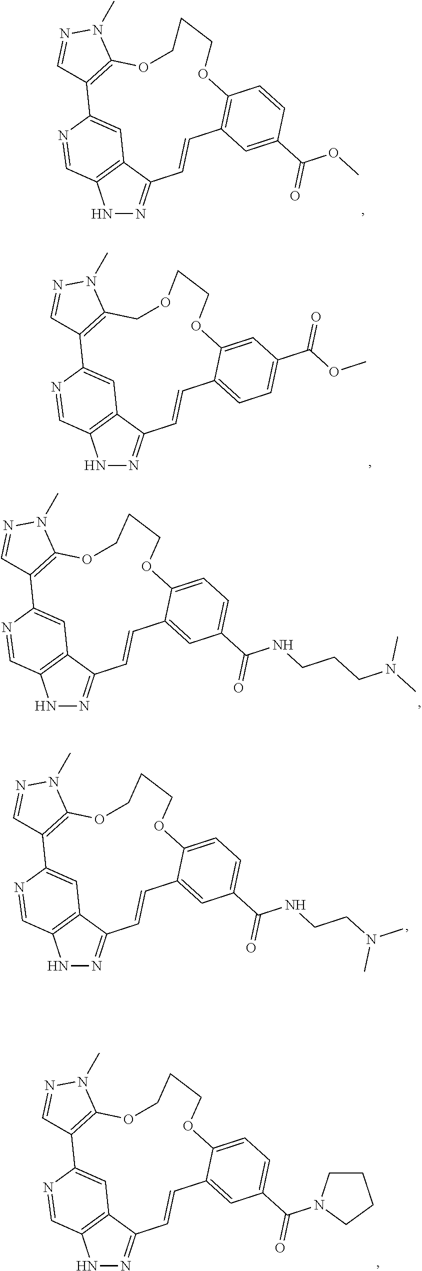 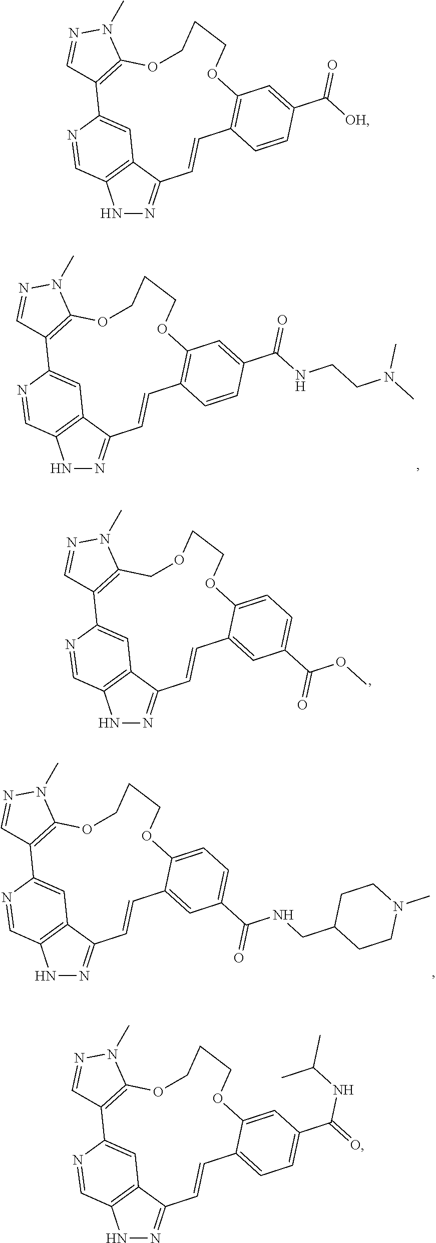 [SMILES: Cn1ncc2c1OCCCOc1cc(C(=O)O)ccc1/C=C/c1n[nH]c3cnc-2cc13 ; CN(C)CCNC(=O)c1ccc2c(c1)OCCCOc1c(cnn1C)-c1cc3c(n[nH]c3cn1)/C=C/2 ; COC(=O)c1ccc2c(c1)/C=C/c1n[nH]c3cnc(cc13)-c1cnn(C)c1COCCO2 ; CN1CCC(CNC(=O)c2ccc3c(c2)/C=C/c2n[nH]c4cnc(cc24)-c2cnn(C)c2OCCCO3)CC1 ; CC(C)NC(=O)c1ccc2c(c1)OCCCOc1c(cnn1C)-c1cc3c(n[nH]c3cn1)/C=C/2] 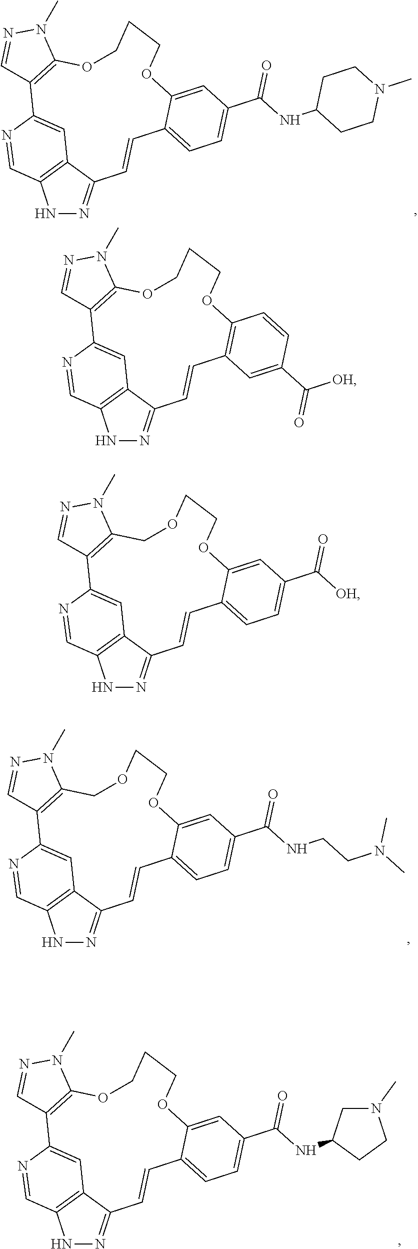 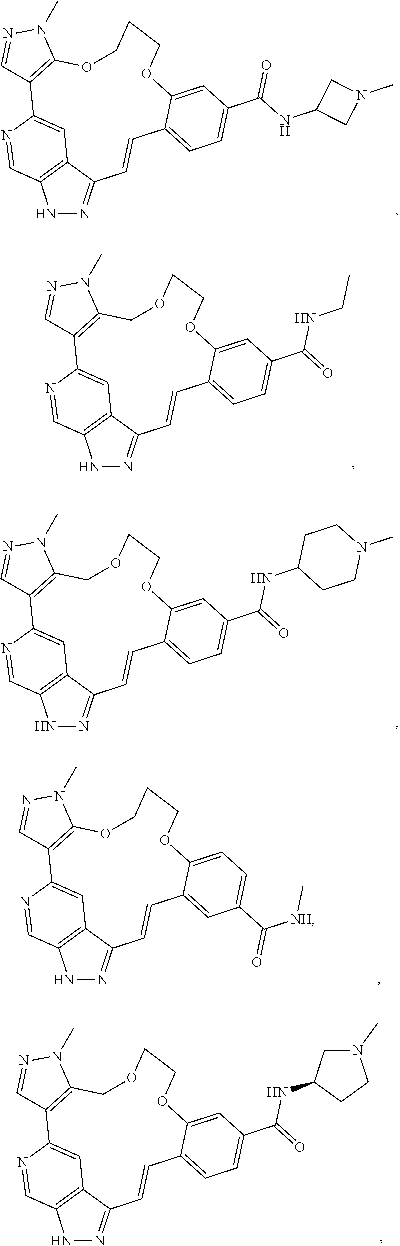 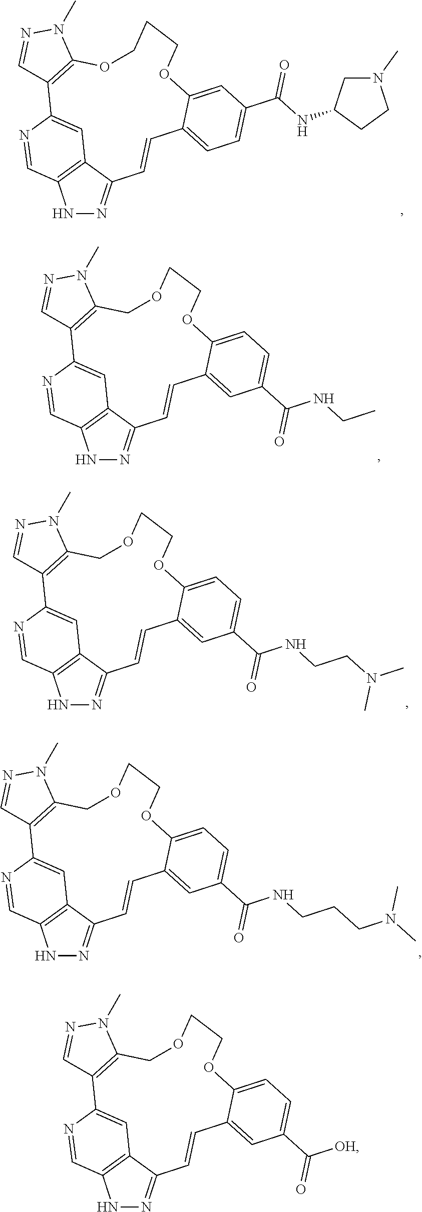 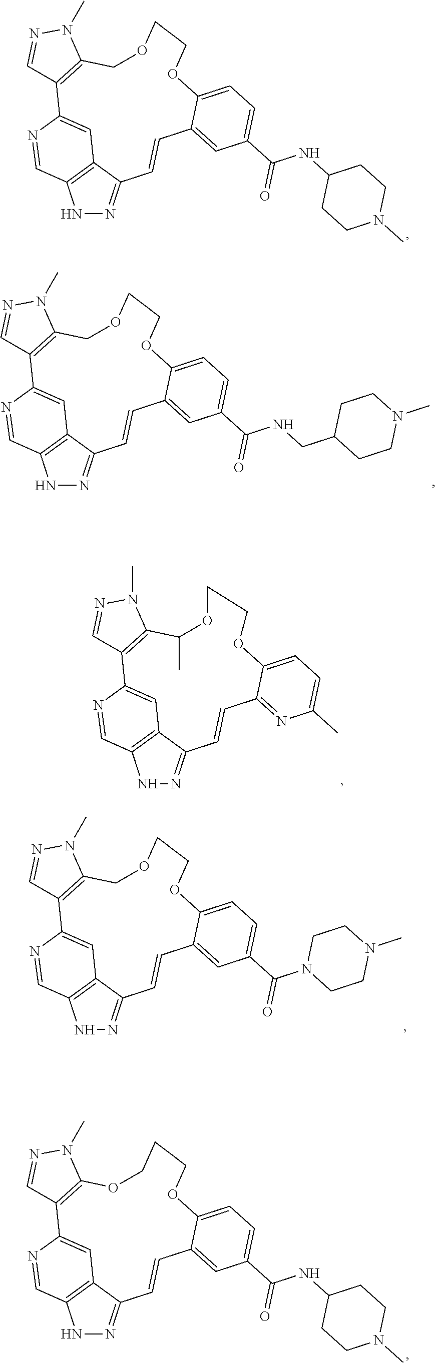 [SMILES: CN1CCC(NC(=O)c2ccc3c(c2)/C=C/c2n[nH]c4cnc(cc24)-c2cnn(C)c2COCCO3)CC1 ; CN1CCC(CNC(=O)c2ccc3c(c2)/C=C/c2n[nH]c4cnc(cc24)-c2cnn(C)c2COCCO3)CC1 ; Cc1ccc2c(n1)/C=C/c1n[nH]c3cnc(cc13)-c1cnn(C)c1C(C)OCCO2 ; CN1CCN(C(=O)c2ccc3c(c2)/C=C/c2n[nH]c4cnc(cc24)-c2cnn(C)c2COCCO3)CC1 ; CN1CCC(NC(=O)c2ccc3c(c2)/C=C/c2n[nH]c4cnc(cc24)-c2cnn(C)c2OCCCO3)CC1] 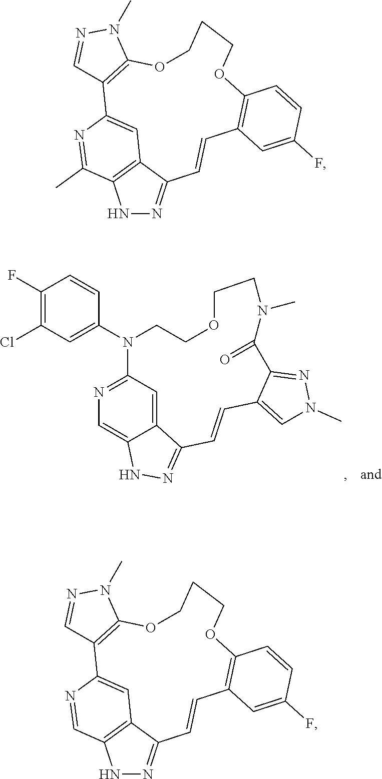 [SMILES: Cc1nc2cc3c(n[nH]c13)/C=C/c1cc(F)ccc1OCCCOc1c-2cnn1C ; CN1CCOCCN(c2ccc(F)c(Cl)c2)c2cc3c(n[nH]c3cn2)/C=C/c2cn(C)nc2C1=O ; Cn1ncc2c1OCCCOc1ccc(F)cc1/C=C/c1n[nH]c3cnc-2cc13]
or a pharmaceutically acceptable salt thereof.

64. The compound of claim 1, selected from the group consisting of 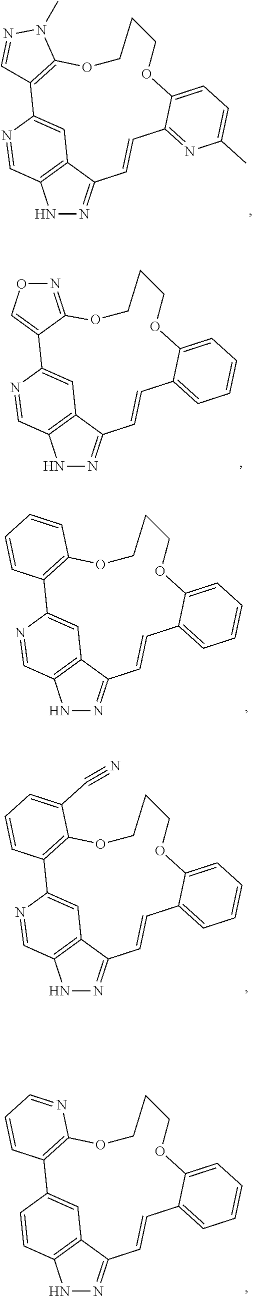 [SMILES: Cc1ccc2c(n1)/C=C/c1n[nH]c3cnc(cc13)-c1cnn(C)c1OCCCO2 ; C1=C/c2n[nH]c3cnc(cc23)-c2conc2OCCCOc2ccccc2/1 ; C1=C/c2n[nH]c3cnc(cc23)-c2ccccc2OCCCOc2ccccc2/1 ; N#Cc1cccc2c1OCCCOc1ccccc1/C=C/c1n[nH]c3cnc-2cc13 ; C1=C/c2n[nH]c3ccc(cc23)-c2cccnc2OCCCOc2ccccc2/1] 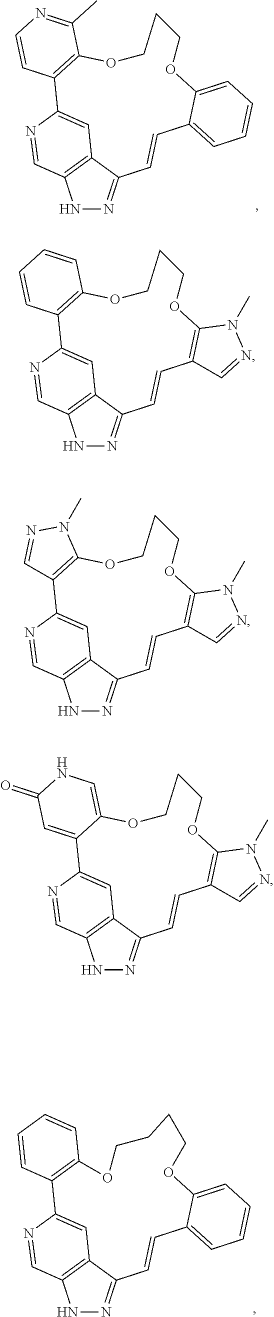 [SMILES: Cc1nccc2c1OCCCOc1ccccc1/C=C/c1n[nH]c3cnc-2cc13 ; Cn1ncc2c1OCCCOc1ccccc1-c1cc3c(n[nH]c3cn1)/C=C/2 ; Cn1ncc2c1OCCCOc1c(cnn1C)-c1cc3c(n[nH]c3cn1)/C=C/2 ; Cn1ncc2c1OCCCOc1c[nH]c(=O)cc1-c1cc3c(n[nH]c3cn1)/C=C/2 ; C1=C/c2n[nH]c3cnc(cc23)-c2ccccc2OCCCCOc2ccccc2/1] 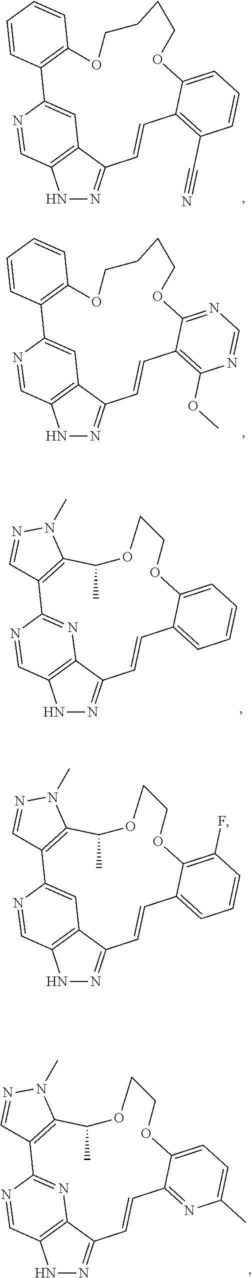 [SMILES: N#Cc1cccc2c1/C=C/c1n[nH]c3cnc(cc13)-c1ccccc1OCCCCO2 ; COc1ncnc2c1/C=C/c1n[nH]c3cnc(cc13)-c1ccccc1OCCCCO2 ; C[C@H]1OCCOc2ccccc2/C=C/c2n[nH]c3cnc(nc23)-c2cnn(C)c21 ; C[C@H]1OCCOc2c(F)cccc2/C=C/c2n[nH]c3cnc(cc23)-c2cnn(C)c21 ; Cc1ccc2c(n1)/C=C/c1n[nH]c3cnc(nc13)-c1cnn(C)c1[C@@H](C)OCCO2] 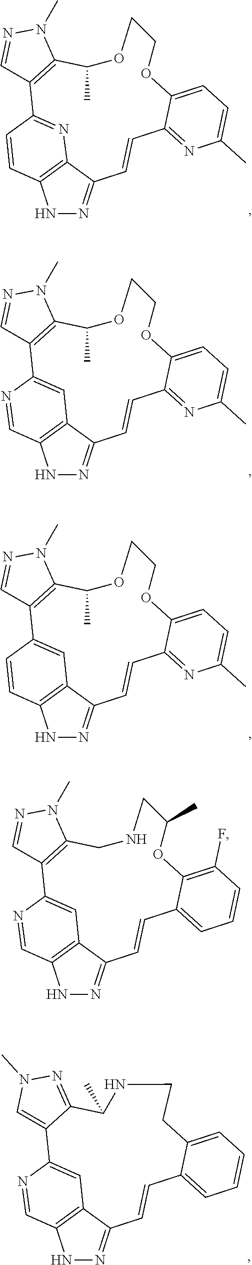 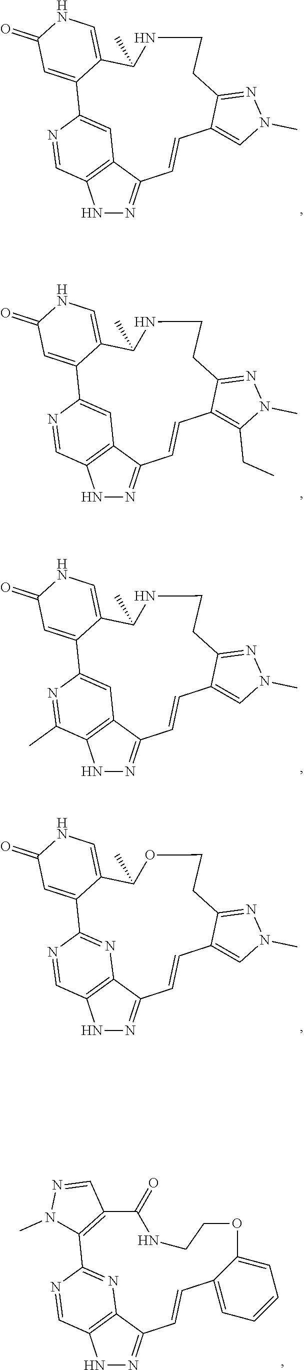 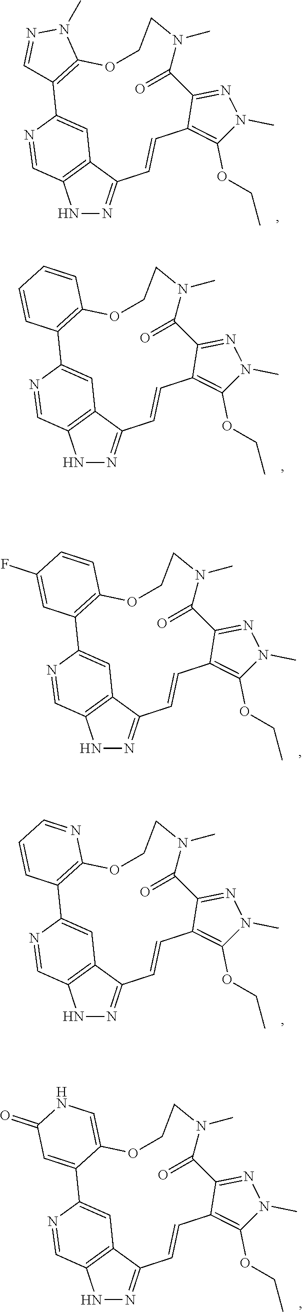 [SMILES: CCOc1c2c(nn1C)C(=O)N(C)CCOc1c(cnn1C)-c1cc3c(n[nH]c3cn1)/C=C/2 ; CCOc1c2c(nn1C)C(=O)N(C)CCOc1ccccc1-c1cc3c(n[nH]c3cn1)/C=C/2 ; CCOc1c2c(nn1C)C(=O)N(C)CCOc1ccc(F)cc1-c1cc3c(n[nH]c3cn1)/C=C/2 ; CCOc1c2c(nn1C)C(=O)N(C)CCOc1ncccc1-c1cc3c(n[nH]c3cn1)/C=C/2 ; CCOc1c2c(nn1C)C(=O)N(C)CCOc1c[nH]c(=O)cc1-c1cc3c(n[nH]c3cn1)/C=C/2] 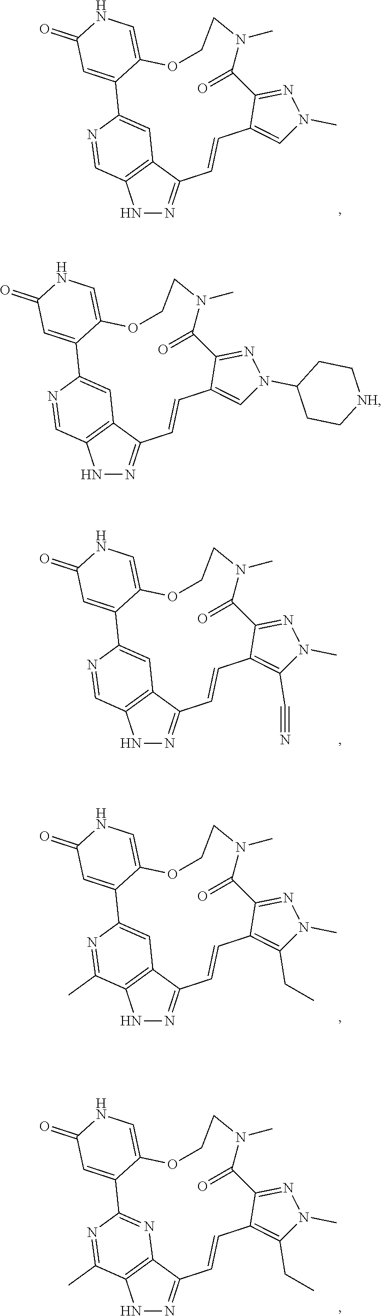 [SMILES: CN1CCOc2c[nH]c(=O)cc2-c2cc3c(n[nH]c3cn2)/C=C/c2cn(C)nc2C1=O ; CN1CCOc2c[nH]c(=O)cc2-c2cc3c(n[nH]c3cn2)/C=C/c2cn(C3CCNCC3)nc2C1=O ; CN1CCOc2c[nH]c(=O)cc2-c2cc3c(n[nH]c3cn2)/C=C/c2c(nn(C)c2C#N)C1=O ; CCc1c2c(nn1C)C(=O)N(C)CCOc1c[nH]c(=O)cc1-c1cc3c(n[nH]c3c(C)n1)/C=C/2 ; CCc1c2c(nn1C)C(=O)N(C)CCOc1c[nH]c(=O)cc1-c1nc(C)c3[nH]nc(c3n1)/C=C/2] 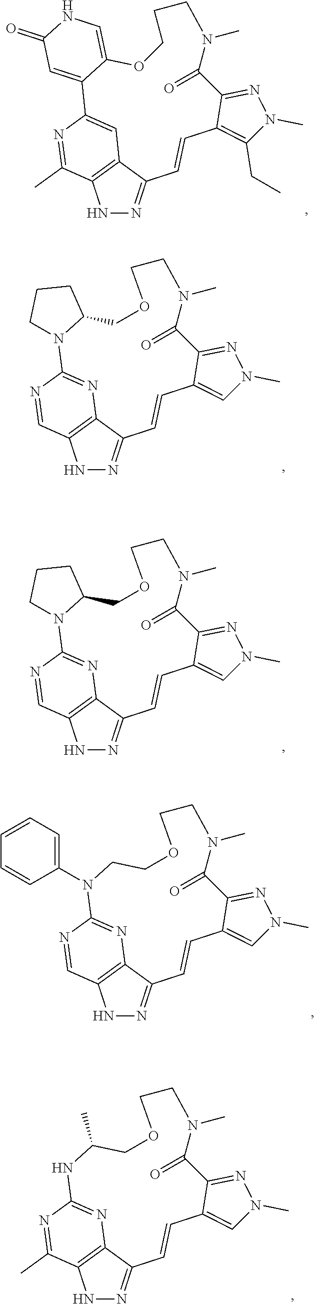 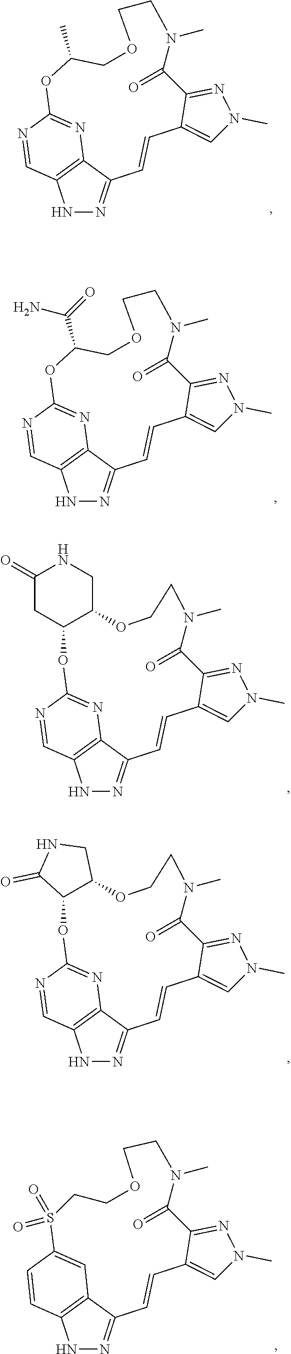 [SMILES: C[C@@H]1COCCN(C)C(=O)c2nn(C)cc2/C=C/c2n[nH]c3cnc(nc23)O1 ; CN1CCOC[C@@H](C(N)=O)Oc2ncc3[nH]nc(c3n2)/C=C/c2cn(C)nc2C1=O ; CN1CCO[C@H]2CNC(=O)C[C@H]2Oc2ncc3[nH]nc(c3n2)/C=C/c2cn(C)nc2C1=O ; CN1CCO[C@H]2CNC(=O)[C@H]2Oc2ncc3[nH]nc(c3n2)/C=C/c2cn(C)nc2C1=O ; CN1CCOCCS(=O)(=O)c2ccc3[nH]nc(c3c2)/C=C/c2cn(C)nc2C1=O] 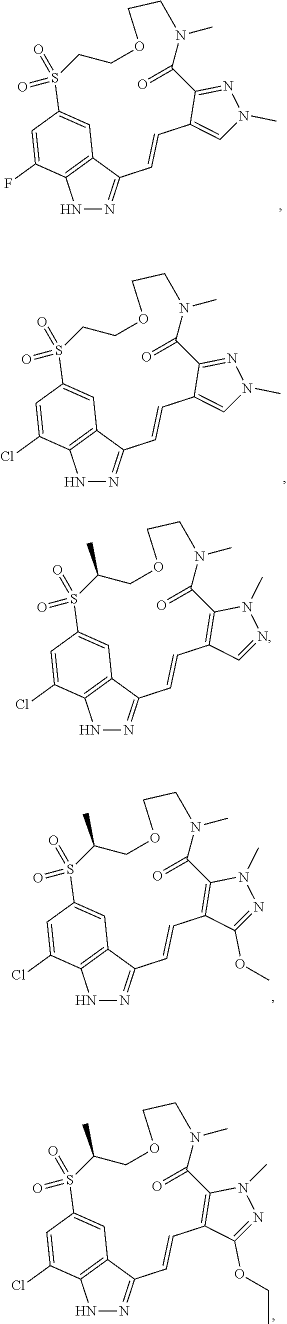 [SMILES: CN1CCOCCS(=O)(=O)c2cc(F)c3[nH]nc(c3c2)/C=C/c2cn(C)nc2C1=O ; CN1CCOCCS(=O)(=O)c2cc(Cl)c3[nH]nc(c3c2)/C=C/c2cn(C)nc2C1=O ; C[C@H]1COCCN(C)C(=O)c2c(cnn2C)/C=C/c2n[nH]c3c(Cl)cc(cc23)S1(=O)=O ; COc1nn(C)c2c1/C=C/c1n[nH]c3c(Cl)cc(cc13)S(=O)(=O)[C@@H](C)COCCN(C)C2=O ; CCOc1nn(C)c2c1/C=C/c1n[nH]c3c(Cl)cc(cc13)S(=O)(=O)[C@@H](C)COCCN(C)C2=O] 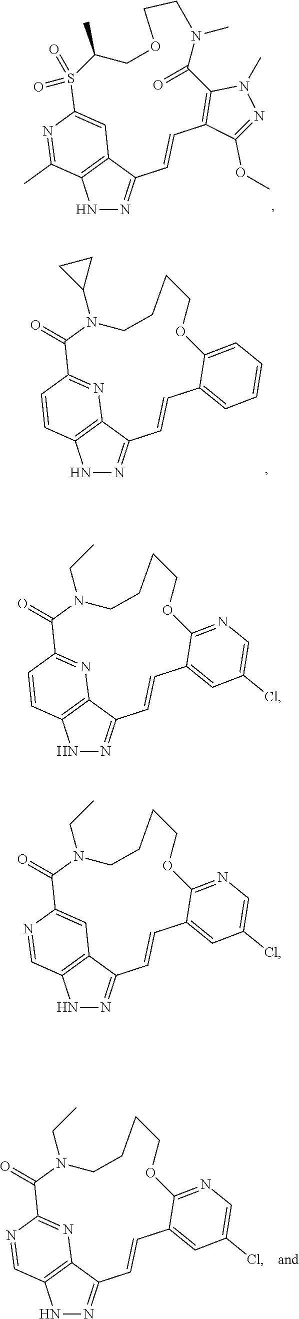 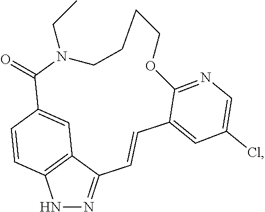 [SMILES: CCN1CCCCOc2ncc(Cl)cc2/C=C/c2n[nH]c3ccc(cc23)C1=O]
or a pharmaceutically acceptable salt thereof.

65. A pharmaceutical composition comprising a compound of any one of the preceding claims, and optionally one or more excipients.

66. A method of treating disease in a subject comprising, administering a therapeutically effective amount of a compound of any one of claims 1 to 64, or a pharmaceutical composition of claim 65.

67. A compound according to any one of claims 1 to 64, for use in a method of treating disease in a subject.

68. Use of a compound according to any one of claims 1 to 64 in the manufacture of a medicament for the treatment of disease in a subject.
